SEQUENCE LISTING (100 sequences)

SEQ ID NO: 1 - DNA, 993 bp, synthetic construct
  source 1..993: mol_type = other DNA; note = Codon-optimized c-Jun
atgacagccaagatggaaaccacattctacgacgacgccctgaacgcctcattcctgccttctgagagcggaccttacggctacagcaatcctaagatcctgaaacagagcatgacccttaacctggctgatcctgttggaagcctgaaacctcacctgagagccaaaaacagcgacctgctcaccagccctgatgtgggcctgctgaagctggcctctccagagctggaacggctgatcatccagagcagcaacggccacatcacaaccacccctacccctacacaattcctgtgccctaagaacgtgaccgacgagcaggagggcttcgccgaaggctttgtgcgggccctggcagaactgcactctcagaacaccctgcctagcgtgacctccgccgcccagcctgtcaacggcgccggaatggtggcccctgccgtggcttctgtggccggcggcagcggcagcggcggattcagcgcctctctgcactctgagcctcctgtctacgccaatctgtctaatttcaaccccggagccctgtccagcggcggcggagctcctagctacggcgctgctggactggccttccccgcccagccccagcaacagcagcagcctccacaccacctgccccagcagatgcccgtgcagcaccctagactgcaggccctgaaggaagaaccccaaacagtgcctgagatgcctggcgagacacctccactgagccccatcgacatggaaagccaggagcggatcaaggccgagagaaagagaatgcggaacagaatcgccgctagcaagtgcagaaagcggaagctggaaagaatcgccagactggaagagaaggtgaagaccctgaaagcccaaaatagcgagctggccagcaccgccaacatgctgcgggaacaggtggcccagctgaagcagaaggtgatgaaccacgtgaactctggttgtcagctgatgctgacccagcagctccagaccttc

SEQ ID NO: 2 - DNA, 993 bp, synthetic construct
  source 1..993: mol_type = other DNA; note = Codon-optimized c-Jun
atgacagccaagatggaaaccaccttctacgacgacgccctcaacgcctccttcctgccttctgagagcggtccttacggctacagcaaccccaagatcctgaagcaaagcatgaccctgaacctggccgaccccgttggctccctgaaacctcacctgagagccaaaaacagcgacctgctgaccagccctgatgtgggcctgctgaagctggcctctccagagctggaaagactgattatccagagcagcaacggccacatcaccacaacacctacccctacacagttcctgtgccctaagaacgtgactgatgagcaggagggctttgccgagggcttcgtgagagccctggctgagctgcattctcagaacaccctgcctagcgtgacctctgccgcccagcctgttaatggcgccggcatggtggcccctgccgtggcctctgtggccggaggcagcggcagcggcggattcagcgcctctctgcacagcgagccccccgtctacgccaacctgagcaatttcaaccctggcgccctgtccagcggcggcggcgccccttcatatggcgctgccggcctggccttccccgctcagccccagcagcagcaacagcctccacaccacctgccccagcagatgcccgtgcagcaccccagactgcaggccctgaaggaagaacctcagaccgtgcccgagatgcctggcgagacccctcctctgagccctatcgacatggaaagccaggagagaatcaaggccgagaggaagcggatgcggaacagaatcgccgccagcaagtgcagaaaaagaaagctggaacggatcgccagactggaggagaaggtgaagacactgaaagcccaaaattctgaactggcctctaccgccaatatgctgcgcgagcaggtggctcaactgaagcagaaggtgatgaaccacgtgaacagcggatgtcagctgatgctgacacagcagctgcagactttt

SEQ ID NO: 3 - DNA, 993 bp, synthetic construct
  source 1..993: mol_type = other DNA; note = Codon-optimized c-Jun
atgaccgccaagatggaaaccaccttctacgacgacgccctgaacgccagctttctgccttctgagtctggcccctacggctacagcaaccccaagatcctgaagcagagcatgaccctgaacctggccgatcctgtgggcagcctgaaacctcacctgagagccaagaacagcgacctgctgacaagccctgatgtgggcctgctgaaactggcctctcctgagctggaacggctgatcatccagagcagcaacggccacatcaccaccacacctacaccaacacagtttctgtgccccaagaacgtgaccgacgagcaagagggattcgccgagggctttgttagagccctggccgaactgcacagccagaataccctgcctagcgtgacatctgccgctcagcctgttaatggcgccggaatggttgctcctgccgtggcttctgttgctggcggatctggatctggcggctttagcgcctctctgcactctgagcctccagtgtacgccaacctgagcaacttcaaccctggcgctcttagctctggtggcggagcaccttcttatggcgctgccggattggcctttcctgctcagcctcagcagcagcaacagcctcctcatcatctgccccagcagatgcctgtgcagcaccctagactgcaggccctgaaagaggaaccccagacagtccctgagatgcccggcgaaacacctcctctgagccccatcgacatggaaagccaagagcggatcaaggccgagcggaagcggatgagaaatagaatcgccgcctccaagtgccggaagaggaagctggaaagaatcgcccggctggaagagaaagtgaaaaccctgaaggcccagaactccgagctggcctctaccgccaacatgctgagagaacaggtggcccagctgaaacagaaagtcatgaaccacgtgaacagcggctgccagctgatgctgacacagcagctgcagaccttc

SEQ ID NO: 4 - DNA, 993 bp, synthetic construct
  source 1..993: mol_type = other DNA; note = Codon-optimized c-Jun
atgactgccaaaatggagactacattctatgacgacgccctcaatgccagttttttgccgagtgaatccggcccctacggctattcaaaccctaagatcctcaagcaatcaatgaccctcaatcttgctgacccagttggctccctgaaaccccatctcagagctaaaaatagtgacctccttacttcccctgatgttggactcctcaaacttgcttctcccgaactcgaacgcttgatcattcaatcttccaacggccacatcacaacaacacccacacccacccagtttctttgcccaaaaaatgtcaccgatgaacaggaaggtttcgcggaaggattcgtccgcgcgctggccgaactgcactcccagaatacacttccttcagttacgtcagccgcccagccagtgaatggtgcgggaatggttgctcctgcggtcgcttctgtcgcagggggctccggttctggcggatttagcgcctctctgcattccgagccacctgtatatgctaatctttctaattttaaccccggagccttgtctagcggcggtggtgcccccagctacggtgctgcaggactcgccttcccagctcaacctcagcagcagcaacaacccccccatcaccttccccaacagatgccagtacaacatccaaggctccaggccctcaaagaggaaccacagacggtgcccgaaatgcctggcgaaactccaccactttcccctattgatatggaatcccaagagcgcatcaaggccgaaagaaagcgaatgcggaatagaatagcagcttcaaaatgtagaaaacggaaattggaacgaatcgcacggttggaagaaaaggtgaagaccttgaaagcccagaacagtgagctcgcctctaccgctaacatgctgcgcgagcaagtcgcacaacttaagcagaaggtgatgaaccatgtgaatagcggatgtcaacttatgctgactcaacagttgcaaaccttt

SEQ ID NO: 5 - DNA, 993 bp, synthetic construct
  source 1..993: mol_type = other DNA; note = Codon-optimized c-Jun
atgaccgcgaaaatggagacaacattttacgatgatgcactgaacgcctcttttctgccaagtgaatccggcccctacggatactcaaaccctaagattctgaaacagtctatgactctcaacctggccgacccagttggcagtctgaagcctcatttgcgagccaagaatagtgatctgctgacctccccagacgtgggactgctgaaactcgcctcacctgaacttgagcgcttgattatacagtcatccaatgggcacatcacaacaacacctactcctacccagtttctgtgccccaaaaacgtcaccgatgagcaggagggattcgcggaaggctttgtgcgcgccctggctgaattgcatagtcagaacactcttcccagcgtaaccagcgccgcccaaccagtgaatggagccggtatggtggctcccgcggtggctagtgttgcgggggggtcaggctctggtgggttcagtgcttctcttcactctgaaccccctgtgtatgccaatctgtctaactttaaccctggggccctctcctctggtgggggtgcccccagctacggagcggccggcctggcctttcctgcccagcctcagcagcagcagcaaccccctcatcatcttccgcagcagatgccagtacagcatccacgcctgcaggctcttaaggaggagccccagacggtgcccgaaatgcccggggaaactccacccttgtcccccattgacatggagtcccaggagcggatcaaggctgaaagaaagaggatgcggaatcgcatcgcagcctctaaatgccgcaagcggaaacttgagaggatcgcgcggttggaggaaaaagtaaaaaccttgaaggcacagaactctgagctggcgagtactgccaacatgctcagagaacaagtcgcacagctgaagcagaaagtgatgaaccatgtgaacagcggttgtcagctgatgctgactcagcagctgcagaccttc

SEQ ID NO: 6 - DNA, 993 bp, synthetic construct
  source 1..993: mol_type = other DNA; note = Codon-optimized c-Jun
atgaccgccaagatggagaccacattctacgatgacgctctgaacgcttcctttctgccttccgagtccggcccctacggctactccaatcccaagattctgaagcagagcatgacactgaatctggctgatcccgtgggatctctgaagcctcatctgagagccaagaattccgatctgctgacaagccccgacgtgggactgctcaaactggccagccccgaactggagaggctcattatccagagctccaacggccacatcaccacaacacctacccctacccagtttctctgtcccaagaacgtgacagacgagcaagagggatttgccgaaggcttcgtgagagccctcgccgaactgcatagccagaacacactgccttccgtgaccagcgctgctcaacccgtgaacggcgctggcatggtcgctcccgccgtcgccagcgtggctggaggaagcggatccggaggcttcagcgcttccctccacagcgaacctcccgtgtacgctaatctgagcaacttcaaccccggcgctctgagcagcggaggaggagctcctagctatggagctgccggactggcttttcccgcccagccccagcagcagcagcagcccccccatcatctgcctcagcagatgcccgtgcagcatcccagactccaagctctgaaggaggagcctcagaccgtccccgagatgcccggcgaaaccccccctctgtcccccatcgacatggaaagccaagagaggatcaaggccgagaggaagaggatgaggaatagaatcgccgccagcaagtgtagaaagaggaagctggagaggatcgccagactggaggagaaggtgaagaccctcaaggctcagaattccgagctggccagcacagccaacatgctgagagagcaagtggcccagctcaagcagaaggtgatgaaccacgtcaacagcggatgccagctgatgctcacccagcagctgcagaccttc

SEQ ID NO: 7 - DNA, 993 bp, synthetic construct
  source 1..993: mol_type = other DNA; note = Codon-optimized c-Jun
atgaccgctaaaatggaaaccactttctatgacgatgccctgaacgcctccttccttccgtccgagtccggaccctacggatactcaaatcctaagatcctcaaacagtcgatgaccctcaacctggccgaccccgtgggatccctgaagccgcacttgcgcgccaagaactccgacctcctgacgagcccagacgtgggcctgctgaagctcgcatcacccgaacttgagcggttgatcattcagtcctccaacggacatatcaccaccactcccaccccaactcagtttctgtgtccgaagaacgtgaccgatgagcaagagggattcgccgagggattcgtgcgggccctggccgagctgcatagccagaacacccttccatccgtgacctcggcggctcagcctgtgaacggcgcgggaatggtcgcgcccgccgtggcctcggtggccgggggcagcggcagcgggggattttccgcgtcgctgcactccgagccgccggtgtacgccaacctgtcaaacttcaaccctggggccctgagctccggcggtggagcaccttcgtacggcgccgctggcctggcgttccccgcgcaaccacagcagcaacagcagccccctcaccacctcccccaacaaatgcctgtgcagcacccgaggctgcaggccctcaaggaagaaccccagactgtgccggaaatgccgggggagactccgccgctgtcccctatcgacatggaatcacaggaacgcattaaggcagagcggaagcgcatgcggaaccggattgccgcctccaagtgccgcaagagaaagctcgaaagaatcgccagattggaagaaaaggtcaagactctgaaggcccagaactctgagctggcatccaccgctaatatgctgagggaacaagtggcccagctgaaacagaaggtcatgaaccacgtcaacagcggttgccagctgatgctgacccagcaactccagacattc

SEQ ID NO: 8 - DNA, 993 bp, synthetic construct
  source 1..993: mol_type = other DNA; note = Codon-optimized c-Jun
atgaccgccaagatggagaccaccttctacgacgacgccctgaacgccagcttcctgcccagcgagagcggaccctacggctactctaaccccaagatcctgaaacagagcatgacactgaatctggccgaccccgtgggcagcctgaagcctcaccttagagccaagaacagcgacctgctgaccagccccgacgtgggcctgctgaagctcgcctctccagagttagagagactgatcatccagtccagcaacggccacatcacaaccaccccaacccctacccagttcctgtgccccaagaacgtgaccgacgagcaggagggcttcgccgagggctttgtgagagccctggccgagttgcactctcagaacaccctgccctccgtgaccagcgccgctcaacctgtgaacggcgcaggaatggttgctcctgccgtggccagcgttgcaggcggatctggaagtggaggcttctccgcctcccttcacagcgagcctcccgtgtacgccaacctgagcaacttcaaccccggcgccctgagcagtggaggaggcgctcccagctatggagcagctggattagccttccccgcccagccacagcagcagcaacagcctccccaccacctgcctcagcaaatgcctgtgcagcaccctcggctgcaggcccttaaggaggagccccagaccgttcctgagatgcctggcgagacccctcccctgagccctatcgacatggagtcccaggagcggatcaaggccgagcggaagcggatgcggaaccggatcgctgcttccaagtgccggaagagaaagctggagagaatcgcccggctggaggagaaggtgaagaccctgaaggcccagaactccgagctggcctccaccgccaacatgctgcgggagcaggttgcacagctgaagcagaaggtcatgaaccacgtgaacagcggctgccagctgatgctgacccagcagctgcagaccttc

SEQ ID NO: 9 - DNA, 1059 bp, synthetic construct
  source 1..1059: mol_type = other DNA; note = Codon-optimized c-Jun
atgacagcgaagatggagacaaccttctatgacgatgctcttaacgcctccttcctgccttccgaaagcgggccctacgggtactctaatcctaagatacttaagcaatcgatgactctcaacctcgctgacccggttggctcactgaaaccacacctgagagctaagaatagtgacctgctcactagtcccgatgtcgggcttctgaagctggcctctcccgagctggagaggcttatcatccaatcatcaaatggccacatcaccactaccccaacaccaactcaattcctttgccctaaaaacgtgaccgacgaacaggaaggcttcgccgagggttttgtccgggccttggccgagctgcattctcaaaatacactgccaagcgtcacttctgcggcgcagccggttaacggagcagggatggtggctcccgccgttgctagcgtggccggcggttccggctccggcggtttctctgcctccttgcattctgagccaccagtctacgcgaacctgtccaactttaatccgggggcgctgagtagcggaggcggcgcccctagctatggggcagctggactggccttcccggcacaaccccaacaacaacagcaaccgccacaccatcttcctcaacaaatgccagtgcaacatccacgcttacaagccctcaaggaggaaccccagaccgtgcctgagatgcccggcgaaaccccgccattgagccctattgacatggaaagtcaagagagaattaaggcagagcgcaagagaatgaggaaccggatcgcagcatctaagtgccgcaaacggaaattggagcggatcgctcgcttggaggagaaggtcaagactctcaaggcccagaactccgagcttgcgagcacagctaatatgctgcgcgagcaggtggcccagttaaaacaaaaggtcatgaaccatgtgaacagcggctgtcagctgatgcttacgcaacagctgcaaacctttggctccggtgcaacgaacttcagcctgctgaagcaggccggagatgttgaggaaaatccaggtccc

SEQ ID NO: 10 - DNA, 993 bp, synthetic construct
  source 1..993: mol_type = other DNA; note = Codon-optimized c-Jun
atgacggccaaaatggagactacgttctacgatgacgcactcaacgcgtccttcctgccctctgagagtggaccctatggctactccaatccaaagatcctgaagcagtctatgaccctcaacctggcggacccggtgggctcccttaagccgcacttgcgcgccaagaactccgacctgctgacctcccctgatgtgggcctcctcaagctcgctagccctgaattggagaggctgatcatccagagctcaaatggccacatcaccaccacacctaccccaacccagttcctgtgcccaaaaaacgtgaccgacgagcaggagggcttcgcggagggcttcgtcagagctctggccgagctgcactcacagaacacgctcccttccgtgacctccgctgcccagccggtcaatggcgctggaatggtggctccggctgtggcctctgttgccggcggctccggctccggaggcttttcagcttctctgcattctgagcccccagtgtacgctaacctgagcaacttcaaccccggggcgctcagctccggtggcggtgccccgagctacggcgcggctgggctggcgttccccgctcagcctcagcagcaacagcaacctccccaccacctgccacagcagatgcctgtgcagcacccacgcctgcaggccttgaaggaggaacctcagactgtgccagagatgcccggcgagaccccacccctgtccccgattgacatggagagccaggagcgcatcaaggcagagcgcaagcgtatgcgcaaccgcatcgcggcctccaagtgccgaaagcgcaagctggagcggattgctcgcctggaggagaaggtgaagaccctgaaggcccagaattccgagctggcctcgaccgccaacatgctacgagaacaggtcgcgcagctgaaacagaaggtcatgaaccatgtcaacagcgggtgccagctgatgttgacccagcagcttcagaccttc

SEQ ID NO: 11 - DNA, 993 bp, Homo sapiens
  source 1..993: mol_type = other DNA; note = Wild-type c-Jun coding region
atgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattt

SEQ ID NO: 12 - DNA, 3257 bp, Homo sapiens
  source 1..3257: mol_type = other DNA; note = Wild-type c-Jun
gctcagagttgcactgagtgtggctgaagcagcgaggcgggagtggaggtgcgcggagtcaggcagacagacagacacagccagccagccaggtcggcagtatagtccgaactgcaaatcttattttcttttcaccttctctctaactgcccagagctagcgcctgtggctcccgggctggtgtttcgggagtgtccagagagcctggtctccagccgcccccgggaggagagccctgctgcccaggcgctgttgacagcggcggaaagcagcggtacccacgcgcccgccgggggaagtcggcgagcggctgcagcagcaaagaactttcccggctgggaggaccggagacaagtggcagagtcccggagccaacttttgcaagcctttcctgcgtcttaggcttctccacggcggtaaagaccagaaggcggcggagagccacgcaagagaagaaggacgtgcgctcagcttcgctcgcaccggttgttgaacttgggcgagcgcgagccgcggctgccgggcgccccctccccctagcagcggaggaggggacaagtcgtcggagtccgggcggccaagacccgccgccggccggccactgcagggtccgcactgatccgctccgcggggagagccgctgctctgggaagtgagttcgcctgcggactccgaggaaccgctgcgcacgaagagcgctcagtgagtgaccgcgacttttcaaagccgggtagcgcgcgcgagtcgacaagtaagagtgcgggaggcatcttaattaaccctgcgctccctggagcgagctggtgaggagggcgcagcggggacgacagccagcgggtgcgtgcgctcttagagaaactttccctgtcaaaggctccggggggcgcgggtgtcccccgcttgccacagccctgttgcggccccgaaacttgtgcgcgcagcccaaactaacctcacgtgaagtgacggactgttctatgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttgaagagagaccgtcgggggctgaggggcaacgaagaaaaaaaataacacagagagacagacttgagaacttgacaagttgcgacggagagaaaaaagaagtgtccgagaactaaagccaagggtatccaagttggactgggttgcgtcctgacggcgcccccagtgtgcacgagtgggaaggacttggcgcgccctcccttggcgtggagccagggagcggccgcctgcgggctgccccgctttgcggacgggctgtccccgcgcgaacggaacgttggacttttcgttaacattgaccaagaactgcatggacctaacattcgatctcattcagtattaaaggggggagggggagggggttacaaactgcaatagagactgtagattgcttctgtagtactccttaagaacacaaagcggggggagggttggggaggggcggcaggagggaggtttgtgagagcgaggctgagcctacagatgaactctttctggcctgccttcgttaactgtgtatgtacatatatatattttttaatttgatgaaagctgattactgtcaataaacagcttcatgcctttgtaagttatttcttgtttgtttgtttgggtatcctgcccagtgttgtttgtaaataagagatttggagcactctgagtttaccatttgtaataaagtatataatttttttatgttttgtttctgaaaattccagaaaggatatttaagaaaatacaataaactattggaaagtactcccctaacctcttttctgcatcatctgtagatactagctatctaggtggagttgaaagagttaagaatgtcgattaaaatcactctcagtgcttcttactattaagcagtaaaaactgttctctattagactttagaaataaatgtacctgatgtacctgatgctatggtcaggttatactcctcctcccccagctatctatatggaattgcttaccaaaggatagtgcgatgtttcaggaggctggaggaaggggggttgcagtggagagggacagcccactgagaagtcaaacatttcaaagtttggattgtatcaagtggcatgtgctgtgaccatttataatgttagtagaaattttacaataggtgcttattctcaaagcaggaattggtggcagattttacaaaagatgtatccttccaatttggaatcttctctttgacaattcctagataaaaagatggcctttgcttatgaatatttataacagcattcttgtcacaataaatgtattcaaataccaa

SEQ ID NO: 13 - protein, 331 aa, Homo sapiens
  source 1..331: note = Wild-type c-Jun
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 14 - protein, 19 aa, synthetic construct
  source 1..19: note = P2A Linker
ATNFSLLKQAGDVEENPGP

SEQ ID NO: 15 - protein, 18 aa, synthetic construct
  source 1..18: note = T2A Linker
EGRGSLLTCGDVEENPGP

SEQ ID NO: 16 - protein, 22 aa, synthetic construct
  source 1..22: note = F2A Linker
VKQTLNFDLLKLAGDVESNPGP

SEQ ID NO: 17 - protein, 20 aa, synthetic construct
  source 1..20: note = E2A Linker
QCTNYALLKLAGDVESNPGP

SEQ ID NO: 18 - protein, 4 aa, synthetic construct
  source 1..4: note = Furin Cleavage Site
RAKR

SEQ ID NO: 19 - protein, 1210 aa, Homo sapiens [sequence omitted]

SEQ ID NO: 20 - protein, 404 aa, Homo sapiens
  source 1..404: note = EGFR Isoform 2
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKCNGIGIGEFKDSLSINATNIKHFKNCTSISGDLHILPVAFRGDSFTHTPPLDPQELDILKTVKEITGLS

SEQ ID NO: 21 - protein, 705 aa, Homo sapiens
  source 1..705: note = EGFR Isoform 3
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGPGNESLKAMLFCLFKLSSCNQSNDGSVSHQSGSPAAQESCLGWIPSLLPSEFQLGWGGCSHLHAWPSASVIITASSCH

SEQ ID NO: 22 - protein, 628 aa, Homo sapiens
  source 1..628: note = EGFR Isoform 4
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGS

SEQ ID NO: 23 - protein, 335 aa, synthetic construct
  source 1..335: note = EGFRt
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 24 - protein, 338 aa, synthetic construct
  source 1..338: note = EGFRopt
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRR

SEQ ID NO: 25 - protein, 6 aa, synthetic construct
  source 1..6: note = Juxtamembrane Domain #5
KSGSGS

SEQ ID NO: 26 - protein, 4 aa, synthetic construct
  source 1..4: note = Juxtamembrane Domain #7
KRSD

SEQ ID NO: 27 - protein, 5 aa, synthetic construct
  source 1..5: note = Juxtamembrane Domain #8
KRSDK

SEQ ID NO: 28 - protein, 5 aa, synthetic construct
  source 1..5: note = Juxtamembrane Domain #9
SGGGG

SEQ ID NO: 29 - protein, 5 aa, synthetic construct
  source 1..5: note = Juxtamembrane Domain #10
SGAGG

SEQ ID NO: 30 - protein, 5 aa, synthetic construct
  source 1..5: note = Juxtamembrane Domain #11
KRADK

SEQ ID NO: 31 - protein, 14 aa, synthetic construct
  source 1..14: note = Juxtamembrane Domain #12
RRRSGGGGSGGGGS

SEQ ID NO: 32 - protein, 11 aa, synthetic construct
  source 1..11: note = Juxtamembrane Domain #13
SGGGGSGGGGS

SEQ ID NO: 33 - protein, 5 aa, synthetic construct
  source 1..5: note = Juxtamembrane Domain #14
  REPEAT 1..5: note = n, wherein n is an integer greater than 1
GGGGS

SEQ ID NO: 34 - protein, 9 aa, synthetic construct
  source 1..9: note = Juxtamembrane Domain #15
RRRHIVRKR

SEQ ID NO: 35 - protein, 8 aa, synthetic construct
  source 1..8: note = Juxtamembrane Domain #16
RRRHIVRK

SEQ ID NO: 36 - protein, 7 aa, synthetic construct
  source 1..7: note = Juxtamembrane Domain #17
RRRHIVR

SEQ ID NO: 37 - protein, 6 aa, synthetic construct
  source 1..6: note = Juxtamembrane Domain #18
RRRHIV

SEQ ID NO: 38 - protein, 5 aa, synthetic construct
  source 1..5: note = Juxtamembrane Domain #19
RRRHI

SEQ ID NO: 39 - protein, 4 aa, synthetic construct
  source 1..4: note = Juxtamembrane Domain #20
RRRH

SEQ ID NO: 40 - protein, 5 aa, synthetic construct
  source 1..5: note = Linker
GGGSG

SEQ ID NO: 41 - protein, 353 aa, Homo sapiens
  source 1..353: note = Human IgA1
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 42 - protein, 340 aa, Homo sapiens
  source 1..340: note = Human IgA2
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 43 - protein, 398 aa, Mus musculus
  source 1..398: note = Mouse IgG2A
KTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGLDLDDVCAEAQDGELDGLWTTITIFISLFLLSVCYSASVTLFKVKWIFSSVVELKQTISPDYRNMIGQGA

SEQ ID NO: 44 - protein, 330 aa, Homo sapiens [sequence omitted]

SEQ ID NO: 45 - protein, 326 aa, Homo sapiens
  source 1..326: note = Human IgG2
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 46 - protein, 377 aa, Homo sapiens
  source 1..377: note = Human IgG3
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 47 - protein, 327 aa, Homo sapiens
  source 1..327: note = Human IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 384 aa, Homo sapiens
  source 1..384: note = Human IgD
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 49 - protein, 428 aa, Homo sapiens
  source 1..428: note = Human IgE
ASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 50 - protein, 453 aa, Homo sapiens
  source 1..453: note = Human IgM
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 51 - protein, 9 aa, synthetic construct
  source 1..9: note = Spacer
KPCPPCKCP

SEQ ID NO: 52 - protein, 24 aa, synthetic construct
  source 1..24: note = EGFR Signal Peptide
MRPSGTAGAALLALLAALCPASRA

SEQ ID NO: 53 - protein, 22 aa, synthetic construct
  source 1..22: note = GM-CSF Signal Peptide
MLLLVTSLLLCELPHPAFLLIP

SEQ ID NO: 54 - protein, 20 aa, synthetic construct
  source 1..20: note = Human Ig Kappa Signal Peptide
MVLQTQVFISLLLWISGAYG

SEQ ID NO: 55 - protein, 16 aa, synthetic construct
  source 1..16: note = Human CD33 Signal Peptide
MPLLLLLPLLWAGALA

SEQ ID NO: 56 - protein, 121 aa, synthetic construct
  source 1..121: note = R12 VH
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISS

SEQ ID NO: 57 - protein, 5 aa, synthetic construct
  source 1..5: note = R12 VH CDR1
AYYMS

SEQ ID NO: 58 - protein, 17 aa, synthetic construct
  source 1..17: note = R12 VH CDR2
TIYPSSGKTYYATWVNG

SEQ ID NO: 59 - protein, 12 aa, synthetic construct
  source 1..12: note = R12 VH CDR3
DSYADDGALFNI

SEQ ID NO: 60 - protein, 112 aa, synthetic construct
  source 1..112: note = R12 VL
ELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 61 - protein, 12 aa, synthetic construct
  source 1..12: note = R12 VL CDR1
TLSSAHKTDTID

SEQ ID NO: 62 - protein, 7 aa, synthetic construct
  source 1..7: note = R12 VL CDR2
GSYTKRP

SEQ ID NO: 63 - protein, 9 aa, synthetic construct
  source 1..9: note = R12 VL CDR3
GADYIGGYV

SEQ ID NO: 64 - protein, 5 aa, synthetic construct
  source 1..5: note = Linker #1
  REPEAT 1..5: note = n, wherein n is an integer between 1 and 100
GGGSG

SEQ ID NO: 65 - protein, 5 aa, synthetic construct
  source 1..5: note = Linker #2
  REPEAT 1..5: note = n, wherein n is an integer between 1 and 100
GGGGS

SEQ ID NO: 66 - protein, 4 aa, synthetic construct
  source 1..4: note = Linker #3
  REPEAT 1..4: note = n, wherein n is an integer between 1 and 100
GGGS

SEQ ID NO: 67 - skipped

SEQ ID NO: 68 - protein, 6 aa, synthetic construct
  source 1..6: note = Linker #5
GSGSGS

SEQ ID NO: 69 - protein, 5 aa, synthetic construct
  source 1..5: note = Linker #6
GGSGG

SEQ ID NO: 70 - protein, 7 aa, synthetic construct
SGGSGGS

SEQ ID NO: 71 - protein, 15 aa, synthetic construct
  source 1..15: note = Linker #8
GGSGGSGGSGGSGGG

SEQ ID NO: 72 - protein, 16 aa, synthetic construct
  source 1..16: note = Linker #9
GGSGGSGGGGSGGGGS

SEQ ID NO: 73 - protein, 18 aa, synthetic construct
GGSGGSGGSGGSGGSGGS

SEQ ID NO: 74 - protein, 15 aa, synthetic construct
  source 1..15: note = Linker #11
GGGGSGGGGSGGGGS

SEQ ID NO: 75 - protein, 28 aa, synthetic construct
  source 1..28: note = CD28 transmembrane domain
MFWVLVVVGGVLACYSLLVTVAFIIFWV

SEQ ID NO: 76 - protein, 42 aa, synthetic construct
  source 1..42: note = 4-1BB costimulatory domain
KRGRKKLLYIFKQPFMRPVQTTQEEDGCSCRFPEEEEGGCEL

SEQ ID NO: 77-80 - skipped (4 entries)

SEQ ID NO: 81 - protein, 4 aa, synthetic construct
  source 1..4: note = Linker #16
GGGG

SEQ ID NO: 82 - protein, 5 aa, synthetic construct
  source 1..5: note = Linker #17
GGGSG

SEQ ID NO: 83 - protein, 248 aa, synthetic construct
  source 1..248: note = R12 scFv
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 84 - protein, 112 aa, synthetic construct
  source 1..112: note = CD3zeta activating domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 21 aa, synthetic construct
  source 1..21: note = p2A
SGATNFSLLKQAGDVEENPGP

SEQ ID NO: 86 - protein, 1198 aa, synthetic construct
  source 1..1198: note = c-jun-anti-ROR1 CAR
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTFGSGATNFSLLKQAGDVEENPGPMVLQTQVFISLLLWISGAYGQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGGGGSGKPCPPCKCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRR

SEQ ID NO: 87 - DNA, 4022 bp, synthetic construct
  source 1..4022: mol_type = other DNA; note = c-jun-anti ROR1
tgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatcagaacctcttacgagtcggctagcgccgccaccatgacagccaagatggaaaccacattctacgacgacgccctgaacgcctcattcctgccttctgagagcggaccttacggctacagcaatcctaagatcctgaaacagagcatgacccttaacctggctgatcctgttggaagcctgaaacctcacctgagagccaaaaacagcgacctgctcaccagccctgatgtgggcctgctgaagctggcctctccagagctggaacggctgatcatccagagcagcaacggccacatcacaaccacccctacccctacacaattcctgtgccctaagaacgtgaccgacgagcaggagggcttcgccgaaggctttgtgcgggccctggcagaactgcactctcagaacaccctgcctagcgtgacctccgccgcccagcctgtcaacggcgccggaatggtggcccctgccgtggcttctgtggccggcggcagcggcagcggcggattcagcgcctctctgcactctgagcctcctgtctacgccaatctgtctaatttcaaccccggagccctgtccagcggcggcggagctcctagctacggcgctgctggactggccttccccgcccagccccagcaacagcagcagcctccacaccacctgccccagcagatgcccgtgcagcaccctagactgcaggccctgaaggaagaaccccaaacagtgcctgagatgcctggcgagacacctccactgagccccatcgacatggaaagccaggagcggatcaaggccgagagaaagagaatgcggaacagaatcgccgctagcaagtgcagaaagcggaagctggaaagaatcgccagactggaagagaaggtgaagaccctgaaagcccaaaatagcgagctggccagcaccgccaacatgctgcgggaacaggtggcccagctgaagcagaaggtgatgaaccacgtgaactctggttgtcagctgatgctgacccagcagctccagaccttcggctccggtgcaacgaacttcagcctgctgaagcaggccggagatgttgaggaaaatccaggtcccatggtcttgcagactcaagtatttatatcccttttgctctggatctctggagcttacggccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgctcgggacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgacaggttcagcggatctagctctggcgccgaccgctacctgatcatccccagcgtgcaggccgatgacgaggccgattactactgtggcgccgactacatcggcggctacgtgttcggcggaggcacccagctgaccgtgaccggtggcggaggttcaggcaaaccgtgccctccgtgcaagtgtcctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtccggagccactaacttctccctgttgaaacaagcaggggatgtcgaagagaatcccgggccaatgcttctcctggtgacaagccttctgctctgtgaattaccacacccagcattcctcctgatcccacgcaaagtgtgcaacggaataggtattggtgaatttaaggactcactctccataaatgctacgaatattaaacacttcaaaaactgcacctccatcagtggcgatctccacatcctgccggtggcatttaggggtgactccttcacacatactcctcctctggacccacaagaactggatattctgaaaaccgtaaaggaaatcacagggtttttgctgattcaagcttggcctgaaaacaggacggacctccatgcctttgagaacctagaaatcatacgcggcaggaccaagcagcatggacagttttctcttgctgtcgtgagcctgaacataacatccttgggattacgctccctcaaggagataagtgatggagatgtgataatttcaggaaacaaaaatttgtgctatgcaaatacaataaactggaaaaaactgtttgggacctccggccagaaaaccaaaattataagcaacagaggcgaaaacagctgcaaggccacaggccaggtctgccatgccttgtgctcccccgagggctgctggggcccggagcccagggattgcgtgtcttgccggaatgtcagccgaggcagggaatgcgtggacaagtgcaaccttctggaaggcgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaacgggcctaagatcccgtccatcgccactgggatggtgggggccctcctcttgctgctggtggtggccctggggatcggcctcttcatgcgccgaaggtga

SEQ ID NO: 88 - protein, 352 aa, synthetic construct
  source 1..352: note = c-Jun after P2A cleavage
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTFGSGATNFSLLKQAGDVEENPG

SEQ ID NO: 89 - protein, 21 aa, synthetic construct
  source 1..21: note = hIgK after P2A cleavage
PMVLQTQVFISLLLWISGAYG

SEQ ID NO: 90 - protein, 133 aa, synthetic construct
  source 1..133: note = CD3z after P2A cleavage
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGATNFSLLKQAGDVEENPGP

SEQ ID NO: 91 - protein, 23 aa, synthetic construct
  source 1..23: note = GMCSFR-alpha-SP after P2A cleavage
PMLLLVTSLLLCELPHPAFLLIP

SEQ ID NO: 92 - DNA, 386 bp, synthetic construct
  source 1..386: mol_type = other DNA; note = MND promotor
tgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggc

SEQ ID NO: 93 - protein, 22 aa, synthetic construct
  source 1..22: note = P2A #2
GSGATNFSLLKQAGDVEENPGP

SEQ ID NO: 94 - skipped

SEQ ID NO: 95 - protein, 958 aa, synthetic construct
  source 1..958: note = c-Jun-anti-NYESO1 TCR
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTFGSGATNFSLLKQAGDVEENPGPMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRGRAKRSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 96 - protein, 307 aa, synthetic construct
  source 1..307: note = NY-ESO1 TCR beta chain
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRG

SEQ ID NO: 97 - protein, 4 aa, synthetic construct
  source 1..4: note = Linker
SGSG

SEQ ID NO: 98 - protein, 271 aa, synthetic construct
  source 1..271: note = NY-ESO1 TCR alpha chain
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 99 - DNA, 2874 bp, synthetic construct
  source 1..2874: mol_type = other DNA; note = cJun NY-ESO1 TCR
atgacagccaagatggaaaccacattctacgacgacgccctgaacgcctcattcctgccttctgagagcggaccttacggctacagcaatcctaagatcctgaaacagagcatgacccttaacctggctgatcctgttggaagcctgaaacctcacctgagagccaaaaacagcgacctgctcaccagccctgatgtgggcctgctgaagctggcctctccagagctggaacggctgatcatccagagcagcaacggccacatcacaaccacccctacccctacacaattcctgtgccctaagaacgtgaccgacgagcaggagggcttcgccgaaggctttgtgcgggccctggcagaactgcactctcagaacaccctgcctagcgtgacctccgccgcccagcctgtcaacggcgccggaatggtggcccctgccgtggcttctgtggccggcggcagcggcagcggcggattcagcgcctctctgcactctgagcctcctgtctacgccaatctgtctaatttcaaccccggagccctgtccagcggcggcggagctcctagctacggcgctgctggactggccttccccgcccagccccagcaacagcagcagcctccacaccacctgccccagcagatgcccgtgcagcaccctagactgcaggccctgaaggaagaaccccaaacagtgcctgagatgcctggcgagacacctccactgagccccatcgacatggaaagccaggagcggatcaaggccgagagaaagagaatgcggaacagaatcgccgctagcaagtgcagaaagcggaagctggaaagaatcgccagactggaagagaaggtgaagaccctgaaagcccaaaatagcgagctggccagcaccgccaacatgctgcgggaacaggtggcccagctgaagcagaaggtgatgaaccacgtgaactctggttgtcagctgatgctgacccagcagctccagaccttcggctccggtgcaacgaacttcagcctgctgaagcaggccggagatgttgaggaaaatccaggtcccatgtctatcggactgctgtgctgtgccgccctgagcctgctgtgggcaggacctgtgaacgcaggagtgacccagacaccaaagttccaggtgctgaagaccggccagagcatgacactgcagtgcgcccaggatatgaatcacgagtacatgtcctggtatcggcaggaccctggcatgggcctgagactgatccactactccgtgggagcaggaatcaccgatcagggagaggtgccaaacggctataacgtgagccggagcaccacagaggacttcccactgagactgctgtccgccgcaccttcccagaccagcgtgtacttttgcgccagctcctatgtgggcaacacaggcgagctgttctttggagagggaagcaggctgaccgtgctggaggatctgcgcaacgtgacaccccctaaggtgtctctgttcgagcccagcaaggccgagatcgccaataagcagaaggccaccctggtgtgcctggcaaggggcttctttcctgatcacgtggagctgtcctggtgggtgaacggcaaggaggtgcactctggcgtgtgcaccgacccacaggcctacaaggagagcaattactcctattgtctgtctagccggctgagagtgtccgccacattttggcacaacccacggaatcacttcagatgccaggtgcagtttcacggcctgtctgaggaggataagtggccagagggaagcccaaagccagtgacccagaacatctccgccgaggcatggggaagggcagactgtggcatcacctccgcctcttatcagcagggcgtgctgagcgccacaatcctgtacgagatcctgctgggcaaggccaccctgtatgccgtgctggtgtccacactggtggtcatggccatggtgaagaggaagaattctaggggccgcgcaaagcgcagcggatccggagcaaccaacttctccctgctgaagcaggccggcgatgtggaggagaatcctggcccaatggagacactgctgggcctgctgatcctgtggctgcagctgcagtgggtgtcctctaagcaggaggtgacccagatccctgccgccctgagcgtgccagagggagagaacctggtgctgaattgctccttcacagattctgccatctacaacctgcagtggtttaggcaggacccaggcaagggactgacctctctgctgctgatccagagctcccagagggagcagaccagcggccggctgaacgcctccctggataagtctagcggcagaagcaccctgtacatcgcagcctcccagcctggcgactctgccacatacctgtgcgccgtgcggccactgtacggaggcagctatatccccaccttcggcagaggcacatccctgatcgtgcacccctacgacatccagaaccccgagcctgccgtgtatcagctgaaggaccctcggtctcaggatagcaccctgtgcctgttcacagactttgattctcagatcaatgtgcccaagaccatggagagcggcacctttatcacagacaagtgcgtgctggatatgaaggccatggactctaagagcaacggcgccatcgcctggagcaatcagacctccttcacatgccaggatatctttaaggagaccaacgccacatacccatcctctgacgtgccctgtgatgccaccctgacagagaagagcttcgagaccgacatgaacctgaattttcagaacctgctggtcatcgtgctgaggatcctgctgctgaaggtggccggctttaatctgctgatgacactgcgcctgtggagctcc

SEQ ID NO: 100 - DNA, 1187 bp, synthetic construct
  source 1..1187: mol_type = other DNA; note = EF1a promoter
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaatcgattg

(19) United States
(12) Patent Application Publication
(10) Pub. No.: US 2023/0313138 A1
(43) Pub. Date: Oct. 5, 2023

(54) METHODS FOR CULTURING CELLS EXPRESSING C-JUN

(71) Applicant: Lyell Immunopharma, Inc., South San Francisco, CA (US)

(72) Inventors: Suman Kumar VODNALA, San Mateo, CA (US); Veena KRISHNAMOORTHY, South San Francisco, CA (US); Spencer PARK, Seattle, WA (US); Queenie VONG, Seattle, WA (US); Blythe SATHER, Seattle, WA (US)

(73) Assignee: Lyell Immunopharma, Inc., South San Francisco, CA (US)

(21) Appl. No.: 18/050,414

(22) Filed: Oct. 27, 2022

Related U.S. Application Data
(60) Provisional application No. 63/339,353, filed on May 6, 2022.
(60) Provisional application No. 63/309,403, filed on Feb. 11, 2022.
(60) Provisional application No. 63/263,233, filed on Oct. 28, 2021.

(51) Int. Cl. C12N 5/0783; C07K 14/82; C07K 14/475; A61K 35/17

(52) U.S. Cl. CPC C12N 5/0636; C07K 14/82; C07K 14/475; A61K 35/17; C12N 2501/2302; C12N 2501/2321; C12N 2501/2307; C12N 2501/2315; C12N 2500/14

(57) ABSTRACT

Disclosed herein are methods of culturing immune cells in a medium comprising at least about 5 mM potassium ion, wherein the medium is capable of increasing the stemness of the immune cells. In some aspects, the immune cells which are cultured using the methods provided herein are modified to overexpress c-Jun and/or comprise one or more exogenous nucleotide sequences encoding a ligand binding protein. In some aspects, the immune cells are administered to a subject in need thereof.

### CROSS-REFERENCE TO RELATED APPLICATIONS

This application claims the priority benefit of U.S. Provisional Application No. 63/263,233, filed on Oct. 28, 2021; 63/309,403, filed on Feb. 11, 2022; and 63/339,353, filed on May 6, 2022; each of which is herein incorporated by reference in its entirety.

### REFERENCE TO SEQUENCE LISTING SUBMITTED ELECTRONICALLY VIA EFS-WEB

The content of the electronically submitted sequence listing (Name: 4385_0790004_Seglisting_ST26.xml, Size: 135,670 bytes; and Date of Creation: Oct. 27, 2022) submitted in this application is incorporated herein by reference in its entirety.

### FIELD OF THE DISCLOSURE

The present disclosure relates to methods of culturing cells, e.g., pluripotent, multipotent, and/or immune cells (e.g., T cells and/or NK cells), that have been modified to express an increased level of a c-Jun protein, e.g., compared to a corresponding cell that has not been modified. As described herein, in some aspects, the immune cells that are cultured using the methods described herein are also modified to comprise an exogenous polynucleotide encoding a protein (e.g., a chimeric binding protein), such that the encoded protein is expressed by the cell. In some aspects, the methods disclosed herein promote enrichment of less-differentiated cells and/or undifferentiated cells in culture, while retaining their effector activity. In some aspects, the culturing methods provided herein can also help increase the expression of a protein of interest (e.g., c-Jun) in a cell. Cells cultured using the methods disclosed herein can be used for various cell therapies, including but not limited to chimeric antigen receptor (CAR) T cell therapy, TCR T cell therapy including neoantigen directed-T cell therapies, and TIL therapy.

### BACKGROUND OF THE DISCLOSURE

Cancer immunotherapy relies on harnessing T cells—the immune system's primary killers of infected and diseased cells—to attack and kill tumor cells. However, the ability of immune cells to target and kill tumor cells is dampened by the presence of various inhibitors of the immune response that are present within the tumor microenvironment. Therefore, while CAR T cells have had various successes in treating certain cancers (e.g., KYMRIAH™ (tisagenlecleucel, Novartis) and YESCARTA™ (axicabtagene ciloleucel, Kite/Gilead) has been approved by the FDA), challenges remain. For instance, the success of CAR T cell immunotherapy is often limited by the extent of CAR T expansion in a recipient's body, which typically requires a large infusion of cells. Additionally, exhaustion and loss of persistence of the transferred CAR T cells have been observed, leading to loss of clinical efficacy and potential relapse.

One means of overcoming T cell exhaustion is to selectively administer T cells having a less-differentiated state. For example, T memory stem cells (T_{SCM}) persist for a greater period in patients following administration than do more differentiated T central memory (T_{CM}) or T effector memory (T_{EM}) cells, and T_{SCM} elicit a more pronounced and prolonged effect on tumor size than more differentiated cells. However, many adoptive cell therapy (ACT) cell preparations comprise an ill-defined mix of immune cells at various states of differentiation, which are ineffective at eradicating solid tumors. To be curative, T cells products with enhanced self-renewing stem/effector properties are needed. As such, there remains a need in the art for methods of efficiently enriching for less differentiated and/or naïve T cells from a mixed population of isolated T cells.

### BRIEF SUMMARY OF THE DISCLOSURE

Provided herein is a method of increasing stemness of immune cells (e.g., human immune cells) during ex vivo or in vitro culture comprising culturing immune cells (e.g., human immune cells) in a medium comprising potassium ion at a concentration higher than 5 mM, wherein the immune cells have been modified to have an increased level of a c-Jun polypeptide as compared to corresponding immune cells that have not been modified to have an increased level of the c-Jun polypeptide. Also provided herein is a method of increasing the yield of immune cells (e.g., human immune cells) during ex vivo or in vitro culture comprising culturing immune cells (e.g., human immune cells) in a medium comprising potassium ion at a concentration higher than 5 mM, wherein the immune cells have been modified to have an increased level of a c-Jun polypeptide as compared to corresponding immune cells that have not been modified to have an increased level of the c-Jun polypeptide. Also provided herein is a method of preparing a population of immune cells (e.g., human immune cells) for immunotherapy comprising culturing immune cells (e.g., human immune cells) in a medium comprising potassium ion at a concentration higher than 5 mM, wherein the immune cells have been modified to have an increased level of a c-Jun polypeptide as compared to corresponding immune cells that have not been modified to have an increased level of the c-Jun polypeptide. Present disclosure also provides a method of increasing stemness of immune cells (e.g., human immune cells) while increasing the yield of immune cells (e.g., human immune cells) during ex vivo or in vitro culture for an immunotherapy comprising culturing immune cells (e.g., human immune cells) in a medium comprising potassium ion at a concentration higher than 5 mM, wherein the immune cells have been modified to have an increased level of a c-Jun polypeptide as compared to corresponding immune cells that have not been modified to have an increased level of the c-Jun polypeptide. Provided herein is a method of expanding a population of stem-like immune cells ex vivo or in vitro comprising culturing immune cells in a medium comprising potassium ion at a concentration higher than 5 mM, wherein the immune cells have been modified to have an increased level of a c-Jun polypeptide as compared to corresponding immune cells that have not been modified to have an increased level of the c-Jun polypeptide.

Provided herein is a method of increasing the production of a cytokine by immune cells in response to an antigen stimulation, wherein the method comprises culturing immune cells in a medium comprising potassium ion at a concentration higher than 5 mM, wherein the immune cells have been modified to have an increased level of a c-Jun polypeptide as compared to corresponding immune cells that have not been modified to have an increased level of the c-Jun polypeptide.

In some aspects, the cytokine comprises IL-2. In some aspects, after the culturing, the production of the cytokine in response to the antigen stimulation is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 25-fold, at least about 30-fold, at least about 35-fold, at least about 40-fold, at least about 45-fold, at least about 50-fold, at least about 75-fold, at least about 100-fold, at least about 200-fold, at least about 300-fold, at least about 400-fold, at least about 500-fold, at least about 750-fold, or at least about 1,000-fold or more, as compared to reference immune cells.

Provided herein is a method of increasing an effector function of immune cells in response to persistent antigen stimulation comprising culturing the immune cells in a medium comprising potassium ion at a concentration higher than 5 mM, wherein the immune cells have been modified to have an increased level of a c-Jun polypeptide as compared to corresponding immune cells that have not been modified to have an increased level of the c-Jun polypeptide.

In some aspects, the immune cells retain effector function for at least one, at least two, or at least three additional rounds of an antigen stimulation assay as compared to reference immune cells. In some aspects, the effector function comprises the ability: (i) to kill target cells (e.g., tumor cells), (ii) to produce a cytokine upon further antigen stimulation, or (iii) both (i) and (ii). In some aspects, the cytokine comprises IFN-γ.

In some aspects, after the culturing, the effector function of the immune cells in response to persistent antigen stimulation is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 25-fold, at least about 30-fold, at least about 35-fold, at least about 40-fold, at least about 45-fold, at least about 50-fold, at least about 75-fold, at least about 100-fold, at least about 200-fold, at least about 300-fold, at least about 400-fold, at least about 500-fold, at least about 750-fold, or at least about 1,000-fold or more, as compared to reference immune cells.

In any of the above methods, in some aspects, the reference immune cells comprise corresponding immune cells that: (i) have been modified to have an increased level of the c-Jun polypeptide and cultured in a medium that does not comprise potassium ion at a concentration higher than 5 mM; (ii) have not been modified to have an increased level of the c-Jun polypeptide and cultured in the medium that comprises potassium ion at a concentration higher than 5 mM; (iii) have not been modified to have an increased level of the c-Jun polypeptide and cultured in a medium that does not comprise potassium ion at a concentration higher than 5 mM; or (iv) any combination of (i) to (iii).

In some aspects, the immune cells have been modified with an exogenous polynucleotide encoding the c-Jun polypeptide, such that after the modification, the immune cells have an increased level of the c-Jun polypeptide as compared to the corresponding immune cells that have not been modified.

In some aspects, the c-Jun polypeptide is endogenous to the immune cells, and wherein the immune cells have been modified with a transcriptional activator that is capable of increasing the expression of the endogenous c-Jun polypeptide. In some aspects, the transcriptional activator is attached to a Cas protein, which has been modified to lack endonuclease activity.

Also provided herein is a method of increasing the expression of a c-Jun polypeptide in an immune cell comprising modifying the immune cell with an exogenous polynucleotide, which encodes the c-Jun polypeptide, in a medium comprising potassium ion at a concentration higher than 5 mM, wherein after the modification the expression of the c-Jun polypeptide in the immune cell is increased compared to a reference cell. As described herein, in some aspects, the reference cell comprises corresponding immune cells that: (i) have been modified to have an increased level of the c-Jun polypeptide and cultured in a medium that does not comprise potassium ion at a concentration higher than 5 mM; (ii) have not been modified to have an increased level of the c-Jun polypeptide and cultured in the medium that comprises potassium ion at a concentration higher than 5 mM; (iii) have not been modified to have an increased level of the c-Jun polypeptide and cultured in a medium that does not comprise potassium ion at a concentration higher than 5 mM; or (iv) any combination of (i) to (iii).

In some aspects, the expression of the c-Jun polypeptide is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 25-fold, at least about 30-fold, at least about 35-fold, at least about 40-fold, at least about 45-fold, at least about 50-fold, at least about 75-fold, at least about 100-fold, at least about 200-fold, at least about 300-fold, at least about 400-fold, at least about 500-fold, at least about 750-fold, or at least about 1,000-fold or more, compared to the reference cells.

Provided herein is a method of preparing immune cells ex vivo or in vitro for immunotherapy comprising modifying immune cells with an exogenous polynucleotide, which encodes a c-Jun polypeptide, in a medium comprising potassium ion at a concentration higher than 5 mM.

Provided herein is a method of preparing immune cells ex vivo or in vitro for immunotherapy comprising modifying immune cells with a transcriptional activator that is capable of increasing the expression of the endogenous c-Jun polypeptide in a medium comprising potassium ion at a concentration higher than 5 mM. In some aspects, the transcriptional activator is attached to a Cas protein, which has been modified to lack endonuclease activity.

In any of the above methods, in some aspects, the c-Jun polypeptide is overexpressed in the immune cells compared to corresponding immune cells that have not been modified.

In some aspects, the c-Jun polypeptide comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the amino acid sequence as set forth in SEQ ID NO: 13.

In some aspects, the exogenous polynucleotide encoding the c-Jun polypeptide comprises: a) a nucleotide sequence having at least 89%, at least 90%, at least about 95%, at least about 96%, at least about 97%, at least about 98%, at least about 99%, or about 100% sequence identity to the nucleic acid sequence as set forth in SEQ ID NO: 1; b) a nucleotide sequence having at least 90%, at least about 95%, at least about 96%, at least about 97%, at least about 98%, at least about 99%, or about 100% sequence identity to the nucleic acid sequence as set forth in SEQ ID NO: 2; c) a nucleotide sequence having at least about 30%, at least about 40%, at least about 50%, at least about 60%, at least about 70%, at least about 80%, at least about 90%, at least about 95%, at least about 96%, at least about 97%, at least about 98%, at least about 99%, or about 100% sequence identity to the nucleic acid sequence as set forth in SEQ ID NO: 4; d) a nucleotide sequence having at least 79%, at least 80%, at least 81%, at least 82%, at least 83%, at least 84%, at least 85%, at least 86%, at least 87%, at least 88%, at least 89%, at least 90%, at least about 95%, at least about 96%, at least about 97%, at least about 98%, at least about 99%, or about 100% sequence identity to the nucleic acid sequence as set forth in SEQ ID NO: 5; e) a nucleotide sequence having at least 88%, at least 89%, at least 90%, at least about 95%, at least about 96%, at least about 97%, at least about 98%, at least about 99%, or about 100% sequence identity to the nucleic acid sequence as set forth in SEQ ID NO: 6; f) a nucleotide sequence having at least 82%, at least 83%, at least 84%, at least 85%, at least 90%, at least about 95%, at least about 96%, at least about 97%, at least about 98%, at least about 99%, or about 100% sequence identity to the nucleic acid sequence as set forth in SEQ ID NO: 7; g) a nucleotide sequence having at least 90%, at least about 95%, at least about 96%, at least about 97%, at least about 98%, at least about 99%, or about 100% sequence identity to the nucleic acid sequence as set forth in SEQ ID NO: 8; h) a nucleotide sequence having at least 55%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the nucleic acid sequence as set forth in SEQ ID NO: 9; or i) a nucleotide sequence having at least 85%, at least 86%, at least 87%, at least 88%, at least 89%, at least about 90%, at least about 95%, at least about 96%, at least about 97%, at least about 98%, at least about 99%, or about 100% sequence identity to the nucleic acid sequence as set forth in SEQ ID NO: 10.

In some aspects, the exogenous polynucleotide encoding the c-Jun polypeptide comprises a nucleotide sequence having at least 89%, at least 90%, at least about 95%, at least about 96%, at least about 97%, at least about 98%, or at least about 99% sequence identity to the nucleic acid sequence as set forth in SEQ ID NO: 1. In some aspects, the nucleotide sequence comprises the nucleic acid sequence as set forth in SEQ ID NO: 1.

In some aspects, the exogenous polynucleotide comprises a nucleotide sequence having at least 90%, at least about 95%, at least about 96%, at least about 97%, at least about 98%, or at least about 99% sequence identity to the nucleic acid sequence as set forth in SEQ ID NO: 2. In some aspects, the nucleotide sequence comprises the nucleic acid sequence as set forth in SEQ ID NO: 2.

In some aspects, the exogenous polynucleotide comprises a nucleotide sequence having at least about 30%, at least about 40%, at least about 50%, at least about 60%, at least about 70%, at least about 80%, at least about 90%, at least about 95%, at least about 96%, at least about 97%, at least about 98%, or at least about 99% sequence identity to the nucleic acid sequence as set forth in SEQ ID NO: 4. In some aspects, the nucleotide sequence comprises the nucleic acid sequence as set forth in SEQ ID NO: 4.

In some aspects, the exogenous polynucleotide comprises a nucleotide sequence having at least 79%, at least 80%, at least 81%, at least 82%, at least 83%, at least 84%, at least 85%, at least 86%, at least 87%, at least 88%, at least 89%, at least 90%, at least about 95%, at least about 96%, at least about 97%, at least about 98%, or at least about 99% sequence identity to the nucleic acid sequence as set forth in SEQ ID NO: 5. In some aspects, the nucleotide sequence comprises the nucleic acid sequence as set forth in SEQ ID NO: 5.

In some aspects, the exogenous polynucleotide comprises a nucleotide sequence having at least 88%, at least 89%, at least 90%, at least about 95%, at least about 96%, at least about 97%, at least about 98%, or at least about 99% sequence identity to the nucleic acid sequence as set forth in SEQ ID NO: 6. In some aspects, the nucleotide sequence comprises the nucleic acid sequence as set forth in SEQ ID NO: 6.

In some aspects, the exogenous polynucleotide comprises a nucleotide sequence having at least 82%, at least 83%, at least 84%, at least 85%, at least 90%, at least about 95%, at least about 96%, at least about 97%, at least about 98%, or at least about 99% sequence identity to the nucleic acid sequence as set forth in SEQ ID NO: 7. In some aspects, the nucleotide sequence comprises the nucleic acid sequence as set forth in SEQ ID NO: 7.

In some aspects, the exogenous polynucleotide comprises a nucleotide sequence having at least 90%, at least about 95%, at least about 96%, at least about 97%, at least about 98%, or at least about 99% sequence identity to the nucleic acid sequence as set forth in SEQ ID NO: 8. In some aspects, the nucleotide sequence comprises the nucleic acid sequence as set forth in SEQ ID NO: 8.

In some aspects, the exogenous polynucleotide comprises a nucleotide sequence having at least 55%, at least about 55%, at least about 60%, at least about 65%, at least about 70%, at least about 75%, at least about 80%, at least about 85%, at least 90%, at least about 95%, at least about 96%, at least about 97%, at least about 98%, or at least about 99% sequence identity to the nucleic acid sequence as set forth in SEQ ID NO: 9. In some aspects, the nucleotide sequence comprises the nucleic acid sequence as set forth in SEQ ID NO: 9.

In some aspects, the exogenous polynucleotide comprises a nucleotide sequence having at least 85%, at least 86%, at least 87%, at least 88%, at least 89%, at least 90%, at least about 95%, at least about 96%, at least about 97%, at least about 98%, or at least about 99% sequence identity to the nucleic acid sequence as set forth in SEQ ID NO: 10. In some aspects, the nucleotide sequence comprises the nucleic acid sequence as set forth in SEQ ID NO: 10.

In some aspects, the immune cells of the methods provided above further comprise a nucleotide sequence encoding a ligand binding protein. In some aspects, the ligand binding protein is selected from a chimeric antigen receptor (CAR), a T cell receptor (TCR), a chimeric antibody-T cell receptor (caTCR), a chimeric signaling receptor (CSR), T cell receptor mimic (TCR mimic), or combinations thereof. In some aspects, the CAR is designed as a standard CAR, a split CAR, an off-switch CAR, an on-switch CAR, a first-generation CAR, a second-generation CAR, a third-generation CAR, or a fourth-generation CAR. In some aspects, the ligand binding protein comprises an antigen-binding domain, a transmembrane domain, a costimulatory domain, an intracellular signaling domain, or combinations thereof.

In some aspects, the antigen-binding domain of the ligand binding protein specifically binds an antigen selected from the group consisting of AFP (alpha-fetoprotein), avP6 or another integrin, BCMA, Braf, B7-H3, B7-H6, CA9 (carbonic anhydrase 9), CCL-1 (C-C motif chemokine ligand 1), CD5, CD19, CD20, CD21, CD22, CD23, CD24, CD30, CD33, CD38, CD40, CD44, CD44v6, CD44v7/8, CD45, CD47, CD56, CD66e, CD70, CD74, CD79a, CD79b, CD98, CD123, CD138, CD171, CD352, CEA (carcinoembryonic antigen), Claudin 18.2, Claudin 6, c-MET, DLL3 (delta-like protein 3), DLL4, ENPP3 (ectonucleotide pyrophosphatase/phosphodiesterase family member 3), EpCAM, EPG-2 (epithelial glycoprotein 2), EPG-40, ephrinB2, EPHa2 (ephrine receptor A2), ERBB dimers, estrogen receptor, ETBR (endothelin B receptor), FAP-α (fibroblast activation protein α), fetal AchR (fetal acetylcholine receptor), FBP (a folate binding protein), FCRL5, FR-α (folate receptor alpha), GCC (guanyl cyclase C), GD2, GD3, GPC2 (glypican-2), GPC3, gp100 (glycoprotein 100), GPNMB (glycoprotein NMB), GPRC5D (G Protein Coupled Receptor 5D), HER2, HER3, HER4, hepatitis B surface antigen, HLA-A1 (human leukocyte antigen A1), HLA-A2 (human leukocyte antigen A2), HMW-MAA (human high molecular weight-melanoma-associated antigen), IGF1R (insulin-like growth factor 1 receptor), Ig kappa, Ig lambda, IL-22Ra (IL-22 receptor alpha), IL-13Ra2 (IL-13 receptor alpha 2), KDR (kinase insert domain receptor), LI cell adhesion molecule (LI-CAM), Liv-1, LRRC8A (leucine rich repeat containing 8 Family member A), Lewis Y, melanoma-associated antigen (MAGE)-A1, MAGE-A3, MAGE-A6, MART-1 (melan A), murine cytomegalovirus (MCMV), MCSP (melanoma-associated chondroitin sulfate proteoglycan), mesothelin, mucin 1 (MUC1), MUC16, MHC/peptide complexes (e.g., HLA-A complexed with peptides derived from AFP, KRAS, NY-ESO, MAGE-A, and WT1), NCAM (neural cell adhesion molecule), Nectin-4, NKG2D (natural killer group 2 member D) ligands, NY-ESO, oncofetal antigen, PD-1, PD-L1, PRAME (preferentially expressed antigen of melanoma), progesterone receptor, PSA (prostate specific antigen), PSCA (prostate stem cell antigen), PSMA (prostate specific membrane antigen), ROR1, ROR2, SIRPa (signal-regulatory protein alpha), SLIT, SLITRK6 (NTRK-like protein 6), STEAPI (six transmembrane epithelial antigen of the prostate 1), survivin, TAG72 (tumor-associated glycoprotein 72), TPBG (trophoblast glycoprotein), Trop-2, VEGFR1 (vascular endothelial growth factor receptor 1), VEGFR2, and antigens from HIV, HBV, HCV, HPV, and other pathogens, and any combination thereof. In some aspects, the antigen-binding domain specifically binds ROR1. In some aspects, the antigen-binding domain specifically binds GPC2.

In some aspects, the costimulatory domain of the ligand-binding domain comprises a costimulatory domain of an interleukin-2 receptor (IL-2R), interleukin-12 receptor (IL-12R), IL-7, IL-21, IL-23, IL-15, CD2, CD3, CD4, CD7, CD8, CD27, CD28, CD30, CD40, 4-1BB/CD137, ICOS, lymphocyte function-associated antigen-1 (LFA-1), LIGHT, NKG2C, OX40, DAP10, or any combination thereof. In some aspects, the costimulatory domain comprises a 4-1BB/CD137 costimulatory domain.

In some aspects, the transmembrane domain of the ligand-binding domain comprises a transmembrane domain of KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rbeta, IL2Rgamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, CD19, CD8, or any combination thereof. In some aspects, the transmembrane domain comprises a CD28 transmembrane domain.

In some aspects, the intracellular signaling domain of a ligand-binding domain comprises an intracellular signaling domain derived from CD3 zeta, FcR gamma, common FcR gamma (FCERIG), Fc gamma RIIa, FcR beta (Fc Epsilon Rib), CD3 gamma, CD3 delta, CD3 epsilon, CD22, CD79a, CD79b, CD278 (also known as ICOS), FcεRI, CD66d, CD32, DAP10, DAP12, or any combination thereof. In some aspects, the intracellular signaling domain comprises a CD3 zeta intracellular signaling domain.

In some aspects, the ligand binding domain is a TCR, wherein the TCR specifically binds a tumor antigen/MHC complex. In some aspects, the tumor antigen is derived from AFP, CD19, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-1Ra, PSCA, PRSS21, VEGFR2, LewisY, CD24, PDGFR-beta, SSEA-4, CD20, folate receptor alpha, ERBB2 (Her2/neu), Kras, Braf, MUC1, MUC16, EGFR, NCAM, prostase, PAP, ELF2M, Ephrin B2, IGF-I receptor, CAIX, LMP2, gp100, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV, HPV E6, E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant (e.g., HRAS, KRAS, NRAS), hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3ε, CD4, CD5, CD7, the extracellular portion of the APRIL protein, neoantigen, or any combinations thereof.

In some aspects, the c-Jun polypeptide is linked to the ligand binding protein by a linker. In some aspects, the linker comprises a cleavable linker. In some aspects, the linker is a P2A linker, a T2A linker, an F2A linker, an E2A linker, a furin cleavage site, or any combination thereof. In some aspects, the linker comprises an amino acid sequence having at least about 80%, at least about 85%, at least about 90%, at least about 95%, at least about 96%, at least about 97%, at least about 98%, or at least about 99% sequence identity to the amino acid sequence as set forth in SEQ ID NO: 14. In some aspects, the linker comprises the amino acid sequence as set forth in SEQ ID NO: 14.

In some aspects, an immune cell of any of the methods provided above further comprise a nucleotide sequence encoding a truncated EGFR (EGFRt), which is expressed in the immune cells. In some aspects, the EGFRt comprises an amino acid sequence having at least about 80%, at least about 85%, at least about 90%, at least about 95%, at least about 96%, at least about 97%, at least about 98%, or at least about 99% sequence identity to the amino acid sequence as set forth in SEQ ID NO: 24. In some aspects, the EGFRt comprises the amino acid sequence as set forth in SEQ ID NO: 24.

In some aspects, the EGFRt is linked to the c-Jun polypeptide and/or the ligand binding protein by a linker. In some aspects, the linker comprises a cleavable linker. In some aspects, the linker is a P2A linker, a T2A linker, an F2A linker, an E2A linker, a furin cleavage site, or any combination thereof. In some aspects, the linker comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 14. In some aspects, linker comprises the amino acid sequence set forth in SEQ ID NO: 14.

In some aspects, the exogenous polynucleotide of the methods provided above comprises a regulatory element, and wherein a vector comprises the exogenous polynucleotide. In some aspects, the vector is a polycistronic expression vector. In some aspects, the vector comprises a viral vector, a mammalian vector, or a bacterial vector. In some aspects, the vector comprises an adenoviral vector, a lentivirus, a Sendai virus vector, a baculoviral vector, an Epstein Barr viral vector, a papovaviral vector, a vaccinia viral vector, a herpes simplex viral vector, a hybrid vector, or an adeno associated virus (AAV) vector. In some aspects, the vector is a lentivirus.

In some aspects, the regulatory element comprises a promoter. In some aspects, the promoter comprises a dl587rev primer-binding site substituted (MND) promoter, EF1a promoter, ubiquitin promoter, or combinations thereof.

In any of the methods provided above, in some aspects, the concentration of potassium ion is higher than about 10 mM, higher than about 15 mM, higher than about 20 mM, higher than about 25 mM, higher than about 30 mM, higher than about 35 mM, higher than about 40 mM, higher than about 45 mM, higher than about 50 mM, higher than about 55 mM, higher than about 60 mM, higher than about 65 mM, higher than about 70 mM, higher than about 75 mM, higher than about 80 mM, higher than about 85 mM, or higher than about 90 mM. In some aspects, the concentration of potassium ion is selected from the group consisting of about 40 mM, about 45 mM, about 50 mM, about 55 mM, about 60 mM, about 65 mM, about 70 mM, about 75 mM, and about 80 mM. In some aspects, the concentration of potassium ion is between about 30 mM and about 80 mM, between about 40 mM and about 80 mM, between about 50 mM and 80 mM, between about 60 mM and about 80 mM, between about 70 mM and about 80 mM, between about 40 mM and about 70 mM, between about 50 mM and about 70 mM, between about 60 mM and about 70 mM, between about 40 mM and about 60 mM, between about 50 mM and about 60 mM, or between about 40 mM and about 50 mM. In some aspects, the concentration of potassium ion is about 50 mM, about 60 mM, or about 70 mM.

In some aspects, the medium further comprises sodium ion. In some aspects, the medium further comprises NaCl. In some aspects, the medium comprises less than about 140 mM, less than about 130 mM, less than about 120 mM, less than about 110 mM, less than about 100 mM, less than about 90 mM, less than about 80 mM, less than about 70 mM, less than about 60 mM, less than about 50 mM, or less than about 40 mM NaCl.

In some aspects, the medium is hypotonic or isotonic. In some aspects, the medium is hypotonic, and wherein the sum of the potassium ion concentration and the sodium ion concentration, multiplied by two is less than 280 mM. In some aspects, the medium is hypotonic, and wherein the sum of the potassium ion concentration and the sodium ion concentration, multiplied by two is more than 240 mM and less than 280 mM. In some aspects, the medium is isotonic, and wherein the sum of the potassium ion concentration and the sodium ion concentration, multiplied by two is more than or equal to 280 mM and less than 300 mM.

In some aspects, the concentration of potassium ion is about 60 mM, and the concentration of NaCl is less than about 80 mM, less than about 75 mM, less than about 70 mM, less than about 65 mM, or less than about 60 mM. In some aspects, the concentration of potassium ion is about 55 mM, and the concentration of NaCl is less than about 85 mM, less than about 80 mM, less than about 75 mM, less than about 70 mM, or less than about 65 mM. In some aspects, the concentration of potassium ion is about 50 mM, and the concentration of NaCl is less than about 90 mM, less than about 85 mM, less than about 80 mM, less than about 75 mM, or less than about 70 mM.

In some aspects, the medium of the methods provided above further comprises one or more cytokines. In some aspects, the one or more cytokines comprise Interleukin-2 (IL-2), Interleukin-7 (IL-7), Interleukin-21 (IL-21), Interleukin-15 (IL-15), or any combination thereof. In some aspects, the one or more cytokines comprise IL-2, IL-7, and IL-15.

In some aspects, the medium comprises IL-2 at a concentration from about 50 IU/mL to about 500 IU/mL. In some aspects, the concentration of IL-2 is about 50 IU/mL, about 60 IU/mL, about 70 IU/mL, about 80 IU/mL, about 90 IU/mL, about 100 IU/mL, about 125 IU/mL, about 150 IU/mL, about 175 IU/mL, about 200 IU/mL, about 225 IU/mL, about 250 IU/mL, about 275 IU/mL, about 300 IU/mL, about 350 IU/mL, about 400 IU/mL, about 450 IU/mL, or about 500 IU/mL. In some aspects, the concentration of IL-2 is between about 100 IU/mL to about 300 IU/mL. In some aspects, the concentration of IL-2 is about 200 IU/mL.

In some aspects, the medium comprises IL-21 at a concentration from about 50 IU/mL to about 500 IU/mL. In some aspects, the concentration of IL-21 is about 50 IU/mL, about 60 IU/mL, about 70 IU/mL, about 80 IU/mL, about 90 IU/mL, about 100 IU/mL, about 125 IU/mL, about 150 IU/mL, about 175 IU/mL, about 200 IU/mL, about 225 IU/mL, about 250 IU/mL, about 275 IU/mL, about 300 IU/mL, about 350 IU/mL, about 400 IU/mL, about 450 IU/mL, or about 500 IU/mL. In some aspects, the concentration of IL-21 is between about 100 IU/mL to about 300 IU/mL. In some aspects, the concentration of IL-21 is about 200 IU/mL.

In some aspects, the medium comprises IL-7 at a concentration from about 500 IU/mL to about 1,500 IU/mL. In some aspects, the concentration of IL-7 is about 500 IU/mL, about 550 IU/mL, about 600 IU/mL, about 650 IU/mL, about 700 IU/mL, about 750 IU/mL, about 800 IU/mL, about 850 IU/mL, about 900 IU/mL, about 950 IU/mL, about 1,000 IU/mL, about 1,050 IU/mL, about 1,100 IU/mL, about 1,150 IU/mL, about 1,200 IU/mL, about 1,250 IU/mL, about 1,300 IU/mL, about 1,350 IU/mL, about 1,400 IU/mL, about 1,450 IU/mL, or about 1,500 IU/mL. In some aspects, the concentration of IL-7 is about 1,000 IU/mL to about 1,400 IU/mL. In some aspects, the concentration of IL-7 is about 1,200 IU/mL.

In some aspects, the medium comprises IL-15 at a concentration from about 50 IU/mL to about 500 IU/mL. In some aspects, the concentration of IL-15 is about 50 IU/mL, about 60 IU/mL, about 70 IU/mL, about 80 IU/mL, about 90 IU/mL, about 100 IU/mL, about 125 IU/mL, about 150 IU/mL, about 175 IU/mL, about 200 IU/mL, about 225 IU/mL, about 250 IU/mL, about 275 IU/mL, about 300 IU/mL, about 350 IU/mL, about 400 IU/mL, about 450 IU/mL, or about 500 IU/mL. In some aspects, the concentration of IL-15 is between about 100 IU/mL to about 300 IU/mL. In some aspects, the concentration of IL-15 is about 200 IU/mL.

In some aspects, the medium further comprises a cell expansion agent. In some aspects, the cell expansion agent comprises a GSK3B inhibitor, an ACLY inhibitor, a PI3K inhibitor, an AKT inhibitor, or any combination thereof. In some aspects, the PI3K inhibitor is selected from hydroxyl citrate, LY294002, pictilisib, CAL101, IC87114, and any combination thereof. In some aspects, the AKT inhibitor is selected from MK2206, A443654, AKTi-VIII, and any combination thereof.

In some aspects, the medium of the methods provided above is capable of: a) increasing the number and/or percentage of less differentiated and/or undifferentiated cells; b) increasing transduction efficiency; c) increasing stem-like immune cells; d) increasing in vivo viability; e) increasing cell potency; f) preventing cell exhaustion; or g) any combination thereof, in the final cell product as compared to the starting immune cells, compared to the immune cells cultured in a medium without the high concentration of potassium ion, and/or the immune cells without the c-Jun polypeptide.

In some aspects, the medium further comprises calcium ion, glucose, or any combination thereof.

In some aspects, the medium further comprises glucose, and wherein the concentration of glucose is more than about 10 mM. In some aspects, the concentration of glucose is from about 10 mM to about 25 mM, from about 10 mM to about 20 mM, from about 15 mM to about 25 mM, from about 15 mM to about 20 mM, from about 15 mM to about 19 mM, from about 15 mM to about 18 mM, from about 15 mM to about 17 mM, from about 15 mM to about 16 mM, from about 16 mM to about 20 mM, from about 16 mM to about 19 mM, from about 16 mM to about 18 mM, from about 16 mM to about 17 mM, from about 17 mM to about 20 mM, from about 17 mM to about 19 mM, or from about 17 mM to about 18 mM. In some aspects, the concentration of glucose is about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, or about 25 mM. In some aspects, the concentration of glucose is about 15.4 mM, about 15.9 mM, about 16.3 mM, about 16.8 mM, about 17.2 mM, or about 17.7 mM.

In some aspects, the medium further comprises calcium ion, and wherein the concentration of calcium ion is more than about 0.4 mM. In some aspects, the concentration of calcium ion is from about 0.4 mM to about 2.8 mM, about 0.4 mM to about 2.5 mM, from about 0.5 mM to about 2.0 mM, from about 1.0 mM to about 2.0 mM, from about 1.1 mM to about 2.0 mM, from about 1.2 mM to about 2.0 mM, from about 1.3 mM to about 2.0 mM, from about 1.4 mM to about 2.0 mM, from about 1.5 mM to about 2.0 mM, from about 1.6 mM to about 2.0 mM, from about 1.6 mM to about 2.8 mM, from about 1.7 mM to about 2.0 mM, from about 1.8 mM to about 2.0 mM, from about 1.2 to about 1.3 mM, from about 1.2 to about 1.4 mM, from about 1.2 to about 1.5 mM, from about 1.2 to about 1.6 mM, from about 1.2 to about 1.7 mM, from about 1.2 to about 1.8 mM, from about 1.3 to about 1.4 mM, from about 1.3 to about 1.5 mM, from about 1.3 to about 1.6 mM, from about 1.3 to about 1.7 mM, from about 1.3 to about 1.8 mM, from about 1.4 to about 1.5 mM, from about 1.4 to about 1.6 mM, from about 1.4 to about 1.7 mM, from about 1.4 to about 1.8 mM, from about 1.5 to about 1.6 mM, from about 1.5 to about 1.7 mM, from about 1.5 to about 1.8 mM, from about 1.6 to about 1.7 mM, from about 1.6 to about 1.8 mM, or from about 1.7 to about 1.8 mM. In some aspects, the concentration of calcium ion is about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, about 2.0 mM, about 2.1 mM, about 2.2. mM, about 2.3 mM, about 2.4 mM, about 2.5 mM, about 2.6 mM, about 2.7 mM, about 2.8 mM, about 2.9 mM, or about 3.0 mM.

In some aspects, the immune cells are CD3+, CD45RO−, CCR7+, CD45RA+, CD62L+, CD27+, CD28+, or TCF7+, or any combination thereof, following the culturing.

Provided herein is a population of human immune cells prepared by any of the methods provided herein. In some aspects, the immune cells are T cells. In some aspects, the T cells comprise CD8⁺ T cells, CD4+ T cells, or both.

Provided herein is a pharmaceutical composition comprising the population of human immune cells described herein.

Provided herein is a composition comprising a population of CD4+ T cells and CD8+ T cells, which have been modified to (a) express a chimeric antigen receptor (CAR) and (b) have an increased level of a c-Jun polypeptide as compared to a corresponding immune cells that have not been modified to have an increased level of the c-Jun polypeptide, wherein (i) at least about 20% of the modified CD4+ T cells are surface positive for CCR7 and CD45RA; (ii) at least about 20% of the modified CD8+ T cells are surface positive for CCR7 and CD45RA; or (iii) both (i) and (ii).

Provided herein is a composition comprising a population of CD4+ T cells, which have been modified to (a) express a chimeric antigen receptor (CAR) and (b) have an increased level of a c-Jun polypeptide as compared to a corresponding immune cells that have not been modified to have an increased level of the c-Jun polypeptide, wherein at least about 20% of the modified CD4+ T cells are surface positive for CCR7 and CD45RA. Provided herein is a composition comprising a population of CD8+ T cells, which have been modified to (a) express a chimeric antigen receptor (CAR) and (b) have an increased level of a c-Jun polypeptide as compared to a corresponding immune cells that have not been modified to have an increased level of the c-Jun polypeptide, wherein at least about 20 percent of the modified CD8+ T cells are surface positive for CCR7 and CD45RA.

Provided herein is a composition comprising a population of CD4+ T cells and CD8+ T cells, which have been modified to (a) express an engineered T cell receptor (TCR) and (b) have an increased level of a c-Jun polypeptide as compared to a corresponding immune cells that have not been modified to have an increased level of the c-Jun polypeptide, wherein (i) at least about 15% of the modified CD4+ T cells are surface positive for CCR7 and CD45RA; (ii) at least about 20% of the modified CD8+ T cells are surface positive for CCR7 and CD45RA; or (iii) both (i) and (ii). Provided herein is a composition comprising a population of CD4+ T cells, which have been modified to (a) express an engineered T cell receptor (TCR) and (b) have an increased level of a c-Jun polypeptide as compared to a corresponding immune cells that have not been modified to have an increased level of the c-Jun polypeptide, wherein at least about 15% of the modified CD4+ T cells are surface positive for CCR7 and CD45RA. Provided herein is a composition comprising a population of CD8+ T cells, which have been modified to (a) express an engineered T cell receptor (TCR) and (b) have an increased level of a c-Jun polypeptide as compared to a corresponding immune cells that have not been modified to have an increased level of the c-Jun polypeptide, wherein at least about 20 percent of the modified CD8+ T cells are surface positive for CCR7 and CD45RA.

Also provided herein is a composition comprising a population of immune cells which have been modified to (a) express an engineered chimeric antigen receptor (CAR) or an engineered T cell receptor (TCR) and (b) have an increased level of a c-Jun polypeptide as compared to a corresponding immune cells that have not been modified to have an increased level of the c-Jun polypeptide, wherein at least about 4% of the cells are progenitor exhausted T cells. Some aspects of the present disclosure is related to a composition comprising a population of immune cells which have been modified to (a) express an engineered chimeric antigen receptor (CAR) or an engineered T cell receptor (TCR) and (b) have an increased level of a c-Jun polypeptide as compared to a corresponding immune cells that have not been modified to have an increased level of the c-Jun polypeptide, wherein between about 4% and about 6% of the cells are progenitor exhausted T cells. Also provided herein is a composition comprising a population of immune cells which have been modified to (a) express an engineered chimeric antigen receptor (CAR) or an engineered T cell receptor (TCR) and (b) have an increased level of a c-Jun polypeptide as compared to a corresponding immune cells that have not been modified to have an increased level of the c-Jun polypeptide, wherein at least about 4% of the cells are progenitor exhausted T cells and at least about 4% of the cells are stem-like T cells.

In some aspects, the population of human immune cells, the pharmaceutical compositions, or the compositions described herein is for treating a subject in need of a therapy. Also provided herein is a use of the population of human immune cells, the pharmaceutical compositions, or the compositions described herein in the manufacture of a medicament for treating or preventing a disease or condition in a subject in need thereof. In some aspects, the disease or condition comprises a cancer.

Provided herein is a use of the population of human immune cells, the pharmaceutical compositions, or the compositions described herein for preventing or reducing exhaustion of a cell useful for a therapy.

Provided herein is a method of treating or preventing a disease or condition in a subject in need thereof comprising administering any of the population of human cells, pharmaceutical compositions, or the compositions described herein. In some aspects, the disease or condition comprises a cancer.

### BRIEF DESCRIPTION OF THE FIGURES

FIGS. 1A, 1B, and 1C show the effect of metabolic reprogramming media (MRM) on c-Jun protein expression level (shown as median fluorescence intensity (MFI)) in transduced T cells from different test groups. The different test groups are as follows: (1) non-transduced T cells cultured in a control medium (i.e., TCM); (2) T cells transduced with control CD19t-R12 CAR (i.e., R12 CAR without c-Jun) and cultured in TCM; (3) T cells transduced with c-Jun-R12 CAR (i.e., R12 CAR with c-Jun) and cultured in TCM; (4) non-transduced T cells cultured in MRM; (5) T cells transduced with control CD19t-R12 CAR and cultured in MRM; and (6) T cells transduced with c-Jun-R12 CAR and cultured in MRM. The T cells (includes both CD4+ and CD8+ T cells) that were transduced were derived from three different donors: donor #1 (FIG. 1A), donor #2 (FIG. 1B), and donor #3 (FIG. 1C).

FIGS. 2A, 2B, 2C, 2D, 2E, and 2F provide comparison of the percentage of stem-like transduced CD4+ T cells (FIGS. 2A, 2B, and 2C—three different donors) and CD8+ T cells (FIGS. 2D, 2E, and 2F—from the three different donors) from the different test groups. The different test groups are the same as those described in FIGS. 1A-1C. As described in Example 2, stem-like cells were identified as CD45RO⁻CCR7⁺CD45RA⁺CD62L⁺CD27⁺CD28⁺TCF7⁺.

FIGS. 2G, 2H, 2I, 2J, 2K, and 2L show the percentage of naïve and stem cell memory T cells from the same three donors for CD4+ T cells (FIGS. 2G, 2H, 2I) and CD8+ T cells (FIGS. 2J, 2K, 2L). As described in Example 2, naïve and stem cell memory T cells were identified as CCR7⁺CD45RA⁺.

FIGS. 3A, 3B, and 3C provide comparison of IL-2 production by T cells transduced and cultured in metabolic reprogramming media (MRM) or in a control medium (i.e., TCM) after primary antigen stimulation. The T cells that were transduced were derived from three different donors: donor #1 (FIG. 3A), donor #2 (FIG. 3B), and donor #3 (FIG. 3C). The different test groups are as follows: (1) T cells transduced with control CD19t-R12 CAR (i.e., R12 CAR without c-Jun) and cultured in TCM (closed circle); (2) T cells transduced with c-Jun-R12 CAR (i.e., R12 CAR with c-Jun) and cultured in TCM (closed square); (3) T cells transduced with control CD19t-R12 CAR and cultured in MRM (open circle); and (4) T cells transduced with c-Jun-R12 CAR and cultured in MRM (open square). The x-axis provides the effector:target (E:T) ratio (i.e. ratio of transduced T cells to target tumor cell).

FIGS. 4A, 4B, and 4C provide comparison of IFN-γ production by T cells transduced and cultured in metabolic reprogramming media (MRM) or in a control medium (i.e., TCM) after multiple rounds of antigen stimulation. As further provided in Example 3, the serial stimulation assay was terminated when the number of transduced T cells required to reseed the subsequent round was not achieved: (i) four rounds of antigen stimulation for donor #1 (FIG. 4A), (ii) three rounds of antigen stimulation for donor #2 (FIG. 4B), and (iii) two rounds of antigen stimulation for donor #3 (FIG. 4C). The different test groups are as follows: (1) T cells transduced with control CD19t-R12 CAR (i.e., R12 CAR without c-Jun) and cultured in TCM (closed circle); (2) T cells transduced with c-Jun-R12 CAR (i.e., R12 CAR with c-Jun) and cultured in TCM (closed square); (3) T cells transduced with control CD19t-R12 CAR and cultured in MRM (open circle); and (3) T cells transduced with c-Jun-R12 CAR and cultured in MRM (open square). The x-axis provides the effector:target (E:T) ratio (i.e. ratio of transduced T cells to target tumor cell).

FIGS. 5A, 5B, 5C, 5D, 5E, and 5F show the ability of the transduced CD8+ T cells to kill target tumor cells after multiple rounds of antigen stimulation. The T cells that were transduced were derived from three different donors: donor #1 (FIGS. 5B and 5E), donor #2 (FIGS. 5C and 5F), and donor #3 (FIGS. 5A and 5D). FIGS. 5A, 5B, and 5C provide results for CD8+ T cells transduced with either the control CD19t-R12 CAR (i.e., R12 CAR without c-Jun; black bars) or the c-Jun-R12 CAR (i.e., R12 CAR with c-Jun; white bars), and cultured in a control medium (i.e., TCM). FIGS. 5D, 5E, and 5F provide results for CD8+ T cells transduced with either the control CD19t-R12 CAR (black bars) or the c-Jun-R12 CAR (white bars), and cultured in metabolic reprogramming media (MRM). The x-axis provides the effector:target (E:T) ratio (i.e. ratio of transduced T cells to target tumor cell).

FIGS. 6A, 6B, and 6C show the effect of metabolic reprogramming media (MRM) on c-Jun protein expression level (shown as median fluorescence intensity (MFI)) in transduced T cells from different test groups. The different test groups are as follows: (1) T cells transduced with control NY-ESO1 TCR (i.e., NY-ESO1 TCR without c-Jun) and cultured in a control medium (TCM) (“Control TCR TCM”); (2) T cells transduced with c-Jun-NY-ESO1 TCR (i.e., NY-ESO1 TCR with c-Jun) and cultured in TCM (“c-Jun-TCR TCM”); (3) T cells transduced with control NY-ESO1 TCR and cultured in MRM (“control TCR MRM”); and (4) T cells transduced with c-Jun-NY-ESO1 TCR and cultured in MRM (“c-Jun-TCR MRM”). The T cells (includes both CD4+ and CD8+ T cells) that were transduced were derived from three different donors: donor #1 (FIG. 6A), donor #2 (FIG. 6B), and donor #3 (FIG. 6C).

FIGS. 7A, 7B, 7C, 7D, 7E, and 7F provide comparison of the percentage of naïve and stem cell memory transduced CD4+ T cells (FIGS. 7A, 7B, and 7C—three different donors) and CD8+ T cells (FIGS. 7D, 7E, and 7F—from the three different donors) from the different test groups. The different test groups are the same as those described in FIGS. 6A-6C. As described in Example 8, naïve and stem cell memory T cells were identified as CCR7⁺CD45RA⁺.

FIGS. 8A, 8B, 8C, 8D, 8E, and 8F provide comparison of IL-2 production by T cells transduced and cultured in metabolic reprogramming media (MRM) or in a control medium (i.e., TCM) during primary antigen stimulation by A375 (FIG. 8A, FIG. 8B and FIG. 8C) and H1703 (FIG. 8D, FIG. 8E and FIG. 8F) target tumor cells. The T cells that were transduced were derived from three different donors: donor #1 (FIG. 8A and FIG. 8D), donor #2 (FIG. 8B and FIG. 8E), and donor #3 (FIG. 8C and FIG. 8F). The different test groups are as follows: (1) T cells transduced with control NY-ESO1 TCR (i.e., NY-ESO1 TCR without c-Jun) and cultured in TCM (closed circle); (2) T cells transduced with c-Jun-NY-ESO1 TCR (i.e., NY-ESO1 TCR with c-Jun) and cultured in TCM (closed square); (3) T cells transduced with control NY-ESO1 TCR and cultured in MRM (open circle); and (4) T cells transduced with c-Jun-NY-ESO1 TCR and cultured in MRM (open square). The effector:target (E:T) ratios (i.e. ratio of transduced T cells to target tumor cell) are denoted at the top of each plot.

FIGS. 9A, 9B, 9C, 9D, 9E, and 9F provide comparison of IFN-γ production by T cells transduced and cultured in metabolic reprogramming media (MRM) or in a control medium (i.e., T_{CM}) during primary antigen stimulation by A375 (FIG. 9A, FIG. 9B and FIG. 9C) and H1703 (FIG. 9D, FIG. 9E and FIG. 9F) target tumor cells. The T cells that were transduced were derived from three different donors: donor #1 (FIG. 9A and FIG. 9D), donor #2 (FIG. 9B and FIG. 9E), and donor #3 (FIG. 9C and FIG. 9F). The different test groups are as follows: (1) T cells transduced with control NY-ESO1 TCR (i.e., NY-ESO1 TCR without c-Jun) and cultured in T_{CM} (closed circle); (2) T cells transduced with c-Jun-NY-ESO1 TCR (i.e., NY-ESO1 TCR with c-Jun) and cultured in TCM (closed square); (3) T cells transduced with control NY-ESO1 TCR and cultured in MRM (open circle); and (4) T cells transduced with c-Jun-NY-ESO1 TCR and cultured in MRM (open square). The effector:target (E:T) ratios (i.e. ratio of transduced T cells to target tumor cell) are denoted at the top of each plot.

FIGS. 10A, 10B, 10C, 10D, 10E, and 10F show the ability of the transduced T cells to kill target tumor cells A375 (FIGS. 10A, 10B, and 10C) or H1703 (FIGS. 10D, 10E, and 10F) through multiple rounds of antigen stimulation. The T cells that were transduced were derived from three different donors: donor #1 (FIGS. 10A and 10D), donor #2 (FIGS. 10B and 10E), and donor #3 (FIGS. 10C and 10F). The different test groups are as follows: (1) non-transduced T cells cultured in a control medium (i.e., TCM) (closed triangle; “Mock—TCM”); (2) non-transduced T cells cultured in MRM (open triangle; “Mock—MRM”); (3) T cells transduced with control NY-ESO1 TCR (i.e., NY-ESO1 TCR without c-Jun) and cultured in TCM (closed circle; “Control TCR—TCM”); (4) T cells transduced with c-Jun-NY-ESO1 TCR (i.e., NY-ESO1 TCR with c-Jun) and cultured in TCM (closed square; “c-JUN-TCR—TCM”); (5) T cells transduced with control NY-ESO1 TCR and cultured in MRM (open circle; “Control TCR—MRM”); and (6) T cells transduced with c-Jun-NY-ESO1 TCR and cultured in MRM (open square; “c-JUN-TCR—MRM”). The effector:target (E:T) ratios (i.e. ratio of transduced T cells to target tumor cell) are 1:4 for A375 and 1:1 for H1703.

FIGS. 11A and 11B provide transcriptome profile of anti-ROR1 CAR T cells following serial antigen stimulation. As further described in Example 6, some of the anti-ROR1 CAR T cells were modified to overexpress c-Jun protein and/or cultured in MRM. The different test groups shown are as follows: (1) T cells transduced with control CD19t-R12 CAR (i.e., R12 CAR without c-Jun) and cultured in control media (gray bars); and (2) T cells transduced with c-Jun-R12 CAR (i.e., R12 CAR with c-Jun) and cultured in MRM (black bars). FIG. 11A shows the proportion of CD8+ T cells that are enriched for stem-like genes at days 7 and 10 of the serial antigen stimulation assay. FIG. 11B shows the proportion of CD8+ T cells that are enriched for T cell terminal exhaustion genes.

FIGS. 12A, 12B, and 12C are bar graphs showing c-Jun expression in T cells transduced with ROR1 CAR and cultured in either TCM or MRM comprising different concentrations of potassium ion. As further described in Example 13, the potassium ion concentration of the different MRMs tested ranged between 40-80 mM (i.e., low to high concentration). The different transduction conditions (or test groups) are as follows: (1) T cells transduced with control CD19t-R12 CAR (i.e., R12 CAR without c-Jun) and cultured in TCM; (2) T cells transduced with c-Jun-R12 CAR (i.e., R12 CAR with c-Jun) and cultured in TCM; (3) T cells transduced with control CD19t-R12 CAR and cultured in MRM of different potassium concentrations; and (4) T cells transduced with c-Jun-R12 CAR and cultured in in MRM of different potassium concentrations. Each of FIGS. 12A, 12B, and 12C provides results for biological replicates of T cells isolated from three independent donors.

FIGS. 13A, 13B, and 13C provide comparison of the percentage of stem-like CD4+ T cells transduced and cultured in either TCM or MRM comprising potassium ion at a concentration ranging from 40-80 mM (i.e., low to high concentration). The different test groups are the same as those described in FIGS. 12A-12C. The stem-like cells were identified with cell surface markers CD45RO⁻CCR7⁺CD45RA⁺CD62L⁺CD27⁺CD28⁺TCF7+. Each of FIGS. 13A, 13B, and 13C provides results for biological replicates of T cells isolated from three independent donors.

FIGS. 14A, 14B, and 14C provide comparison of the percentage of stem-like CD8+ T cells transduced and cultured in either TCM or MRM comprising potassium ion at a concentration ranging from 40-80 mM (i.e., low to high concentration). The different test groups are the same as those described in FIGS. 12A-12C. The stem-like cells were identified with cell surface markers CD45RO⁻CCR7⁺CD45RA⁺CD62L⁺CD27⁺CD28⁺TCF7+. Each of FIGS. 14A, 14B, and 14C provides results for biological replicates of T cells isolated from three independent donors.

FIGS. 15A, 15B, 15C, 15D, 15E, 15F, 15G, 15H, and 15I show IFN-γ (FIGS. 15A, 15B, and 15C), IL-2 (FIGS. 15D, 15E, and 15F), and TNF-α (FIGS. 15G, 15H, and 15I) production by anti-ROR1 CAR T cells after primary antigen stimulation at an effector to target (E:T) ratio of 1:1. As further described in Example 13, the T cells (isolated from three separate donors) were transduced and cultured in either T_{CM} or MRM comprising potassium ion at a concentration ranging from 40-80 mM (i.e., low to high concentration). The T cells were transduced with the following: (1) T cells control CD19t-R12 CAR (i.e., R12 CAR without c-Jun) (closed circle); or (2) c-Jun-R12 CAR (i.e., R12 CAR with c-Jun) (closed square).

FIGS. 16A, 16B, 16C, 16D, 16E, 16F, 16G, 16H, and 16I show IFN-γ (FIGS. 16A, 16B, and 16C), IL-2 (FIGS. 16D, 16E, and 16F), and TNF-α (FIGS. 16G, 16H, and 16I) production by anti-ROR1 CAR T cells after primary antigen stimulation at an effector to target (E:T) ratio of 1:4. As further described in Example 13, the T cells (isolated from three independent donors) were transduced and cultured in either TCM or MRM comprising potassium ion at a concentration ranging from 40-80 mM (i.e., low to high concentration). The T cells were transduced with the following: (1) T cells control CD19t-R12 CAR (i.e., R12 CAR without c-Jun) (closed circle); or (2) c-Jun-R12 CAR (i.e., R12 CAR with c-Jun) (closed square).

FIGS. 17A, 17B, 17C, and 17D show the ability of the anti-ROR1 CAR CD8+ T cells (transduced and cultured either in TCM or MRM comprising varying concentration of potassium ion) to kill target tumor cells after multiple rounds of antigen stimulation. As further described in Example 13, the transduced T cells were stimulated with the antigen at an effector:target (E:T) ratio of 1:1 (FIGS. 17A and 17B) or 1:4 (FIGS. 17C and 17D). FIGS. 17A and 17C provide the results for CD8+ T cells transduced with the control CD19t-R12 CAR (i.e., R12 CAR without c-Jun) and cultured in TCM or MRM comprising potassium ion at a concentration ranging from 40-80 mM (i.e., low to high concentration). FIGS. 17B and 17D provide the results for CD8+ T cells transduced with the c-Jun-R12-CAR (i.e., R12 CAR with c-Jun) and cultured in TCM or MRM comprising potassium ion at a concentration ranging from 40-80 mM (i.e., low to high concentration). The tumor viability percentage was calculated using the area under the curve (AUC) from IncuCyte killing curves (the lower the bar, the higher the cytotoxicity). In each of FIGS. 17A-17D, tumor only cells and non-transduced (“mock”) T cells were used as controls.

FIGS. 18A, 18B, 18C, 18D, and 18E provide transcriptome profiles of anti-ROR1 CAR T cells (with or without c-Jun overexpression) after adoptive transfer into tumor-bearing mice. As further described in Example 12, tumor bearing mice were treated with either c-Jun ROR1 CAR T cells cultured in MRM (c-Jun R12 CAR MRM) or control ROR1 CAR T cells cultured in MRM (control R12 CAR MRM). And, then the adoptively transferred transduced T cells were isolated from the tumors and single cell RNA-seq analysis was performed. FIG. 18A provides a UMAP of CD8⁺ T cells from all samples from both treatment groups. FIG. 18B shows the proportions of CD8⁺ T cells that are enriched for T cell terminal exhaustion genes. FIG. 18C shows the proportions of CD8⁺ T cells that are enriched for T cell progenitor exhaustion genes. FIG. 18D shows the proportions of CD8⁺ T cells that are enriched for stem-like genes. FIG. 18E shows the proportions of CD8+ T cells that are enriched for T cell activation related genes.

### DETAILED DESCRIPTION OF THE DISCLOSURE

The efficacy of cellular immunotherapy is dependent on a number of factors including the persistence, multipotency, and asymmetric cell division of the cell product that is infused into the patient. The media used in culturing and/or engineering of the cells used for cell therapy can profoundly affect the metabolic, epigenetic, and phenotypic attributes of these cells thereby affecting their therapeutic potential.

The present disclosure is directed to methods of culturing cells, cells prepared by the methods, and/or compositions or kits for the cell culturing methods. In some aspects, the disclosure provides methods of generating a population of immune cells, e.g., T cells or NK cells (e.g., modified to express an increased level of a c-Jun protein), for adoptive cell therapy (ACT), wherein the immune cells, e.g., T cells or NK cells, have a less differentiated state and retain the ability to proliferate. In some aspects, the immune cells, e.g., T cells or NK cells (e.g., modified to express an increased level of a c-Jun protein), have a less differentiated state and maintain the ability to target and kill tumor cells. In some aspects, the immune cells, e.g., T cells or NK cells (e.g., modified to express an increased level of a c-Jun protein), have a less differentiated state, retain the ability to proliferate, and maintain the ability to target and kill tumor cells. In some aspects, immune cells, e.g., T cells or NK cells (e.g., modified to express an increased level of a c-Jun protein), cultured according to the methods disclosed herein, have increased efficacy in ACT, as compared to cells cultured according to conventional methods, e.g., in a medium having less than 5 mM potassium ion. In some aspects, immune cells, e.g., T cells or NK cells (e.g., modified to express an increased level of a c-Jun protein), cultured according to the methods disclosed herein, have increased persistence upon administration to a subject in ACT, as compared to immune cells cultured according to conventional methods, e.g., in a medium having less than 5 mM potassium ion. Such increased persistence refers to the ability of the immune cell, e.g., T cells or NK cells (e.g., modified to express an increased level of a c-Jun protein), to infiltrate and function in the tumor microenvironment, ability to resist or delay the onset of exhaustion, and the persistence of stemness to ensure continued expansion and durability of response. In some aspects, immune cells, e.g., T cells or NK cells (e.g., modified to express an increased level of a c-Jun protein), cultured according to the methods disclosed herein, are stem-like cells. Such cells are capable of self-renewal, proliferation and differentiation. In some aspects, immune cells, e.g., T cells or NK cells (e.g., modified to express an increased level of a c-Jun protein), cultured according to the methods disclosed herein, are stem-like cells which also express effector-like markers. In some aspects, immune cells, e.g., T cells or NK cells, cultured according to the methods disclosed herein, are stem-like cells which also maintain the ability to target and kill tumor cells.

The cell culturing methods of the present disclosure are capable of increasing multipotency and/or pluripotency of the cultured cells or increasing transduction efficiency when the cells are being transduced with a vector. In some aspects, the culturing methods are capable of reducing and/or preventing cell exhaustion when the cells are cultured and/or the cells are used in therapy in vivo. In some aspects, the culturing methods are also capable of increasing in vivo viability, in vivo persistence, in vivo effector function, or any combination thereof. In some aspects, the culturing methods disclosed herein are capable of enriching oligoclonal or polyclonal tumor reactive stem-like T-cells and/or CD8⁺ TILs. In some aspects, the culturing methods disclosed herein are capable of preserving clonal diversity of the TILs derived from cancer patients.

In some aspects, the disclosure is directed to methods of culturing cells, e.g., immune cells, e.g., T cells or NK cells (e.g., modified to express an increased level of a c-Jun protein), comprising placing the cells in metabolic reprogramming medium comprising potassium at a concentration of at least about 5 mM (e.g., higher than 5 mM), wherein the medium is not hypertonic, e.g., hypotonic or isotonic. Some aspects of the present disclosure are directed to methods of culturing cells, e.g., immune cells, e.g., T cells or NK cells (e.g., modified to express an increased level of a c-Jun protein), comprising placing the cells in a medium comprising potassium at a concentration higher than 40 mM, e.g., about 40 mM-80 mM, e.g., about 50 mM-80 mM. In some aspects, the immune cells comprise T cells, tumor-infiltrating lymphocytes (TILs), natural killer (NK) cells, regulatory T (T_{reg}) cells, or any combination thereof.

Some aspects of the present disclosure are directed to a method of increasing the yield of immune cells, e.g., T cells or NK cells (e.g., modified to express an increased level of a c-Jun protein), during ex vivo or in vitro culturing while increasing stemness of the immune cells comprising culturing the cells in metabolic reprogramming medium comprising potassium ion at a concentration between 40 mM and 80 mM and NaCl at a concentration between 30 mM and 100 mM, wherein the total concentration of potassium ion and NaCl is between 110 and 140 mM. Some aspects of the present disclosure are directed to a method of preparing a population of immune cells, e.g., T cells or NK cells (e.g., modified to express an increased level of a c-Jun protein), for immunotherapy comprising culturing the cells in a medium comprising potassium ion at a concentration between 40 mM and 80 mM and NaCl at a concentration between 30 mM and 100 mM, wherein the total concentration of potassium ion and NaCl is between 110 and 140 mM. Some aspects of the present disclosure are directed to a method of increasing stemness of immune cells, e.g., T cells or NK cells (e.g., modified to express an increased level of a c-Jun protein), during ex vivo or in vitro culturing comprising culturing immune cells, e.g., T cells or NK cells (e.g., modified to express an increased level of a c-Jun protein), in a medium comprising potassium ion at a concentration between 40 mM and 80 mM and NaCl at a concentration between 30 mM and 100 mM, wherein the total concentration of potassium ion and NaCl is between 110 and 140 mM. In some aspects the immune cells are T cells.

In some aspects, the medium is hypotonic. In some aspects, the medium is isotonic. In certain aspects, the medium further comprises interleukin (IL)-2, IL-21, IL-7, IL-15, or any combination thereof. In some aspects, the medium comprises IL-2, IL-7 and IL-15. In some aspects, the medium comprises IL-2 and IL-21. In some aspects, the medium further comprises sodium ion, calcium ion, glucose, or any combination thereof.

As described herein, in some aspects, modifying immune cells (e.g., T cells or NK cells) to overexpress c-Jun (e.g., with an exogenous nucleotide sequence encoding c-Jun and/or a transcriptional activator that is capable of increasing the expression of the endogenous c-Jun polypeptide) in a medium comprising potassium ion at a concentration higher than 5 mM can further improve one or more properties of the immune cells compared to a reference method, in which: (i) the immune cells are modified but not cultured in the medium comprising potassium at a concentration higher than 5 mM; (ii) the immune cells are not modified but cultured in a medium comprising potassium at a concentration higher than 5 mM; or (iii) both (i) and (ii). Additional aspects of such methods are provided throughout the present disclosure.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

Throughout the disclosure, the term “a” or “an” entity refers to one or more of that entity; for example, “a chimeric polypeptide,” is understood to represent one or more chimeric polypeptides. As such, the terms “a” (or “an”), “one or more,” and “at least one” can be used interchangeably herein. In addition, “or” is used to mean an open list of the components in the list. For example, “wherein X comprises A or B” means X comprises A, X comprises B, X comprises A and B, or X comprises A or B and any other components.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range, unless otherwise explicitly stated.

Abbreviations used herein are defined throughout the present disclosure. Various aspects of the disclosure are described in further detail in the following subsections.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% (e.g., a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value)). For example, “about 55 mM,” as used herein, includes 49.5 mM to 60.5 mM. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As used herein, the term “approximately,” as applied to one or more values of interest, refers to a value that is similar to a stated reference value. In some aspects, the term “approximately”, like the term “about”, refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As described herein, any concentration range, percentage range, ratio range, or integer range is to be understood to include the value of any integer within the recited range and, when appropriate, fractions thereof (such as one tenth and one hundredth of an integer), unless otherwise indicated.

The term “control media,” “conventional culture media,” or “reference culture media” as used herein refers to any media in comparison to a metabolic reprogramming media (MRM) disclosed herein. Control media can comprise the same components as the metabolic reprogramming media except certain ion concentrations, e.g., potassium ion. In some aspects, metabolic reprogramming media described herein are prepared from control media by adjusting one or more ion concentrations, e.g., potassium ion concentration, as described herein. In some aspects, control media comprise basal media, e.g., CTS™ OPTMIZER™. In some aspects, control media thus comprises one or more additional components, including, but not limited to, amino acids, glucose, glutamine, T cell stimulators, antibodies, substituents, etc. that are also added to the metabolic reprogramming media, but control media have certain ion concentrations different from the metabolic reprogramming media. Unless indicated otherwise, the terms “media” and “medium” can be used interchangeably.

The term “culturing” as used herein refers to the controlled growth of cells ex vivo and/or in vitro. As used herein, “culturing” includes the growth of cells, e.g., immune cells, e.g., one or more engineered immune cell disclosed herein, during cell expansion, or cell engineering (e.g., transduction with a construct for expressing a CAR, a TCR, or a TCRm). In some aspects, the cultured cells are obtained from a subject, e.g., a human subject/patient. In some aspects, the cultured cells comprise immune cells obtained from a human subject/patient. In some aspects, the cultured cells comprise one or more engineered immune cell disclosed herein. In some aspects, the cultured cells comprise T cells or NK cells obtained from a human subject/patient. In some aspects, the T cells and/or NK cells are purified prior to the culture. In some aspects, the T cells and/or NK cells are tumor-infiltrating T cells and/or NK cells. In some aspects, the cultured cells comprise one or more engineered immune cell disclosed herein.

The term “expand” or “expansion,” as used herein in reference to immune cell culture refers to the process of stimulating or activating the cells and culturing the cells. The expansion process can lead to an increase in the proportion or the total number of desired cells, e.g., an increase in the proportion or total number of less differentiated immune cells, in a population of cultured cells, after the cells are stimulated or activated and cultured. Expansion does not require that all cell types in a population of cultured cells are increased in number. Rather, in some aspects, only a subset of cells in a population of cultured cells are increased in number during expansion, while the number of other cell types may not change or may decrease.

As used herein, the term “yield” refers to the total number of cells following a culture method or a portion thereof. In some aspects, the term “yield” refers to a particular population of cells, e.g., stem-like T cells in a population of T cells. The yield can be determined using any methods, including, but not limited to, estimating the yield based on a representative sample.

As used herein, the term “metabolic reprogramming media,” “metabolic reprogramming medium,” or “MRM,” refers to a medium of the present disclosure, wherein the medium has an increased potassium concentration. In some aspects, the metabolic reprogramming media comprises potassium ion at a concentration higher than 5 mM. In some aspects, the metabolic reprogramming media comprises potassium ion at a concentration higher than 40 mM. In some aspects, the MRM comprises potassium ion at a concentration between about 40 mM and about 80 mM. In some aspects, the metabolic reprogramming media comprises a concentration of potassium ion of at least about 10 mM, at least about 15 mM, at least about 20 mM, at least about 25 mM, at least about 30 mM, at least about 35 mM, at least about 40 mM, at least about 45 mM, at least about 50 mM, at least about 55 mM, at least about 60 mM, at least about 65 mM, at least about 70 mM, at least about 75 mM, at least about 80 mM, at least about 85 mM, at least about 90 mM, at least about 95 mM, or at least about 100 mM. In some aspects, the MRM comprises potassium ion at a concentration of about 40 mM. In some aspects, the MRM comprises potassium ion at a concentration of about 50 mM. In some aspects, the MRM comprises potassium ion at a concentration of about 60 mM. In some aspects, the MRM comprises potassium ion at a concentration of about 70 mM. In some aspects, the MRM comprises potassium ion at a concentration of about 80 mM. In some aspects, the metabolic reprogramming media comprises about 40 mM to about 80 mM NaCl, about 40 mM to about 90 mM KCl, about 0.5 mM to about 2.8 mM calcium, and about 10 mM to about 24 mM glucose. In some aspects, the MRM comprises about 40 mM to about 80 mM NaCl, about 40 mM to about 80 mM potassium ion, about 0.5 mM to about 2.8 mM calcium, and about 10 mM to about 24 mM glucose. In some aspects, the MRM comprises about 55 mM to about 90 mM NaCl and about 40 mM to about 80 mM potassium ion.

In some aspects, the metabolic reprogramming media further comprises an osmolality of about 250 to about 300 mOsmol.

As used herein, the term “higher than” means greater than but not equal to. For example, “higher than 5 mM” means any amount that is more than 5 mM, but which does not include 5 mM.

As used herein, the term “tonicity” refers to the calculated effective osmotic pressure gradient across a cell membrane, represented by the sum of the concentration of potassium ion and the concentration of sodium chloride (NaCl), multiplied by two. Tonicity can be expressed in terms of the osmolality (mOsm/kg) or osmolarity (mOsm/L) of the solution, e.g., the media. Osmolality and osmolarity are measurements of the solute osmotic concentration of a solvent per mass (osmolality) and per volume (osmolarity). As used herein, an isotonic medium has a tonicity of about 280 mOsm/L (e.g., ([K+]+[NaCl])×2=280).

As used herein, a hypotonic solution has a tonicity of less than 280 mOsm/L (e.g., ([K+]+[NaCl])×2<280). In some aspects, a hypotonic medium has a tonicity from at least about 210 mOsm/L to less than about 280 mOsm/L. In some aspects, a hypotonic medium has a tonicity from at least about 220 mOsm/L to less than about 280 mOsm/L. In some aspects, a hypotonic medium has a tonicity from at least about 230 mOsm/L to less than about 280 mOsm/L. In some aspects, a hypotonic medium has a tonicity from at least about 240 mOsm/L to less than about 280 mOsm/L. In some aspects, a hypotonic medium described herein has a tonicity of about 250 mOsm/L.

As used herein, a hypertonic solution has a tonicity of greater than 300 mOsm/L (e.g., ([K+]+[NaCl])×2>300). In some aspects, a hypertonic medium described herein has a tonicity of about 320 mOsm/L. In some aspects, the tonicity of the solution, e.g., medium is adjusted by increasing or decreasing the concentration of potassium ions and NaCl. In some aspects, the tonicity of a medium can be maintained by offsetting the increase of one solute with a decrease in a second solute. For example, increasing the concentration of potassium ion in a medium without changing the concentration of sodium ions can increase the tonicity of the medium. However, if the concentration of potassium ions is increased and the concentration of sodium ions is decreased, the tonicity of the original medium can be maintained.

As used herein, the terms “potassium,” “potassium ion,” “potassium cation,” and “K+” are used interchangeably to refer to elemental potassium. Elemental potassium exists in solution as a positive ion. However, it would be readily apparent to a person of ordinary skill in the art that standard means of preparing a solution comprising potassium ion include diluting a potassium containing salt (e.g., KCl) into a solution. As such, a solution, e.g., a medium, comprising a molar (M) concentration of potassium ion, can be described as comprising an equal molar (M) concentration of a salt comprising potassium.

As used herein, the terms “sodium ion” and “sodium cation” are used interchangeably to refer to elemental sodium. Elemental sodium exists in solution as a monovalent cation. However, it would be readily apparent to a person of ordinary skill in the art that standard means of preparing a solution comprising sodium ion include diluting a sodium-containing salt (e.g., NaCl) into a solution. As such, a solution, e.g., a medium, comprising a molar (M) concentration of sodium ion, can be described as comprising an equal molar (M) concentration of a salt comprising sodium.

As used herein, the terms “calcium ion” and “calcium cation” are used interchangeably to refer to elemental calcium. Elemental calcium exists in solution as a divalent cation. However, it would be readily apparent to a person of ordinary skill in the art that standard means of preparing a solution comprising calcium ion include diluting a calcium-containing salt (e.g., CaCl₂)) into a solution. As such, a solution, e.g., a medium, comprising a molar (M) concentration of calcium ion, can be described as comprising an equal molar (M) concentration of a salt comprising calcium.

As used herein, the term “immune cell” refers to a cell of the immune system. In some aspects, the immune cell is selected from a T lymphocyte (“T cell”), B lymphocyte (“B cell”), natural killer (NK) cell, macrophage, eosinophil, mast cell, dendritic cell or neutrophil. As used herein, a “population” of cells refers to a collection of more than one cell, e.g., a plurality of cells. In some aspects, the population of cells comprises more than one immune cell, e.g., a plurality of immune cells. In some aspects, the population of cells is comprises a heterogeneous mixture of cells, comprising multiple types of cells, e.g., a heterogeneous mixture of immune cells and non-immune cells. In some aspects, the population of cells comprises a plurality of T cells.

As used herein, the term “reference immune cell” (or “reference cell”) refers to a cell which has not been modified and/or cultured using the methods provided herein. For example, in some aspects, a reference cell comprises a cell (e.g., corresponding immune cell) that has not been modified as described herein (e.g., with any of the c-Jun nucleotide sequences and/or transcriptional activators provided herein). In some aspects, a reference cell comprises such a cell (which has not been modified as described herein) cultured in a medium of the present disclosure (e.g., comprising potassium ion at a concentration higher than 5 mM). In some aspects, a reference cell comprises such a cell (which has not been modified as described herein) cultured in a medium that does not comprise potassium ion at a concentration higher than 5 mM (i.e., reference medium). In some aspects, a reference cell comprises a cell which has been modified as described herein (e.g., with any of the c-Jun nucleotide sequences and/or transcriptional activators provided herein) but cultured in the reference medium. Accordingly, unless indicated otherwise, reference cell can comprise any of the following: (1) a cell (e.g., corresponding immune cell) which has not been modified as described herein; (2) a cell (e.g., corresponding immune cell) which has neither been modified as described herein nor cultured in a medium of the present disclosure; (3) a cell (e.g., corresponding immune cell) which has not been modified as described herein but cultured in a medium of the present disclosure; (4) a cell (e.g., corresponding immune cell) which has been modified as described herein but cultured in a reference medium; or (5) any combination of (1) to (4). Based on at least the present disclosure, it will be apparent to those skilled in the arts the scope of the term “reference cell” when used herein.

As used herein, the terms “T cell” and “T lymphocyte” are interchangeable and refer to any lymphocytes produced or processed by the thymus gland. Non-limiting classes of T cells include effector T cells and T helper (Th) cells (such as CD4+ or CD8+ T cells). In some aspects, the T cell is a Th1 cell. In some aspects, the T cell is a Th2 cell. In some aspects, the T cell is a Tc17 cell. In some aspects, the T cell is a Th17 cell. In some aspects, the T cell is a T_{reg} cell. In some aspects, the T cell is a tumor-infiltrating cell (TIL).

As used herein, the term “memory” T cells refers to T cells that have previously encountered and responded to their cognate antigen (e.g., in vivo, in vitro, or ex vivo) or which have been stimulated, e.g., with an anti-CD3 antibody (e.g., in vitro or ex vivo). Immune cells having a “memory-like” phenotype upon secondary exposure, such memory T cells can reproduce to mount a faster and stronger immune response than during the primary exposure. In some aspects, memory T cells comprise central memory T cells (T_{CM} cells), effector memory T cells (T_{EM} cells), tissue resident memory T cells (T_{RM} cells), stem cell-like memory T cells (T_{SCM} cells), or any combination thereof.

As used herein, the term “stem cell-like memory T cells,” “T memory stem cells,” or “T_{SCM} cells” refers to memory T cells that express CD95, CD45RA, CCR7, and CD62L and are endowed with the stem cell-like ability to self-renew and the multipotent capacity to reconstitute the entire spectrum of memory and effector T cell subsets.

As used herein, the term “central memory T cells” or “T_{CM} cells” refers to memory T cells that express CD45RO, CCR7, and CD62L. Central memory T cells are generally found within the lymph nodes and in peripheral circulation.

As used herein, the term “effector memory T cells” or “T_{EM} cells” refers to memory T cells that express CD45RO but lack expression of CCR7 and CD62L. Because effector memory T cells lack lymph node-homing receptors (e.g., CCR7 and CD62L), these cells are typically found in peripheral circulation and in non-lymphoid tissues.

As used herein, the term “tissue resident memory T cells” or “T_{RM} cells” refers to memory T cells that do not circulate and remain resident in peripheral tissues, such as skin, lung, and gastrointestinal tract. In some aspects, tissue resident memory T cells are also effector memory T cells.

As used herein, the term “naïve T cells” or “T_{N} cells” refers to T cells that express CD45RA, CCR7, and CD62L, but which do not express CD95. T_{N} cells represent the most undifferentiated cell in the T cell lineage. The interaction between a T_{N} cell and an antigen presenting cell (APC) induces differentiation of the T_{N} cell towards an activated TEFF cell and an immune response.

As used herein, the term “stemness,” “stem cell-like,” “stem-like,” or “less-differentiated” refers to an immune cell (e.g., a T cell, an NK cell, or a TIL), that expresses markers consistent with a more naïve phenotype. For example, a less differentiated T cell can express one or more marker characteristic of a T_{N} or a T_{SCM} cell. In some aspects, a “less-differentiated” or “stem-like” T cell expresses CD45RA, CCR7, and CD62L. In some aspects, a “less-differentiated” or “stem-like” T cell expresses CD45RA, CCR7, CD62L, and TCF7. In some aspects, a “less-differentiated” or “stem-like” T cell does not express CD45RO or is CD45RO^{low}. In some aspects, the methods disclosed herein promote immune cells (e.g., T cells and/or NK cells) having a less-differentiated phenotype. Without being bound by any particular mechanism, in some aspects, the methods disclosed herein block, inhibit, or limit differentiation of less-differentiated immune cells (e.g., T cells and/or NK cells), resulting in an increased number of stem-like cells in culture. For example, it is generally thought that to effectively control tumors, adoptive transfer of less-differentiated immune cells, e.g., T cells and/or NK cells, with a stem cell-like memory or central memory phenotype are preferred. See Gattinoni, L., et al., J. Clin. Invest. 115:1616-1626 (2005), Gattinoni, L., et al. Nat Med 15(7):808-814 (2009), Lynn, R. C., et al., Nature 576(7786): 293-300 (2019); Gattinoni, L., et al. Nat Rev 12:671-684 (2012), Klebanoff, C., et al., J. Immunother 35(9):651-670 (2012) and Gattinoni, L., et al., Nat Med 17(10): 1290-1297 (2011).

Stemness is characterized by the capacity to self-renew, the multipotency, and the persistence of proliferative potential. In some aspects, stemness is characterized by a particular gene signature, e.g., a combined pattern of expression across a multitude of genes. In some aspects, the stem-like cells can be identified by a transcriptome analysis, e.g., using sternness gene signatures disclosed herein. In some aspects, the gene signature comprises one or more genes selected from ACTN1, DSC1, TSHZ2, MYB, LEF1, TIMD4, MAL, KRT73, SESN3, CDCA7L, LOC283174, TCF7, SLC16A10, LASS6, UBE2E2, IL7R, GCNT4, TAF4B, SULT1B1, SELP, KRT72, STXBP1, TCEA3, FCGBP, CXCR5, GPA33, NELL2, APBA2, SELL, VIPR1, FAM153B, PPFIBP2, FCER1G, GJB6, OCM2, GCET2, LRRN1, IL6ST, LRRC16A, IGSF9B, EFHA2, LOC129293, APP, PKIA, ZC3H12D, CHMP7, KIAA0748, SLC22A17, FLJ13197, NRCAM, C5orf13, GIPC3, WNT7A, FAM117B, BEND5, LGMN, FAM63A, FAM153B, ARHGEF11, RBM11, RIC3, LDLRAP1, PELI1, PTK2, KCTD12, LMO7, CEP68, SDK2, MCOLN3, ZNF238, EDAR, FAM153C, FAAH2, BCL9, C17orf48, MAP1D, ZSWIM1, SORBS3, IL4R, SERPINF1, C16orf45, SPTBN1, KCNQ1, LDHB, BZW2, NBEA, GAL3ST4, CRTC3, MAP3K1, HLA-DOA, RAB43, SGTB, CNN3, CWH43, KLHL3, PIM2, RGMB, C16orf74, AEBP1, SNORD 115-11, SNORD 115-11, GRAP, and any combination thereof (see, e.g., Gattinoni et al., Nature Medicine 17(10):1290-97 (2011)). In some aspects, the gene signature comprises one or more gene selected from NOG, TIMD4, MYB, UBE2E2, FCER1G, HAVCR1, FCGBP, PPFIBP2, TPST1, ACTN1, IGF1R, KRT72, SLC16A10, GJB6, LRRN1, PRAGMIN, GIPC3, FLNB, ARRB1, SLC7A8, NUCB2, LRRC7, MYO15B, MAL, AEBP1, SDK2, BZW2, GAL3ST4, PITPNM2, ZNF496, FAM117B, C16orf74, TDRD6, TSPAN32, C18orf22, C3orf44, LOC129293, ZC3H12D, MLXIP, C7orf10, STXBP1, KCNQ1, FLJ13197, LDLRAP1, RAB43, RIN3, SLC22A17, AGBL3, TCEA3, NCRNA00185, FAM153B, FAM153C, VIPR1, MMP19, HBS1L, EEF2K, SNORA5C, UBASH3A, FLJ43390, RP6-213H19.1, INPP5A, PIM2, TNFRSF10D, SNRK, LOC100128288, PIGV, LOC100129858, SPTBN1, PROS1, MMP28, HES1, CACHD1, NSUN5C, LEF1, TTTY14, SNORA54, HSF2, C16orf67, NSUN5B, KIAA1257, NRG2, CAD, TARBP1, STRADB, MT1F, TMEM41B, PDHX, KDM6B, LOC100288322, UXS1, LGMN, NANOS2, PYGB, RASGRP2, C14orf80, XPO6, SLC24A6, FAM113A, MRM1, FBXW8, NDUFS2, KCTD12, and any combination thereof (see, e.g., Gattinoni, L., et al., Nat Med 17(10): 1290-1297 (2011)). In some aspects, the gene signature comprises one or more gene selected from SELL, CCR7, S1PR1, KLF3, TCF7, GPR183, SC5D, FAAH2, LTB, SESN3, MAL, TSHZ2, LEF1, AP3M2, SLC2A3, ICAM2, PLAC8, SCML1, IL7R, ABLIM1, RASGRP2, TRABD2A, SATB1, ALG13, ARID5A, BACH2, PABPC1, GPCPD1, NELL2, TAF4B, FCMR, ARRDC2, C1orf162, FAM177A1, ANKRD12, TXK, SORL1, AQP3, ADTRP, FXYD7, CD28, P2RY8, CRYBG1, TNFSF8, BEX2, PGAP1, PTGER4, MAML2, BEX3, PCSK1N, INPP4B, AC119396.1, CXCR5, LINC00402, CCR4, IL6R, ZBTB10, ITGA6, ARMH1, RILPL2, FOXP1, TESPA1, YPEL5, LPAR6, CMSS1, RIPOR2, ZNF331, EMP3, GIMAP7, WDR74, RIC3, CYSLTR1, ITGB1, CD5, SAMHD1, SERINC5, and any combination thereof (see e.g., Caushi et al., Nature 596: 126-132 (2021)).

As used herein, the term “effector-like” or “effector cell-like” refers to tumor cell killing capacity and cytokine polyfunctionality, e.g., ability of a cell to produce inflammatory cytokines and/or cytotoxic molecules. In some aspects, an effector-like cell is characterized by specific markers expressed by the cell. In some aspects, those effector-like markers comprise one or more of pSTAT5+, STAT5+, pSTAT3+, and STAT3+. In some aspects, the effector-like marker comprises a STAT target selected from the group consisting of AKT1, AKT2, AKT3, BCL2L1, CBL, CBLB, CBLC, CCND1, CCND2, CCND3, CISH, CLCF1, CNTF, CNTFR, CREBBP, CRLF2, CSF2, CSF2RA, CSF2RB, CSF3, CSF3R, CSH1, CTF1, EP300, EPO, EPOR, GH1, GH2, GHR, GRB2, IFNA1, IFNA10, IFNA13, IFNA14, IFNA16, IFNA17, IFNA2, IFNA21, IFNA4, IFNA5, IFNA6, IFNA7, IFNA8, IFNAR1, IFNAR2, IFNB1, IFNE, IFNG, IFNGR1, IFNGR2, IFNK, IFNL1, IFNL2, IFNL3, IFNLR1, IFNW1, IL10, IL10RA, IL10RB, IL11, IL1IRA, IL12A, IL12B, IL12RB1, IL12RB2, IL13, IL13RA1, IL13RA2, IL15, IL15RA, IL19, IL2, IL20, IL20RA, IL20RB, IL21, IL21R, IL22, IL22RA1, IL22RA2, IL23A, IL23R, IL24, IL26, IL2RA, IL2RB, IL2RG, IL3, IL3RA, IL4, IL4R, IL5, IL5RA, IL6, IL6R, IL6ST, IL7, IL7R, IL9, IL9R, IRF9, JAK1, JAK2, JAK3, LEP, LEPR, LIF, LIFR, MPL, MYC, OSM, OSMR, PIAS1, PIAS2, PIAS3, PIAS4, PIK3CA, PIK3CB, PIK3CD, PIK3CG, PIK3R1, PIK3R2, PIK3R3, PIK3R5, PIM1, PRL, PRLR, PTPN11, PTPN6, SOCS1, SOCS2, SOCS3, SOCS4, SOCS5, SOCS7, SOS1, SOS2, SPRED1, SPRED2, SPRY1, SPRY2, SPRY3, SPRY4, STAM, STAM2, STAT1, STAT2, STAT3, STAT4, STAT5A, STAT5B, STAT6, TPO, TSLP, TYK2, and any combination thereof. In some aspects, the effector-like cells are characterized by a transcriptome analysis. In some aspects, the effector-like marker comprises a marker disclosed in Kaech et al., Cell 111:837-51 (2002); Tripathi et al., J. Immunology 185:2116-24 (2010); and/or Johnnidis et al., Science Immunology 6:eabe3702 (Jan. 15, 2021), each of which is incorporated by reference herein in its entirety.

In some aspects, the effector-like cells are characterized using an effector-associated gene set described in Gattinoni, L., et al., Nat Med 17(10):1290-97 (2011). In some aspects, the gene signature for effector-like cells comprises one or more genes selected from MTCH2, RAB6C, KIAA0195, SETD2, C2orf24, NRD1, GNA13, COPA, SELT, TNIP1, CBFA2T2, LRP10, PRKCI, BRE, ANKS1A, PNPLA6, ARL6IP1, WDFY1, MAPK1, GPR153, SHKBP1, MAP1LC3B2, PIP4K2A, HCN3, GTPBP1, TLN1, C4orf34, KIF3B, TCIRG1, PPP3CA, ATG4D, TYMP, TRAF6, C17orf76, WIPF1, FAM108A1, MYL6, NRM, SPCS2, GGT3P, GALK1, CLIP4, ARL4C, YWHAQ, LPCAT4, ATG2A, IDS, TBC1D5, DMPK, ST6GALNAC6, REEP5, ABHD6, KIAA0247, EMB, TSEN54, SPIRE2, PIWIL4, ZSCAN22, ICAM1, CHD9, LPIN2, SETD8, ZC3H12A, ULBP3, IL15RA, HLA-DQB2, LCP1, CUP, RUNX3, TMEM43, REEP4, MEF2D, ABL1, TMEM39A, PCBP4, PLCD1, CHST12, RASGRP1, C1orf58, C11orf63, C6orf129, FHOD1, DKFZp434F142, PIK3CG, ITPR3, BTG3, C4orf50, CNNM3, IFI16, AK1, CDK2AP1, REL, BCL2L1, MVD, TTC39C, PLEKHA2, FKBP11, EML4, FANCA, CDCA4, FUCA2, MFSD10, TBCD, CAPN2, IQGAP1, CHST11, PIK3R1, MYOSA, KIR2DL3, DLG3, MXD4, RALGDS, S1PR5, WSB2, CCR3, TIPARP, SP140, CD151, SOX13, KRTAP5-2, NF1, PEA15, PARP8, RNF166, UEVLD, LIMK1, CACNB1, TMX4, SLC6A6, LBA1, SV2A, LLGL2, IRF1, PPP2R5C, CD99, RAPGEF1, PPP4R1, OSBPL7, FOXP4, SLA2, TBC1D2B, ST7, JAZF1, GGA2, PI4K2A, CD68, LPGAT1, STX11, ZAK, FAM160B1, RORA, C8orf80, APOBEC3F, TGFBI, DNAJC1, GPR114, LRP8, CD69, CMI, NAT13, TGFB1, FLJ00049, ANTXR2, NR4A3, IL12RB1, NTNG2, RDX, MLLT4, GPRIN3, ADCY9, CD300A, SCD5, ABI3, PTPN22, LGALS1, SYTL3, BMPR1A, TBK1, PMAIP1, RASGEFlA, GCNT1, GABARAPLI, STOM, CALHM2, ABCA2, PPP1R16B, SYNE2, PAM, C12orf75, CLCF1, MXRA7, APOBEC3C, CLSTN3, ACOT9, HIP1, LAG3, TNFAIP3, DCBLD1, KLF6, CACNB3, RNF19A, RAB27A, FADS3, DLG5, APOBEC3D, TNFRSF1B, ACTN4, TBKBP1, ATXN1, ARAP2, ARHGEF12, FAM53B, MAN1A1, FAM38A, PLXNC1, GRLF1, SRGN, HLA-DRB5, B4GALT5, WIPI1, PTPRJ, SLFN11, DUSP2, ANXA5, AHNAK, NEO1, CLIC1, EIF2C4, MAP3K5, IL2RB, PLEKHG1, MYO6, GTDC1, EDARADD, GALM, TARP, ADAM8, MSC, HNRPLL, SYT11, ATP2B4, NHSL2, MATK, ARHGAP18, SLFN12L, SPATS2L, RAB27B, PIK3R3, TP53INP1, MBOAT1, GYG1, KATNAL1, FAM46C, ZC3HAV1L, ANXA2P2, CTNNA1, NPC1, C3AR1, CRIM1, SH2D2A, ERN1, YPEL1, TBX21, SLC1A4, FASLG, PHACTR2, GALNT3, ADRB2, PIK3AP1, TLR3, PLEKHA5, DUSP10, GNAO1, PTGDR, FRMD4B, ANXA2, EOMES, CADM1, MAF, TPRG1, NBEAL2, PPP2R2B, PELO, SLC4A4, KLRF1, FOSL2, RGS2, TGFBR3, PRF1, MYO1F, GAB3, C17orf66, MICAL2, CYTH3, TOX, HLA-DRA, SYNE1, WEE1, PYHIN1, F2R, PLD1, THBS1, CD58, FAS, NETO2, CXCR6, ST6GALNAC2, DUSP4, AUTS2, C1orf2l, KLRG1, TNIP3, GZMA, PRR5L, PRDM1, ST8SIA6, PLXND1, PTPRM, GFPT2, MYBL1, SLAMF7, FLJ16686, GNLY, ZEB2, CST7, IL18RAP, CCL5, KLRD1, KLRB1, and any combination thereof (see, e.g., Gattinoni, L., et al., Nat Med 17(10):1290-97 (2011).

As further described herein (see, e.g., Example 12), in some aspects, the characteristics of a cell (e.g., T cells and/or NK cells) can be assessed using transcriptome analysis by comparing the upregulation and/or downregulation of different sets of genes associated with T cell activation (also referred to herein as “TACT genes”), T cell progenitor exhaustion (also referred to herein as “TPE genes”), T cell terminal exhaustion (also referred to herein as “TTE genes”).

In some aspects, the terminally exhausted T cells are characterized using a TTE-associated gene set described in Oliveira et al., Nature 596: 119-125 (2021). In some aspects, the gene signature for TTE cells comprises one or more or all of the genes selected from: KRT86, RDH10, ACP5, CXCR6, HMOX1, LAYN, CLIC3, HAVCR2, AC243829.4, PRF1, SLC2A8, CHST12, GALNT2, ENTPD1, LAG3, GZMB, PDCD1, CARD16, CTLA4, SLA2, CD27, RALA, VCAM1, SYNGR2, NKG7, LSP1, CCL5, RARRES3, CD7, CTSW, MTSS1, PTMS, BATF, KIR2DL4, AKAP5, CD38, RAB27A, GZMH, IGFLR1, ATP8B4, CD63, HOPX, TNFRSF18, ADGRG1, PLPP1, CSF1, TNFSF10, SNAP47, LINC01871, MYO1E, ZBED2, AHI1, ABI3, FASLG, TYMP, ZBTB38, CTSB, PLSCR1, AFAP1L2, ITGAE, TNS3, DUSP16, CASP1, CARS, DUSP5, IFIT1, SLC1A4, GOLIM4, RSAD2, DNPH1, NBL1, ACOT9, ABHD6, OAS1, SLC27A2, ZBP1, CD200R1, OAS3, CMPK2, TNFSF4, POLR1E, CADM1, HELZ2, SYTL2, AGPAT2, UBE2F, GIMAP6, ZBTB32, RIN3, PLEKHFI, CHPF, PACSIN2, ABCB1, SPATS2L, USP18, TMEM9, KLRC1, MPST. In some aspects, progenitor exhausted T cells (TPE) are characterized using a TPE-associated gene set described in Oliveira et al., Nature 596: 119-125 (2021). In some aspects, the gene signature for TPE cells comprises one or more or all of the genes selected from: FXYD6, CAV1, GNG4, XCL1, CRTAM, CXCL13, GEM, XCL2, FXYD2, HLA-DRA, LANCL2, RASSF4, BAG3, HSPA1B, HLA-DQA1, HSPB1, FABP5, MS4A6A, SERPINHI, HLA-DPA1, HLA-DRB1, HSPA1A, RGS2, DRAIC, CD74, HSPD1, HSPA6, HSPE1, CD82, TOX, CD200, HLA-DPB1, NR4A2, VCAM1, BEX3, AIF1, DNAJA1, HSPH1, DNAJB1, HIPK2, LHFPL6, HLA-DMA, GK, TSHZ2, LPL, C16orf45, ZFAND2A, CD80, ETV1, NMB, DEDD2, CMC1, PON2, SEMA4A, ENC1, GRAMD1A, MYL6B, BCAT1, ARMH1, TIAM1, PIKFYVE, MRPL18, INPP5F, LMCD1, SESN3, CCDC6, KIAA1324, CHN1, ANKRD10, CD70, PRRG4, TNFSF4, CORO1B, DNAJB4, MAGEH1, ICAM1, GGT1, NINJ2, BLVRA, FAAH2, TOX2, SLK, CCDC141, ATF3, INPP1, FAM3C, GADD45G, APP, MAL, SIT1, DRAM1, CLECL1, MDFIC, PMCH, HLA-DMB, PHF6, AFAP1L2, BTN2A2, CCL4L2. In some aspects, activated T cells (TACT) are characterized using a TACT-associated gene set described in Oliveira et al., Nature 596: 119-125 (2021). In some aspects, the gene signature for activated T cells comprises one or more or all of the genes selected from: EGR1, HSPA6, FOS, HSPA1B, GADD45B, NR4A1, FOSB, ATF3, DNAJB1, DUSP1, JUNB, CD69, NR4A2, NFKBIA, PPP1R15A, KLF6, DNAJA1, JUN, SRSF7, SLC2A3, ZFP36L1, IER2, HSPA1A, EIF4A2, ID1, IFRD1, CCNL1, RSRP1, SERTAD1, DEDD2, KLF10, AL118516.1, KLF2, ZFAND2A, CLK1, RSRC2, IER3, BTG2, MYLIP, MAFF, CSRNP1, ID2, ZC3H12A, BAG3, SNHG12, TNF, DDIT4, SGK1, SNHG15, DNAJB4, NR4A3, NFKBID, SCML1, RASD1, ATF4, AREG, RASGEF1B, AC020916.1, DDIT3, SNHG8, CITED2, TXNIP, TOB1, PIM2, SOCS3, GADD45G, RGS16, TIPARP, NFKBIZ, CCL4, CD83, PPP1R10, CCL4L2, SESN2, CHMPIB, LEF1, CSKMT, HEXIMI, HSPA2, MRPL18, RBKS, CD55, ARRDC2, SC5D, FAM53C, ATP2B1-AS1, IFNG, MYC, TSC22D2, SERPINHI, LRIF1, ARRDC3, ILF3-DT, INTS6, ZNF10, PRMT9, ATM, SELL, AC243960.1.

As used herein, the term “basal” media refers to any starting media that is supplemented with one or more of the additional elements disclosed herein, e.g., potassium, sodium, calcium, glucose, IL-2, IL-7, IL-15, IL-21, or any combination thereof. The basal media can be any media for culturing immune cells, e.g., T cells and/or NK cells. In some aspects, the basal media comprises a balanced salt solution (e.g., PBS, DPBS, HBSS, EBSS), Dulbecco's Modified Eagle's Medium (DMEM), Click's medium, Minimal Essential Medium (MEM), Basal Medium Eagle (BME), F-10, F-12, RPMI 1640, Glasgow Minimal Essential Medium (GMEM), alpha Minimal Essential Medium (alpha MEM), Iscove's Modified Dulbecco's Medium (IMDM), M199, OPTMIZER™ Pro, OPTMIZER™ CTS™ T-Cell Expansion Basal Medium (ThermoFisher), OPTMIZER™, OPTMIZER™ Complete, IMMUNOCULT™ XF (STEMCELL™ Technologies), AIM V™, TEXMACS™ medium, PRIME-XV® T cell CDM, X-VIVO™ 15 (Lonza), TRANSACT™ TIL expansion medium, or any combination thereof. In some aspects, the basal medium is serum free. In some aspects, the basal media comprises PRIME-XV® T cell CDM. In some aspects, the basal media comprises OPTMIZER™. In some aspects, the basal media comprises OPTMIZER™ Pro. In some aspects, the basal medium further comprises immune cell serum replacement (ICSR). For example, in some aspects, the basal medium comprises OPTMIZER™ Complete supplemented with ICSR, AIM V™ supplemented with ICSR, IMMUNOCULT™ XF supplemented with ICSR, RPMI supplemented with ICSR, TEXMACS™ supplemented with ICSR, or any combination thereof. In some aspects, suitable basal media include Click's medium, OPTMIZER™ (CTS™) medium, STEMLINE® T cell expansion medium (Sigma-Aldrich), AIM V™ medium (CTS™), TEXMACS™ medium (Miltenyi Biotech), IMMUNOCULT™ medium (Stem Cell Technologies), PRIME-XV® T-Cell Expansion XSFM (Irvine Scientific), Iscoves medium, and/or RPMI-1640 medium. In some aspects, the basal media comprises NaCl free CTS™ OPTMIZER™. In some aspects, the basal media comprises one or more sodium salt in addition to the NaCl.

As used herein, the term “cytokine” refers to small, secreted proteins released by cells that have a specific effect on the interactions and communications between cells. Non-limiting examples of cytokines include interleukins (e.g., interleukin (IL)-1, IL-2, IL-4, IL-7, IL-9, IL-13, IL-15, IL-3, IL-5, IL-6, IL-11, IL-10, IL-20, IL-14, IL-16, IL-17, IL-21 and IL-23), interferons (IFN; e.g., IFN-α, IFN-β, and IFN-7), tumor necrosis factor (TNF) family members, and transforming growth factor (TGF) family members. Some aspects of the present disclosure are directed to methods of culturing and/or expanding immune cells, e.g., T cells and/or NK cells or one or more engineered immune cell disclosed herein, in a medium comprising a cytokine. In some aspects, the cytokine is an interleukin. In some aspects, the cytokine comprises IL-2, IL-7, IL-15, IL-21 or any combination thereof. IL-2 (UniProtKB—P60568) is produced by T cells in response to antigenic or mitogenic stimulation. IL-2 is known to stimulate T cell proliferation and other activities crucial to regulation of the immune response. IL-7 (UniProtKB—P13232) is a hematopoietic growth factor capable of stimulating the proliferation of lymphoid progenitors. IL-7 is believed to play a role in proliferation during certain stages of B-cell maturation. IL-15 (UniProtKB—P40933), like IL-2, is a cytokine that stimulates the proliferation of T-lymphocytes. IL-21 (UniProtKB—Q9HBE4) is a cytokine with immunoregulatory activity. IL-21 is thought to promote the transition between innate and adaptive immunity and to induce the production of IgG1 and IgG3 in B-cells. IL-21 may also play a role in proliferation and maturation of natural killer (NK) cells in synergy with IL-15, and IL-21 may regulate proliferation of mature B- and T-cells in response to activating stimuli. In synergy with IL-15 and IL-18, IL-15 also stimulates interferon gamma production in T-cells and NK cells, and IL-21 may also inhibit dendritic cell activation and maturation during a T-cell-mediated immune response.

As used herein, “administering” refers to the physical introduction of a therapeutic agent or a composition comprising a therapeutic agent to a subject, using any of the various methods and delivery systems. The different routes of administration for a therapeutic agent described herein (e.g., an immune cell or a population of immune cells modified to express an increased level of a c-Jun polypeptide, and cultured as described herein) include intravenous, intraperitoneal, intramuscular, subcutaneous, spinal or other parenteral routes of administration, for example by injection or infusion.

The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intratumoral, intracapsular, intraorbital, intracardiac, intradermal, transtracheal, intratracheal, pulmonary, subcutaneous, subcuticular, intraarticular, subcapsular, subarachnoid, intraventricular, intravitreal, epidural, and intrasternal injection and infusion, as well as in vivo electroporation.

Alternatively, a therapeutic agent described herein (e.g., an immune cell modified to express an increased level of a c-Jun polypeptide, and cultured as described herein) can be administered via a non-parenteral route, such as a topical, epidermal, or mucosal route of administration, for example, intranasally, orally, vaginally, rectally, sublingually, or topically. Administering can also be performed, for example, once, a plurality of times, and/or over one or more extended periods.

As used herein, “cell engineering” or “cell modification” (including derivatives thereof) refers to the targeted modification of a cell, e.g., an immune cell disclosed herein. In some aspects, the cell engineering comprises viral genetic engineering, non-viral genetic engineering, introduction of receptors to allow for tumor specific targeting (e.g., a chimeric binding protein) introduction of one or more endogenous genes that improve T cell function, introduction of one or more synthetic genes that improve immune cell, e.g., T cell, function (e.g., a polynucleotide encoding a c-Jun polypeptide, such that the immune cell exhibits increased c-Jun expression compared to a corresponding cell that has not been modified), or any combination thereof. As further described elsewhere in the present disclosure, in some aspects, a cell can be engineered or modified with a transcription activator (e.g., CRISPR/Cas system-based transcription activator), wherein the transcription activator is capable of inducing and/or increasing the endogenous expression of a protein of interest (e.g., c-Jun).

As used herein, the term “antigen” refers to any natural or synthetic immunogenic substance, such as a protein, peptide, or hapten. As used herein, the term “cognate antigen” refers to an antigen which an immune cell (e.g., T cell) recognizes and thereby, induces the activation of the immune cell (e.g., triggering intracellular signals that induce effector functions, such as cytokine production, and/or for proliferation of the cell). In some aspects, the antigen comprises a tumor antigen. In some aspects, the antigen comprises a neoantigen.

A “cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein comprises primary, metastatic and recurrent cancers. Unless indicated otherwise, the terms “cancer” and “tumor” can be used interchangeably.

The term “hematological malignancy” or “hematological cancer” refers to mammalian cancers and tumors of the hematopoietic and lymphoid tissues. Non-limiting examples of hematological malignancies include those affecting tissues of the blood, bone marrow, lymph nodes, and lymphatic system, including acute lymphoblastic leukemia (ALL), chronic lymphocytic lymphoma (CLL), small lymphocytic lymphoma (SLL), acute myelogenous leukemia (AML), chronic myelogenous leukemia (CIVIL), acute monocytic leukemia (AMoL), Hodgkin's lymphoma, and non-Hodgkin's lymphomas. Hematological malignancies are also referred to as “liquid tumors.” Liquid tumor cancers include, but are not limited to, leukemias, myelomas, and lymphomas, as well as other hematological malignancies.

A “solid tumor,” as used herein, refers to an abnormal mass of tissue. Solid tumors may be benign or malignant. Non-limiting examples of solid tumors include sarcomas, carcinomas, and lymphomas, such as cancers of the lung, breast, prostate, colon, rectum, and bladder. The tissue structure of a solid tumor includes interdependent tissue compartments including the parenchyma (cancer cells) and the supporting stromal cells in which the cancer cells are dispersed, and which may provide a supporting microenvironment.

In some aspects, the cancer is selected from adrenal cortical cancer, advanced cancer, anal cancer, aplastic anemia, bile duct cancer, bladder cancer, bone cancer, bone metastasis, brain tumors, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, non-small cell lung cancer, small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment. In some aspects, the cancer is selected from chondrosarcoma, fibrosarcoma, lymphosarcoma, melanosarcoma, myxosarcoma, myxoid/round cell liposarcoma, osteosarcoma, Abemethy's sarcoma, adipose sarcoma, liposarcoma, alveolar soft part sarcoma, ameloblastic sarcoma, botryoid sarcoma, chloroma sarcoma, choriocarcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma. In some aspects, the cancer is selected from acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma. In some aspects, the cancer is selected from acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum. In some aspects, the cancer is selected from Leukemia, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, esophageal cancer, genitourinary tract cancer, malignant hypercalcemia, cervical cancer, endometrial cancer, adrenal cortical cancer, prostate cancer, Müllerian cancer, ovarian cancer, peritoneal cancer, fallopian tube cancer, or uterine papillary serous carcinoma. In some aspects, the cancer is selected from metastatic melanoma, non-small cell lung cancer, myeloma, esophageal cancer, synovial sarcoma, gastric cancer, breast cancer, hepatocellular cancer, head and neck cancer, ovarian cancer, prostate cancer, bladder cancer, or any combination thereof.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, NKT cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4+ or CD8+ T cell, or the inhibition of a Treg cell. As used herein, the terms “T cell” and “T lymphocytes” are interchangeable and refer to any lymphocytes produced or processed by the thymus gland. In some aspects, a T cell is a CD4+ T cell. In some aspects, a T cell is a CD8+ T cell. In some aspects, a T cell is a NKT cell.

As used herein, the term “anti-tumor immune response” refers to an immune response against a tumor antigen.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject,” “patient,” “individual,” and “host” are used interchangeably herein. As used herein, the phrase “subject in need thereof” includes subjects, such as mammalian subjects, that would benefit, e.g., from administration of immune cells, e.g., modified to express an increased level of a c-Jun polypeptide, and cultured using the methods provided herein, as described herein to control tumor growth.

The term “therapeutically effective amount” or “therapeutically effective dosage” refers to an amount of an agent (e.g., an immune cell modified to express an increased level of a c-Jun polypeptide and cultured as described herein) that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations.

The effective amount of the composition (e.g., immune cells as described herein, e.g., modified to express an increased level of a c-Jun polypeptide and cultured as described herein) can, for example, (i) reduce the number of cancer cells; (ii) reduce tumor size; (iii) inhibit, delay, slow to some extent and can stop cancer cell infiltration into peripheral organs; (iv) inhibit (i.e., slow to some extent and can stop tumor metastasis); (v) inhibit tumor growth; (vi) prevent or delay occurrence and/or recurrence of tumor; and/or (vii) relieve to some extent one or more of the symptoms associated with the cancer.

In some aspects, a “therapeutically effective amount” is the amount of a composition disclosed herein (e.g., an immune cell modified to express an increased level of a c-Jun polypeptide, and cultured as described herein), which is clinically proven to effect a significant decrease in cancer or slowing of progression (regression) of cancer, such as an advanced solid tumor. The ability of a therapeutic agent of the present disclosure (e.g., an immune cell modified and cultured as described herein) to promote disease regression can be evaluated using a variety of methods known to the skilled practitioner, such as in human subjects during clinical trials, in animal model systems predictive of efficacy in humans, or by assaying the activity of the agent in in vitro assays.

The terms “effective” and “effectiveness” with regard to a treatment include both pharmacological effectiveness and physiological safety. Pharmacological effectiveness refers to the ability of a composition disclosed herein (e.g., immune cells modified and cultured as described herein) to promote cancer regression in the patient. Physiological safety refers to the level of toxicity, or other adverse physiological effects at the cellular, organ, and/or organism level (adverse effects) resulting from administration of a composition disclosed herein (e.g., immune cells modified and cultured as described herein).

The terms “chimeric antigen receptor” and “CAR,” as used herein, refer to a set of polypeptides, typically two in the simplest form, which when in an immune effector cell, provides the cell with specificity for a target cell, typically a cancer cell, and with intracellular signal generation. In some aspects, a CAR comprises at least an extracellular antigen-binding domain, a transmembrane domain and a cytoplasmic signaling domain (also referred to herein as “an intracellular signaling domain”) comprising a functional signaling domain derived from a stimulatory molecule and/or costimulatory molecule as defined below. In some aspects, the set of polypeptides are in the same polypeptide chain, e.g., comprise a chimeric fusion protein. In some aspects, the set of polypeptides are not contiguous with each other, e.g., are in different polypeptide chains. In some aspects, the set of polypeptides include a dimerization switch that, upon the presence of a dimerization molecule, can couple the polypeptides to one another, e.g., can couple an antigen-binding domain to an intracellular signaling domain. In some aspects, the stimulatory molecule of the CAR is the zeta chain associated with the T cell receptor complex (e.g., CD3 zeta). In some aspects, the cytoplasmic signaling domain comprises a primary signaling domain (e.g., a primary signaling domain of CD3-zeta). In some aspects, the cytoplasmic signaling domain further comprises one or more functional signaling domains derived from at least one costimulatory molecule as defined below. In some aspects, the costimulatory molecule is chosen from the costimulatory molecules described herein, e.g., 4-1BB (i.e., CD137), CD27, and/or CD28.

In some aspects, the CAR comprises a chimeric fusion protein comprising an antigen-binding domain, a transmembrane domain, and an intracellular signaling domain comprising a functional signaling domain derived from a stimulatory molecule, wherein the antigen-binding domain and the transmembrane domain are linked by a CAR spacer. In some aspects, the CAR comprises a chimeric fusion protein comprising an antigen-binding domain linked to a transmembrane domain via a CAR spacer and an intracellular signaling domain comprising a functional signaling domain derived from a costimulatory molecule and a functional signaling domain derived from a stimulatory molecule. In some aspects, the CAR comprises a chimeric fusion protein comprising an antigen-binding domain linked to a transmembrane domain via a CAR spacer and an intracellular signaling domain comprising two functional signaling domains derived from one or more costimulatory molecule(s) and a functional signaling domain derived from a stimulatory molecule. In some aspects, the CAR comprises a chimeric fusion protein comprising an antigen-binding domain linked to a transmembrane domain via a CAR spacer and an intracellular signaling domain comprising at least two functional signaling domains derived from one or more costimulatory molecule(s) and a functional signaling domain derived from a stimulatory molecule. In some aspects, the CAR comprises an optional leader sequence at the amino-terminus (N-terminus) of the CAR. In some aspects, the CAR further comprises a leader sequence at the N-terminus of the antigen-binding domain, wherein the leader sequence is optionally cleaved from the antigen-binding domain (e.g., a scFv) during cellular processing and localization of the CAR to the cellular membrane.

The antigen-specific extracellular domain of a chimeric antigen receptor recognizes and specifically binds an antigen, typically a surface-expressed antigen of a malignancy. An antigen-specific extracellular domain specifically binds an antigen when, for example, it binds the antigen with an affinity constant or affinity of interaction (K_{D}) between about 0.1 pM to about 10 μM, for example, about 0.1 pM to about 1 μM or about 0.1 pM to about 100 nM. Methods for determining the affinity of interaction are known in the art. An antigen-specific extracellular domain suitable for use in a CAR of the present disclosure can be any antigen-binding polypeptide, a wide variety of which are known in the art. In some aspects, the antigen-binding domain is a single chain Fv (scFv). Other antibody-based recognition domains such as cAb VHH (camelid antibody variable domains) and humanized versions thereof, IgNAR VH (shark antibody variable domains) and humanized versions thereof, sdAb VH (single domain antibody variable domains), and “camelized” antibody variable domains are also suitable for use in a CAR of the present disclosure. In some aspects, T cell receptor (TCR) based recognition domains, such as single chain TCR (scTv, i.e., single chain two-domain TCR containing VαVβ) are also suitable for use in the chimeric binding proteins of the present disclosure.

As used herein, the term “T cell receptor” or “TCR” refers to a heterodimer composed of 2 different transmembrane polypeptide chains: an α chain and a β chain, each consisting of a constant region, which anchors the chain inside the T-cell surface membrane, and a variable region, which recognizes and binds to the antigen presented by MHCs. The TCR complex is associated with 6 polypeptides forming 2 heterodimers, CD3γε and CD36ε, and 1 homodimer CD3ζ, which together forms the CD3 complex. T-cell receptor-engineered T-cell therapy utilizes the modification of T cells that retain these complexes to specifically target the antigens expressed by particular tumor cells. As used herein, the term “TCR” includes naturally occurring TCRs and engineered TCRs.

As used herein, an “engineered TCR” or “engineered T-cell receptor” refers to a T-cell receptor (TCR) engineered to specifically bind with a desired affinity to a major histocompatibility complex (MHC)/peptide target antigen that is selected, cloned, and/or subsequently introduced into a population of immune cells, e.g., T cells and/or NK cells.

A “TCR mimic” or a “TCRm” refers to a type of engineered chimeric TCR comprising an antigen binding domain (e.g., derived from an antibody) that recognize epitopes comprising both the peptide and the MHC-I molecule, similar to the recognition of such complexes by the TCR on T cells. The TCR mimic further comprises a T cell receptor module (TCRM) capable of recruiting at least one TCR-associated signaling molecule. Exemplary TCR mimics are described for example in U.S. Pat. No. 10,822,413, which is incorporated herein by reference in its entirety.

The terms “nucleic acids,” “nucleic acid molecules, “nucleotides,” “nucleotide(s) sequence,” and “polynucleotide” can be used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein. As described herein, in some aspects, a polynucleotide of the present disclosure can comprise a single nucleotide sequence encoding a single protein (e.g., codon-optimized c-Jun nucleotide sequence) (“monocistronic”). In some aspects, a polynucleotide of the present disclosure is polycistronic (i.e., comprises two or more cistrons). In some aspects, each of the cistrons of a polycistronic polynucleotide can encode for a protein disclosed herein (e.g., c-Jun protein, chimeric binding protein, or EGFRt). In some aspects, each of the cistrons can be translated independently of one another.

As used herein, the term “polypeptide” encompasses both peptides and proteins, unless indicated otherwise. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

As used herein, the term “fragment” of a polypeptide (e.g., a c-Jun polypeptide) refers to an amino acid sequence of a polypeptide that is shorter than the naturally-occurring sequence, N- and/or C-terminally deleted or any part of the polypeptide deleted in comparison to the naturally occurring polypeptide. Thus, a fragment does not necessarily need to have only N- and/or C-terminal amino acids deleted. A polypeptide in which internal amino acids have been deleted with respect to the naturally occurring sequence is also considered a fragment.

As used herein, the term “functional fragment” refers to a polypeptide fragment that retains polypeptide function. Accordingly, in some aspects, a functional fragment of an Ig hinge, retains the ability to position an antigen-binding domain (e.g., an scFv) in a chimeric binding protein at a distance from a target epitope (e.g., a tumor antigen) such that the antigen-binding domain (e.g., an scFv) can effectively interact with the target epitope (e.g., a tumor antigen). Similarly, in some aspects, a c-Jun functional fragment is a fragment that when expressed in an immune cell (e.g., CAR T cell), results in an immune cell with, e.g., at least about 20%, at least about 25%, at least about 30%, at least about 35%, at least about 40%, at least about 45%, at least about 50%, at least 55%, at least about 60%, at least about 65%, at least about 70%, at least about 75% at least about 80%, at least about 85%, at least about 90%, at least about 95%, at least about 99%, or about 100% of the activity of a reference immune cell expressing a corresponding full length c-Jun. Non-limiting examples of such activity are further described elsewhere in the present disclosure.

A “recombinant” polypeptide or protein refers to a polypeptide or protein produced via recombinant DNA technology. Recombinantly produced polypeptides and proteins expressed in engineered host cells are considered isolated for the purpose of the disclosure, as are native or recombinant polypeptides which have been separated, fractionated, or partially or substantially purified by any suitable technique. The polypeptides encoded by the polynucleotides disclosed herein (e.g., chimeric binding protein, c-Jun, and/or EGFRt) can be recombinantly produced using methods known in the art. In some aspects, the polypeptides encoded by the polynucleotides of the present disclosure (e.g., chimeric binding protein, c-Jun, and/or EGFRt) are produced by cells, e.g., T cells, following transfection with at least one polynucleotide or vector encoding the polypeptides described here.

As used herein, a “coding region,” “coding sequence,” or “translatable sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide.

The terms “complementary” and “complementarity” refer to two or more oligomers (i.e., each comprising a nucleobase sequence), or between an oligomer and a target gene, that are related with one another by Watson-Crick base-pairing rules. For example, the nucleobase sequence “T-G-A (5′ to 3′),” is complementary to the nucleobase sequence “A-C-T (3′ to 5′).” Complementarity can be “partial,” in which less than all of the nucleobases of a given nucleobase sequence are matched to the other nucleobase sequence according to base pairing rules. For example, in some aspects, complementarity between a given nucleobase sequence and the other nucleobase sequence can be about 70%, about 75%, about 80%, about 85%, about 90%, or about 95%. Accordingly, in some aspects, the term “complementary” refers to at least about 80%, at least about 85%, at least about 90%, at least about 91%, at least about 92%, at least about 93%, at least about 94%, at least about 95%, at least about 96%, at least about 97%, at least about 98%, or at least about 99% match or complementarity to a target nucleic acid sequence (e.g., c-Jun encoding nucleic acid sequence). Or, there can be “complete” or “perfect” (100%) complementarity between a given nucleobase sequence and the other nucleobase sequence to continue the example. In some aspects, the degree of complementarity between nucleobase sequences has significant effects on the efficiency and strength of hybridization between the sequences.

The term “expression” as used herein refers to a process by which a polynucleotide produces a gene product, for example, a c-Jun polypeptide. It includes, without limitation, transcription of the polynucleotide into messenger RNA (mRNA) and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

As used herein, the term “identity” refers to the overall monomer conservation between polymeric molecules, e.g., between polynucleotide molecules. The term “identical” without any additional qualifiers, e.g., polynucleotide A is identical to polynucleotide B, implies the polynucleotide sequences are 100% identical (100% sequence identity). Describing two sequences as, e.g., “70% identical,” is equivalent to describing them as having, e.g., “70% sequence identity.”

Calculation of the percent identity of two polypeptide or polynucleotide sequences, for example, can be performed by aligning the two sequences for optimal comparison purposes (e.g., gaps can be introduced in one or both of a first and a second polypeptide or polynucleotide sequences for optimal alignment and non-identical sequences can be disregarded for comparison purposes). In some aspects, the length of a sequence aligned for comparison purposes is at least about 30%, at least about 40%, at least about 50%, at least about 60%, at least about 70%, at least about 80%, at least about 90%, at least about 95%, or about 100% of the length of the reference sequence. The amino acids at corresponding amino acid positions, or bases in the case of polynucleotides, are then compared.

When a position in the first sequence is occupied by the same amino acid or nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs that can be used to align different sequences (e.g., polynucleotide sequences) are available from various sources. One suitable program to determine percent sequence identity is bl2seq, part of the BLAST suite of programs available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at worldwideweb.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In some aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at worldwidewebtcoffee.org, and alternatively available, e.g., from the EBI. It will also be appreciated that the final alignment used to calculate percent sequence identity can be curated either automatically or manually.

As used herein, the terms “isolated,” “purified,” “extracted,” and grammatical variants thereof are used interchangeably and refer to the state of a preparation of desired composition of the present disclosure that has undergone one or more processes of purification. In some aspects, isolating or purifying as used herein is the process of removing, including partially removing (e.g., a fraction), a composition of the present disclosure (e.g., a modified immune cell expressing an increased level of a c-Jun protein) from a sample containing contaminants.

In some aspects, an isolated composition has no detectable undesired activity or, alternatively, the level or amount of the undesired activity is at or below an acceptable level or amount. In some aspects, an isolated composition has an amount and/or concentration of desired composition of the present disclosure, at or above an acceptable amount and/or concentration and/or activity. In some aspects, the isolated composition is enriched as compared to the starting material from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.9999%, or greater than 99.9999% as compared to the starting material.

In some aspects, isolated preparations are substantially free of residual biological products. In some aspects, the isolated preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites.

The term “linked” as used herein refers to a first amino acid sequence or polynucleotide sequence covalently or non-covalently joined to a second amino acid sequence or polynucleotide sequence, respectively. The first amino acid or polynucleotide sequence can be directly joined or juxtaposed to the second amino acid or polynucleotide sequence or alternatively an intervening sequence can covalently join the first sequence to the second sequence. The term “linked” means not only a fusion of a first polynucleotide sequence to a second polynucleotide sequence at the 5′-end or the 3′-end, but also includes insertion of the whole first polynucleotide sequence (or the second polynucleotide sequence) into any two nucleotides in the second polynucleotide sequence (or the first polynucleotide sequence, respectively). The first polynucleotide sequence can be linked to a second polynucleotide sequence by a phosphodiester bond or a linker. The linker can be, e.g., a polynucleotide.

“Treatment” or “therapy” (including any grammatical derivatives thereof) of a subject refers to any type of intervention or process performed on, or the administration of an active agent to, a subject with the objective of reversing, alleviating, ameliorating, inhibiting, slowing down, or preventing the onset, progression, development, severity, or recurrence of a symptom, complication, condition, or biochemical indicia associated with a disease. In some aspects, the terms refers to inducing an immune response in a subject against an antigen.

The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

As used herein, the term “promoter” refers to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

As used herein, the terms “ug” and “uM“are used interchangeably with” g“and” M,” respectively.

Various aspects of the disclosure are described in further detail in the following subsections.

### II. Methods of the Disclosure

II.A. Metabolic Reprogramming Media

Some aspects of the present disclosure are directed to methods of culturing immune cells, e.g., T cells and/or NK cells (e.g., modified to express an increased level of a c-Jun protein), in a culture condition (e.g., media), wherein the culture condition (e.g., certain ion concentrations, tonicity of the media, cytokines, and/or any combination thereof) is capable of reducing, limiting or preventing the differentiation of the immune cells, e.g., T cells and/or NK cells (e.g., modified to express an increased level of a c-Jun protein), thereby affecting or improving their use in cell therapy, e.g., adoptive cell therapy. In some aspects, the immune cells, e.g., T cells and/or NK cells (e.g., modified to express an increased level of a c-Jun protein), are cultured in a metabolic reprogramming media (MRM) disclosed herein. In some aspects, the immune cells, e.g., T cells and/or NK cells (e.g., modified to express an increased level of a c-Jun protein), cultured in MRM have a higher proportion of stem-like cells as compared to cells cultured using conventional methods, e.g., in a medium having less than 5 mM potassium ion. In some aspects, the immune cells, e.g., T cells and/or NK cells (e.g., modified to express an increased level of a c-Jun protein), cultured in MRM have a higher proportion of effector-like cells as compared to cells cultured using conventional methods, e.g., in a medium having less than 5 mM potassium ion. In some aspects, the immune cells, e.g., T cells and/or NK cells (e.g., modified to express an increased level of a c-Jun protein), cultured in MRM have a higher proportion of both stem-like and effector-like cells as compared to cells cultured using conventional methods, e.g., in a medium having less than 5 mM potassium ion. In some aspects, the immune cells, e.g., T cells and/or NK cells (e.g., modified to express an increased level of a c-Jun protein), cultured in MRM have a higher proliferative potential as compared to cells cultured using conventional methods, e.g., in a medium having less than 5 mM potassium ion.

Some aspects of the present disclosure are directed to methods of preparing a population of immune cells, e.g., T cells and/or NK cells (e.g., modified to express an increased level of a c-Jun protein), comprising culturing the cells in a medium comprising potassium ion at a concentration higher than 5 mM (e.g., a metabolic reprogramming medium disclosed herein). Some aspects of the present disclosure are directed to methods of preparing a population of T cells, comprising culturing the T cells (e.g., modified to express an increased level of a c-Jun protein) in a medium comprising potassium ion at a concentration higher than 5 mM (e.g., a metabolic reprogramming medium disclosed herein). In some aspects, the present disclosure provides methods of preparing immune cells, e.g., T cells and/or NK cells (e.g., modified to express an increased level of a c-Jun protein), comprising culturing the cells in a medium comprising potassium ion at a concentration higher than 5 mM (e.g., higher than 40 mM, e.g., between 40 mM and 80 mM, e.g., between 55 mM and 70 mM), are capable of preserving a stem-like phenotype (e.g., minimal differentiation) of the cultured cells. In some aspects, the present disclosure provides methods of preparing T cells, comprising culturing the T cells (e.g., modified to express an increased level of a c-Jun protein) in a medium comprising potassium ion at a concentration higher than 5 mM (e.g., higher than 40 mM, e.g., between 40 mM and 80 mM, e.g., between 55 mM and 70 mM), are capable of preserving a stem-like phenotype (e.g., minimal differentiation) of the cultured T cells. In some aspects, the cultured cells have more stem-like phenotypes (e.g., less differentiated) than cells grown in a medium having a lower potassium concentration. In some aspects, the medium further comprises interleukin (IL)-2, IL-21, IL-7, IL-15, or any combination thereof. In some aspects, the medium further comprises sodium ion (e.g., NaCl), calcium ion, glucose, or any combination thereof.

In some aspects, a population of immune cells, e.g., T cells and/or NK cells (e.g., modified to express an increased level of a c-Jun protein), cultured using the methods disclosed herein, exhibits an increased number of stem-like cells relative to a population of cells cultured using conventional methods, e.g., in a medium having less than 5 mM potassium ion. In some aspects, a population of T cells (e.g., modified to express an increased level of a c-Jun protein), cultured using the methods disclosed herein, exhibits an increased number of stem-like T cells relative to a population of T cells cultured using conventional methods, e.g., in a medium having less than 5 mM potassium ion. In some aspects, the immune cells, e.g., T cells and/or NK cells (e.g., modified to express an increased level of a c-Jun protein), exhibit increased expression of markers characteristic of stem-like cells relative to the starting population of immune cells (i.e., prior to the culturing). In some aspects, the T cells (e.g., modified to express an increased level of a c-Jun protein), exhibit increased expression of markers characteristic of stem-like cells relative to the starting population of T cells (i.e., prior to the culturing). In some aspects, the starting population of immune cells comprises immune cells (e.g., T cells and/or NK cells) obtained from a human subject. In some aspects, the starting population of immune cells comprises T cells obtained from a human subject. In some aspects, the starting population of immune T cells comprises T_{N} cells, T_{SCM} cells, T_{CM} cells, T_{EM} cells, or any combination thereof. In some aspects, the starting population of immune cells comprises T cells prior to modification as described herein (e.g., transfection with a polynucleotide encoding a c-Jun protein and/or with a transcriptional activator that is capable of increasing the expression of endogenous c-Jun protein).

Increased cell multipotency can be measured using any methods known in the art. In some aspects, cell stemness is measured by antibody staining followed by gated flow cytometry. In some aspects, the cell stemness is measured by autophagy flux. In some aspects, the cell stemness is measured by glucose uptake. In some aspects, the cell stemness is measured by fatty acid uptake. In some aspects, the cell stemness is measured by mitochondrial biomass. In some aspects, the cell stemness is measured by RNA quantification/expression analysis (e.g., microarray, qPCR (taqman), RNA-Seq., single-cell RNA-Seq., or any combinations thereof). In some aspects, the cell stemness is measured by transcripts that are linked to a metabolism assay (e.g., a seahorse metabolism assay, analysis of extracellular acidification rate (ECAR); analysis of oxygen consumption rate (OCR); analysis of spare respiratory capacity; and/or analysis of mitochondrial membrane potential). In some aspects, stemness is measured using one or more in vivo or in vitro functional assays (e.g., assaying cell persistence, antitumor capacity, antitumor clearance, viral clearance, multipotency, cytokine release, cell killing, or any combination thereof).

In some aspects, the differentiation status of the immune cells, e.g., T cells and/or NK cells (e.g., modified to express an increased level of a c-Jun protein), is characterized by increased numbers of cells expressing markers typical of less differentiated cells. In some aspects, the differentiation status of the T cells is characterized by increased numbers of cells expressing markers typical of less differentiated T cells. In some aspects, an increase in the number of stem-like cells is characterized by increased numbers of T cells expressing markers typical of T_{N} and/or T_{SCM} cells. In some aspects, an increase in the number of stem-like T cells is characterized by increased numbers of cells expressing markers typical of T_{SCM} cells. In some aspects, the T cell population exhibits an increased number of cells that express CD45RA. In some aspects, the T cell population exhibits an increased number of cells that express CCR7. In some aspects, the T cell population exhibits an increased number of cells that express CD62L. In some aspects, the T cell population exhibits an increased number of cells that express CD28. In some aspects, the T cell population exhibits an increased number of cells that express CD95. In some aspects, the cells are CD45RO^{low}. In some aspects, the cells do not express CD45RO. In some aspects, the cell population exhibits an increased number of cells (e.g., CD4+ and/or CD8+ T cells) that are CD45RA⁺ and CCR7⁺. In some aspects, the cell population exhibits an increased number of cells that are CD45RA⁺, CCR7⁺, and CD62L⁺. In some aspects, the cell population exhibits an increased number of cells that are CD95⁺, CD45RA⁺, CCR7⁺, and CD62L⁺. In some aspects, the cell population exhibits an increased number of cells that express TCF7. In some aspects, the T cell population exhibits an increased number of cells that are CD45RA⁺, CCR7⁺, CD62L⁺, and TCF7⁺. In some aspects, the T cell population exhibits an increased number of cells that are CD95⁺, CD45RA⁺, CCR7⁺, CD62L⁺, and TCF7⁺. In some aspects, the T cell population exhibits an increased number of cells that are CD3⁺, CD45RA⁺, CCR7⁺, CD62L⁺, and TCF7⁺. In some aspects, the T cell population exhibits an increased number of cells that are CD3⁺, CD95⁺, CD45RA⁺, CCR7⁺, CD62L⁺, and TCF7⁺. In some aspects, the cells express CD27. In some aspects, the T cell population exhibits an increased number of cells that are CD27⁺, CD3⁺, CD45RA⁺, CCR7⁺, CD62L⁺, and TCF7⁺. In some aspects, the T cell population exhibits an increased number of cells that are CD27⁺, CD3⁺, CD95⁺, CD45RA⁺, CCR7⁺, CD62L⁺, and TCF7⁺. In some aspects, the T cell population exhibits an increased number of cells that are CD39⁻ and CD69⁻. In some aspects the T cell population exhibits an increased number of cells that are TCF7⁺ and CD39⁻. In some aspects, the cell population exhibits an increased number of T_{SCM} cells. In some aspects, the cell population exhibits an increased number of T_{N} cells. In some aspects, the cell population exhibits an increased number of T_{SCM} and T_{N} cells. In some aspects, the cell population exhibits an increased number of stem-like T cells. In some aspects, the T cells are CD4+ cells; in some aspects, the T cells are CD8+ cells. In some aspects, the T cells comprise both CD4+ T cells and CD8+ T cells.

In some aspects, the number of stem-like cells in the culture is increased by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 100%, relative to the number of stem-like cells prior to culture with MRM. In some aspects, the number of stem-like cells in the culture is increased by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 15-fold, or at least about 20-fold, relative to the number of stem-like cells prior to culture with MRM.

In some aspects, following culture of T cells (e.g., modified to express an increased level of a c-Jun protein) according to the methods disclosed herein, stem-like T cells constitute at least about 1%, at least about 2%, at least about 3%, at least about 4%, at least about 5%, at least about 10%, or at least about 15% of the total number of CD8⁺ T cells in the culture. In some aspects, the stem-like T cells (e.g., CD45RA⁺ and CCR7⁺) constitute at least about 20% of the CD8⁺ T cells. In some aspects, the stem-like T cells (e.g., CD45RA⁺ and CCR7⁺) constitute at least about 15% of the CD8⁺ T cells. In some aspects, following culture of T cells (e.g., modified to express an increased level of a c-Jun protein) according to the methods disclosed herein, stem-like T cells constitute at least about 1%, at least about 2%, at least about 3%, at least about 4%, at least about 5%, at least about 10%, or at least about 15% of the total number of CD4⁺ T cells in the culture. In some aspects, the stem-like T cells (e.g., CD45RA⁺ and CCR7⁺) constitute at least about 20% of the CD4⁺ T cells.

As described herein, in some aspects, the culturing methods of the present disclosure can be used to modify T cells (e.g., CD8⁺ T cells and/or CD4⁺ T cells) to (a) express a ligand binding protein (e.g., CAR or engineered TCRs) and (b) have an increased level of a c-Jun protein. Accordingly, in some aspects, after the culturing, CD8⁺ T cells express a CAR and have increased level of a c-Jun protein and at least about 20% of the modified CD8⁺ T cells are stem-like T cells (e.g., CD45RA⁺ and CCR7⁺). In some aspects, after the culturing, CD8⁺ T cells express an engineered TCR and have increased level of a c-Jun protein and at least about 15% of the modified CD8⁺ T cells are stem-like T cells (e.g., CD45RA⁺ and CCR7⁺). In some aspects, after the culturing, CD4⁺ T cells express a CAR and have increased level of a c-Jun protein and at least about 20% of the modified CD4⁺ T cells are stem-like T cells (e.g., CD45RA⁺ and CCR7⁺). In some aspects, after the culturing, CD4⁺ T cells express an engineered TCR and have increased level of a c-Jun protein and at least about 15% of the modified CD4⁺ T cells are stem-like T cells (e.g., CD45RA⁺ and CCR7⁺).

In some aspects, following culture of T cells (e.g., modified to express an increased level of a c-Jun protein) according to the methods disclosed herein, stem-like T cells constitute at least about 10% to at least about 70% of the total number of T cells in the culture. In some aspects, following culture of T cells (e.g., modified to express an increased level of a c-Jun protein) according to the methods disclosed herein, stem-like T cells constitute at least about 10%, at least about 20%, at least about 30%, at least about 40%, at least about 50%, at least about 60%, or at least about 70% of the total number of CD8⁺ T cells in the culture. In some aspects, following culture of T cells (e.g., modified to express an increased level of a c-Jun protein) according to the methods disclosed herein, stem-like T cells constitute at least about 10%, at least about 20%, at least about 30%, at least about 40%, at least about 50%, at least about 60%, or at least about 70% of the total number of CD4⁺ T cells in the culture.

In some aspects, following culture of T cells (e.g., modified to express an increased level of a c-Jun protein) according to the methods disclosed herein, at least about 10% to at least about 40% of the total number of T cells in the culture are CD39⁻/CD69⁻ T cells. In some aspects, following culture of T cells (e.g., modified to express an increased level of a c-Jun protein) according to the methods disclosed herein, at least about 10%, at least about 15%, at least about 20%, at least about 25%, at least about 30%, at least about 35%, or at least about 40% of the total number of T cells in the culture are CD39⁻/CD69⁻ T cells.

In some aspects, following culture of T cells (e.g., modified to express an increased level of a c-Jun protein) according to the methods disclosed herein, at least about 10% to at least about 70% of the total number of T cells in the culture are CD39⁻/TCF7⁺ T cells. In some aspects, following culture of T cells (e.g., modified to express an increased level of a c-Jun protein) according to the methods disclosed herein, at least about 10%, at least about 15%, at least about 20%, at least about 25%, at least about 30%, at least about 35%, or at least about 40% of the total number of T cells in the culture are CD39⁻/TCF7⁺ T cells. In some aspects, the T cells are CD4⁺ T cells. In some aspects, the T cells are CD8⁺ T cells.

In some aspects, following culture of T cells (e.g., modified to have an increased level of a c-Jun protein) according to the methods disclosed herein, at least about 10% to at least about 70% of the total number of T cells are CD45RA⁺ and CCR7⁺ T cells. In some aspects, following culture of T cells (e.g., modified to have an increased level of a c-Jun protein) according to the methods disclosed herein, at least about 10%, at least about 15%, at least about 20%, at least about 25%, at least about 30%, at least about 35%, or at least about 40% of the total number of T cells in the culture are CD45RA⁺ and CCR7⁺ T cells. In some aspects, the T cells are CD4⁺ T cells. In some aspects, the T cells are CD8⁺ T cells. In some aspects, the T cells comprise both CD4⁺ T cells and CD8⁺ T cells.

In some aspects, the immune cells, e.g., engineered immune cells (e.g., T cells and/or NK cells modified to comprise an increased level of a c-Jun protein) of the present disclosure, cultured according to the methods disclosed herein, exhibit increased transduction efficiency.

In some aspects, a greater percentage of cells express a target transgene, e.g., encoding a ligand binding protein, following transduction, wherein the cells are cultured according to the methods disclosed herein as compared to cells similarly transduced and cultured using conventional methods, (e.g., in media containing less than 5 mM K⁺). In certain aspects, a greater percentage of cells cultured according to the methods disclosed herein express a ligand binding protein following lentiviral transduction of the cells, as compared to similarly transduced cells cultured using conventional methods, e.g., in media containing less than 5 mM K⁺. In some aspects, transduction efficiency is increased at least about 1.5-fold relative to similarly transduced cells cultured using conventional methods, e.g., in media containing less than 5 mM K⁺. In some aspects, transduction efficiency is increased at least about 2-fold relative to similarly transduced cells cultured using conventional methods, e.g., in media containing less than 5 mM K⁺. As used herein, the term “transduction efficiency” refers to: (i) the amount of material (e.g., exogenous polynucleotide) that can be physically introduced into a cell within a defined period of time; (ii) the amount of time it takes to physically introduce a given amount of material into a cell; (iii) the level to which a target material, e.g., an exogenous polynucleotide, i.e., a transgene, is taken up by a population of cells (e.g., the percentage of cells that express the transgene); or (iv) any combination of (i)-(iii). In some aspects, by increasing transduction efficiency, the culturing methods provided herein can allow for a greater amount of an exogenous nucleotide sequence to be introduced into a cell and/or decrease the amount of time required to introduce a given amount of an exogenous nucleotide sequence. Not to be bound by any one theory, in some aspects, such an effect can increase the expression of the encoded protein (e.g., c-Jun polypeptide) in the modified immune cell.

In some aspects, the immune cells, e.g., T cells and/or NK cells, are transduced before culturing according to the methods disclosed herein. In some aspects, the immune cells, e.g., T cells and/or NK cells, are transduced after culturing according to the methods disclosed herein. In some aspects, the immune cells, e.g., T cells and/or NK cells, are cultured according to the methods disclosed herein, e.g., by contacting the immune cells with an APC-MS in a medium comprising at least 5 mM potassium ion (e.g., higher than 5 mM, e.g., between about 40 mM to about 80 mM), prior to, during, and after transduction.

In certain aspects, the immune cells are transduced using a viral vector. In some aspects, the vector comprises a lentiviral vector, adenoviral vector, adeno-associated viral vector, vaccinia vector, herpes simplex viral vector, and Epstein-Barr viral vector. In some aspects, the viral vector comprises a retrovirus. In some aspects, the viral vector comprises a lentivirus. In some aspects, the viral vector comprises an AAV.

In some aspects, the immune cells are transduced using a non-viral method. In some aspects, the non-viral method includes the use of a transposon. In some aspects, use of a non-viral method of delivery permits reprogramming of immune cells, e.g., T cells and/or NK cells, and direct infusion of the cells into the subject. In some aspects, the polynucleotide can be inserted into the genome of a target cell (e.g., a T cell) or a host cell (e.g., a cell for recombinant expression of the encoded proteins) by using CRISPR/Cas systems and genome edition alternatives such as zinc-finger nucleases (ZFNs), transcription activator-like effector nucleases (TALENs), and meganucleases (MNs).

In some aspects, upon adoptive transfer of the immune cells, e.g., T cells and/or NK cells (e.g., modified to express an increased level of a c-Jun protein), optionally expressing a ligand binding protein, cultured according to the methods disclosed herein, the transferred cells exhibit decreased cell exhaustion, as compared to cells cultured using conventional methods, e.g., in media containing less than 5 mM K⁺. In some aspects, upon adoptive transfer of the T cells (e.g., modified to express an increased level of a c-Jun protein), optionally expressing a ligand binding protein, cultured according to the methods disclosed herein, the transferred T cells exhibit decreased cell exhaustion, as compared to T cells cultured using conventional methods, e.g., in media containing less than 5 mM K⁺.

In some aspects, upon adoptive transfer of the cells cultured according to the methods disclosed herein, the transferred cells persist for a longer period of time in vivo, as compared to cells cultured using conventional methods, e.g., in media containing less than 5 mM K⁺. In some aspects, the transferred cells, e.g., T cells and/or NK cells, have a greater in vivo efficacy, e.g., tumor-killing activity, as compared to cells cultured using conventional methods, e.g., in media containing less than 5 mM K⁺. In some aspects, a lower dose of the cells cultured according to the methods disclosed herein is needed to elicit a response, e.g., decreased tumor volume, in a subject as compared to cells cultured using conventional methods, e.g., in media containing less than 5 mM K⁺.

In some aspects, the immune cells (e.g., T cells and/or NK cells) are cultured according to the methods disclosed herein, e.g., in a medium comprising at least 5 mM potassium ion (e.g., higher than 5 mM, e.g., between about 40 mM to about 80 mM), immediately upon isolation from a subject. In some aspects, the immune cells, e.g., T cells and/or NK cells, are cultured according to the methods disclosed herein during expansion of the cells. In some aspects, the immune cells, e.g., T cells and/or NK cells, are cultured according to the methods disclosed herein during engineering of the cells, e.g., during transduction with a construct encoding a transgene, e.g., a ligand binding protein. In some aspects, the immune cells, e.g., T cells and/or NK cells, are cultured according to the methods disclosed herein following engineering of the cells, e.g., following transduction with a construct encoding a transgene, e.g., a ligand binding protein. In some aspects, the immune cells, e.g., T cells and/or NK cells, are cultured according to the methods disclosed herein throughout expansion and engineering. In some aspects, the immune cells, e.g., T cells and/or NK cells, are cultured according to the methods disclosed herein throughout viral genetic engineering. In some aspects, the immune cells, e.g., T cells and/or NK cells, are cultured according to the methods disclosed herein throughout non-viral genetic engineering. In some aspects, the immune cells, e.g., T cells and/or NK cells, are cultured according to the methods disclosed herein during introduction of ligand binding proteins to the immune cell (e.g., T cells and/or NK cells) to allow for tumor specific targeting (e.g., a CAR, TCR or a TCR mimic). In some aspects, the immune cells, e.g., T cells and/or NK cells, are cultured according to the methods disclosed herein throughout introduction of one or more endogenous genes that improve T cell function (e.g., c-Jun). In some aspects, the immune cells, e.g., T cells and/or NK cells, are cultured according to the methods disclosed herein throughout introduction of one or more synthetic genes that improve T cell function (e.g., exogenous polynucleotide encoding a c-Jun protein, or exogenous polynucleotide encoding a CAR, TCR, caTCR, CSR, or TCR mimic).

In some aspects, the immune cells, e.g., T cells and/or NK cells, are cultured according to the methods disclosed herein, e.g., in a medium comprising at least 5 mM potassium ion (e.g., higher than 5 mM, e.g., between about 40 mM to about 80 mM), for the entirety of ex vivo culture, e.g., from the time the immune cells, e.g., T cells and/or NK cells, are isolated from a subject, through growing, expansion, engineering, and until administration into a subject in need of adoptive cell therapy. In some aspects, the T cells are cultured according to the methods disclosed herein, e.g., in a medium comprising at least 5 mM potassium ion (e.g., higher than 5 mM, e.g., between about 40 mM to about 80 mM), for the entirety of ex vivo culture, e.g., from the time the T cells are isolated from a subject, through growing, expansion, engineering, and until administration into a subject in need of adoptive cell therapy. In some aspects, the immune cells, e.g., T cells and/or NK cells, are cultured according to the methods disclosed herein for the duration of expansion. In some aspects, the immune cells, e.g., T cells and/or NK cells, are cultured according to the methods disclosed herein until the total number of viable immune cells, e.g., T cells and/or NK cells, is at least about 10⁴, at least about 5×10⁴, at least about 10⁵, at least about 5×10⁵, at least about 10⁶, at least about 5×10⁶, at least about 1×10⁷, at least about 5×10⁷, at least about 1×10⁸, at least about 5×10⁸, at least about 1×10⁹, at least about 5×10⁹, at least about 1×10¹⁰, at least about 5×10¹⁰, at least about 1×10¹¹, at least about 5×10¹¹, at least about 1×10¹², or at least about 5×10¹² total cells. In some aspects, the T cells are cultured according to the methods disclosed herein until the total number of viable T cells is at least about 10⁴, at least about 5×10⁴, at least about 10⁵, at least about 5×10⁵, at least about 10⁶, at least about 5×10⁶, at least about 1×10⁷, at least about 5×10⁷, at least about 1×10⁸, at least about 5×10⁸, at least about 1×10⁹, at least about 5×10⁹, at least about 1×10¹⁰, at least about 5×10¹⁰, at least about 1×10¹¹, at least about 5×10¹¹, at least about 1×10¹², or at least about 5×10¹² total T cells.

In some aspects, the medium further comprises a cell expansion agent. As used herein, a “cell expansion agent” refers to an agent, e.g., small molecule, polypeptide, or any combination thereof, that promotes the in vitro and/or ex vivo growth and proliferation of cultured cells, e.g., immune cells (e.g., T cells and/or NK cells). In some aspects, the cell expansion agent comprises a PI3K inhibitor. In some aspects, the medium further comprises an AKT inhibitor. In some aspects, the medium further comprises a PI3K inhibitor and an AKT inhibitor. In some aspects, the PI3K inhibitor comprises LY294002. In some aspects, the PI3K inhibitor comprises IC87114. In some aspects, the PI3K inhibitor comprises idelalisib (see, e.g., Peterson et al., Blood Adv. 2(3):210-23 (2018)). In some aspects, the medium further comprises a GSK3B inhibitor. In some aspects, the GSK3B inhibitor comprises TWS119. In some aspects, the medium further comprises an ACLY inhibitor. In some aspects, the ACLY inhibitor comprises potassium hydroxycitrate tribasic monohydrate. In some aspects, the PI3K inhibitor comprises hydroxyl citrate. In some aspects, the PI3K inhibitor comprises pictilisib. In some aspects, the PI3K inhibitor comprises CAL-101. In some aspects, the AKT inhibitor comprises MK2206, A443654, or AKTi-VIII (CAS 612847-09-3).

In some aspects, the metabolic reprogramming media comprises a mitochondrial fuel. In some aspects, the metabolic reprogramming media comprises O-Acetyl-L-carnitine hydrochloride. In some aspects, the metabolic reprogramming media comprises at least about 0.1 mM, at least about 0.5 mM, at least about 1.0 mM, at least about 5 mM, or at least about 10 mM O-Acetyl-L-carnitine hydrochloride. In some aspects, the metabolic reprogramming media comprises at least about 1.0 mM O-Acetyl-L-carnitine hydrochloride.

In some aspects, the metabolic reprogramming media further comprises one or more of (i) one or more cell expansion agents, (ii) sodium ion (e.g., NaCl), (iii) one or more saccharides, (iv) calcium ion, and (v) one or more cytokines.

II.A.1. Potassium

Some aspects of the disclosure are directed to methods of culturing immune cells, e.g., T cells and/or NK cells, in a medium comprising an increased concentration of potassium ion (e.g., greater than about 5 mM, greater than about 40 mM, greater than about 45 mM, greater than about 50 mM, greater than about 55 mM, greater than about 60 mM, greater than about 65 mM, or greater than about 70 mM), i.e., a metabolic reprogramming medium disclosed herein, relative to a control medium. In some aspects, the metabolic reprogramming medium comprises at least about 5 mM to at least about 100 mM potassium ion, at least about 5 mM to at least about 90 mM potassium ion, at least about 5 mM to at least about 80 mM potassium ion, at least about 5 mM to at least about 75 mM potassium ion, at least about 5 mM to at least about 70 mM potassium ion, at least about 5 mM to at least about 65 mM potassium ion, at least about 5 mM to at least about 60 mM potassium ion, at least about 5 mM to at least about 55 mM potassium ion, at least about 5 mM to at least about 50 mM potassium ion, at least about 5 mM to at least about 45 mM potassium ion, at least about 5 mM to at least about 40 mM potassium ion, at least about 10 mM to at least about 80 mM potassium ion, at least about 10 mM to at least about 75 mM potassium ion, at least about 10 mM to at least about 70 mM potassium ion, at least about 10 mM to at least about 65 mM potassium ion, at least about 10 mM to at least about 60 mM potassium ion, at least about 10 mM to at least about 55 mM potassium ion, at least about 10 mM to at least about 50 mM potassium ion, at least about 10 mM to at least about 45 mM potassium ion, at least about 10 mM to at least about 40 mM potassium ion, at least about 20 mM to at least about 80 mM potassium ion, at least about 20 mM to at least about 75 mM potassium ion, at least about 20 mM to at least about 70 mM potassium ion, at least about 20 mM to at least about 65 mM potassium ion, at least about 20 mM to at least about 60 mM potassium ion, at least about 20 mM to at least about 55 mM potassium ion, at least about 20 mM to at least about 50 mM potassium ion, at least about 20 mM to at least about 45 mM potassium ion, at least about 20 mM to at least about 40 mM potassium ion, at least about 30 mM to at least about 80 mM potassium ion, at least about 30 mM to at least about 75 mM potassium ion, at least about 30 mM to at least about 70 mM potassium ion, at least about 30 mM to at least about 65 mM potassium ion, at least about 30 mM to at least about 60 mM potassium ion, at least about 30 mM to at least about 55 mM potassium ion, at least about 30 mM to at least about 50 mM potassium ion, at least about 30 mM to at least about 45 mM potassium ion, at least about 30 mM to at least about 40 mM potassium ion, at least about 40 mM to at least about 80 mM potassium ion, at least about 40 mM to at least about 75 mM potassium ion, at least about 40 mM to at least about 70 mM potassium ion, at least about 40 mM to at least about 65 mM potassium ion, at least about 40 mM to at least about 60 mM potassium ion, at least about 40 mM to at least about 55 mM potassium ion, at least about 40 mM to at least about 50 mM potassium ion, at least about 40 mM to at least about 45 mM potassium ion, at least about 45 mM to at least about 80 mM potassium ion, at least about 45 mM to at least about 75 mM potassium ion, at least about 45 mM to at least about 70 mM potassium ion, at least about 45 mM to at least about 65 mM potassium ion, at least about 45 mM to at least about 60 mM potassium ion, at least about 45 mM to at least about 55 mM potassium ion, at least about 45 mM to at least about 50 mM potassium ion, at least about 50 mM to at least about 80 mM potassium ion, at least about 50 mM to at least about 75 mM potassium ion, at least about 50 mM to at least about 70 mM potassium ion, at least about 50 mM to at least about 65 mM potassium ion, at least about 50 mM to at least about 60 mM potassium ion, or at least about 50 mM to at least about 55 mM potassium ion.

In some aspects, the metabolic reprogramming medium comprises at least about 5 mM, at least about 10 mM, at least about 15 mM, at least about 20 mM, at least about 25 mM, at least about 30 mM, at least about 35 mM, at least about 40 mM, at least about 45 mM, at least about 50 mM, at least about 55 mM, at least about 60 mM, at least about 65 mM, at least about 70 mM, at least about 75 mM, or at least about 80 mM potassium ion. In some aspects, the metabolic reprogramming medium comprises at least about 5 mM potassium ion. In some aspects, the metabolic reprogramming medium comprises at least about 10 mM potassium ion. In some aspects, the metabolic reprogramming medium comprises at least about 15 mM potassium ion. In some aspects, the metabolic reprogramming medium comprises at least about 20 mM potassium ion. In some aspects, the metabolic reprogramming medium comprises at least about 25 mM potassium ion. In some aspects, the metabolic reprogramming medium comprises at least about 30 mM potassium ion. In some aspects, the metabolic reprogramming medium comprises at least about 35 mM potassium ion. In some aspects, the metabolic reprogramming medium comprises at least about 40 mM potassium ion. In some aspects, the metabolic reprogramming medium comprises at least about 45 mM potassium ion. In some aspects, the metabolic reprogramming medium comprises at least about 50 mM potassium ion. In some aspects, the metabolic reprogramming medium comprises at least about 55 mM potassium ion. In some aspects, the metabolic reprogramming medium comprises at least about 60 mM potassium ion. In some aspects, the metabolic reprogramming medium comprises at least about 65 mM potassium ion. In some aspects, the metabolic reprogramming medium comprises at least about 70 mM potassium ion. In some aspects, the metabolic reprogramming medium comprises at least about 75 mM potassium ion. In some aspects, the metabolic reprogramming medium comprises at least about 80 mM potassium ion. In some aspects, the MRM comprises between about 40 mM to about 80 mM potassium ion (e.g., between 40-80 mM).

In some aspects, the metabolic reprogramming medium comprises an increased concentration of potassium ion, e.g., at least about 5 mM potassium ion, and the medium is hypotonic. In some aspects, the metabolic reprogramming medium comprises potassium ion at a concentration between about 40 mM and about 80 mM and NaCl at a concentration between about 30 mM and about 100 mM, wherein the total concentration of potassium ion and NaCl is between about 110 and about 140 mM.

In some aspects, the concentration of potassium ion in a metabolic reprogramming medium of the present disclosure is about 5 mM to about 100 mM. In some aspects, the concentration of potassium ion in a metabolic reprogramming medium of the present disclosure is about 5 mM to about 100 mM, wherein the medium is hypotonic. In some aspects, the concentration of potassium ion in a metabolic reprogramming medium of the present disclosure is about 5 mM to about 90 mM, about 5 mM to about 80 mM, about 5 mM to about 70 mM, about 5 mM to about 60 mM, or about 5 mM to about 50 mM. In some aspects, the concentration of potassium ion in a metabolic reprogramming medium of the present disclosure is about 5 mM to about 90 mM, about 5 mM to about 80 mM, about 5 mM to about 70 mM, about 5 mM to about 60 mM, or about 5 mM to about 50 mM, wherein the medium is hypotonic. In some aspects, the concentration of potassium ion in a metabolic reprogramming medium of the present disclosure is about 25 mM to about 100 mM. In some aspects, the concentration of potassium ion in a metabolic reprogramming medium of the present disclosure is about 25 mM to about 100 mM, wherein the medium is hypotonic. In some aspects, the concentration of potassium ion in a metabolic reprogramming medium of the present disclosure is about 25 mM to about 90 mM, about 25 mM to about 80 mM, about 25 mM to about 70 mM, about 25 mM to about 60 mM, or about 25 mM to about 50 mM. In some aspects, the concentration of potassium ion in a metabolic reprogramming medium of the present disclosure is about 25 mM to about 90 mM, about 25 mM to about 80 mM, about 25 mM to about 70 mM, about 25 mM to about 60 mM, or about 25 mM to about 50 mM, wherein the medium is hypotonic. In some aspects, the concentration of potassium ion in a metabolic reprogramming medium of the present disclosure is about 40 mM to about 100 mM. In some aspects, the concentration of potassium ion in a metabolic reprogramming medium of the present disclosure is about 40 mM to about 100 mM, wherein the medium is hypotonic. In some aspects, the concentration of potassium ion is about 40 mM to about 90 mM, about 40 mM to about 85 mM, about 40 mM to about 80 mM, about 40 mM to about 75 mM, about 40 mM to about 70 mM, about 40 mM to about 65 mM, about 40 mM to about 60 mM, about 40 mM to about 55 mM, or about 40 mM to about 50 mM. In some aspects, the concentration of potassium ion is about 40 mM to about 90 mM, about 40 mM to about 85 mM, about 40 mM to about 80 mM, about 40 mM to about 75 mM, about 40 mM to about 70 mM, about 40 mM to about 65 mM, about 40 mM to about 60 mM, about 40 mM to about 55 mM, or about 40 mM to about 50 mM, wherein the medium is hypotonic. In some aspects, the concentration of potassium ion is between about 40 mM to about 80 mM, wherein the medium is hypotonic. In some aspects, the concentration of potassium ion is about 50 mM to about 90 mM, about 50 mM to about 85 mM, about 50 mM to about 80 mM, about 50 mM to about 75 mM, about 50 mM to about 70 mM, about 50 mM to about 65 mM, about 50 mM to about 60 mM, or about 50 mM to about 55 mM. In some aspects, the concentration of potassium ion is about 50 mM to about 90 mM, about 50 mM to about 85 mM, about 50 mM to about 80 mM, about 50 mM to about 75 mM, about 50 mM to about 70 mM, about 50 mM to about 65 mM, about 50 mM to about 60 mM, or about 50 mM to about 55 mM, and wherein the medium is hypotonic. In some aspects, the metabolic reprogramming medium comprises at least about 50 mM potassium ion and less than about 90 mM NaCl. In some aspects, the total concentration of potassium ion and NaCl is between 110 mM and 140 mM.

In some aspects, the concentration of potassium ion in a metabolic reprogramming medium of the present disclosure is about 50 mM to about 120 mM. In some aspects, the concentration of potassium ion is about 50 mM to about 115 mM, about 50 mM to about 110 mM, about 50 mM to about 105 mM, about 50 mM to about 100 mM, about 50 mM to about 95 mM, about 50 mM to about 90 mM, about 50 mM to about 85 mM, about 50 mM to about 80 mM, about 50 mM to about 75 mM, about 50 mM to about 70 mM, about 50 mM to about 65 mM, about 50 mM to about 60 mM, or about 50 mM to about 55 mM. In some aspects, the medium is hypotonic. In some aspects, the medium comprises at least about 50 mM to about 120 mM potassium ion and less than about 90 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 55 mM to about 120 mM. In some aspects, the concentration of potassium ion is about 55 mM to about 115 mM, about 55 mM to about 110 mM, about 55 mM to about 105 mM, about 55 mM to about 100 mM, about 55 mM to about 95 mM, about 55 mM to about 90 mM, about 55 mM to about 85 mM, about 55 mM to about 80 mM, about 55 mM to about 75 mM, about 55 mM to about 70 mM, about 55 mM to about 65 mM, or about 55 mM to about 60 mM. In some aspects, the medium is hypotonic. In some aspects, the metabolic reprogramming medium comprises at least about 55 mM to about 120 mM potassium ion and less than about 85 mM to about 20 mM NaCl. In some aspects, the total concentration of potassium ion and NaCl in a metabolic reprogramming medium of the present disclosure is between 110 mM and 140 mM.

In some aspects, the concentration of potassium ion in a metabolic reprogramming medium of the present disclosure is about 60 mM to about 120 mM. In some aspects, the concentration of potassium ion is about 60 mM to about 115 mM, about 60 mM to about 110 mM, about 60 mM to about 105 mM, about 60 mM to about 100 mM, about 60 mM to about 95 mM, about 60 mM to about 90 mM, about 60 mM to about 85 mM, about 60 mM to about 80 mM, about 60 mM to about 75 mM, about 60 mM to about 70 mM, or about 60 mM to about 65 mM. In some aspects, the medium is hypotonic. In some aspects, the metabolic reprogramming medium comprises at least about 60 mM to about 120 mM potassium ion and less than about 80 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 65 mM to about 120 mM. In some aspects, the concentration of potassium ion is about 65 mM to about 115 mM, about 65 mM to about 110 mM, about 65 mM to about 105 mM, about 65 mM to about 100 mM, about 65 mM to about 95 mM, about 65 mM to about 90 mM, about 65 mM to about 85 mM, about 65 mM to about 80 mM, about 65 mM to about 75 mM, or about 65 mM to about 70 mM. In some aspects, the medium is hypotonic. In some aspects, the metabolic reprogramming medium comprises at least about 65 mM to about 120 mM potassium ion and less than about 75 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 70 mM to about 120 mM. In some aspects, the concentration of potassium ion is about 70 mM to about 115 mM, about 70 mM to about 110 mM, about 70 mM to about 105 mM, about 70 mM to about 100 mM, about 70 mM to about 95 mM, about 70 mM to about 90 mM, about 70 mM to about 85 mM, about 70 mM to about 80 mM, or about 70 mM to about 75 mM. In some aspects, the medium is hypotonic. In some aspects, the metabolic reprogramming medium comprises at least about 70 mM to about 120 mM potassium ion and less than about 70 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 75 mM to about 120 mM. In some aspects, the concentration of potassium ion is about 75 mM to about 115 mM, about 75 mM to about 110 mM, about 75 mM to about 105 mM, about 75 mM to about 100 mM, about 75 mM to about 95 mM, about 75 mM to about 90 mM, about 75 mM to about 85 mM, or about 75 mM to about 80 mM. In some aspects, the medium is hypotonic. In some aspects, the metabolic reprogramming medium comprises at least about 75 mM to about 120 mM potassium ion and less than about 65 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 80 mM to about 120 mM. In some aspects, the concentration of potassium ion is about 80 mM to about 115 mM, about 80 mM to about 110 mM, about 80 mM to about 105 mM, about 80 mM to about 100 mM, about 80 mM to about 95 mM, about 80 mM to about 90 mM, or about 80 mM to about 85 mM. In some aspects, the medium is hypotonic. In some aspects, the metabolic reprogramming medium comprises at least about 80 mM to about 120 mM potassium ion and less than about 60 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 85 mM to about 120 mM. In some aspects, the concentration of potassium ion is about 85 mM to about 115 mM, about 85 mM to about 110 mM, about 85 mM to about 105 mM, about 85 mM to about 100 mM, about 85 mM to about 95 mM, or about 85 mM to about 90 mM. In some aspects, the medium is hypotonic. In some aspects, the metabolic reprogramming medium comprises at least about 85 mM to about 120 mM potassium ion and less than about 65 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 90 mM to about 120 mM. In some aspects, the concentration of potassium ion is about 90 mM to about 115 mM, about 90 mM to about 110 mM, about 90 mM to about 105 mM, about 90 mM to about 100 mM, or about 90 mM to about 95 mM. In some aspects, the medium is hypotonic. In some aspects, the metabolic reprogramming medium comprises at least about 90 mM to about 120 mM potassium ion and less than about 50 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 95 mM to about 120 mM. In some aspects, the concentration of potassium ion is about 95 mM to about 115 mM, about 95 mM to about 110 mM, about 95 mM to about 105 mM, or about 95 mM to about 100 mM. In some aspects, the medium is hypotonic. In some aspects, the metabolic reprogramming medium comprises at least about 95 mM to about 120 mM potassium ion and less than about 55 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 100 mM to about 120 mM. In some aspects, the concentration of potassium ion is about 100 mM to about 115 mM, about 100 mM to about 110 mM, or about 100 mM to about 105 mM. In some aspects, the medium is hypotonic. In some aspects, the metabolic reprogramming medium comprises at least about 100 mM to about 120 mM potassium ion and less than about 50 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 105 mM to about 120 mM. In some aspects, the concentration of potassium ion is about 105 mM to about 115 mM, or about 105 mM to about 110 mM. In some aspects, the medium is hypotonic. In some aspects, the metabolic reprogramming medium comprises at least about 105 mM to about 120 mM potassium ion and less than about 35 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 110 mM to about 120 mM. In some aspects, the concentration of potassium ion is about 110 mM to about 115 mM. In some aspects, the medium is hypotonic. In some aspects, the metabolic reprogramming medium comprises at least about 110 mM to about 120 mM potassium ion and less than about 30 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 50 mM to about 90 mM. In some aspects, the concentration of potassium ion is about 50 mM to about 80 mM. In some aspects, the concentration of potassium ion is about 60 mM to about 90 mM. In some aspects, the concentration of potassium ion is about 60 mM to about 80 mM. In some aspects, the concentration of potassium ion is about 70 mM to about 90 mM. In some aspects, the concentration of potassium ion is about 70 mM to about 80 mM. In some aspects, the concentration of potassium ion is about 80 mM to about 90 mM.

In some aspects, the concentration of potassium ion in a metabolic reprogramming medium of the present disclosure is about 50 mM to about 90 mM, and the concentration of NaCl is less than about 90 mM to about 50 mM. In some aspects, the concentration of potassium ion is about 50 mM to about 80 mM, and the concentration of NaCl is less than about 90 mM to about 60 mM. In some aspects, the concentration of potassium ion is about 60 mM to about 90 mM, and the concentration of NaCl is less than about 90 mM to about 60 mM. In some aspects, the concentration of potassium ion is about 60 mM to about 80 mM, and the concentration of NaCl is less than about 80 mM to about 60 mM. In some aspects, the concentration of potassium ion is about 70 mM to about 90 mM, and the concentration of NaCl is less than about 70 mM to about 50 mM. In some aspects, the concentration of potassium ion is about 70 mM to about 80 mM, and the concentration of NaCl is less than about 70 mM to about 60 mM. In some aspects, the concentration of potassium ion is about 80 mM to about 90 mM, and the concentration of NaCl is less than about 60 mM to about 50 mM. In some aspects, the total concentration of potassium ion and NaCl is between 110 mM and 140 mM.

In some aspects, the concentration of potassium ion in a metabolic reprogramming medium of the present disclosure is about 50 mM to about 55 mM. In some aspects, the concentration of potassium ion is about 50 mM to about 55 mM, and the concentration of NaCl is less than about 90 to about 85. In some aspects, the concentration of potassium ion is about 55 mM to about 60 mM. In some aspects, the concentration of potassium ion is about 55 mM to about 60 mM, and the concentration of NaCl is less than about 85 to about 80. In some aspects, the concentration of potassium ion is about 60 mM to about 65 mM. In some aspects, the concentration of potassium ion is about 60 mM to about 65 mM, and the concentration of NaCl is less than about 80 mM to about 75 mM. In some aspects, the concentration of potassium ion is about 65 mM to about 70 mM. In some aspects, the concentration of potassium ion is about 65 mM to about 70 mM, and the concentration of NaCl is less than about 75 mM to about 70 mM. In some aspects, the concentration of potassium ion is about 70 mM to about 75 mM. In some aspects, the concentration of potassium ion is about 70 mM to about 75 mM, and the concentration of NaCl is less than about 70 mM to about 65 mM. In some aspects, the concentration of potassium ion is about 75 mM to about 80 mM. In some aspects, the concentration of potassium ion is about 75 mM to about 80 mM, and the concentration of NaCl is less than about 65 mM to about 60 mM. In some aspects, the concentration of potassium ion is about 80 mM to about 85 mM. In some aspects, the concentration of potassium ion is about 80 mM to about 85 mM, and the concentration of NaCl is less than about 60 mM to about 55 mM. In some aspects, the concentration of potassium ion is about 85 mM to about 90 mM. In some aspects, the concentration of potassium ion is about 85 mM to about 90 mM, and the concentration of NaCl is less than about 55 mM to about 50 mM. In some aspects, the concentration of potassium ion is about 90 mM to about 95 mM. In some aspects, the concentration of potassium ion is about 90 mM to about 95 mM, and the concentration of NaCl is less than about 50 to about 45. In some aspects, the concentration of potassium ion is about 95 mM to about 100 mM. In some aspects, the concentration of potassium ion is about 95 mM to about 100 mM, and the concentration of NaCl is less than about 45 mM to about 40 mM. In some aspects, the concentration of potassium ion is about 100 mM to about 105 mM. In some aspects, the concentration of potassium ion is about 100 mM to about 105 mM, and the concentration of NaCl is less than about 40 mM to about 35 mM. In some aspects, the concentration of potassium ion is about 105 mM to about 110 mM. In some aspects, the concentration of potassium ion is about 105 mM to about 110 mM, and the concentration of NaCl is less than about 35 to about 30. In some aspects, the concentration of potassium ion is about 110 mM to about 115 mM. In some aspects, the concentration of potassium ion is about 110 mM to about 115 mM, and the concentration of NaCl is less than about 30 mM to about 25 mM. In some aspects, the concentration of potassium ion is about 115 mM to about 120 mM. In some aspects, the concentration of potassium ion is about 115 mM to about 120 mM, and the concentration of NaCl is less than about 25 mM to about 20 mM. In some aspects, the total concentration of potassium ion and NaCl is between 110 mM and 140 mM.

In some aspects, the concentration of potassium ion is about 40 mM to about 90 mM, wherein the medium is hypotonic or isotonic. In some aspects, the concentration of potassium ion is about 40 mM to about 80 mM, wherein the medium is hypotonic or isotonic. In some aspects, the concentration of potassium ion is about 40 mM to about 70 mM, wherein the medium is hypotonic or isotonic. In some aspects, the concentration of potassium ion is about 50 mM to about 90 mM, wherein the medium is hypotonic or isotonic. In some aspects, the concentration of potassium ion is about 50 mM to about 80 mM, wherein the medium is hypotonic or isotonic. In some aspects, the concentration of potassium ion is about 50 mM to about 70 mM, wherein the medium is hypotonic or isotonic. In some aspects, the concentration of potassium ion is about 55 mM to about 90 mM, wherein the medium is hypotonic or isotonic. In some aspects, the concentration of potassium ion is about 55 mM to about 80 mM, wherein the medium is hypotonic or isotonic. In some aspects, the concentration of potassium ion is about 55 mM to about 70 mM, wherein the medium is hypotonic or isotonic. In some aspects, the concentration of potassium ion is about 60 mM to about 90 mM, wherein the medium is hypotonic or isotonic. In some aspects, the concentration of potassium ion is about 60 mM to about 80 mM, wherein the medium is hypotonic or isotonic. In some aspects, the concentration of potassium ion is about 60 mM to about 70 mM, wherein the medium is hypotonic or isotonic. In some aspects, the concentration of potassium ion is about 65 mM to about 90 mM, wherein the medium is hypotonic or isotonic. In some aspects, the concentration of potassium ion is about 65 mM to about 80 mM, wherein the medium is hypotonic or isotonic. In some aspects, the concentration of potassium ion is about 65 mM to about 70 mM, wherein the medium is hypotonic or isotonic.

In some aspects, the concentration of potassium ion is higher than about 4 mM, wherein the medium is hypotonic or isotonic. In some aspects, the concentration of potassium ion is about 4 mM, wherein the medium is hypotonic or isotonic. In some aspects, the concentration of potassium ion is higher than about 5 mM, wherein the medium is hypotonic or isotonic. In some aspects, the concentration of potassium ion is about 5 mM, wherein the medium is hypotonic or isotonic. In some aspects, the concentration of potassium ion is higher than about 6 mM, wherein the medium is hypotonic or isotonic. In some aspects, the concentration of potassium ion is about 6 mM, wherein the medium is hypotonic or isotonic. In some aspects, the concentration of potassium ion is higher than about 7 mM, wherein the medium is hypotonic or isotonic. In some aspects, the concentration of potassium ion is about 7 mM, wherein the medium is hypotonic or isotonic. In some aspects, the concentration of potassium ion is higher than about 8 mM, wherein the medium is hypotonic or isotonic. In some aspects, the concentration of potassium ion is about 8 mM, wherein the medium is hypotonic or isotonic. In some aspects, the concentration of potassium ion is higher than about 9 mM, wherein the medium is hypotonic or isotonic. In some aspects, the concentration of potassium ion is about 9 mM, wherein the medium is hypotonic or isotonic.

In some aspects, the concentration of potassium ion is higher than about 10 mM, wherein the medium is hypotonic or isotonic. In some aspects, the concentration of potassium ion is about 10 mM, wherein the medium is hypotonic or isotonic. In some aspects, the concentration of potassium ion is higher than about 11 mM, wherein the medium is hypotonic or isotonic. In some aspects, the concentration of potassium ion is about 11 mM, wherein the medium is hypotonic or isotonic. In some aspects, the concentration of potassium ion is higher than about 12 mM, wherein the medium is hypotonic or isotonic. In some aspects, the concentration of potassium ion is about 12 mM, wherein the medium is hypotonic or isotonic. In some aspects, the concentration of potassium ion is higher than about 13 mM, wherein the medium is hypotonic or isotonic. In some aspects, the concentration of potassium ion is about 13 mM, wherein the medium is hypotonic. In some aspects, the concentration of potassium ion is higher than about 14 mM, wherein the medium is hypotonic or isotonic. In some aspects, the concentration of potassium ion is about 14 mM, wherein the medium is hypotonic or isotonic. In some aspects, the concentration of potassium ion is higher than about 15 mM, wherein the medium is hypotonic or isotonic. In some aspects, the concentration of potassium ion is about 15 mM, wherein the medium is hypotonic or isotonic. In some aspects, the concentration of potassium ion is higher than about 16 mM, wherein the medium is hypotonic or isotonic. In some aspects, the concentration of potassium ion is about 16 mM, wherein the medium is hypotonic or isotonic. In some aspects, the concentration of potassium ion is higher than about 17 mM, wherein the medium is hypotonic or isotonic. In some aspects, the concentration of potassium ion is about 17 mM, wherein the medium is hypotonic or isotonic. In some aspects, the concentration of potassium ion is higher than about 18 mM, wherein the medium is hypotonic or isotonic. In some aspects, the concentration of potassium ion is about 18 mM, wherein the medium is hypotonic or isotonic. In some aspects, the concentration of potassium ion is higher than about 19 mM, wherein the medium is hypotonic or isotonic. In some aspects, the concentration of potassium ion is about 19 mM, wherein the medium is hypotonic or isotonic.

In some aspects, the concentration of potassium ion is higher than about 20 mM, wherein the medium is hypotonic or isotonic. In some aspects, the concentration of potassium ion is about 20 mM, wherein the medium is hypotonic or isotonic. In some aspects, the concentration of potassium ion is higher than about 21 mM, wherein the medium is hypotonic or isotonic. In some aspects, the concentration of potassium ion is about 21 mM, wherein the medium is hypotonic or isotonic. In some aspects, the concentration of potassium ion is higher than about 22 mM, wherein the medium is hypotonic or isotonic. In some aspects, the concentration of potassium ion is about 22 mM, wherein the medium is hypotonic or isotonic. In some aspects, the concentration of potassium ion is higher than about 23 mM, wherein the medium is hypotonic or isotonic. In some aspects, the concentration of potassium ion is about 23 mM, wherein the medium is hypotonic. In some aspects, the concentration of potassium ion is higher than about 24 mM, wherein the medium is hypotonic or isotonic. In some aspects, the concentration of potassium ion is about 24 mM, wherein the medium is hypotonic or isotonic. In some aspects, the concentration of potassium ion is higher than about 25 mM, wherein the medium is hypotonic or isotonic. In some aspects, the concentration of potassium ion is about 25 mM, wherein the medium is hypotonic or isotonic. In some aspects, the concentration of potassium ion is higher than about 26 mM, wherein the medium is hypotonic or isotonic. In some aspects, the concentration of potassium ion is about 26 mM, wherein the medium is hypotonic or isotonic. In some aspects, the concentration of potassium ion is higher than about 27 mM, wherein the medium is hypotonic or isotonic. In some aspects, the concentration of potassium ion is about 27 mM, wherein the medium is hypotonic or isotonic. In some aspects, the concentration of potassium ion is higher than about 28 mM, wherein the medium is hypotonic or isotonic. In some aspects, the concentration of potassium ion is about 28 mM, wherein the medium is hypotonic or isotonic. In some aspects, the concentration of potassium ion is higher than about 29 mM, wherein the medium is hypotonic or isotonic. In some aspects, the concentration of potassium ion is about 29 mM, wherein the medium is hypotonic or isotonic.

In some aspects, the concentration of potassium ion is higher than about 30 mM, wherein the medium is hypotonic or isotonic. In some aspects, the concentration of potassium ion is about 30 mM, wherein the medium is hypotonic or isotonic. In some aspects, the concentration of potassium ion is higher than about 31 mM, wherein the medium is hypotonic or isotonic. In some aspects, the concentration of potassium ion is about 31 mM, wherein the medium is hypotonic or isotonic. In some aspects, the concentration of potassium ion is higher than about 32 mM, wherein the medium is hypotonic or isotonic. In some aspects, the concentration of potassium ion is about 32 mM, wherein the medium is hypotonic or isotonic. In some aspects, the concentration of potassium ion is higher than about 33 mM, wherein the medium is hypotonic or isotonic. In some aspects, the concentration of potassium ion is about 33 mM, wherein the medium is hypotonic. In some aspects, the concentration of potassium ion is higher than about 34 mM, wherein the medium is hypotonic or isotonic. In some aspects, the concentration of potassium ion is about 34 mM, wherein the medium is hypotonic or isotonic. In some aspects, the concentration of potassium ion is higher than about 35 mM, wherein the medium is hypotonic or isotonic. In some aspects, the concentration of potassium ion is about 35 mM, wherein the medium is hypotonic or isotonic. In some aspects, the concentration of potassium ion is higher than about 36 mM, wherein the medium is hypotonic or isotonic. In some aspects, the concentration of potassium ion is about 36 mM, wherein the medium is hypotonic or isotonic. In some aspects, the concentration of potassium ion is higher than about 37 mM, wherein the medium is hypotonic or isotonic. In some aspects, the concentration of potassium ion is about 37 mM, wherein the medium is hypotonic or isotonic. In some aspects, the concentration of potassium ion is higher than about 38 mM, wherein the medium is hypotonic or isotonic. In some aspects, the concentration of potassium ion is about 38 mM, wherein the medium is hypotonic or isotonic. In some aspects, the concentration of potassium ion is higher than about 39 mM, wherein the medium is hypotonic or isotonic. In some aspects, the concentration of potassium ion is about 39 mM, wherein the medium is hypotonic or isotonic.

In some aspects, the concentration of potassium ion is higher than about 40 mM, wherein the medium is hypotonic or isotonic. In some aspects, the concentration of potassium ion is about 40 mM, wherein the medium is hypotonic or isotonic. In some aspects, the concentration of potassium ion is higher than about 41 mM, wherein the medium is hypotonic or isotonic. In some aspects, the concentration of potassium ion is about 41 mM, wherein the medium is hypotonic or isotonic. In some aspects, the concentration of potassium ion is higher than about 42 mM, wherein the medium is hypotonic or isotonic. In some aspects, the concentration of potassium ion is about 42 mM, wherein the medium is hypotonic or isotonic. In some aspects, the concentration of potassium ion is higher than about 43 mM, wherein the medium is hypotonic or isotonic. In some aspects, the concentration of potassium ion is about 43 mM, wherein the medium is hypotonic. In some aspects, the concentration of potassium ion is higher than about 44 mM, wherein the medium is hypotonic or isotonic. In some aspects, the concentration of potassium ion is about 44 mM, wherein the medium is hypotonic or isotonic. In some aspects, the concentration of potassium ion is higher than about 45 mM, wherein the medium is hypotonic or isotonic. In some aspects, the concentration of potassium ion is about 45 mM, wherein the medium is hypotonic or isotonic. In some aspects, the concentration of potassium ion is higher than about 46 mM, wherein the medium is hypotonic or isotonic. In some aspects, the concentration of potassium ion is about 46 mM, wherein the medium is hypotonic or isotonic. In some aspects, the concentration of potassium ion is higher than about 47 mM, wherein the medium is hypotonic or isotonic. In some aspects, the concentration of potassium ion is about 47 mM, wherein the medium is hypotonic or isotonic. In some aspects, the concentration of potassium ion is higher than about 48 mM, wherein the medium is hypotonic or isotonic. In some aspects, the concentration of potassium ion is about 48 mM, wherein the medium is hypotonic or isotonic. In some aspects, the concentration of potassium ion is higher than about 49 mM, wherein the medium is hypotonic or isotonic. In some aspects, the concentration of potassium ion is about 49 mM, wherein the medium is hypotonic or isotonic.

In some aspects, the metabolic reprogramming medium comprising a high concentration of potassium ion is prepared by adding a sufficient amount of a potassium salt in a medium. In some aspects, non-limiting examples of potassium salt include potassium aminetrichloroplatinate, potassium aquapentachlororuthenate, potassium bis(oxalato)platinate(II) dihydrate, potassium bisulfate, potassium borohydride, potassium bromide, potassium carbonate, potassium chloride, potassium chromate, potassium dichromate, potassium dicyanoargentate, potassium dicyanoaurate, potassium fluoride, potassium fluorosulfate, potassium hexachloroiridate, potassium hexachloroosmate, potassium hexachloropalladate, potassium hexachloroplatinate, potassium hexachlororhenate, potassium hexacyanochromate, potassium hexacyanoferrate, potassium hexacyanoruthenate(II) hydrate, potassium hexafluoroantimonate, potassium hexafluoronickelate, potassium hexafluorophosphate, potassium hexafluorotitanate, potassium hexafluorozirconate, potassium hexahydroxoantimonate, potassium hexaiodoplatinate, potassium hexaiodorhenate, potassium hydroxide, potassium iodate, potassium iodide, potassium manganate, potassium metavanadate, potassium molybdate, potassium nitrate, potassium nitrosodisulfonate, potassium osmate(VI) dihydrate, potassium pentachloronitrosylruthenate, potassium perchlorate, potassium perrhenate, potassium perruthenate, potassium persulfate, potassium phosphate dibasic, potassium phosphate monobasic, potassium pyrophosphate, potassium selenocyanate, potassium selenocyanate, potassium stannate trihydrate, potassium sulfate, potassium tellurate hydrate, potassium tellurite, potassium tetraborate tetrahydrate, potassium tetrabromoaurate, potassium tetrabromopalladate, potassium tetrachloropalladate, potassium tetrachloroplatinate, potassium tetracyanopalladate, potassium tetracyanoplatinate, potassium tetrafluoroborate, potassium tetranitroplatinate, potassium tetrathionate, potassium p-toluenethiosulfonate, potassium hydroxycitrate tribasic monohydrate, or any combination thereof. In certain aspects, the potassium salt comprises potassium chloride (KCl). In certain aspects, the potassium salt comprises potassium gluconate. In certain aspects, the potassium salt comprises potassium citrate. In certain aspects, the potassium salt comprises potassium hydroxycitrate.

II.A.2. Sodium

Some aspects of the present disclosure are directed to methods of culturing immune cells in a medium comprising (i) potassium ion at a concentration of at least about 5 mM (e.g., higher than 5 mM, e.g., between about 40 mM and about 80 mM) and (ii) sodium ion (e.g., NaCl) at a concentration of less than about 115 mM. In some aspects, the medium is hypotonic or isotonic. In some aspects, the target concentration of sodium (e.g., NaCl) is reached by starting with a basal medium comprising a higher concentration of sodium ion (e.g., NaCl), and diluting the solution to reach the target concentration of sodium ion (e.g., NaCl). In some aspects, the target concentration of sodium ion (e.g., NaCl) is reached by adding one or more sodium salts (e.g., more NaCl). Non-limiting examples of sodium salts include sodium (meta)periodate, sodium arsenyl tartrate hydrate, sodium azide, sodium benzyloxide, sodium bromide, sodium carbonate, sodium chloride, sodium chromate, sodium cyclohexanebutyrate, sodium ethanethiolate, sodium fluoride, sodium fluorophosphate, sodium formate, sodium hexachloroiridate(III) hydrate, sodium hexachloroiridate(IV) hexahydrate, sodium hexachloroplatinate(IV) hexahydrate, sodium hexachlororhodate(III), sodium hexafluoroaluminate, sodium hexafluoroantimonate(V), sodium hexafluoroarsenate(V), sodium hexafluoroferrate(III), sodium hexafluorophosphate, sodium hexafluorosilicate, sodium hexahydroxyplatinate(IV), sodium hexametaphosphate, sodium hydrogen difluoride, sodium hydrogen sulfate, sodium hydrogencyanamide, sodium hydroxide, sodium iodide, sodium metaborate tetrahydrate, sodium metasilicate nonahydrate, sodium metavanadate, sodium molybdate, sodium nitrate, sodium nitrite, sodium oxalate, sodium perborate monohydrate, sodium percarbonate, sodium perchlorate, sodium periodate, sodium permanganate, sodium perrhenate, sodium phosphate, sodium pyrophosphate, sodium selenate, sodium selenite, sodium stannate, sodium sulfate, sodium tellurite, sodium tetraborate, sodium tetrachloroaluminate, sodium tetrachloroaurate(III), sodium tetrachloropalladate(II), sodium tetrachloroplatinate(II), sodium thiophosphate tribasic, sodium thiosulfate, sodium thiosulfate pentahydrate, sodium yttrium oxyfluoride, Trisodium trimetaphosphate, or any combination thereof. In some aspects, the sodium salt comprises sodium chloride (NaCl). In some aspects, the sodium salt comprises sodium gluconate. In some aspects, the sodium salt comprises sodium bicarbonate. In some aspects, the sodium salt comprises sodium hydroxycitrate. In some aspects, the sodium salt comprises sodium phosphate.

In some aspects, the concentration of the sodium ion (e.g., NaCl) in a metabolic reprogramming medium of the present disclosure is less than that of the basal medium. In some aspects, the concentration of the sodium ion (e.g., NaCl) is reduced as the concentration of potassium ion is increased. In some aspects, the concentration of the sodium ion (e.g., NaCl) is from about 25 mM to about 115 mM. In some aspects, the concentration of the sodium (e.g., NaCl) ion is from about 25 mM to about 100 mM, about 30 mM to about 40 mM, about 30 mM to about 50 mM, about 30 mM to about 60 mM, about 30 mM to about 70 mM, about 30 mM to about 80 mM, about 40 mM to about 50 mM, about 40 mM to about 60 mM, about 40 mM to about 70 mM, about 40 mM to about 80 mM, about 50 mM to about 55 mM, about 50 mM to about 60 mM, about 50 mM to about 65 mM, about 50 mM to about 70 mM, about 50 mM to about 75 mM, about 50 mM to about 80 mM, about 55 mM to about 60 mM, about 55 mM to about 65 mM, about 55 mM to about 70 mM, about 55 mM to about 75 mM, about 55 mM to about 80 mM, about 60 mM to about 65 mM, about 60 mM to about 70 mM, about 60 mM to about 75 mM, about 60 mM to about 80 mM, about 70 mM to about 75 mM, about 70 mM to about 80 mM, or about 75 mM to about 80 mM. In some aspects, the concentration of the sodium ion (e.g., NaCl) is from about 40 mM to about 80 mM. In some aspects, the concentration of the sodium ion (e.g., NaCl) is from about 50 mM to about 85 mM. In some aspects, the concentration of the sodium ion (e.g., NaCl) is from about 55 mM to about 80 mM. In some aspects, the concentration of the sodium ion (e.g., NaCl) is from about 30 mM to about 35 mM. In some aspects, the concentration of the sodium ion (e.g., NaCl) is from about 35 mM to about 40 mM. In some aspects, the concentration of the sodium ion (e.g., NaCl) is from about 40 mM to about 45 mM. In some aspects, the concentration of the sodium ion (e.g., NaCl) is from about 45 mM to about 50 mM. In some aspects, the concentration of the sodium ion (e.g., NaCl) is from about 50 mM to about 55 mM. In some aspects, the concentration of the sodium ion (e.g., NaCl) is from about 55 mM to about 60 mM. In some aspects, the concentration of the sodium ion (e.g., NaCl) is from about 60 mM to about 65 mM. In some aspects, the concentration of the sodium ion (e.g., NaCl) is from about 65 mM to about 70 mM. In some aspects, the concentration of the sodium ion (e.g., NaCl) is from about 70 mM to about 75 mM. In some aspects, the concentration of the sodium ion (e.g., NaCl) is from about 75 mM to about 80 mM. In some aspects, the concentration of the sodium ion (e.g., NaCl) is from about 80 mM to about 85 mM.

In some aspects, the concentration of the sodium ion (e.g., NaCl) is about 30 mM, about 35 mM, about 40 mM, about 45 mM, about 50 mM, about 55 mM, about 60 mM, about 65 mM, about 70 mM, about 75 mM, about 80 mM, about 85 mM, or about 90 mM. In certain aspects, the concentration of sodium ion (e.g., NaCl) is about 40 mM. In some aspects, the concentration of sodium ion (e.g., NaCl) is about 45 mM. In some aspects, the concentration of sodium ion (e.g., NaCl) is about 50 mM. In some aspects, the concentration of sodium ion (e.g., NaCl) is about 55 mM. In some aspects, the concentration of sodium ion (e.g., NaCl) is about 55.6 mM. In some aspects, the concentration of sodium ion (e.g., NaCl) is about 59.3 mM. In some aspects, the concentration of sodium ion (e.g., NaCl) is about 60 mM. In some aspects, the concentration of sodium ion (e.g., NaCl) is about 63.9 mM. In some aspects, the concentration of sodium ion (e.g., NaCl) is about 65 mM. In some aspects, the concentration of sodium ion (e.g., NaCl) is about 67.6 mM. In some aspects, the concentration of sodium ion (e.g., NaCl) is about 70 mM. In some aspects, the concentration of sodium ion (e.g., NaCl) is about 72.2 mM. In some aspects, the concentration of sodium ion (e.g., NaCl) is about 75 mM. In some aspects, the concentration of sodium ion (e.g., NaCl) is about 76 mM. In some aspects, the concentration of sodium ion (e.g., NaCl) is about 80 mM. In some aspects, the concentration of sodium ion (e.g., NaCl) is about 80.5 mM. In some aspects, the metabolic reprogramming medium comprises about 40 mM to about 90 mM potassium ion and about 40 mM to about 80 mM sodium ion (e.g., NaCl).

In some aspects, the metabolic reprogramming medium comprises about 50 mM to about 75 mM potassium ion and about 80 mM to about 90 mM sodium ion (e.g., NaCl). In some aspects, the metabolic reprogramming medium comprises about 55 mM to about 75 mM potassium ion and about 80 mM to about 90 mM sodium ion (e.g., NaCl). In some aspects, the metabolic reprogramming medium comprises about 60 mM to about 75 mM potassium ion and about 80 mM to about 90 mM sodium ion (e.g., NaCl). In some aspects, the metabolic reprogramming medium comprises about 65 mM to about 75 mM potassium ion and about 80 mM to about 85 mM sodium ion (e.g., NaCl). In some aspects, the metabolic reprogramming medium comprises about 65 mM potassium ion and about 80 mM to about 85 mM sodium ion (e.g., NaCl). In some aspects, the metabolic reprogramming medium comprises about 66 mM potassium ion and about 80 mM to about 85 mM sodium ion (e.g., NaCl). In some aspects, the metabolic reprogramming medium comprises about 67 mM potassium ion and about 80 mM to about 85 mM sodium ion (e.g., NaCl). In some aspects, the metabolic reprogramming medium comprises about 68 mM potassium ion and about 80 mM to about 85 mM sodium ion (e.g., NaCl). In some aspects, the metabolic reprogramming medium comprises about 69 mM potassium ion and about 80 mM to about 85 mM sodium ion (e.g., NaCl). In some aspects, the metabolic reprogramming medium comprises about 70 mM potassium ion and about 80 mM to about 85 mM sodium ion (e.g., NaCl). In some aspects, the metabolic reprogramming medium comprises about 71 mM potassium ion and about 80 mM to about 85 mM sodium ion (e.g., NaCl). In some aspects, the metabolic reprogramming medium comprises about 72 mM potassium ion and about 80 mM to about 85 mM sodium ion (e.g., NaCl). In some aspects, the metabolic reprogramming medium comprises about 73 mM potassium ion and about 80 mM to about 85 mM sodium ion (e.g., NaCl). In some aspects, the metabolic reprogramming medium comprises about 74 mM potassium ion and about 80 mM to about 85 mM sodium ion (e.g., NaCl). In some aspects, the metabolic reprogramming medium comprises about 75 mM potassium ion and about 80 mM to about 85 mM sodium ion (e.g., NaCl). In some aspects, the metabolic reprogramming medium comprises about 65 mM potassium ion and about 80 mM sodium ion (e.g., NaCl). In some aspects, the metabolic reprogramming medium comprises about 65 mM potassium ion and about 85 mM sodium ion (e.g., NaCl). In some aspects, the metabolic reprogramming medium comprises about 65 mM potassium ion and about 90 mM sodium ion (e.g., NaCl). In some aspects, the metabolic reprogramming medium comprises about 70 mM potassium ion and about 80 mM sodium ion (e.g., NaCl). In some aspects, the metabolic reprogramming medium comprises about 70 mM potassium ion and about 85 mM sodium ion (e.g., NaCl). In some aspects, the metabolic reprogramming medium comprises about 70 mM potassium ion and about 90 mM sodium ion (e.g., NaCl). In some aspects, the metabolic reprogramming medium comprises about 75 mM potassium ion and about 80 mM sodium ion (e.g., NaCl). In some aspects, the metabolic reprogramming medium comprises about 75 mM potassium ion and about 85 mM sodium ion (e.g., NaCl). In some aspects, the metabolic reprogramming medium comprises about 75 mM potassium ion and about 90 mM sodium ion (e.g., NaCl).

In some aspects, the metabolic reprogramming medium comprises about 40 mM to about 90 mM potassium ion and about 30 mM to about 109 mM NaCl, wherein the concentration of NaCl (mM) is equal to or lower than (135−potassium ion concentration, meaning 135 minus the concentration of potassium ion). In some aspects, the metabolic reprogramming medium comprises about 40 mM potassium ion and less than or equal to about 95 mM NaCl (e.g., about 95 mM, about 94 mM, about 93 mM, about 92 mM, about 91 mM, about 90 mM, about 85 mM, about 80 mM, about 75 mM, about 70 mM, about 65 mM, about 60 mM, about 55 mM, or about 50 mM NaCl). In some aspects, the metabolic reprogramming medium comprises about 45 mM potassium ion and less than or equal to about 90 mM NaCl (e.g., about 90 mM, about 89 mM, about 88 mM, about 87 mM, about 86 mM, about 85 mM, about 80 mM, about 75 mM, about 70 mM, about 65 mM, about 60 mM, about 55 mM, or about 50 mM NaCl). In some aspects, the metabolic reprogramming medium comprises about 50 mM potassium ion and less than or equal to about 85 mM NaCl (e.g., about 85 mM, about 84 mM, about 83 mM, about 82 mM, about 81 mM, about 80 mM, about 75 mM, about 70 mM, about 65 mM, about 60 mM, about 55 mM, or about 50 mM NaCl). In some aspects, the metabolic reprogramming medium comprises about 55 mM potassium ion and less than or equal to about 80 mM NaCl (e.g., about 80 mM, about 79 mM, about 78 mM, about 77 mM, about 76 mM, about 75 mM, about 70 mM, about 65 mM, about 60 mM, about 55 mM, or about 50 mM NaCl). In some aspects, the metabolic reprogramming medium comprises about 60 mM potassium ion and less than or equal to about 75 mM NaCl (e.g., about 75 mM, about 74 mM, about 73 mM, about 72 mM, about 71 mM, about 70 mM, about 65 mM, about 60 mM, about 55 mM, or about 50 mM NaCl). In some aspects, the metabolic reprogramming medium comprises about 65 mM potassium ion and less than or equal to about 70 mM NaCl (e.g., about 70 mM, about 69 mM, about 68 mM, about 67 mM, about 66 mM, about 65 mM, about 60 mM, about 55 mM, or about 50 mM NaCl). In some aspects, the metabolic reprogramming medium comprises about 70 mM potassium ion and less than or equal to about 70 mM NaCl (e.g., about 65 mM, about 64 mM, about 63 mM, about 62 mM, about 61 mM, about 60 mM, about 55 mM, or about 50 mM NaCl). In some aspects, the metabolic reprogramming medium comprises about 75 mM potassium ion and less than or equal to about 60 mM NaCl (e.g., about 60 mM, about 59 mM, about 58 mM, about 57 mM, about 56 mM, about 55 mM, about 50 mM, about 45 mM, or about 40 mM NaCl). In some aspects, the metabolic reprogramming medium comprises about 80 mM potassium ion and less than or equal to about 55 mM NaCl (e.g., about 55 mM, about 54 mM, about 53 mM, about 52 mM, about 51 mM, about 50 mM, about 45 mM, about 40 mM, or about 35 mM NaCl). In some aspects, the metabolic reprogramming medium comprises about 85 mM potassium ion and less than or equal to about 50 mM NaCl (e.g., about 50 mM, about 49 mM, about 48 mM, about 47 mM, about 46 mM, about 45 mM, about 40 mM, about 35 mM, or about 30 mM NaCl). In some aspects, the metabolic reprogramming medium comprises about 90 mM potassium ion and less than or equal to about 45 mM NaCl (e.g., about 45 mM, about 44 mM, about 43 mM, about 42 mM, about 41 mM, about 40 mM, about 35 mM, about 30 mM, or about 25 mM NaCl). In some aspects, the metabolic reprogramming medium comprises about 70 mM potassium ion and about 60 mM NaCl. In some aspects, the metabolic reprogramming medium comprises about 70 mM potassium ion and about 61 mM NaCl. In some aspects, the metabolic reprogramming medium comprises about 70 mM potassium ion and about 62 mM NaCl.

In some aspects, the medium comprises about 50 mM potassium ion and about 75 mM NaCl. In some aspects, the medium is hypotonic. In some aspects, the medium is isotonic.

Some aspects of the present disclosure are directed to methods of culturing immune cells (e.g., T cells and/or NK cells) in a medium comprising (i) potassium ion at a concentration higher than 5 mM and (ii) NaCl at a concentration of less than about 135 mM. Some aspects of the present disclosure are directed to methods of culturing immune cells, e.g., T cells and/or NK cells, in a medium comprising (i) potassium ion at a concentration higher than 40 mM and (ii) NaCl at a concentration of less than about 100 mM. Some aspects of the present disclosure are directed to methods of culturing immune cells, e.g., T cells and/or NK cells, in a medium comprising (i) potassium ion at a concentration higher than 50 mM and (ii) NaCl at a concentration of less than about 90 mM. Some aspects of the present disclosure are directed to methods of culturing immune cells, e.g., T cells and/or NK cells, in a medium comprising (i) potassium ion at a concentration higher than 55 mM and (ii) NaCl at a concentration of less than about 70 mM. Some aspects of the present disclosure are directed to methods of culturing immune cells, e.g., T cells and/or NK cells, in a medium comprising (i) potassium ion at a concentration higher than 60 mM and (ii) NaCl at a concentration of less than about 70 mM. Some aspects of the present disclosure are directed to methods of culturing immune cells (e.g., T cells and/or NK cells) in a medium comprising (i) potassium ion at a concentration between about 40 mM to about 80 mM and (ii) NaCl at a concentration between about 40 mM to about 80 mM. Some aspects of the present disclosure are directed to methods of culturing immune cells (e.g., T cells and/or NK cells) in a medium comprising (i) potassium ion at a concentration between about 40 mM to about 80 mM and (ii) NaCl at a concentration between about 55 mM to about 90 mM.

II.A.3. Tonicity

In some aspects of the present disclosure, the tonicity of the metabolic reprogramming medium (e.g., (concentration of potassium ion and concentration of NaCl)×2) is adjusted based on the concentration of potassium ion and/or NaCl. In some aspects, the tonicity of the metabolic reprogramming medium is lower than that of the basal medium. In some aspects, the tonicity of the metabolic reprogramming medium is higher than that of the basal medium. In some aspect, the tonicity of the medium is the same as that of the basal medium. The tonicity of the metabolic reprogramming medium can be affected by modifying the concentration of potassium ion and/or NaCl in the media. In some aspects, increased potassium ion concentration is paired with an increase or a decrease in the concentration of NaCl. In some aspects, this pairing affects the tonicity of the metabolic reprogramming medium. In some aspects, the concentration of potassium ion is increased while the concentration of NaCl, is decreased.

In some aspects, the medium useful for the present media is prepared based on the function of potassium ion and tonicity. For example, in some aspects, if the medium useful for the present disclosure is hypotonic (e.g., less than 280 mOsm) and comprises at least about 50 mM of potassium ion, a concentration of NaCl that is sufficient to maintain the medium as hypotonic can be determined based on the following formula: NaCl concentration=(desired tonicity (280)/2)−potassium ion concentration. (i.e., the concentration of NaCl (mM) is equal to or lower than (140−potassium ion concentration)). In some aspects, a hypotonic medium disclosed herein comprises a total concentration of potassium ion and NaCl between 110 mM and 140 mM. Therefore, for hypotonic medium, the concentration of potassium ion can be set at a concentration between 50 mM and 90 mM, and the NaCl concentration can be between 90 mM and 50 mM, or lower, so long as the total concentration of potassium ion and NaCl is between 110 mM and 140 mM. In some aspects, a hypotonic medium disclosed herein comprises a total concentration of potassium ion and NaCl between 115 mM and 140 mM. In some aspects, the hypotonic medium disclosed herein comprises a total concentration of potassium ion and NaCl between 120 mM and 140 mM.

In some aspects, the metabolic reprogramming medium is isotonic (between 280 mOsm and 300 mOsm) and comprises a concentration of potassium ion between about 50 mM and 70 mM. The corresponding concentration of NaCl can be again calculated based on the formula: NaCl concentration=(desired tonicity/2)−potassium ion concentration. For example, if the concentration of potassium is 50 mM and the desired tonicity is 300 mOsm, the NaCl concentration can be 100 mM.

In some aspects, the metabolic reprogramming medium is isotonic. In some aspects, the metabolic reprogramming medium has a tonicity of about 280 mOsm/L. In some aspects, the metabolic reprogramming medium has a tonicity of 280 mOsm/L. In some aspects, the metabolic reprogramming medium has a tonicity of 280 mOsm/L±1 mOsm/L. In some aspects, the metabolic reprogramming medium has a tonicity of 280 mOsm/L±2 mOsm/L. In some aspects, the metabolic reprogramming medium has a tonicity of 280 mOsm/L±3 mOsm/L. In some aspects, the metabolic reprogramming medium has a tonicity of 280 mOsm/L±4 mOsm/L. In some aspects, the metabolic reprogramming medium has a tonicity of 280 mOsm/L±5 mOsm/L. In some aspects, the metabolic reprogramming medium has a tonicity of 280 mOsm/L±6 mOsm/L. In some aspects, the metabolic reprogramming medium has a tonicity of 280 mOsm/L±7 mOsm/L. In some aspects, the MRM has a tonicity of 280 mOsm/L±8 mOsm/L. In some aspects, the metabolic reprogramming medium has a tonicity of 280 mOsm/L±9 mOsm/L. In some aspects, the metabolic reprogramming medium has a tonicity of 280 mOsm/L±10 mOsm/L. In some aspects, the metabolic reprogramming medium has a tonicity of about 280 mOsm/L to about 285 mOsm/L, about 280 mOsm/L to about 290 mOsm/L, about 280 mOsm/L to about 295 mOsm/L, about 280 mOsm/L to about 300 mOsm/L, about 280 mOsm/L to about 305 mOsm/L, about 280 mOsm/L to about 310 mOsm/L, about 280 mOsm/L to about 315 mOsm/L, or about 280 mOsm/L to less than 320 mOsm/L. In some aspects, the metabolic reprogramming medium has a tonicity of about 285 mOsm/L, about 290 mOsm/L, about 295 mOsm/L, about 300 mOsm/L, about 305 mOsm/L, about 310 mOsm/L, or about 315 mOsm/L.

In some aspects, the metabolic reprogramming medium is hypotonic. In some aspects, the metabolic reprogramming medium has a tonicity lower than about 280 mOsm/L. In some aspects, the metabolic reprogramming medium has a tonicity lower than about 280 mOsm/L; as measured by adding the potassium ion concentration and the NaCl concentration, and multiplying by two. In some aspects, the metabolic reprogramming medium has a tonicity lower than 280 mOsm/L. In some aspects, the metabolic reprogramming medium has a tonicity lower than 280 mOsm/L; as measured by adding the potassium ion concentration and the NaCl concentration, and multiplying by two. In some aspects, the metabolic reprogramming medium has a tonicity lower than 275 mOsm/L. In some aspects, the metabolic reprogramming medium has a tonicity lower than 275 mOsm/L; as measured by adding the potassium ion concentration and the NaCl concentration, and multiplying by two; as measured by adding the potassium ion concentration and the NaCl concentration, and multiplying by two. In some aspects, the metabolic reprogramming medium has a tonicity lower than 270 mOsm/L. In some aspects, the metabolic reprogramming medium has a tonicity lower than 270 mOsm/L; as measured by adding the potassium ion concentration and the NaCl concentration, and multiplying by two. In some aspects, the metabolic reprogramming medium has a tonicity lower than 265 mOsm/L. In some aspects, the metabolic reprogramming medium has a tonicity lower than 265 mOsm/L; as measured by adding the potassium ion concentration and the NaCl concentration, and multiplying by two. In some aspects, the metabolic reprogramming medium has a tonicity lower than 260 mOsm/L. In some aspects, the metabolic reprogramming medium has a tonicity lower than 260 mOsm/L; as measured by adding the potassium ion concentration and the NaCl concentration, and multiplying by two. In some aspects, the metabolic reprogramming medium has a tonicity lower than 265 mOsm/L. In some aspects, the metabolic reprogramming medium has a tonicity lower than 265 mOsm/L; as measured by adding the potassium ion concentration and the NaCl concentration, and multiplying by two. In some aspects, the metabolic reprogramming medium has a tonicity lower than 260 mOsm/L. In some aspects, the metabolic reprogramming medium has a tonicity lower than 260 mOsm/L; as measured by adding the potassium ion concentration and the NaCl concentration, and multiplying by two. In some aspects, the metabolic reprogramming medium has a tonicity lower than 255 mOsm/L. In some aspects, the metabolic reprogramming medium has a tonicity lower than 255 mOsm/L; as measured by adding the potassium ion concentration and the NaCl concentration, and multiplying by two. In some aspects, the metabolic reprogramming medium has a tonicity lower than about 250 mOsm/L. In some aspects, the metabolic reprogramming medium has a tonicity lower than about 250 mOsm/L; as measured by adding the potassium ion concentration and the NaCl concentration, and multiplying by two. In some aspects, the metabolic reprogramming medium has a tonicity lower than about 245 mOsm/L. In some aspects, the metabolic reprogramming medium has a tonicity lower than about 245 mOsm/L; as measured by adding the potassium ion concentration and the NaCl concentration, and multiplying by two. In some aspects, the metabolic reprogramming medium has a tonicity lower than about 240 mOsm/L. In some aspects, the metabolic reprogramming medium has a tonicity lower than about 240 mOsm/L; as measured by adding the potassium ion concentration and the NaCl concentration, and multiplying by two. In some aspects, the metabolic reprogramming medium has a tonicity lower than about 235 mOsm/L. In some aspects, the metabolic reprogramming medium has a tonicity lower than about 235 mOsm/L; as measured by adding the potassium ion concentration and the NaCl concentration, and multiplying by two. In some aspects, the metabolic reprogramming medium has a tonicity lower than about 230 mOsm/L. In some aspects, the metabolic reprogramming medium has a tonicity lower than about 230 mOsm/L; as measured by adding the potassium ion concentration and the NaCl concentration, and multiplying by two. In some aspects, the metabolic reprogramming medium has a tonicity lower than about 225 mOsm/L. In some aspects, the metabolic reprogramming medium has a tonicity lower than about 225 mOsm/L. In some aspects, the tonicity is higher than about 220 mOsm/L; as measured by adding the potassium ion concentration and the NaCl concentration, and multiplying by two. In some aspects, the metabolic reprogramming medium has a tonicity from about 230 mOsm/L to about 280 mOsm/L. In some aspects, the metabolic reprogramming medium has a tonicity from about 240 mOsm/L to about 280 mOsm/L.

In some aspects, the metabolic reprogramming medium has an osmolality lower than about 220 mOsm/L. In some aspects, the metabolic reprogramming medium has an osmolality lower than about 215 mOsm/L. In some aspects, the metabolic reprogramming medium has an osmolality lower than about 210 mOsm/L. In some aspects, the metabolic reprogramming medium has an osmolality lower than about 205 mOsm/L. In some aspects, the metabolic reprogramming medium has an osmolality lower than about 200 mOsm/L.

In some aspects, the metabolic reprogramming medium has a tonicity from about 100 mOsm/L to about 280 mOsm/L, about 125 mOsm/L to about 280 mOsm/L, about 150 mOsm/L to about 280 mOsm/L, about 175 mOsm/L to about 280 mOsm/L, about 200 mOsm/L to about 280 mOsm/L, about 210 mOsm/L to about 280 mOsm/L, about 220 mOsm/L to about 280 mOsm/L, about 225 mOsm/L to about 280 mOsm/L, about 230 mOsm/L to about 280 mOsm/L, about 235 mOsm/L to about 280 mOsm/L, about 240 mOsm/L to about 280 mOsm/L, about 245 mOsm/L to about 280 mOsm/L, about 250 mOsm/L to about 280 mOsm/L, about 255 mOsm/L to about 280 mOsm/L, about 260 mOsm/L to about 280 mOsm/L, about 265 mOsm/L to about 280 mOsm/L, about 270 mOsm/L to about 280 mOsm/L, or about 275 mOsm/L to about 280 mOsm/L. In some aspects, the metabolic reprogramming medium has a tonicity from about 250 mOsm/L to about 270 mOsm/L. In some aspects, the metabolic reprogramming medium has a tonicity from about 250 mOsm/L to about 255 mOsm/L, about 250 mOsm/L to about 260 mOsm/L, about 250 mOsm/L to about 265 mOsm/L, about 255 mOsm/L to about 260 mOsm/L, about 255 mOsm/L to about 265 mOsm/L, about 255 mOsm/L to about 265 mOsm/L, about 260 mOsm/L to about 265 mOsm/L, or about 254 mOsm/L to about 263 mOsm/L. In some aspects, the metabolic reprogramming medium has a tonicity from about 254 mOsm/L to about 255 mOsm/L. In some aspects, the metabolic reprogramming medium has a tonicity from about 255 mOsm/L to about 256 mOsm/L. In some aspects, the metabolic reprogramming medium has a tonicity from about 256 mOsm/L to about 257 mOsm/L. In some aspects, the metabolic reprogramming medium has a tonicity from about 257 mOsm/L to about 258 mOsm/L. In some aspects, the metabolic reprogramming medium has a tonicity from about 258 mOsm/L to about 259 mOsm/L. In some aspects, the metabolic reprogramming medium has a tonicity from about 260 mOsm/L to about 261 mOsm/L. In some aspects, the metabolic reprogramming medium has a tonicity from about 261 mOsm/L to about 262 mOsm/L. In some aspects, the metabolic reprogramming medium has a tonicity from about 262 mOsm/L to about 263 mOsm/L. In some aspects, the metabolic reprogramming medium has a tonicity from about 263 mOsm/L to about 264 mOsm/L. In some aspects, the metabolic reprogramming medium has a tonicity from about 264 mOsm/L to about 265 mOsm/L. In some aspects, the metabolic reprogramming medium has a tonicity from about 220 mOsm/L to about 280 mOsm/L.

In some aspects, the metabolic reprogramming medium has a tonicity of about 100 mOsm/L, about 125 mOsm/L, about 150 mOsm/L, about 175 mOsm/L, about 200 mOsm/L, about 210 mOsm/L, about 220 mOsm/L, about 225 mOsm/L, about 230 mOsm/L, about 235 mOsm/L, about 240 mOsm/L, about 245 mOsm/L, about 250 mOsm/L, about 255 mOsm/L, about 260 mOsm/L, about 265 mOsm/L, about 270 mOsm/L, or about 275 mOsm/L.

In some aspects, the metabolic reprogramming medium has a tonicity of about 250 mOsm/L. In some aspects, the metabolic reprogramming medium has a tonicity of about 262.26 mOsm/L. In some aspects, the metabolic reprogramming medium has a tonicity of about 260 mOsm/L. In some aspects, the metabolic reprogramming medium has a tonicity of about 259.7 mOsm/L. In some aspects, the metabolic reprogramming medium has a tonicity of about 257.5 mOsm/L. In some aspects, the metabolic reprogramming medium has a tonicity of about 257.2 mOsm/L. In some aspects, the metabolic reprogramming medium has a tonicity of about 255.2 mOsm/L. In some aspects, the metabolic reprogramming medium has a tonicity of about 254.7. In some aspects, the metabolic reprogramming medium has a tonicity of about 255 mOsm/L. In some aspects, the metabolic reprogramming medium has a tonicity of about 260 mOsm/L. In some aspects, the MRM comprises (i) potassium ion at a concentration higher than 5 mM, (ii) NaCl at a concentration between about 40 mM to about 80 mM, and (iii) a tonicity of about 250-260 mOsm/L. In some aspects, the MRM comprises (i) potassium ion at a concentration between about 40 mM to about 80 mM, (ii) NaCl at a concentration between about 40 mM to about 80 mM, and (iii) a tonicity of about 250-260 mOsm/L. In some aspects, the MRM comprises (i) potassium ion at a concentration between about 40 mM to about 80 mM, (ii) NaCl at a concentration between about 55 mM to about 90 mM, and (iii) a tonicity of about 250-260 mOsm/L.

In some aspects, the metabolic reprogramming medium comprises about 50 mM potassium ion and (i) about 80.5 mM NaCl; (ii) about 17.7 mM glucose; and (iii) about 1.9 mM calcium ion.

In some aspects, the metabolic reprogramming medium comprises about 50 mM potassium ion and (i) about 80.5 mM NaCl; (ii) about 24 mM glucose; and (iii) about 2.8 mM calcium ion.

In some aspects, the metabolic reprogramming medium comprises about 40 mM potassium ion and (i) about 88.9 mM NaCl; (ii) about 24 mM glucose; and (iii) about 2.8 mM calcium ion.

In some aspects, the metabolic reprogramming medium comprises about 60 mM potassium ion and (i) about 72.2 mM NaCl; (ii) about 24 mM glucose; and (iii) about 2.8 mM calcium ion.

In some aspects, the metabolic reprogramming medium comprises about 70 mM potassium ion and (i) about 63.9 mM NaCl; (ii) about 24 mM glucose; and (iii) about 2.8 mM calcium ion.

In some aspects, the metabolic reprogramming medium comprises about 80 mM potassium ion and (i) about 55.6 mM NaCl; (ii) about 24 mM glucose; and (iii) about 2.8 mM calcium ion.

In some aspects, the metabolic reprogramming medium comprises about 50 mM potassium ion and (i) about 80.5 mM NaCl; (ii) about 17.7 mM glucose; and (iii) about 1.8 mM calcium ion.

In some aspects, the metabolic reprogramming medium comprises about 55 mM potassium ion and (i) about 76 mM NaCl; (ii) about 17.2 mM glucose; and (iii) about 1.7 mM calcium ion.

In some aspects, the metabolic reprogramming medium comprises about 60 mM potassium ion and (i) about 72.2 mM NaCl; (ii) about 16.8 mM glucose; and (iii) about 1.6 mM calcium ion.

In some aspects, the metabolic reprogramming medium comprises about 65 mM potassium ion and (i) about 67.6 mM NaCl; (ii) about 16.3 mM glucose; and (iii) about 1.5 mM calcium ion.

In some aspects, the metabolic reprogramming medium comprises about 70 mM potassium ion and (i) about 63.9 mM NaCl; (ii) about 15.9 mM glucose; and (iii) about 1.4 mM calcium ion.

In some aspects, the metabolic reprogramming medium comprises about 75 mM potassium ion and (i) about 59.3 mM NaCl; (ii) about 15.4 mM glucose; and (iii) about 1.3 mM calcium ion.

In some aspects, the metabolic reprogramming medium comprises about 80 mM potassium ion and (i) about 55.6 mM NaCl; (ii) about 15 mM glucose; and (iii) about 1.2 mM calcium ion.

The tonicity of the metabolic reprogramming medium can be adjusted, e.g., to an isotonic or hypotonic state disclosed herein, at any point. In some aspects, the tonicity of the metabolic reprogramming medium can be adjusted, e.g., to an isotonic or hypotonic state disclosed herein, before the cells are added to the metabolic reprogramming medium. In some aspects, the cells are cultured in the hypotonic or isotonic medium prior to cell engineering, e.g., prior to transduction with a construct expressing a CAR, TCR or TCR mimic. In some aspects, the cells are cultured in the hypotonic or isotonic medium during cell engineering, e.g., during transduction with a construct expressing a CAR, TCR or TCR mimic. In some aspects the cells are cultured in the hypotonic or isotonic medium after cell engineering, e.g., after transduction with a construct expressing a CAR, TCR or TCR mimic. In some aspects, the cells are cultured in the hypotonic or isotonic medium throughout cell expansion.

II.A.4. Saccharides

Some aspects of the present disclosure are directed to methods of culturing immune cells, e.g., T cells and/or NK cells, in a medium comprising (i) potassium ion at a concentration of at least about 5 mM (e.g., higher than 5 mM, e.g., between about 40 mM and about 80 mM) and (ii) a saccharide. In some aspects, the medium is hypotonic or isotonic.

In some aspects, the target concentration of the saccharide is reached by starting with a basal medium comprising a higher concentration of the saccharide, and diluting the solution to reach the target concentration of the saccharide. In some aspects, the target concentration of the saccharide is reached by raising the concentration of the saccharide by adding the saccharide until the desired concentration is reached. In some aspects, the saccharide is a monosaccharide, a disaccharide, or a polysaccharide. In some aspects, the saccharide is selected from glucose, fructose, galactose, mannose, maltose, sucrose, lactose, trehalose, or any combination thereof. In certain aspects, the saccharide is glucose. In some aspects, the medium comprises (i) potassium ion at a concentration of at least about 5 mM and (ii) glucose. In some aspects, the medium comprises (i) potassium ion at a concentration higher than 40 mM and (ii) glucose. In some aspects, the medium comprises (i) potassium ion at a concentration of at least about 5 mM and (ii) mannose. In some aspects, the medium comprises (i) potassium ion at a concentration of at least about 50 mM and (ii) mannose. In some aspects, the medium is hypotonic. In some aspects, the medium is isotonic. In some aspects, the medium comprises (i) potassium ion at a concentration higher than 40 mM and (ii) glucose; wherein the total concentration of potassium ion and NaCl is between 110 mM and 140 mM. In some aspects, the medium comprises (i) potassium ion at a concentration higher than 50 mM and (ii) glucose; wherein the total concentration of potassium ion and NaCl is between 110 mM and 140 mM. In some aspects, the medium comprises (i) potassium ion at a concentration of at least about 40 mM and (ii) mannose; wherein the total concentration of potassium ion and NaCl is between 110 mM and 140 mM. In some aspects, the medium comprises (i) potassium ion at a concentration of at least about 50 mM and (ii) mannose; wherein the total concentration of potassium ion and NaCl is between 110 mM and 140 mM.

In some aspects, the metabolic reprogramming medium comprises (i) potassium ion at a concentration higher than 5 mM and (ii) glucose. In some aspects, the metabolic reprogramming medium comprises (i) potassium ion at a concentration of at least about 30 mM to at least about 100 mM and (ii) glucose. In some aspects, the metabolic reprogramming medium comprises (i) potassium ion at a concentration higher than 40 mM and (ii) glucose. In some aspects, the metabolic reprogramming medium comprises (i) potassium ion at a concentration higher than 5 mM and (ii) mannose. In some aspects, the metabolic reprogramming medium comprises (i) potassium ion at a concentration of at least about 30 mM to at least about 100 mM and (ii) mannose. In some aspects, the metabolic reprogramming medium comprises (i) potassium ion at a concentration of higher than 40 mM and (ii) mannose. In some aspects, the metabolic reprogramming medium comprises (i) potassium ion at a concentration of at least about 50 mM and (ii) mannose. In some aspects, the metabolic reprogramming medium is hypotonic. In some aspects, the medium is isotonic. In some aspects, the metabolic reprogramming medium comprises (i) potassium ion at a concentration higher than 40 mM and (ii) glucose; wherein the total concentration of potassium ion and NaCl is between 110 mM and 140 mM. In some aspects, the metabolic reprogramming medium comprises (i) potassium ion at a concentration higher than 50 mM and (ii) glucose; wherein the total concentration of potassium ion and NaCl is between 110 mM and 140 mM. In some aspects, the metabolic reprogramming medium comprises (i) potassium ion at a concentration of at least about 40 mM and (ii) mannose; wherein the total concentration of potassium ion and NaCl is between 110 mM and 140 mM. In some aspects, the metabolic reprogramming medium comprises (i) potassium ion at a concentration of at least about 50 mM and (ii) mannose; wherein the total concentration of potassium ion and NaCl is between 110 mM and 140 mM.

In some aspects, the concentration of the saccharide, e.g., glucose, is about 10 mM to about 24 mM. In some aspects, the concentration of the saccharide, e.g., glucose, is less than about 4.29 g/L. In some aspects, the concentration of the saccharide, e.g., glucose, is less than about 24 mM. In some aspects, the concentration of the saccharide, e.g., glucose, is more than about 5 mM. In some aspects, the concentration of the saccharide, e.g., glucose, is about 5 mM. In some aspects, the concentration of the saccharide, e.g., glucose, is from about 5 mM to about 20 mM. In some aspects, the concentration of the saccharide, e.g., glucose, is from about 10 mM to about 20 mM. In some aspects, the concentration of the saccharide, e.g., glucose, is from about 10 mM to about 25 mM, about 10 mM to about 20 mM, about 10 mM to about 5 mM, about 15 mM to about 25 mM, about 15 mM to about 20 mM, about 15 mM to about 19 mM, about 15 mM to about 18 mM, about 15 mM to about 17 mM, about 15 mM to about 16 mM, about 16 mM to about 20 mM, about 16 mM to about 19 mM, about 16 mM to about 18 mM, about 16 mM to about 17 mM, about 17 mM to about 20 mM, about 17 mM to about 19 mM, or about 17 mM to about 18 mM. In some aspects, the concentration of the saccharide, e.g., glucose, is from about 5 mM to about 20 mM. In some aspects, the concentration of the saccharide, e.g., glucose, is from about 10 mM to about 20 mM. In some aspects, the concentration of the saccharide, e.g., glucose, is from about 10 mM to about 15 mM. In some aspects, the concentration of the saccharide, e.g., glucose, is from about 14 mM to about 14.5 mM. In some aspects, the concentration of the saccharide, e.g., glucose, is from about 14.5 mM to about 15 mM. In some aspects, the concentration of the saccharide, e.g., glucose, is from about 15 mM to about 15.5 mM. In some aspects, the concentration of the saccharide, e.g., glucose, is from about 15.5 mM to about 16 mM. In some aspects, the concentration of the saccharide, e.g., glucose, is from about 16 mM to about 16.5 mM. In some aspects, the concentration of the saccharide, e.g., glucose, is from about 16.5 mM to about 17 mM. In some aspects, the concentration of the saccharide, e.g., glucose, is from about 17 mM to about 17.5 mM. In some aspects, the concentration of the saccharide, e.g., glucose, is from about 17.5 mM to about 18 mM.

In some aspects, the concentration of the saccharide, e.g., glucose, is about 5 mM, about 6 mM, about 7 mM, about 8 mM, about 9 mM, about 10 mM, is about 10.5 mM, about 11 mM, about 11.5 mM, about 12 mM, about 12.5 mM, about 13 mM, about 13.5 mM, about 14 mM, about 14.5 mM, about 15 mM, about 15.5 mM, about 16 mM, about 16.5 mM, about 17 mM, about 17.5 mM, about 18 mM, about 18.5 mM, about 19 mM, about 19.5 mM, about 20 mM, about 20.5 mM, about 21 mM, about 22 mM, about 23 mM, about 24 mM, or about 25 mM.

In some aspects, a medium useful for the present disclosure comprises (i) potassium ion at a concentration higher than 5 mM (e.g., between about 40 mM to about 80 mM), (ii) NaCl at a concentration between about 40 mM to about 80 mM, and (iii) glucose. In some aspects, a medium useful for the present disclosure comprises (i) potassium ion at a concentration higher than 5 mM (e.g., between about 40 mM to about 80 mM), (ii) NaCl at a concentration between about 40 mM to about 80 mM, (iii) glucose, and (iv) a tonicity of about 250-260 mOsm/L. In some aspects, a medium useful for the present disclosure comprises (i) potassium ion at a concentration higher than 5 mM (e.g., between about 40 mM to about 80 mM), (ii) NaCl at a concentration between about 40 mM to about 80 mM, (iii) glucose at a concentration between about 10 mM to about 24 mM, and (iv) a tonicity of about 250-260 mOsm/L.

II.A.5. Calcium

Some aspects of the present disclosure are directed to methods of culturing immune cells, e.g., T cells and/or NK cells, in a medium comprising (i) potassium ion at a concentration of at least about 5 mM (e.g., higher than 5 mM, e.g., between about 40 mM and about 80 mM) and (ii) calcium ion. In some aspects, the medium is hypotonic or isotonic.

In some aspects, the target concentration of calcium is reached by starting with a basal medium comprising a higher concentration of calcium ion, and diluting the solution to reach the target concentration of calcium ion. In some aspects, the target concentration of calcium is reached by raising the concentration of calcium ion by adding one or more calcium salts. Non-limiting examples of calcium salts include calcium bromide, calcium carbonate, calcium chloride, calcium cyanamide, calcium fluoride, calcium hydride, calcium hydroxide, calcium iodate, calcium iodide, calcium nitrate, calcium nitrite, calcium oxalate, calcium perchlorate tetrahydrate, calcium phosphate monobasic, calcium phosphate tribasic, calcium sulfate, calcium thiocyanate tetrahydrate, hydroxyapatite, or any combination thereof. In some aspects, the calcium salt comprises calcium chloride (CaCl₂)). In some aspects, the calcium salt comprises calcium gluconate.

In some aspects, the concentration of the calcium ion is less than that of the basal medium. In some aspects, the concentration of the calcium ion is greater than that of the basal medium. In some aspects, the concentration of calcium ion is more than about 0.4 mM. In some aspects, the concentration of calcium ion is less than about 2.8 mM. In some aspects, the concentration of calcium ion is less than about 2.5 mM. In some aspects, the concentration of calcium ion is less than about 2.0 mM. In some aspects, the concentration of calcium ion is less than about 1.9 mM. In some aspects, the concentration of calcium ion is less than about 1.8 mM. In some aspects, the concentration of calcium ion is less than about 1.7 mM. In some aspects, the concentration of calcium ion is less than about 1.6 mM. In some aspects, the concentration of calcium ion is less than about 1.5 mM. In some aspects, the concentration of calcium ion is less than about 1.4 mM. In some aspects, the concentration of calcium ion is less than about 1.3 mM. In some aspects, the concentration of calcium ion is less than about 1.2 mM. In some aspects, the concentration of calcium ion is less than about 1.1 mM. In some aspects, the concentration of calcium ion is less than about 1.0 mM.

In some aspects, the concentration of calcium ion is from about 0.4 mM to about 2.8 mM, about 0.4 mM to about 2.7 mM, about 0.4 mM to about 2.5 mM, about 0.5 mM to about 2.0 mM, about 1.0 mM to about 2.0 mM, about 1.1 mM to about 2.0 mM, about 1.2 mM to about 2.0 mM, about 1.3 mM to about 2.0 mM, about 1.4 mM to about 2.0 mM, about 1.5 mM to about 2.0 mM, about 1.6 mM to about 2.0 mM, about 1.7 mM to about 2.0 mM, about 1.8 mM to about 2.0 mM, about 0.8 to about 0.9 mM, about 0.8 to about 1.0 mM, about 0.8 to about 1.1 mM, about 0.8 to about 1.2 mM, about 0.8 to about 1.3 mM, about 0.8 to about 1.4 mM, about 0.8 to about 1.5 mM, about 0.8 to about 1.6 mM, about 0.8 to about 1.7 mM, about 0.8 to about 1.8 mM, about 0.8 to about 1.9 mM, about 0.9 to about 1.0 mM, about 0.9 to about 1.1 mM, about 0.9 to about 1.2 mM, about 0.9 to about 1.3 mM, about 0.9 to about 1.4 mM, about 0.9 to about 1.5 mM, about 0.9 to about 1.6 mM, about 0.9 to about 1.7 mM, about 0.9 to about 1.8 mM, about 0.9 to about 1.9 mM, about 1.0 to about 1.1 mM, about 1.0 to about 1.2 mM, about 1.0 to about 1.3 mM, about 1.0 to about 1.4 mM, about 1.0 to about 1.5 mM, about 1.0 to about 1.6 mM, about 1.0 to about 1.7 mM, about 1.0 to about 1.8 mM, about 1.0 to about 1.9 mM, about 1.1 to about 1.2 mM, about 1.1 to about 1.3 mM, about 1.1 to about 1.4 mM, about 1.1 to about 1.5 mM, about 1.1 to about 1.6 mM, about 1.1 to about 1.7 mM, about 1.1 to about 1.8 mM, about 1.1 to about 1.9 mM, about 1.2 to about 1.3 mM, about 1.2 to about 1.4 mM, about 1.2 to about 1.5 mM, about 1.2 to about 1.6 mM, about 1.2 to about 1.7 mM, about 1.2 to about 1.8 mM, about 1.2 to about 1.9 mM, about 1.3 to about 1.4 mM, about 1.3 to about 1.5 mM, about 1.3 to about 1.6 mM, about 1.3 to about 1.7 mM, about 1.3 to about 1.8 mM, about 1.3 to about 1.9 mM, about 1.4 to about 1.5 mM, about 1.4 to about 1.6 mM, about 1.4 to about 1.7 mM, about 1.4 to about 1.8 mM, about 1.4 to about 1.9 mM, about 1.5 to about 1.6 mM, about 1.5 to about 1.7 mM, about 1.5 to about 1.8 mM, about 1.5 to about 1.9 mM, about 1.6 to about 1.7 mM, about 1.6 to about 1.8 mM, about 1.6 to about 1.9 mM, about 1.7 to about 1.8 mM, about 1.7 to about 1.9 mM, or about 1.8 to about 1.9 mM.

In some aspects, the concentration of calcium ion is from about 0.8 mM to about 1.8 mM. In some aspects, the concentration of calcium ion is from about 0.9 mM to about 1.8 mM. In some aspects, the concentration of calcium ion is from about 1.0 mM to about 1.8 mM. In some aspects, the concentration of calcium ion is from about 1.1 mM to about 1.8 mM. In some aspects, the concentration of calcium ion is from about 1.2 mM to about 1.8 mM. In some aspects, the concentration of calcium ion is from about 0.8 mM to about 1.8 mM. In some aspects, the concentration of calcium ion is from about 0.8 mM to about 0.9 mM. In some aspects, the concentration of calcium ion is from about 0.9 mM to about 1.0 mM. In some aspects, the concentration of calcium ion is from about 1.0 mM to about 1.1 mM. In some aspects, the concentration of calcium ion is from about 1.1 mM to about 1.2 mM. In some aspects, the concentration of calcium ion is from about 1.2 mM to about 1.3 mM. In some aspects, the concentration of calcium ion is from about 1.3 mM to about 1.4 mM. In some aspects, the concentration of calcium ion is from about 1.4 mM to about 1.5 mM. In some aspects, the concentration of calcium ion is from about 1.5 mM to about 1.6 mM. In some aspects, the concentration of calcium ion is from about 1.7 mM to about 1.8 mM. In some aspects, the concentration of calcium ion is from about 1.8 mM to about 1.9 mM.

In some aspects, the concentration of calcium ion is about 0.6 mM, about 0.7 mM, about 0.8 mM, about 0.9 mM, about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, or about 2.0 mM. In some aspects, the concentration of calcium ion is about 0.6 mM. In some aspects, the concentration of calcium ion is about 0.7 mM. In some aspects, the concentration of calcium ion is about 0.8 mM. In some aspects, the concentration of calcium ion is about 0.9 mM. In some aspects, the concentration of calcium ion is about 1.0 mM. In some aspects, the concentration of calcium ion is about 1.1 mM. In some aspects, the concentration of calcium ion is about 1.2 mM. In some aspects, the concentration of calcium ion is about 1.3 mM. In some aspects, the concentration of calcium ion is about 1.4 mM. In some aspects, the concentration of calcium ion is about 1.5 mM. In some aspects, the concentration of calcium ion is about 1.6 mM. In some aspects, the concentration of calcium ion is about 1.7 mM. In some aspects, the concentration of calcium ion is about 1.8 mM. In some aspects, the concentration of calcium ion is about 1.9 mM.

In some aspects, a medium useful for the present disclosure comprises: (i) potassium ion at a concentration between about 40 mM to about 80 mM and (ii) calcium at a concentration between about 0.5 mM to about 2.8 mM. In some aspects, the medium comprises: (i) potassium ion at a concentration between about 40 mM to about 80 mM, (ii) NaCl at a concentration between about 40 mM to about 80 mM, and (iii) calcium at a concentration between about 0.5 mM to about 2.8 mM. In some aspects, the medium comprises: (i) potassium ion at a concentration between about 40 mM to about 80 mM, (ii) NaCl at a concentration between about 40 mM to about 80 mM, (iii) glucose at a concentration between about 10 mM to about 24 mM, and (iv) calcium at a concentration between about 0.5 mM to about 2.8 mM. In some aspects, the medium comprises: (i) potassium ion at a concentration between about 40 mM to about 80 mM, (ii) NaCl at a concentration between about 40 mM to about 80 mM, (iii) glucose at a concentration between about 10 mM to about 24 mM, (iv) calcium at a concentration between about 0.5 mM to about 2.8 mM, and (v) a tonicity of about 250-260 mOsm/L.

II.A.6. Cytokines

In some aspects, the metabolic reprogramming medium comprises a cytokine. In some aspects, the medium is hypotonic. In some aspects, the medium is isotonic. In some aspects, the medium is hypertonic. In some aspects, the cytokine is selected from IL-2, IL-7, IL-15, IL-21, and any combination thereof. In some aspects, the metabolic reprogramming medium does not comprise IL-2. In some aspects, the metabolic reprogramming medium comprises IL-2 and IL-21. In some aspects, the metabolic reprogramming medium comprises IL-2, IL-21, and IL-15.

The cytokine can be added to the medium at any point. In some aspects, the cytokine is added to the medium before the immune cells, e.g., T cells and/or NK cells, are added to the medium. In some aspects, the immune cells, e.g., T cells and/or NK cells, are cultured in the medium comprising (i) potassium at a concentration disclosed herein (e.g., higher than 5 mM, e.g., between about 40 mM and about 80 mM), and (ii) a cytokine prior to cell engineering, e.g., prior to transduction with a construct encoding a ligand binding protein. In some aspects, the immune cells, e.g., T cells and/or NK cells, are cultured in the medium comprising (i) potassium at a concentration disclosed herein (e.g., higher than 5 mM, e.g., between about 40 mM and about 80 mM), and (ii) a cytokine during cell engineering, e.g., during transduction with a ligand binding protein. In some aspects, the immune cells, e.g., T cells and/or NK cells, are cultured in the medium comprising (i) potassium at a concentration disclosed herein (e.g., higher than 5 mM, e.g., between about 40 mM and about 80 mM), and (ii) a cytokine after cell engineering, e.g., after transduction with a construct encoding polypeptide ligand binding protein. In some aspects, the immune cells, e.g., T cells and/or NK cells, are cultured in the medium comprising (i) potassium at a concentration disclosed herein (e.g., higher than 5 mM, e.g., between about 40 mM and about 80 mM), and (ii) a cytokine throughout cell expansion.

In some aspects, the metabolic reprogramming medium comprises (i) at least about 5 mM potassium ion and (ii) IL-2. In some aspects, the metabolic reprogramming medium comprises (i) more than 40 mM potassium ion and (ii) IL-2. In some aspects, the metabolic reprogramming medium comprises (i) at least about 50 mM potassium ion and (ii) IL-2. In some aspects, the metabolic reprogramming medium comprises (i) at least about 5 mM potassium ion and (ii) IL-7. In some aspects, the metabolic reprogramming medium comprises (i) more than 40 mM potassium ion and (ii) IL-7. In some aspects, the metabolic reprogramming medium comprises (i) at least about 50 mM potassium ion and (ii) IL-7. In some aspects, the metabolic reprogramming medium comprises (i) at least about 5 mM potassium ion and (ii) IL-15. In some aspects, the metabolic reprogramming medium comprises (i) more than 40 mM potassium ion and (ii) IL-15. In some aspects, the metabolic reprogramming medium comprises (i) at least about 50 mM potassium ion and (ii) IL-15. In some aspects, the metabolic reprogramming medium comprises (i) at least about 5 mM potassium ion and (ii) IL-21. In some aspects, the metabolic reprogramming medium comprises (i) more than 40 mM potassium ion and (ii) IL-21. In some aspects, the metabolic reprogramming medium comprises (i) at least about 50 mM potassium ion and (ii) IL-21. In some aspects, the metabolic reprogramming medium comprises (i) at least about 5 mM potassium ion and (ii) IL-2, and the metabolic reprogramming medium does not comprise IL-7. In some aspects, the metabolic reprogramming medium comprises (i) more than 40 mM potassium ion and (ii) IL-2, and the metabolic reprogramming medium does not comprise IL-7. In some aspects, the metabolic reprogramming medium comprises (i) at least about 50 mM potassium ion and (ii) IL-2, and the metabolic reprogramming medium does not comprise IL-7. In some aspects, the metabolic reprogramming medium comprises (i) at least about 5 mM potassium ion and (ii) IL-2, and the metabolic reprogramming medium does not comprise IL-15. In some aspects, the metabolic reprogramming medium comprises (i) more than 40 mM potassium ion and (ii) IL-2, and the metabolic reprogramming medium does not comprise IL-15. In some aspects, the metabolic reprogramming medium comprises (i) at least about 50 mM potassium ion and (ii) IL-2, and the metabolic reprogramming medium does not comprise IL-15. In some aspects, the metabolic reprogramming medium comprises (i) at least about 5 mM potassium ion and (ii) IL-2, and the metabolic reprogramming medium does not comprise IL-7 and IL-15. In some aspects, the metabolic reprogramming medium comprises (i) more than 40 mM potassium ion and (ii) IL-2, and the metabolic reprogramming medium does not comprise IL-7 and IL-15. In some aspects, the metabolic reprogramming medium comprises (i) at least about 50 mM potassium ion and (ii) IL-2, and the metabolic reprogramming medium does not comprise IL-7 and IL-15. In some aspects, the metabolic reprogramming medium comprises (i) at least about 5 mM potassium ion and (ii) IL-2 and IL-21. In some aspects, the metabolic reprogramming medium comprises (i) more than 40 mM potassium ion and (ii) IL-2 and IL-21. In some aspects, the metabolic reprogramming medium comprises (i) at least about 50 mM potassium ion and (ii) IL-2 and IL-21. In some aspects, the metabolic reprogramming medium comprises (i) at least about 5 mM potassium ion and (ii) IL-7 and IL-21. In some aspects, the metabolic reprogramming medium comprises (i) more than 40 mM potassium ion and (ii) IL-7 and IL-21. In some aspects, the metabolic reprogramming medium comprises (i) at least about 50 mM potassium ion and (ii) IL-7 and IL-21. In some aspects, the metabolic reprogramming medium comprises (i) at least about 5 mM potassium ion and (ii) IL-15 and IL-21. In some aspects, the metabolic reprogramming medium comprises (i) more than 40 mM potassium ion and (ii) IL-15 and IL-21. In some aspects, the metabolic reprogramming medium comprises (i) at least about 50 mM potassium ion and (ii) IL-15 and IL-21. In some aspects, the metabolic reprogramming medium is hypotonic. In some aspects, the metabolic reprogramming medium is isotonic. In some aspects, the metabolic reprogramming medium further comprises NaCl, wherein the total concentration of potassium ion and NaCl is from 110 mM to 140 mM.

In some aspects, the metabolic reprogramming medium described herein (e.g., comprising potassium ion at a concentration greater than 5 mM) comprises between about 50 IU/mL to about 500 IU/mL of IL-2. In some aspects, the metabolic reprogramming medium comprises about 50 IU/mL, about 60 IU/mL, about 70 IU/mL, about 80 IU/mL, about 90 IU/mL, about 100 IU/mL, about 125 IU/mL, about 150 IU/mL, about 175 IU/mL, about 200 IU/mL, about 225 IU/mL, about 250 IU/mL, about 275 IU/mL, about 300 IU/mL, about 350 IU/mL, about 400 IU/mL, about 450 IU/mL, or about 500 IU/mL of IL-2.

Therefore, in some aspects, the metabolic reprogramming medium comprises (i) potassium ion at a concentration higher than 5 mM and (ii) about 50 IU/mL of IL-2. In some aspects, the metabolic reprogramming medium comprises (i) potassium ion at a concentration higher than 5 mM and (ii) about 60 IU/mL of IL-2. In some aspects, the metabolic reprogramming medium comprises (i) potassium ion at a concentration higher than 5 mM and (ii) about 70 IU/mL of IL-2. In some aspects, the metabolic reprogramming medium comprises (i) potassium ion at a concentration higher than 5 mM and (ii) about 80 IU/mL of IL-2. In some aspects, the metabolic reprogramming medium comprises (i) potassium ion at a concentration higher than 5 mM and (ii) about 90 IU/mL of IL-2. In some aspects, the metabolic reprogramming medium comprises (i) potassium ion at a concentration higher than 5 mM and (ii) about 100 IU/mL of IL-2. In some aspects, the metabolic reprogramming medium comprises (i) potassium ion at a concentration higher than 5 mM and (ii) about 125 IU/mL of IL-2. In some aspects, the metabolic reprogramming medium comprises (i) potassium ion at a concentration higher than 5 mM and (ii) about 150 IU/mL of IL-2. In some aspects, the metabolic reprogramming medium comprises (i) potassium ion at a concentration higher than 5 mM and (ii) about 175 IU/mL of IL-2. In some aspects, the metabolic reprogramming medium comprises (i) potassium ion at a concentration higher than 5 mM and (ii) about 200 IU/mL of IL-2. In some aspects, the metabolic reprogramming medium comprises (i) potassium ion at a concentration higher than 5 mM and (ii) about 225 IU/mL of IL-2. In some aspects, the metabolic reprogramming medium comprises (i) potassium ion at a concentration higher than 5 mM and (ii) about 250 IU/mL of IL-2. In some aspects, the metabolic reprogramming medium comprises (i) potassium ion at a concentration higher than 5 mM and (ii) about 275 IU/mL of IL-2. In some aspects, the metabolic reprogramming medium comprises (i) potassium ion at a concentration higher than 5 mM and (ii) about 300 IU/mL of IL-2. In some aspects, the metabolic reprogramming medium comprises (i) potassium ion at a concentration higher than 5 mM and (ii) about 350 IU/mL of IL-2. In some aspects, the metabolic reprogramming medium comprises (i) potassium ion at a concentration higher than 5 mM and (ii) about 400 IU/mL of IL-2. In some aspects, the metabolic reprogramming medium comprises (i) potassium ion at a concentration higher than 5 mM and (ii) about 450 IU/mL of IL-2. In some aspects, the metabolic reprogramming medium comprises (i) potassium ion at a concentration higher than 5 mM and (ii) about 500 IU/mL of IL-2. In some aspects, the metabolic reprogramming medium comprising potassium ion and IL-2 further comprises NaCl at a concentration less than about 115 nM.

In some aspects, the metabolic reprogramming medium comprises at least about 0.1 ng/mL IL-2. In some aspects, the metabolic reprogramming medium comprises from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL IL-2.

In some aspects, the metabolic reprogramming medium comprises at least about 0.1 ng/mL, at least about 0.5 ng/mL, at least about 1 ng/mL, at least about 2 ng/mL, at least about 3 ng/mL, at least about 4 ng/mL, at least about 5 ng/mL, at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, at least about 15 ng/mL, at least about 16 ng/mL, at least about 17 ng/mL, at least about 18 ng/mL, at least about 19 ng/mL, or at least about 20 ng/mL IL-2. In some aspects, the metabolic reprogramming medium comprises at least about 1.0 ng/mL IL-2. In some aspects, the metabolic reprogramming medium comprises at least about 2.0 ng/mL IL-2. In some aspects, the metabolic reprogramming medium comprises at least about 3.0 ng/mL IL-2. In some aspects, the metabolic reprogramming medium comprises at least about 4.0 ng/mL IL-2. In some aspects, the metabolic reprogramming medium comprises at least about 5.0 ng/mL IL-2. In some aspects, the metabolic reprogramming medium comprises at least about 6.0 ng/mL IL-2. In some aspects, the metabolic reprogramming medium comprises at least about 7.0 ng/mL IL-2. In some aspects, the metabolic reprogramming medium comprises at least about 8.0 ng/mL IL-2. In some aspects, the metabolic reprogramming medium comprises at least about 9.0 ng/mL IL-2. In some aspects, the metabolic reprogramming medium comprises at least about 10 ng/mL IL-2.

In some aspects, the metabolic reprogramming medium comprises at least about 0.1 ng/mL IL-2. In some aspects, the metabolic reprogramming medium comprises from about 50 ng/mL to about 600 ng/mL, about 50 ng/mL to about 500 ng/mL, about 50 ng/mL to about 450 ng/mL, about 50 ng/mL to about 400 ng/mL, about 50 ng/mL to about 350 ng/mL, about 50 ng/mL to about 300 ng/mL, about 100 ng/mL to about 600 ng/mL, about 100 ng/mL to about 500 ng/mL, about 100 ng/mL to about 450 ng/mL, about 100 ng/mL to about 400 ng/mL, about 100 ng/mL to about 350 ng/mL, about 100 ng/mL to about 300 ng/mL, about 200 ng/mL to about 500 ng/mL, about 200 ng/mL to about 450 ng/mL, about 200 ng/mL to about 400 ng/mL, about 200 ng/mL to about 350 ng/mL, about 200 ng/mL to about 300 ng/mL, about 250 ng/mL to about 350 ng/mL, about 300 ng/mL to about 600 ng/mL, about 300 ng/mL to about 500 ng/mL, about 300 ng/mL to about 450 ng/mL, about 300 ng/mL to about 400 ng/mL, about 300 ng/mL to about 350 ng/mL, about 250 ng/mL to about 300 ng/mL, or about 275 ng/mL to about 325 ng/mL IL-2.

In some aspects, the metabolic reprogramming medium comprises at least about 50 ng/mL, at least about 60 ng/mL, at least about 70 ng/mL, at least about 80 ng/mL, at least about 90 ng/mL, at least about 100 ng/mL, at least about 110 ng/mL, at least about 120 ng/mL, at least about 130 ng/mL, at least about 140 ng/mL, at least about 150 ng/mL, at least about 160 ng/mL, at least about 170 ng/mL, at least about 180 ng/mL, at least about 190 ng/mL, at least about 200 ng/mL, at least about 210 ng/mL, at least about 220 ng/mL, at least about 230 ng/mL, at least about 240 ng/mL, at least about 250 ng/mL, at least about 260 ng/mL, at least about 270 ng/mL, at least about 280 ng/mL, at least about 290 ng/mL, at least about 300 ng/mL, at least about 310 ng/mL, at least about 320 ng/mL, at least about 330 ng/mL, at least about 340 ng/mL, at least about 350 ng/mL, at least about 360 ng/mL, at least about 370 ng/mL, at least about 380 ng/mL, at least about 390 ng/mL, at least about 400 ng/mL, at least about 410 ng/mL, at least about 420 ng/mL, at least about 430 ng/mL, at least about 440 ng/mL, at least about 450 ng/mL, at least about 460 ng/mL, at least about 470 ng/mL, at least about 480 ng/mL, at least about 490 ng/mL, at least about 500 ng/mL, at least about 510 ng/mL, at least about 520 ng/mL, at least about 530 ng/mL, at least about 540 ng/mL, at least about 550 ng/mL, at least about 560 ng/mL, at least about 570 ng/mL, at least about 580 ng/mL, at least about 590 ng/mL, or at least about 600 ng/mL IL-2. In some aspects, the metabolic reprogramming medium comprises at least about 50 ng/mL IL-2. In some aspects, the metabolic reprogramming medium comprises at least about 60 ng/mL IL-2. In some aspects, the metabolic reprogramming medium comprises at least about 70 ng/mL IL-2. In some aspects, the metabolic reprogramming medium comprises at least about 73.6 ng/mL IL-2. In some aspects, the metabolic reprogramming medium comprises at least about 75 ng/mL IL-2. In some aspects, the metabolic reprogramming medium comprises at least about 80 ng/mL IL-2. In some aspects, the metabolic reprogramming medium comprises at least about 90 ng/mL IL-2. In some aspects, the metabolic reprogramming medium comprises at least about 100 ng/mL IL-2. In some aspects, the metabolic reprogramming medium comprises at least about 200 ng/mL IL-2. In some aspects, the metabolic reprogramming medium comprises at least about 300 ng/mL IL-2. In some aspects, the metabolic reprogramming medium comprises at least about 400 ng/mL IL-2. In some aspects, the metabolic reprogramming medium comprises at least about 500 ng/mL IL-2. In some aspects, the metabolic reprogramming medium comprises at least about 600 ng/mL IL-2.

In some aspects, the metabolic reprogramming medium described herein (e.g., comprising potassium ion at a concentration greater than 5 mM) comprises between about 50 IU/mL to about 500 IU/mL of IL-21. In some aspects, the culture medium comprises about 50 IU/mL, about 60 IU/mL, about 70 IU/mL, about 80 IU/mL, about 90 IU/mL, about 100 IU/mL, about 125 IU/mL, about 150 IU/mL, about 175 IU/mL, about 200 IU/mL, about 225 IU/mL, about 250 IU/mL, about 275 IU/mL, about 300 IU/mL, about 350 IU/mL, about 400 IU/mL, about 450 IU/mL, or about 500 IU/mL of IL-21.

In some aspects, the metabolic reprogramming medium comprises (i) potassium ion at a concentration higher than 5 mM and (ii) about 50 IU/mL of IL-21. In some aspects, the metabolic reprogramming medium comprises (i) potassium ion at a concentration higher than 5 mM and (ii) about 60 IU/mL of IL-21. In some aspects, the metabolic reprogramming medium comprises (i) potassium ion at a concentration higher than 5 mM and (ii) about 70 IU/mL of IL-21. In some aspects, the metabolic reprogramming medium comprises (i) potassium ion at a concentration higher than 5 mM and (ii) about 80 IU/mL of IL-21. In some aspects, the metabolic reprogramming medium comprises (i) potassium ion at a concentration higher than 5 mM and (ii) about 90 IU/mL of IL-21. In some aspects, the metabolic reprogramming medium comprises (i) potassium ion at a concentration higher than 5 mM and (ii) about 100 IU/mL of IL-21. In some aspects, the metabolic reprogramming medium comprises (i) potassium ion at a concentration higher than 5 mM and (ii) about 125 IU/mL of IL-21. In some aspects, the metabolic reprogramming medium comprises (i) potassium ion at a concentration higher than 5 mM and (ii) about 150 IU/mL of IL-21. In some aspects, the metabolic reprogramming medium comprises (i) potassium ion at a concentration higher than 5 mM and (ii) about 175 IU/mL of IL-21. In some aspects, the metabolic reprogramming medium comprises (i) potassium ion at a concentration higher than 5 mM and (ii) about 200 IU/mL of IL-21. In some aspects, the metabolic reprogramming medium comprises (i) potassium ion at a concentration higher than 5 mM and (ii) about 225 IU/mL of IL-21. In some aspects, the metabolic reprogramming medium comprises (i) potassium ion at a concentration higher than 5 mM and (ii) about 250 IU/mL of IL-21. In some aspects, the metabolic reprogramming medium comprises (i) potassium ion at a concentration higher than 5 mM and (ii) about 275 IU/mL of IL-21. In some aspects, the metabolic reprogramming medium comprises (i) potassium ion at a concentration higher than 5 mM and (ii) about 300 IU/mL of IL-21. In some aspects, the metabolic reprogramming medium comprises (i) potassium ion at a concentration higher than 5 mM and (ii) about 350 IU/mL of IL-21. In some aspects, the metabolic reprogramming medium comprises (i) potassium ion at a concentration higher than 5 mM and (ii) about 400 IU/mL of IL-21. In some aspects, the metabolic reprogramming medium comprises (i) potassium ion at a concentration higher than 5 mM and (ii) about 450 IU/mL of IL-21. In some aspects, the metabolic reprogramming medium comprises (i) potassium ion at a concentration higher than 5 mM and (ii) about 500 IU/mL of IL-21. In some aspects, the metabolic reprogramming medium comprising potassium ion and IL-21 further comprises NaCl at a concentration less than about 115 nM.

In some aspects, the metabolic reprogramming medium comprises at least about 0.1 ng/mL IL-21. In some aspects, the metabolic reprogramming medium comprises from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL IL-21.

In some aspects, the metabolic reprogramming medium comprises at least about 0.1 ng/mL, at least about 0.5 ng/mL, at least about 1 ng/mL, at least about 2 ng/mL, at least about 3 ng/mL, at least about 4 ng/mL, at least about 5 ng/mL, at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, at least about 15 ng/mL, at least about 16 ng/mL, at least about 17 ng/mL, at least about 18 ng/mL, at least about 19 ng/mL, or at least about 20 ng/mL IL-21. In some aspects, the metabolic reprogramming medium comprises at least about 1.0 ng/mL IL-21. In some aspects, the metabolic reprogramming medium comprises at least about 2.0 ng/mL IL-21. In some aspects, the metabolic reprogramming medium comprises at least about 3.0 ng/mL IL-21. In some aspects, the metabolic reprogramming medium comprises at least about 4.0 ng/mL IL-21. In some aspects, the metabolic reprogramming medium comprises at least about 5.0 ng/mL IL-21. In some aspects, the metabolic reprogramming medium comprises at least about 6.0 ng/mL IL-21. In some aspects, the metabolic reprogramming medium comprises at least about 7.0 ng/mL IL-21. In some aspects, the metabolic reprogramming medium comprises at least about 8.0 ng/mL IL-21. In some aspects, the metabolic reprogramming medium comprises at least about 9.0 ng/mL IL-21. In some aspects, the metabolic reprogramming medium comprises at least about 10 ng/mL IL-21. In some aspects, the metabolic reprogramming medium comprises at least about 10 ng/mL IL-21. In some aspects, the metabolic reprogramming medium comprises at least about 15 ng/mL IL-21. In some aspects, the metabolic reprogramming medium comprises at least about 20 ng/mL IL-21. In some aspects, the metabolic reprogramming medium comprises at least about 25 ng/mL IL-21. In some aspects, the metabolic reprogramming medium comprises at least about 30 ng/mL IL-21. In some aspects, the metabolic reprogramming medium comprises at least about 35 ng/mL IL-21.

In some aspects, the metabolic reprogramming medium described herein (e.g., comprising potassium ion at a concentration greater than 5 mM) comprises between about 500 IU/mL to about 1,500 IU/mL of IL-7. In some aspects, the culture medium comprises about 500 IU/mL, about 550 IU/mL, about 600 IU/mL, about 650 IU/mL, about 700 IU/mL, about 750 IU/mL, about 800 IU/mL, about 850 IU/mL, about 900 IU/mL, about 950 IU/mL, about 1,000 IU/mL, about 1,050 IU/mL, about 1,100 IU/mL, about 1,150 IU/mL, about 1,200 IU/mL, about 1,250 IU/mL, about 1,300 IU/mL, about 1,350 IU/mL, about 1,400 IU/mL, about 1,450 IU/mL, or about 1,500 IU/mL of IL-7.

In some aspects, the metabolic reprogramming medium useful for the present disclosure comprises (i) potassium ion at a concentration higher than 5 mM and (ii) about 500 IU/mL of IL-7. In some aspects, the metabolic reprogramming medium comprises (i) potassium ion at a concentration higher than 5 mM and (ii) about 550 IU/mL of IL-7. In some aspects, the metabolic reprogramming medium comprises (i) potassium ion at a concentration higher than 5 mM and (ii) about 600 IU/mL of IL-7. In some aspects, the metabolic reprogramming medium comprises (i) potassium ion at a concentration higher than 5 mM and (ii) about 650 IU/mL of IL-7. In some aspects, the metabolic reprogramming medium comprises (i) potassium ion at a concentration higher than 5 mM and (ii) about 700 IU/mL of IL-7. In some aspects, the metabolic reprogramming medium comprises (i) potassium ion at a concentration higher than 5 mM and (ii) about 750 IU/mL of IL-7. In some aspects, the metabolic reprogramming medium comprises (i) potassium ion at a concentration higher than 5 mM and (ii) about 800 IU/mL of IL-7. In some aspects, the metabolic reprogramming medium comprises (i) potassium ion at a concentration higher than 5 mM and (ii) about 850 IU/mL of IL-7. In some aspects, the metabolic reprogramming medium comprises (i) potassium ion at a concentration higher than 5 mM and (ii) about 900 IU/mL of IL-7. In some aspects, the metabolic reprogramming medium comprises (i) potassium ion at a concentration higher than 5 mM and (ii) about 950 IU/mL of IL-7. In some aspects, the metabolic reprogramming medium comprises (i) potassium ion at a concentration higher than 5 mM and (ii) about 1,000 IU/mL of IL-7. In some aspects, the metabolic reprogramming medium comprises (i) potassium ion at a concentration higher than 5 mM and (ii) about 1,050 IU/mL of IL-7. In some aspects, the metabolic reprogramming medium comprises (i) potassium ion at a concentration higher than 5 mM and (ii) about 1,100 IU/mL of IL-7. In some aspects, the metabolic reprogramming medium comprises (i) potassium ion at a concentration higher than 5 mM and (ii) about 1,150 IU/mL of IL-7. In some aspects, the metabolic reprogramming medium comprises (i) potassium ion at a concentration higher than 5 mM and (ii) about 1,200 IU/mL of IL-7. In some aspects, the metabolic reprogramming medium comprises (i) potassium ion at a concentration higher than 5 mM and (ii) about 1,250 IU/mL of IL-7. In some aspects, the metabolic reprogramming medium comprises (i) potassium ion at a concentration higher than 5 mM and (ii) about 1,300 IU/mL of IL-7. In some aspects, the metabolic reprogramming medium comprises (i) potassium ion at a concentration higher than 5 mM and (ii) about 1,350 IU/mL of IL-7. In some aspects, the metabolic reprogramming medium comprises (i) potassium ion at a concentration higher than 5 mM and (ii) about 1,400 IU/mL of IL-7. In some aspects, the metabolic reprogramming medium comprises (i) potassium ion at a concentration higher than 5 mM and (ii) about 1,450 IU/mL of IL-7. In some aspects, the metabolic reprogramming medium comprises (i) potassium ion at a concentration higher than 5 mM and (ii) about 1,500 IU/mL of IL-7. In some aspects, the metabolic reprogramming medium comprising potassium ion and IL-7 further comprises NaCl at a concentration less than about 115 nM.

In some aspects, the metabolic reprogramming medium comprises at least about 0.1 ng/mL IL-7. In some aspects, the metabolic reprogramming medium comprises from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL IL-7.

In some aspects, the metabolic reprogramming medium comprises at least about 0.1 ng/mL, at least about 0.5 ng/mL, at least about 1 ng/mL, at least about 2 ng/mL, at least about 3 ng/mL, at least about 4 ng/mL, at least about 5 ng/mL, at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, at least about 15 ng/mL, at least about 16 ng/mL, at least about 17 ng/mL, at least about 18 ng/mL, at least about 19 ng/mL, or at least about 20 ng/mL IL-7. In some aspects, the metabolic reprogramming medium comprises at least about 1.0 ng/mL IL-7. In some aspects, the metabolic reprogramming medium comprises at least about 2.0 ng/mL IL-7. In some aspects, the metabolic reprogramming medium comprises at least about 3.0 ng/mL IL-7. In some aspects, the metabolic reprogramming medium comprises at least about 4.0 ng/mL IL-7. In some aspects, the metabolic reprogramming medium comprises at least about 5.0 ng/mL IL-7. In some aspects, the metabolic reprogramming medium comprises at least about 6.0 ng/mL IL-7. In some aspects, the metabolic reprogramming medium comprises at least about 7.0 ng/mL IL-7. In some aspects, the metabolic reprogramming medium comprises at least about 8.0 ng/mL IL-7. In some aspects, the metabolic reprogramming medium comprises at least about 9.0 ng/mL IL-7. In some aspects, the metabolic reprogramming medium comprises at least about 10 ng/mL IL-7.

In some aspects, the metabolic reprogramming medium described herein (e.g., comprising potassium ion at a concentration greater than 5 mM) comprises between about 50 IU/mL to about 500 IU/mL of IL-15. In some aspects, the culture medium comprises about 50 IU/mL, about 60 IU/mL, about 70 IU/mL, about 80 IU/mL, about 90 IU/mL, about 100 IU/mL, about 125 IU/mL, about 150 IU/mL, about 175 IU/mL, about 200 IU/mL, about 225 IU/mL, about 250 IU/mL, about 275 IU/mL, about 300 IU/mL, about 350 IU/mL, about 400 IU/mL, about 450 IU/mL, or about 500 IU/mL of IL-15.

Therefore, in some aspects, the metabolic reprogramming medium comprises (i) potassium ion at a concentration higher than 5 mM and (ii) about 50 IU/mL of IL-15. In some aspects, the metabolic reprogramming medium comprises (i) potassium ion at a concentration higher than 5 mM and (ii) about 60 IU/mL of IL-15. In some aspects, the metabolic reprogramming medium comprises (i) potassium ion at a concentration higher than 5 mM and (ii) about 70 IU/mL of IL-15. In some aspects, the metabolic reprogramming medium comprises (i) potassium ion at a concentration higher than 5 mM and (ii) about 80 IU/mL of IL-15. In some aspects, the metabolic reprogramming medium comprises (i) potassium ion at a concentration higher than 5 mM and (ii) about 90 IU/mL of IL-15. In some aspects, the metabolic reprogramming medium comprises (i) potassium ion at a concentration higher than 5 mM and (ii) about 100 IU/mL of IL-15. In some aspects, the metabolic reprogramming medium comprises (i) potassium ion at a concentration higher than 5 mM and (ii) about 125 IU/mL of IL-15. In some aspects, the metabolic reprogramming medium comprises (i) potassium ion at a concentration higher than 5 mM and (ii) about 150 IU/mL of IL-15. In some aspects, the metabolic reprogramming medium comprises (i) potassium ion at a concentration higher than 5 mM and (ii) about 175 IU/mL of IL-15. In some aspects, the metabolic reprogramming medium comprises (i) potassium ion at a concentration higher than 5 mM and (ii) about 200 IU/mL of IL-15. In some aspects, the metabolic reprogramming medium comprises (i) potassium ion at a concentration higher than 5 mM and (ii) about 225 IU/mL of IL-15. In some aspects, the metabolic reprogramming medium comprises (i) potassium ion at a concentration higher than 5 mM and (ii) about 250 IU/mL of IL-15. In some aspects, the metabolic reprogramming medium comprises (i) potassium ion at a concentration higher than 5 mM and (ii) about 275 IU/mL of IL-15. In some aspects, the metabolic reprogramming medium comprises (i) potassium ion at a concentration higher than 5 mM and (ii) about 300 IU/mL of IL-15. In some aspects, the metabolic reprogramming medium comprises (i) potassium ion at a concentration higher than 5 mM and (ii) about 350 IU/mL of IL-15. In some aspects, the metabolic reprogramming medium comprises (i) potassium ion at a concentration higher than 5 mM and (ii) about 400 IU/mL of IL-15. In some aspects, the metabolic reprogramming medium comprises (i) potassium ion at a concentration higher than 5 mM and (ii) about 450 IU/mL of IL-15. In some aspects, the metabolic reprogramming medium comprises (i) potassium ion at a concentration higher than 5 mM and (ii) about 500 IU/mL of IL-15. In some aspects, the metabolic reprogramming medium comprising potassium ion and IL-15 further comprises NaCl at a concentration less than about 115 nM.

In some aspects, the metabolic reprogramming medium comprises at least about 0.1 ng/mL IL-15. In some aspects, the metabolic reprogramming medium comprises from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL IL-15.

In some aspects, the metabolic reprogramming medium comprises at least about 0.1 ng/mL, at least about 0.2 ng/mL, at least about 0.3 ng/mL, at least about 0.4 ng/mL, at least about 0.5 ng/mL, at least about 0.6 ng/mL, at least about 0.7 ng/mL, at least about 0.8 ng/mL, at least about 0.9 ng/mL, at least about 1 ng/mL, at least about 2 ng/mL, at least about 3 ng/mL, at least about 4 ng/mL, at least about 5 ng/mL, at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, at least about 15 ng/mL, at least about 16 ng/mL, at least about 17 ng/mL, at least about 18 ng/mL, at least about 19 ng/mL, or at least about 20 ng/mL IL-15. In some aspects, the metabolic reprogramming medium comprises at least about 1.0 ng/mL IL-15. In some aspects, the metabolic reprogramming medium comprises at least about 2.0 ng/mL IL-15. In some aspects, the metabolic reprogramming medium comprises at least about 3.0 ng/mL IL-15. In some aspects, the metabolic reprogramming medium comprises at least about 4.0 ng/mL IL-15. In some aspects, the metabolic reprogramming medium comprises at least about 5.0 ng/mL IL-15. In some aspects, the metabolic reprogramming medium comprises at least about 6.0 ng/mL IL-15. In some aspects, the metabolic reprogramming medium comprises at least about 7.0 ng/mL IL-15. In some aspects, the metabolic reprogramming medium comprises at least about 8.0 ng/mL IL-15. In some aspects, the metabolic reprogramming medium comprises at least about 9.0 ng/mL IL-15. In some aspects, the metabolic reprogramming medium comprises at least about 10 ng/mL IL-15. In some aspects, the metabolic reprogramming medium further comprises NaCl, wherein the total concentration of potassium ion and NaCl is from 110 mM to 140 mM.

In some aspects, the metabolic reprogramming medium comprises at least about 30 mM to at least about 100 mM potassium ion, about 300 ng/mL IL-2, and about 0.4 ng/mL IL-15. In some aspects, the metabolic reprogramming medium comprises more than 40 mM potassium ion, about 300 ng/mL IL-2, and about 0.4 ng/mL IL-15. In some aspects, the metabolic reprogramming medium comprises at least about 45 mM potassium ion, about 300 ng/mL IL-2, and about 0.4 ng/mL IL-15. In some aspects, the metabolic reprogramming medium comprises at least about 50 mM potassium ion, about 300 ng/mL IL-2, and about 0.4 ng/mL IL-15. In some aspects, the metabolic reprogramming medium comprises at least about 55 mM potassium ion, about 300 ng/mL IL-2, and about 0.4 ng/mL IL-15. In some aspects, the metabolic reprogramming medium comprises at least about 60 mM potassium ion, about 300 ng/mL IL-2, and about 0.4 ng/mL IL-15. In some aspects, the metabolic reprogramming medium comprises at least about 65 mM potassium ion, about 300 ng/mL IL-2, and about 0.4 ng/mL IL-15. In some aspects, the metabolic reprogramming medium comprises at least about 70 mM potassium ion, about 300 ng/mL IL-2, and about 0.4 ng/mL IL-15. In some aspects, the metabolic reprogramming medium comprises at least about 75 mM potassium ion, about 300 ng/mL IL-2, and about 0.4 ng/mL IL-15. In some aspects, the metabolic reprogramming medium comprises at least about 80 mM potassium ion, about 300 ng/mL IL-2, and about 0.4 ng/mL IL-15. In some aspects, the metabolic reprogramming medium comprises at least about 85 mM potassium ion, about 300 ng/mL IL-2, and about 0.4 ng/mL IL-15. In some aspects, the metabolic reprogramming medium comprises at least about 90 mM potassium ion, about 300 ng/mL IL-2, and about 0.4 ng/mL IL-15. In some aspects, the metabolic reprogramming medium comprises (i) at least about 70 mM potassium ion, (ii) about 60 mM NaCl, (iii) about 1.4 mM calcium, (iv) about 16 mM glucose, (v) about 300 ng/mL IL-2, and (vi) about 0.4 ng/mL IL-15.

II.A.7. Basal Media

In some aspects, the basal medium comprises a balanced salt solution (e.g., PBS, DPBS, HBSS, EBSS), Dulbecco's Modified Eagle's Medium (DMEM), Minimal Essential Medium (MEM), Basal Medium Eagle (BME), F-10, F-12, RPMI 1640, Glasgow Minimal Essential Medium (GMEM), alpha Minimal Essential Medium (alpha MEM), Iscove's Modified Dulbecco's Medium (IMDM), M199, OPTMIZER™ CTS™ T-Cell Expansion Basal Medium (ThermoFisher), OPTMIZER™ Complete, IMMUNOCULT™ XF (STEMCELL™ Technologies), IMMUNOCULT™, AIM V, TEXMACS™ medium, PRIME-XV® T cell CDM, X-VIVO™ 15 (Lonza), TRANSACT™ TIL expansion medium, or any combination thereof. In some aspects, the basal medium comprises PRIME-XV T cell CDM. In some aspects, the basal medium comprises OPTMIZER™. In some aspects, the basal medium comprises OPTMIZER™ Pro. In some aspects, the basal medium is serum free. In some aspects, the basal medium further comprises immune cell serum replacement (ICSR). For example, in some aspects, the basal medium comprises OPTMIZER™ Complete supplemented with ICSR, AIM V supplemented with ICSR, IMMUNOCULT™ XF supplemented with ICSR, RPMI supplemented with ICSR, TEXMACS™ supplemented with ICSR, or any combination thereof. In particular aspects, the basal medium comprises OPTMIZER™ complete.

In some aspects, the medium, e.g., the MRM, further comprises about 2.5% serum supplement (CTS™ Immune Cell SR, Thermo Fisher), 2 mM L-glutamine, 2 mM L-glutamax, MEM Non-Essential Amino Acids Solution, Pen-strep, 20 μg/ml Fungin™, sodium pyruvate, or any combination thereof. In some aspects, the medium further comprises O-Acetyl-L-carnitine hydrochloride. In some aspects, the medium further comprises a kinase inhibitor.

In some aspects, the medium further comprises a CD3 agonist. In some aspects, the CD3 agonist is an anti-CD3 antibody. In some aspects, the anti-CD3 antibody comprises OKT-3.

In some aspects, the medium further comprises a CD28 agonist. In some aspects, the CD28 agonist is an anti-CD28 antibody. In some aspects, the medium further comprises a CD27 ligand (CD27L). In some aspects, the medium further comprises a 4-1BB ligand (4-1BBL).

In some aspects, the present disclosure includes a cell culture comprising the medium disclosed herein, a cell bag comprising the medium disclosed herein, or a bioreactor comprising the medium disclosed herein.

II.B. Source and Activation of Cells

The immune cells of the present disclosure (which can be modified and cultured using the methods described herein), including primary T cells, can be obtained from a number of tissue sources, including peripheral blood mononuclear cells (PBMCs), bone marrow, lymph node tissue, cord blood, thymus tissue, tissue from a site of infection, ascites, pleural effusion, spleen tissue, and/or tumor tissue. Leukocytes, including PBMCs, can be isolated from other blood cells by well-known techniques, e.g., FICOLL™ separation and leukapheresis. Leukapheresis products typically contain lymphocytes (including T and B cells), monocytes, granulocytes, and other nucleated white blood cells. T cells can be further isolated from other leukocytes, for example, by centrifugation through a PERCOLL™ gradient or by counterflow centrifugal elutriation. A specific subpopulation of T cells, such as CD3⁺, CD25⁺, CD28⁺, CD4⁺, CD8⁺, CD45RA⁺, GITR⁺, and/or CD45RO⁺ T cells, can be further isolated by positive or negative selection techniques (e.g., using fluorescence-based or magnetic-based cell sorting). For example, T cells can be isolated by incubation with any of a variety of commercially available antibody-conjugated beads, such as Dynabeads®, CELLection™, DETACHaBEAD™ (Thermo Fisher) or MACS® cell separation products (Miltenyi Biotec), for a time period sufficient for positive selection of the desired T cells or negative selection for removal of unwanted cells.

In some instances, autologous T cells are obtained from a cancer patient directly following cancer treatment. It has been observed that following certain cancer treatments, in particular those that impair the immune system, the quality of T cells collected shortly after treatment can have an improved ability to expand ex vivo and/or to engraft after being engineered ex vivo.

Whether prior to or after genetic modification (e.g., using any of the modification methods described herein), T cells can be activated and expanded generally using methods as described, for example, in U.S. Pat. Nos. 5,858,358; 5,883,223; 6,352,694; 6,534,055; 6,797,514; 6,867,041; 6,692,964; 6,887,466; 6,905,680; 6,905,681; 6,905,874; 7,067,318; 7,144,575; 7,172,869; 7,175,843; 7,232,566; 7,572,631; and 10,786,533, each of which is expressly incorporated by reference herein in its entirety. Generally, T cells can be expanded in vitro or ex vivo by contact with a surface having attached thereto an agent that stimulates a CD3/TCR complex associated signal and a ligand that stimulates a costimulatory molecule on the surface of the T cells. In some aspects, T cell populations can be stimulated, such as by contact with an anti-CD3 antibody or antigen-binding fragment thereof, or an anti-CD3 antibody immobilized on a surface or by contact with a protein kinase C activator (e.g., bryostatins) in conjunction with a calcium ionophore. For co-stimulation of an accessory molecule on the surface of the T cells, a ligand that binds the accessory molecule can be used. For example, a population of T cells can be contacted with an anti-CD3 antibody and an anti-CD28 antibody under conditions appropriate for stimulating proliferation of the T cells. To stimulate proliferation of either CD4⁺ T cells or CD8⁺ T cells, an anti-CD3 antibody and an anti-CD28 antibody can be employed. In some aspects, the T cells are activated and expanded using e.g., DYNABEADS™, or commercial nanoparticles, e.g., TRANSACT™ (Miltenyi Biotech) or other known activation agents.

In some aspects, the methods described herein comprise contacting human immune cells (e.g., T cells and/or NK cells modified to express an increased level of a c-Jun protein) with programmable cell-signaling scaffolds (PCS) in a medium comprising potassium ion at a concentration higher than 5 mM (e.g., a metabolic reprogramming medium), as described herein. Non-limiting examples of programmable cell-signaling scaffolds (PCS) are described in WO2018/013797 and Chung et al. (Nature Biotechnology 36(2): 160-169 (2018), the contents of which are incorporated herein by reference in their entirety. In some aspects, the programmable cell-signaling scaffolds of the disclosure comprise a first layer comprising high surface area mesoporous silica micro rods (MSRs); a second layer comprising lipids coating the first layer; and a plurality of functional molecules loaded onto the scaffold. In some aspects, the functional molecules include, but are not limited to, a stimulatory molecule that activates T cells (T cell activating molecules). In some aspects, a stimulatory molecule activates T cells by engaging and/or clustering components of the T cell receptor complex. In some aspects, the stimulatory molecule comprises an anti-CD3 antibody or antigen-binding portion thereof. In some aspects, the functional molecules includes one or more co-stimulatory molecules which bind specifically to one or more co-stimulatory antigens. Representative examples of co-stimulatory molecules include, but are not limited to, molecules that specifically bind to CD28, 4-1BB (CD137), OX40 (CD134), CD27 (TNFRSF7), GITR (CD357), and/or CD30 (TNFRSF8). Such scaffolds are capable of mimicking functions commonly associated with antigen-presenting cells (APCs), which allows the scaffolds to elicit various functions on target cells, e.g., eliciting effector functions of T cells. As contemplated herein, in some aspects, the scaffolds mediate these effects via either direct or indirect interactions between the cell surface molecules residing in target cells (e.g., T cells) and the various functional molecules presented by the scaffolds. In some aspects, the scaffold modulates survival of target cells (e.g., T cells), growth of targeted cells (e.g., T cells), and/or function of target cells (e.g., T cells) through the physical or chemical characteristics of a scaffold itself.

II.C. Cells

The present disclosure also provides a modified cell which expresses an increased level of a c-Jun polypeptide compared to a reference cell (e.g., corresponding cell that has not been modified to have increased level of the c-Jun polypeptide). In some aspects, a cell does not naturally express a c-Jun protein, but has been modified to express the c-Jun protein. In some aspects, a cell is naturally capable of expressing a c-Jun protein, but has been modified to express an increased level of c-Jun protein. In some aspects, a cell is naturally capable of expressing a c-Jun protein, but has been modified to increase the expression of the endogenous c-Jun protein. Unless indicated otherwise, “c-Jun overexpression” (or derivatives thereof) comprises any of such modified cells. As described herein, any suitable methods known in the art can be used to modify the cells described herein.

In some aspects, a cell useful for the present disclosure has been modified to comprise an exogenous nucleotide sequence encoding a protein of interest, such that the encoded protein is expressed in the cell. As described herein, in some aspects, after the modification, the expression of the encoded protein is increased compared to a reference cell (e.g., corresponding cell that has not been modified to comprise the exogenous nucleotide sequence). In some aspects, a cell described herein has been modified to comprise multiple exogenous nucleotide sequence encoding different proteins of interest (e.g., a chimeric binding protein, c-Jun polypeptide, and/or EGFRt). Where multiple exogenous nucleotide sequences are involved, in some aspects, the multiple exogenous nucleotide sequences can be part of a single polycistronic polynucleotide.

In some aspects, a cell described herein has been modified with a transcriptional activator, which is capable of inducing and/or increasing the endogenous expression of a protein of interest (e.g., c-Jun) in the cell. As described herein, in some aspects, after the modification, the endogenous expression of the protein is increased compared to a reference cell (e.g., corresponding cell that has not been modified with the transcriptional activator). As used herein, the term “transcriptional activator” refers to a protein that increases the transcription of a gene or set of genes (e.g., by binding to enhancers or promoter-proximal elements of a nucleic acid sequence and thereby, inducing its transcription). Non-limiting examples of such transcriptional activators that can be used with the present disclosure include: Transcription Activator-like Effector (TALE)-based transcriptional activator, zinc finger protein (ZFP)-based transcriptional activator, Clustered Regularly Interspaced Short Palindromic Repeats (CRISPR)/CRISPR-associated protein (Cas) system-based transcriptional activator, or a combination thereof. See, e.g., Kabadi et al., Methods 69(2): 188-197 (September 2014), which is incorporated herein by reference in its entirety.

In some aspects, a cell described herein has been modified with a CRISPR/Cas-system-based transcriptional activator, such as CRISPR activation (CRISPRa). See, e.g., Nissim et al., Molecular Cell 54: 1-13 (May 2014), which is incorporated herein by reference in its entirety. CRISPRa is a type of CRISPR tool that comprises the use of modified Cas proteins that lacks endonuclease activity but retains the ability to bind to its guide RNA and the target DNA nucleic acid sequence. Non-limiting examples of such modified Cas proteins which can be used with the present disclosure are known in the art. See, e.g., Pandelakis et al., Cell Systems 10(1): 1-14 (January 2020), which is incorporated herein by reference in its entirety. In some aspects, the modified Cas protein comprises a modified Cas9 protein (also referred to in the art as “dCas9”). In some aspects, the modified Cas protein comprises a modified Cas12a protein. In some aspects, a modified Cas protein that is useful for the present disclosure is bound to a guide polynucleotide (e.g., small guide RNA) (“modified Cas-guide complex”), wherein the guide polynucleotide comprises a recognition sequence that is complementary to a region of a nucleic acid sequence encoding a protein of interest (e.g., c-Jun). In some aspects, the guide polynucleotide comprises a recognition sequence that is complementary to the promoter region of an endogenous nucleic acid sequence encoding a protein of interest. In some aspects, one or more transcriptional activators are attached to the modified Cas-guide complex (e.g., the N- and/or C-terminus of the modified Cas protein), such that when the modified Cas-guide complex is introduced into a cell, the one or more transcription activators can bind to a regulatory element (e.g., promoter region) of a nucleic acid sequence, and thereby induce and/or increase the expression of the encoded protein (e.g., c-Jun). In some aspects, the one or more transcription activators can bind to a regulatory element (e.g., promoter region) of an endogenous gene, and thereby induce and/or increase the expression of the encoded protein (e.g., c-Jun). Non-limiting Illustrative examples of common general activators that can be used include the omega subunit of RNAP, VP16, VP64 and p65. See, e.g., Kabadi and Gersbach, Methods 69: 188-197 (2014), which is incorporated herein by reference in its entirety.

In some aspects, one or more transcriptional repressors (e.g., Kruppel-associated box domain (KRAB)) can be attached to the modified Cas-guide complex (e.g., the N- and/or C-terminus of the modified Cas protein), such that when introduced into a cell, the one or more transcriptional repressors can repress or reduce the transcription of a gene, e.g., such as those that can interfere with the expression of c-Jun (e.g., Bach2). See, e.g., US20200030379A1 and Yang et al., J Transl Med 19:459 (2021), each of which is incorporated herein by reference in its entirety. In some aspects, a modified Cas protein useful for the present disclosure can be attached to both one or more transcriptional activators and one or more transcriptional repressors.

Not to be bound by any one theory, in some aspects, the use of such modified Cas proteins can allow for the conditional transcription and expression of a gene of interest. For example, in some aspects, a cell (e.g., T cells) is modified to comprise a ligand binding protein (e.g., CAR or TCR described herein), which is linked to a protease (e.g., tobacco etch virus (TEV)) and a single guide RNA (sgRNA) targeting the promoter region of c-Jun. In some aspects, the cell is modified to further comprise a linker for activation of T cells (LAT), complexed to the modified Cas protein attached to a transcriptional activator (e.g., dCas9-Vβ64-p65-Rta transcriptional activator (VPR)) via a linker (e.g., TEV-cleavable linker). Upon activation of the ligand binding protein, the modified Cas protein is released for nuclear localization and conditionally and reversibly induces the expression of c-Jun. Yang et al., J Immunother Cancer 9(Suppl2): A164 (2021), which is herein incorporated by reference in its entirety.

As will be apparent to those skilled in the art, in some aspects, a cell described herein has been modified using a combination of multiple approaches. For instance, in some aspects, a cell has been modified to comprise (i) an exogenous nucleotide sequence encoding one or more proteins (e.g., a chimeric binding protein and an EGFRt) and (ii) an exogenous transcriptional activator (e.g., CRISPRa) that increases expression of an endogenous protein (e.g., c-Jun). In some aspects, a cell has been modified to comprise (i) an exogenous nucleotide sequence encoding a first protein (e.g., a chimeric binding protein) and (ii) an exogenous nucleotide sequence encoding a second protein (e.g., a c-Jun protein). In some aspects, the modified cell can further comprise an exogenous nucleotide sequence encoding a third protein (e.g., EGFRt). As described herein, in some aspects, the exogenous nucleotide sequences encoding the first, second, and third proteins can be part of a single polycistronic vector.

Unless indicated otherwise, the one or more exogenous nucleotide sequences and/or transcriptional activators can be introduced into a cell using any suitable methods known in the art. Non-limiting examples of suitable methods for delivering one or more exogenous nucleotide sequences to a cell include: transfection (also known as transformation and transduction), electroporation, non-viral delivery, viral transduction, lipid nanoparticle delivery, and combinations thereof.

In some aspects, the immune cells of the present disclosure (which can be modified and cultured using the methods described herein) are isolated from a human subject, e.g., prior to culturing in vitro or ex vivo. In some aspects, the immune cells are isolated from a human subject for allogeneic cell therapy. In some aspects, the immune cells are isolated from a human subject for autologous cell therapy. In some aspects, the immune cells are T cells (e.g., CD4+ T cells and/or CD8+ T cells). In some aspects, the immune cells are NK cells. In some aspects, the immune cells are Tregs.

In some aspects, the cells, e.g., T cells and/or NK cells, are engineered before culturing according to the methods disclosed herein. In some aspects, the cells, e.g., T cells and/or NK cells, are engineered after culturing according to the methods disclosed herein. In some aspects, the cells, e.g., T cells and/or NK cells, are cultured according to the methods disclosed herein, e.g., in a hypotonic or isotonic medium comprising at least 5 mM potassium ion (e.g., higher than 5 mM, e.g., between about 40 mM to about 80 mM), prior to, during, and after cell engineering. In some aspects, the cells, e.g., T cells and/or NK cells, are engineered to express a chimeric antigen receptor (CAR). In some aspects, the cells, e.g., T cells and/or NK cells, are engineered to express an engineered T cell receptor (TCR). In certain aspects, culturing the cells, e.g., T cells and/or NK cells, under the conditions disclosed herein, e.g., in a hypotonic or isotonic medium comprising at least about 5 mM potassium ion, results in higher transduction efficiency. In some aspects, transduction efficiency is at least about 2-fold greater in cells, e.g., T cells and/or NK cells, cultured in hypotonic or isotonic medium comprising at least about 60 mM potassium ion, according to the methods disclosed herein, as compared to cells, e.g., T cells and/or NK cells, cultured in medium comprising 4 mM potassium ion or less. In some aspects, transduction efficiency is at least about 2.5-fold greater in cells, e.g., T cells and/or NK cells, cultured in hypotonic or isotonic medium comprising at least about 65 mM potassium ion, according to the methods disclosed herein, as compared to cells, e.g., T cells and/or NK cells, cultured in medium comprising 4 mM potassium ion or less.

As is apparent from the present disclosure, in some aspects, immune cells useful for the present disclosure (e.g., modified and cultured using the methods provided herein) comprise any suitable immune cells that are known in the art. Additionally, as further described elsewhere in the present disclosure, immune cells of the present disclosure have been modified, such that they differ from the corresponding immune cells that naturally exist in nature. For instance, immune cells described herein have been modified to express one or more proteins that help confer the distinct properties of the immune cells. Specifically, in some aspects, the modified immune cells provided herein express an increased level of a c-Jun protein compared to a reference cell (e.g., corresponding immune cells that have not been modified as described herein). In some aspects, the modified immune cells described herein also express a chimeric binding protein (e.g., CAR) that is not naturally expressed in the immune cells. As is apparent from the present disclosure, in some aspects, a chimeric binding protein can be expressed in a cell by modifying the cell with an exogenous polynucleotide encoding the chimeric binding protein. Additional proteins that can be encoded by the exogenous polynucleotide and thus, expressed in the immune cells are described elsewhere in the present disclosure. Non-limiting disclosures relating to such polynucleotides are provided below.

### II.C.1. c-Jun Encoding Polynucleotides

As described herein, in some aspects, immune cells described herein (e.g., modified and cultured using the methods provided herein) comprise, or are capable of expressing, a c-Jun protein. Where the immune cells are capable of naturally expressing the c-Jun protein, in some aspects, expression of the endogenous c-Jun protein is induced thereby resulting in increased or overexpression of the protein. In inducing the expression (or overexpression) of the c-Jun protein in a cell, in some aspects, the c-Jun protein is exogenously added. In some aspects, the c-Jun protein is recombinantly expressed in the cell. For instance, in some aspects, a cell described herein has been modified or engineered (e.g., genetically) to comprise an exogenous polynucleotide which comprises a nucleotide sequence encoding a c-Jun protein (also referred to herein as “c-Jun nucleotide sequence”), such that the expression of the c-Jun protein in the modified cell is increased compared to a reference cell (e.g., corresponding cell that was not modified to comprise the exogenous polynucleotide). In some aspects, a cell has been modified with a transcriptional activator (e.g., CRISPR/Cas-system-based transcription activator, e.g., CRISPRa), such that the expression of the endogenous c-Jun protein is increased compared to a reference cell (e.g., corresponding cell that has not been modified with the transcriptional activator).

In some aspects, due to the modification (e.g., introduction of the exogenously introduced c-Jun nucleotide sequence and/or transcriptional activator), the engineered cells overexpress, i.e., express a higher level (e.g., at least about 10, 20, 30, 40, 50, 60, 70, 80, 90, or 100% more, or at least about 1.5-, 2-, 3-, 4-, 5-, or 10-fold more) of, a c-Jun protein than corresponding cells without such a modification (“reference cell”). The terms “express increased levels [or amounts] of,” “overexpress,” or have “increased expression of” (and similar forms of the phrase used herein), are used interchangeably.

In some aspects, the engineered (or modified) cells described herein express at least about 2-100 fold more, about 5-50 fold more, about 5-40 fold more, about 5-30 fold more, about 5-20 fold more, about 8-20 fold more, or about 10-20 fold more c-Jun protein than the reference cell. In some aspects, the expression of the c-Jun protein in a modified cell described herein is increased by at least about 0.5-fold, by at least about 1-fold, by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 12-fold, by at least about 14-fold, by at least about 16-fold, by at least about 18-fold, by at least about 20-fold, by at least about 25-fold, by at least about 30-fold, by at least about 35-fold, by at least about 40-fold, by at least about 45-fold, or by at least about 50-fold, compared to the expression of the c-Jun protein in the reference cell.

Additionally, as described herein, in some aspects, a culture medium of the present disclosure (e.g., comprising potassium ion at a concentration higher than 5 mM) can also help further increase the expression of the c-Jun protein (or any other protein of interest) in the modified cells. Accordingly, in some aspects, when cultured using the methods provided herein, the expression of the c-Jun protein in the modified cells (e.g., resulting from the introduction of an exogenous nucleotide sequence encoding a c-Jun protein and/or a transcriptional activator that is capable of increasing the expression of the endogenous c-Jun protein) is further increased by at least 0.5-fold, by at least about 1-fold, by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 12-fold, by at least about 14-fold, by at least about 16-fold, by at least about 18-fold, by at least about 20-fold, by at least about 25-fold, by at least about 30-fold, by at least about 35-fold, by at least about 40-fold, by at least about 45-fold, or by at least about 50-fold, compared to the expression of the c-Jun protein in a reference cell. Accordingly, in some aspects, methods provided herein comprise modifying immune cells (e.g., T cells) with an exogenous polynucleotide, which encodes a c-Jun polypeptide, in a medium comprising potassium ion at a concentration higher than 5 mM, wherein after the modification the expression of the c-Jun polypeptide in the immune cell is increased compared to a reference cell. In some aspects, the immune cells can be modified with the exogenous polynucleotide in a separate medium and then subsequently transferred and cultured in the medium comprising the potassium ion at a concentration higher than 5 mM.

As described herein, in some aspects, the reference cell can comprise any of the following: (i) a corresponding cell that has not been modified and not cultured in the culture medium (i.e., does not comprise potassium ion at a concentration higher than 5 mM, e.g., TCM); (ii) a corresponding cell that has been modified but not cultured in the culture medium; (iii) a corresponding cell that has not been modified but cultured in the culture medium; or (iv) any combination of (i), (ii), and (iii).

As is apparent from the present disclosure, in some aspects, immune cells described herein (e.g., cultured using the methods provided herein) have been modified to express one or more additional transgenes in combination with an increased amount of c-Jun protein. For instance, in some aspects, an immune cell useful for the present disclosure has been modified to comprise: (i) a first exogenous nucleotide sequence encoding a c-Jun polypeptide and (ii) a second exogenous nucleotide sequence encoding a chimeric binding protein. In some aspects, the first and second nucleotide sequences are part of a single polynucleotide (referred to herein as a “polycistronic polynucleotide”). Non-limiting examples of such polycistronic polynucleotides are described further below. As described herein, in some aspects, such modification of the immune cells occurs in a medium comprising potassium ion at a concentration higher than 5 mM. In some aspects, the immune cells are modified in a reference medium (e.g., medium that does not comprise potassium ion at a concentration higher than 5 mM) and then cultured in a medium comprising potassium ion at a concentration higher than 5 mM. In some aspects, the T cells can be cultured in the medium comprising potassium ion at a concentration higher than 5 mM prior to the modification. In some aspects, the T cells that are modified can be further cultured in the medium comprising potassium ion at a concentration higher than 5 mM after the modification. In some aspects, the immune cells are cultured in a medium comprising potassium ion at a concentration higher than 5 mM prior to, during, and after the modification with the exogenous nucleotide sequence encoding one or more transgenes, such as those described herein.

c-Jun is an oncogenic transcription factor belonging to the activator protein-1 (AP-1) family. It interacts with various proteins (e.g., c-Fos) to form dimeric complexes that modulate a diverse range of cellular signaling pathways, including cell proliferation and tumor progression. Accordingly, increased c-Jun expression has been observed in certain cancers, and there has been much interest in developing c-Jun antagonists to treat such cancer. See, e.g., Brennan, A., et al., J Exp Clin Cancer Res 39(1): 184 (September 2020).

In humans, the c-Jun protein is encoded by the JUN gene, which is located on chromosome 1 (nucleotides 58,780,791 to 58,784,047 of GenBank Accession No. NC_000001.11, minus strand orientation). Synonyms of the JUN gene, and the encoded protein thereof, are known and include “Jun proto-oncogene, AP-1 transcription factor subunit,” “v-Jun avian sarcoma virus 17 oncogene homolog,” “transcription factor AP-1,” “Jun oncogene,” “AP-1,” “Jun activation domain binding protein,” “p39”, and “enhancer-binding protein AP1.” The wild-type human c-Jun protein sequence is 331 amino acids in length. The amino acid and nucleic acid sequences of the wild-type human c-Jun are provided in Tables 1 and 2, respectively.

The wild type human c-Jun (UniProt identifier: P05412-1) protein sequence is 331 amino acids in length (SEQ ID NO: 13). The amino acid and nucleic acid sequences are shown in Table 1 and 2, respectively.

TABLE 1
c-Jun Protein Sequence

| | |
|---|---|
| Wild-type human | MTAKMETTFYDDALNASFLPSESGPYGYSNPKILKQSMTLNLADPVGSLKPHLRAKNSDL |
| c-Jun (UniProt: | LTSPDVGLLKLASPELERLIIQSSNGHITTTPTPTQFLCPKNVTDEQEGFAEGFVRALAE |
| P05412-1) (SEQ | LHSQNTLPSVTSAAQPVNGAGMVAPAVASVAGGSGSGGFSASLHSEPPVYANLSNFNPGA |
| ID NO: 13) | LSSGGGAPSYGAAGLAFPAQPQQQQQPPHHLPQQMPVQHPRLQALKEEPQTVPEMPGETP |
| | PLSPIDMESQERIKAERKRMRNRIAASKCRKRKLERIARLEEKVKTLKAQNSELASTANM |
| | LREQVAQLKQKVMNHVNSGCQLMLTQQLQTF |

TABLE 2
c-Jun Nucleic Acid Sequence

| | |
|---|---|
| Wild-type JUN | gctcagagttgcactgagtgtggctgaagcagcgaggcgggagtggaggtgcgcggagt |
| (GenBank | caggcagacagacagacacagccagccagccaggtcggcagtatagtccgaactgcaaa |
| Accession No. | tcttattttcttttcaccttctctctaactgcccagagctagcgcctgtggctcccggg |
| NM_002228.4) | ctggtgtttcgggagtgtccagagagcctggtctccagccgcccccgggaggagagagc |
| (SEQ ID NO: 12) | tgctgcccaggcgctgttgacagcggcggaaagcagcggtacccacgcgcccgccgggg |
| * coding region is | gaagtcggcgagcggctgcagcagcaaagaactttcccggctgggaggaccggagacaa |
| bolded and | gtggcagagtcccggagccaacttttgcaagcctttcctgcgtcttaggcttctccacg |
| capitalized (SEQ | gcggtaaagaccagaaggcggcggagagccacgcaagagaagaaggacgtgcgctcagc |
| ID NO: 11) | ttcgctcgcaccggttgttgaacttgggcgagcgcgagccgcggctgccgggcgccccc |
| | tccccctagcagcggaggaggggacaagtcgtcggagtccgggcggccaagacccgccg |
| | ccggccggccactgcagggtccgcactgatccgctccgcggggagagccgctgctctgg |
| | gaagtgagttcgcctgcggactccgaggaaccgctgcgcacgaagagcgctcagtgagt |
| | gaccgcgacttttcaaagccgggtagcgcgcgcgagtcgacaagtaagagtgcgggagg |
| | catcttaattaaccctgcgctccctggagcgagctggtgaggagggcgcagcggggacg |
| | acagccagcgggtgcgtgcgctcttagagaaactttccctgtcaaaggctccggggggc |
| | gcgggtgtcccccgcttgccacagccctgttgcggccccgaaacttgtgcgcgcagccc |
| | aaactaacctcacgtgaagtgacggactgttctATGACTGCAAAGATGGAAACGACCTT |
| | CTATGACGATGCCCTCAACGCCTCGTTCCTCCCGTCCGAGAGCGGACCTTATGGCTACA |
| | GTAACCCCAAGATCCTGAAACAGAGCATGACCCTGAACCTGGCCGACCCAGTGGGGAGC |
| | CTGAAGCCGCACCTCCGCGCCAAGAACTCGGACCTCCTCACCTCGCCCGACGTGGGGCT |
| | GCTCAAGCTGGCGTCGCCCGAGCTGGAGCGCCTGATAATCCAGTCCAGCAACGGGCACA |
| | TCACCACCACGCCGACCCCCACCCAGTTCCTGTGCCCCAAGAACGTGACAGATGAGCAG |
| | GAGGGCTTCGCCGAGGGCTTCGTGCGCGCCCTGGCCGAACTGCACAGCCAGAACACGCT |
| | GCCCAGCGTCACGTCGGCGGCGCAGCCGGTCAACGGGGCAGGCATGGTGGCTCCCGCGG |
| | TAGCCTCGGTGGCAGGGGGCAGCGGCAGCGGCGGCTTCAGCGCCAGCCTGCACAGCGAG |
| | CCGCCGGTCTACGCAAACCTCAGCAACTTCAACCCAGGCGCGCTGAGCAGCGGCGGCGG |
| | GGCGCCCTCCTACGGCGCGGCCGGCCTGGCCTTTCCCGCGCAACCCCAGCAGCAGCAGC |
| | AGCCGCCGCACCACCTGCCCCAGCAGATGCCCGTGCAGCACCCGCGGCTGCAGGCCCTG |
| | AAGGAGGAGCCTCAGACAGTGCCCGAGATGCCCGGCGAGACACCGCCCCTGTCCCCCAT |
| | CGACATGGAGTCCCAGGAGCGGATCAAGGCGGAGAGGAAGCGCATGAGGAACCGCATCG |
| | CTGCCTCCAAGTGCCGAAAAAGGAAGCTGGAGAGAATCGCCCGGCTGGAGGAAAAAGTG |
| | AAAACCTTGAAAGCTCAGAACTCGGAGCTGGCGTCCACGGCCAACATGCTCAGGGAACA |
| | GGTGGCACAGCTTAAACAGAAAGTCATGAACCACGTTAACAGTGGGTGCCAACTCATGC |
| | TAACGCAGCAGTTGCAAACATTTtgaagagagaccgtcgggggctgaggggcaacgaag |
| | aaaaaaaataacacagagagacagacttgagaacttgacaagttgcgacggagagaaaa |
| | aagaagtgtccgagaactaaagccaagggtatccaagttggactgggttgcgtcctgac |
| | ggcgcccccagtgtgcacgagtgggaaggacttggcgcgccctcccttggcgtggagcc |
| | aacgttggacttttcgttaacattgaccaagaactgcatggacctaacattcgatctca |
| | ttcagtattaaaggggggagggggagggggttacaaactgcaatagagactgtagattg |
| | cttctgtagtactccttaagaacacaaagcggggggagggttggggaggggcggcagga |
| | gggaggtttgtgagagcgaggctgagcctacagatgaactctttctggcctgccttcgt |
| | taactgtgtatgtacatatatatattttttaatttgatgaaagctgattactgtcaata |
| | aacagcttcatgcctttgtaagttatttcttgtttgtttgtttgggtatcctgcccagt |
| | gttgtttgtaaataagagatttggagcactctgagtttaccatttgtaataaagtatat |
| | aatttttttatgttttgtttctgaaaattccagaaaggatatttaagaaaatacaataa |
| | actattggaaagtactcccctaacctcttttctgcatcatctgtagatactagctatct |
| | aggtggagttgaaagagttaagaatgtcgattaaaatcactctcagtgcttcttactat |
| | taagcagtaaaaactgttctctattagactttagaaataaatgtacctgatgtacctga |
| | tgctatggtcaggttatactcctcctcccccagctatctatatggaattgcttaccaaa |
| | ggatagtgcgatgtttcaggaggctggaggaaggggggttgcagtggagagggacagcc |
| | cactgagaagtcaaacatttcaaagtttggattgtatcaagtggcatgtgctgtgacca |
| | tttataatgttagtagaaattttacaataggtgcttattctcaaagcaggaattggtgg |
| | cagattttacaaaagatgtatccttccaatttggaatcttctctttgacaattcctaga |
| | taaaaagatggcctttgcttatgaatatttataacagcattcttgtcacaataaatgta |
| | ttcaaataccaa |

In some aspects, the immune cells disclosed herein have been modified to comprise an exogenous nucleotide sequence encoding a wild-type c-Jun protein, such as the wild-type nucleotide sequence set forth in SEQ ID NO: 12. Alternatively, in some aspects, the immune cells described herein are modified to comprise an exogenous nucleotide sequence encoding a mutant c-Jun protein, which retains the ability to prevent and/or reduce exhaustion in the immune cells. In some aspects, a mutant c-Jun protein, which can be expressed on the immune cells disclosed herein, comprises at least about 70% (e.g., at least about 75%, at least about 80%, at least about 85%, at least about 90%, at least about 95%, at least about 96%, at least about 97%, at least about 98%, or at least about 99%) sequence identity with the C-terminal amino acid residues (e.g., C-terminal 50, 75, 100, 150, 200, or 250 or more residues), the C-terminal portion (e.g., quarter, third, or half) or C-terminal domains (e.g., epsilon, bZIP, and amino acids C-terminal thereof) of a wildtype c-Jun (i.e., SEQ ID NO: 13). In some aspects, the N-terminal amino acid residues (e.g., N-terminal 50, 75, 100, or 150 or more), the N-terminal portion (e.g., quarter, third, or half) or N-terminal domains (e.g., delta, transactivation domain, and amino acids N-terminal thereof) of a wildtype c-Jun (i.e., SEQ ID NO: 13) are deleted, mutated, or otherwise inactivated. In some aspects, the c-Jun is a mutant human c-Jun, optionally comprising an inactivating mutation in its transactivation domain or delta domain. In some aspects, the c-Jun mutant comprises S63A and S73A mutations. In some aspects, the c-Jun mutant comprises a deletion between residues 2 and 102 as compared to the wild-type c-Jun (SEQ ID NO: 13). In some aspects, the c-Jun mutant comprises a deletion between residues 30 and 50 as compared to the wild-type c-Jun (SEQ ID NO: 13). In some aspects, the mutant c-Jun comprises (i) S63A and S73A mutations or (ii) a deletion between residues 2 and 102 or between residues 30 and 50 as compared to wild-type c-Jun (SEQ ID NO: 13). Non-limiting examples of mutant c-Jun proteins that are useful for the present disclosure are provided in US 2019/0183932 A1 and US 2017/0037376 A1, each of which is incorporated herein by reference in its entirety.

In some aspects, an immune cell described herein has been modified to comprise an exogenous nucleotide sequence encoding a c-Jun polypeptide, wherein the exogenous nucleotide sequence has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to any one of the nucleic acid sequences set forth in SEQ ID NOs: 1 to 11. In some aspects, an exogenous polynucleotide encoding a c-Jun polypeptide comprises the nucleic acid sequence set forth in any one of SEQ ID NOs: 1 to 11.

In some aspects, an exogenous polynucleotide encoding a c-Jun polypeptide has at least about 80%, at least about 85%, at least about 90%, at least about 95%, at least about 96%, at least about 97%, at least about 98%, or at least about 99% sequence identity to the nucleic acid sequence set forth in SEQ ID NO: 1. In some aspects, an exogenous polynucleotide encoding a c-Jun polypeptide has at least 89%, at least 90%, at least 91%, at least 92%, at least 93%, at least 94%, at least 95%, at least 96%, at least 97%, at least 98%, or at least 99% sequence identity to the nucleic acid sequence set forth in SEQ ID NO: 1. In some aspects, the exogenous polynucleotide comprises the nucleic acid sequence set forth in SEQ ID NO: 1.

In some aspects, an exogenous polynucleotide encoding a c-Jun polypeptide has at least about 80%, at least about 85%, at least about 90%, at least about 95%, at least about 96%, at least about 97%, at least about 98%, or at least about 99% sequence identity to the nucleic acid sequence set forth in SEQ ID NO: 2. In some aspects, an exogenous polynucleotide encoding a c-Jun polypeptide has at least 90%, at least 91%, at least 92%, at least 93%, at least 94%, at least 95%, at least 96%, at least 97%, at least 98%, or at least 99% sequence identity to the nucleic acid sequence set forth in SEQ ID NO: 2. In some aspects, the exogenous polynucleotide comprises the nucleic acid sequence set forth in SEQ ID NO: 2.

In some aspects, an exogenous polynucleotide encoding a c-Jun polypeptide has at least about 80%, at least about 85%, at least about 90%, at least about 95%, at least about 96%, at least about 97%, at least about 98%, or at least about 99% sequence identity to the nucleic acid sequence set forth in SEQ ID NO: 3. In some aspects, an exogenous polynucleotide encoding a c-Jun polypeptide has at least 87%, at least 88%, at least 89%, at least 90%, at least 91%, at least 92%, at least 93%, at least 94%, at least 95%, at least 96%, at least 97%, at least 98%, or at least 99% sequence identity to the nucleic acid sequence set forth in SEQ ID NO: 3. In some aspects, the exogenous polynucleotide comprises the nucleic acid sequence set forth in SEQ ID NO: 3.

In some aspects, an exogenous polynucleotide encoding a c-Jun polypeptide has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 4. In some aspects, an exogenous polynucleotide encoding a c-Jun polypeptide has at least 96%, at least 97%, at least 98%, or at least 99% to the nucleic acid sequence set forth in SEQ ID NO: 4. In some aspects, the exogenous polynucleotide comprises the nucleic acid sequence set forth in SEQ ID NO: 4.

In some aspects, an exogenous polynucleotide encoding a c-Jun polypeptide has at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 5. In some aspects, an exogenous polynucleotide encoding a c-Jun polypeptide has at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the nucleic acid sequence set forth in SEQ ID NO: 5. In some aspects, the exogenous polynucleotide comprises the nucleic acid sequence set forth in SEQ ID NO: 5.

In some aspects, an exogenous polynucleotide encoding a c-Jun polypeptide has at least about 80%, at least 85%, at least 90%, at least about 95%, at least about 96%, at least about 97%, at least about 98%, or at least about 99% sequence identity to the nucleic acid sequence set forth in SEQ ID NO: 6. In some aspects, an exogenous polynucleotide encoding a c-Jun polypeptide has at least 88%, at least 89%, at least 90%, at least 91%, at least 92%, at least 93%, at least 94%, at least 95%, at least 96%, at least 97%, at least 98%, or at least 99% sequence identity to the nucleic acid sequence set forth in SEQ ID NO: 6. In some aspects, the exogenous polynucleotide comprises the nucleic acid sequence set forth in SEQ ID NO: 6.

In some aspects, an exogenous polynucleotide encoding a c-Jun polypeptide has at least about 80%, at least about 85%, at least about 90%, at least about 95%, at least about 96%, at least about 97%, at least about 98%, or at least about 99% sequence identity to the nucleic acid sequence set forth in SEQ ID NO: 7. In some aspects, an exogenous polynucleotide encoding a c-Jun polypeptide has at least 88%, at least 89%, at least 90%, at least 91%, at least 92%, at least 93%, at least 94%, at least 95%, at least 96%, at least 97%, at least 98%, or at least 99% sequence identity to the nucleic acid sequence set forth in SEQ ID NO: 7. In some aspects, the exogenous polynucleotide comprises the nucleotide sequence set forth in SEQ ID NO: 7.

In some aspects, an exogenous polynucleotide encoding a c-Jun polypeptide has at least about 80%, at least about 85%, at least about 90%, at least about 95%, at least about 96%, at least about 97%, at least about 98%, or at least about 99% sequence identity to the nucleic acid sequence set forth in SEQ ID NO: 8. In some aspects, an exogenous polynucleotide encoding a c-Jun polypeptide has at least 90%, at least 91%, at least 92%, at least 93%, at least 94%, at least 95%, at least 96%, at least 97%, at least 98%, or at least 99% sequence identity to the nucleic acid sequence set forth in SEQ ID NO: 8. In some aspects, the exogenous polynucleotide comprises the nucleotide sequence set forth in SEQ ID NO: 8.

In some aspects, an exogenous polynucleotide encoding a c-Jun polypeptide has at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 9. In some aspects, an exogenous polynucleotide encoding a c-Jun polypeptide has at least 88%, at least 89%, at least 90%, at least 91%, at least 92%, at least 93%, at least 94%, at least 95%, at least 96%, at least 97%, at least 98%, or at least 99% sequence identity to the nucleic acid sequence set forth in SEQ ID NO: 9. In some aspects, the exogenous polynucleotide comprises the nucleotide sequence set forth in SEQ ID NO: 9.

In some aspects, an exogenous polynucleotide encoding a c-Jun polypeptide has at least about 85%, at least about 90%, at least about 95%, at least about 96%, at least about 97%, at least about 98%, or at least about 99% sequence identity to the nucleic acid sequence set forth in SEQ ID NO: 10. In some aspects, an exogenous polynucleotide encoding a c-Jun polypeptide has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the nucleic acid sequence set forth in SEQ ID NO: 10. In some aspects, the exogenous nucleotide comprises the nucleotide sequence set forth in SEQ ID NO: 10.

Exemplary c-Jun nucleotide sequences are provided in Table 3 (below).

TABLE 3
c-Jun Nucleotide Sequences

| | |
|---|---|
| c-Jun nucleotide | atgacagccaagatggaaaccacattctacgacgacgccctgaacgcctcattcctgc |
| sequence #1 (SEQ | cttctgagagcggaccttacggctacagcaatcctaagatcctgaaacagagcatgac |
| ID NO: 1) | ccttaacctggctgatcctgttggaagcctgaaacctcacctgagagccaaaaacagc |
| | gacctgctcaccagccctgatgtgggcctgctgaagctggcctctccagagctggaac |
| | ggctgatcatccagagcagcaacggccacatcacaaccacccctacccctacacaatt |
| | cctgtgccctaagaacgtgaccgacgagcaggagggcttcgccgaaggctttgtgcgg |
| | gccctggcagaactgcactctcagaacaccctgcctagcgtgacctccgccgcccagc |
| | ctgtcaacggcgccggaatggtggcccctgccgtggcttctgtggccggcggcagcgg |
| | cagcggcggattcagcgcctctctgcactctgagcctcctgtctacgccaatctgtct |
| | aatttcaaccccggagccctgtccagcggcggcggagctcctagctacggcgctgctg |
| | gactggccttccccgcccagccccagcaacagcagcagcctccacaccacctgcccca |
| | gcagatgcccgtgcagcaccctagactgcaggccctgaaggaagaaccccaaacagtg |
| | cctgagatgcctggcgagacacctccactgagccccatcgacatggaaagccaggagc |
| | ggatcaaggccgagagaaagagaatgcggaacagaatcgccgctagcaagtgcagaaa |
| | gcggaagctggaaagaatcgccagactggaagagaaggtgaagaccctgaaagcccaa |
| | aatagcgagctggccagcaccgccaacatgctgcgggaacaggtggcccagctgaagc |
| | agaaggtgatgaaccacgtgaactctggttgtcagctgatgctgacccagcagctcca |
| | gaccttc |
| c-Jun nucleotide | atgacagccaagatggaaaccaccttctacgacgacgccctcaacgcctccttcctgc |
| sequence #2 (SEQ | cttctgagagcggtccttacggctacagcaaccccaagatcctgaagcaaagcatgac |
| ID NO: 2) | cctgaacctggccgaccccgttggctccctgaaacctcacctgagagccaaaaacagc |
| | gacctgctgaccagccctgatgtgggcctgctgaagctggcctctccagagctggaaa |
| | gactgattatccagagcagcaacggccacatcaccacaacacctacccctacacagtt |
| | cctgtgccctaagaacgtgactgatgagcaggagggctttgccgagggcttcgtgaga |
| | gccctggctgagctgcattctcagaacaccctgcctagcgtgacctctgccgcccagc |
| | ctgttaatggcgccggcatggtggcccctgccgtggcctctgtggccggaggcagcgg |
| | cagcggcggattcagcgcctctctgcacagcgagccccccgtctacgccaacctgagc |
| | aatttcaaccctggcgccctgtccagcggcggcggcgccccttcatatggcgctgccg |
| | gcctggccttccccgctcagccccagcagcagcaacagcctccacaccacctgcccca |
| | gcagatgcccgtgcagcaccccagactgcaggccctgaaggaagaacctcagaccgtg |
| | cccgagatgcctggcgagacccctcctctgagccctatcgacatggaaagccaggaga |
| | gaatcaaggccgagaggaagcggatgcggaacagaatcgccgccagcaagtgcagaaa |
| | aagaaagctggaacggatcgccagactggaggagaaggtgaagacactgaaagcccaa |
| | aattctgaactggcctctaccgccaatatgctgcgcgagcaggtggctcaactgaagc |
| | agaaggtgatgaaccacgtgaacagcggatgtcagctgatgctgacacagcagctgca |
| | gactttt |
| c-Jun nucleotide | atgaccgccaagatggaaaccaccttctacgacgacgccctgaacgccagctttctgc |
| sequence #3 (SEQ | cttctgagtctggcccctacggctacagcaaccccaagatcctgaagcagagcatgac |
| ID NO: 3) | cctgaacctggccgatcctgtgggcagcctgaaacctcacctgagagccaagaacagc |
| | gacctgctgacaagccctgatgtgggcctgctgaaactggcctctcctgagctggaac |
| | ggctgatcatccagagcagcaacggccacatcaccaccacacctacaccaacacagtt |
| | tctgtgccccaagaacgtgaccgacgagcaagagggattcgccgagggctttgttaga |
| | gccctggccgaactgcacagccagaataccctgcctagcgtgacatctgccgctcagc |
| | ctgttaatggcgccggaatggttgctcctgccgtggcttctgttgctggcggatctgg |
| | atctggcggctttagcgcctctctgcactctgagcctccagtgtacgccaacctgagc |
| | aacttcaaccctggcgctcttagctctggtggcggagcaccttcttatggcgctgccg |
| | gattggcctttcctgctcagcctcagcagcagcaacagcctcctcatcatctgcccca |
| | gcagatgcctgtgcagcaccctagactgcaggccctgaaagaggaaccccagacagtc |
| | cctgagatgcccggcgaaacacctcctctgagccccatcgacatggaaagccaagagc |
| | ggatcaaggccgagcggaagcggatgagaaatagaatcgccgcctccaagtgccggaa |
| | gaggaagctggaaagaatcgcccggctggaagagaaagtgaaaaccctgaaggcccag |
| | aactccgagctggcctctaccgccaacatgctgagagaacaggtggcccagctgaaac |
| | agaaagtcatgaaccacgtgaacagcggctgccagctgatgctgacacagcagctgca |
| | gaccttc |
| c-Jun nucleotide | atgactgccaaaatggagactacattctatgacgacgccctcaatgccagttttttgc |
| sequence #4 (SEQ | cgagtgaatccggcccctacggctattcaaaccctaagatcctcaagcaatcaatgac |
| ID NO: 4) | cctcaatcttgctgacccagttggctccctgaaaccccatctcagagctaaaaatagt |
| | gacctccttacttcccctgatgttggactcctcaaacttgcttctcccgaactcgaac |
| | gcttgatcattcaatcttccaacggccacatcacaacaacacccacacccacccagtt |
| | tctttgcccaaaaaatgtcaccgatgaacaggaaggtttcgcggaaggattcgtccgc |
| | gcgctggccgaactgcactcccagaatacacttccttcagttacgtcagccgcccagc |
| | cagtgaatggtgcgggaatggttgctcctgcggtcgcttctgtcgcagggggctccgg |
| | ttctggcggatttagcgcctctctgcattccgagccacctgtatatgctaatctttct |
| | aattttaaccccggagccttgtctagcggcggtggtgcccccagctacggtgctgcag |
| | gactcgcetteccagctcaacctcagcagcagcaacaacccccccatcaccttcccca |
| | acagatgccagtacaacatccaaggctccaggccctcaaagaggaaccacagacggtg |
| | cccgaaatgcctggcgaaactccaccactttcccctattgatatggaatcccaagagc |
| | gcatcaaggccgaaagaaagcgaatgcggaatagaatagcagcttcaaaatgtagaaa |
| | acggaaattggaacgaatcgcacggttggaagaaaaggtgaagaccttgaaagcccag |
| | aacagtgagctcgcctctaccgctaacatgctgcgcgagcaagtcgcacaacttaagc |
| | agaaggtgatgaaccatgtgaatagcggatgtcaacttatgctgactcaacagttgca |
| | aaccttt |
| c-Jun nucleotide | atgaccgcgaaaatggagacaacattttacgatgatgcactgaacgcctcttttctgc |
| sequence #5 (SEQ | caagtgaatccggcccctacggatactcaaaccctaagattctgaaacagtctatgac |
| ID NO: 5) | tctcaacctggccgacccagttggcagtctgaagcctcatttgcgagccaagaatagt |
| | gatctgctgacctccccagacgtgggactgctgaaactcgcctcacctgaacttgagc |
| | gcttgattatacagtcatccaatgggcacatcacaacaacacctactcctacccagtt |
| | tctgtgccccaaaaacgtcaccgatgagcaggagggattcgcggaaggctttgtgcgc |
| | gccctggctgaattgcatagtcagaacactcttcccagcgtaaccagcgccgcccaac |
| | cagtgaatggagccggtatggtggctcccgcggtggctagtgttgcgggggggtcagg |
| | ctctggtgggttcagtgcttctcttcactctgaaccccctgtgtatgccaatctgtct |
| | aactttaaccctggggccctctcctctggtgggggtgcccccagctacggagcggccg |
| | gcctggcctttcctgcccagcctcagcagcagcagcaaccccctcatcatcttccgca |
| | gcagatgccagtacagcatccacgcctgcaggctcttaaggaggagccccagacggtg |
| | cccgaaatgcccggggaaactccacccttgtcccccattgacatggagtcccaggagc |
| | ggatcaaggctgaaagaaagaggatgcggaatcgcatcgcagcctctaaatgccgcaa |
| | gcggaaacttgagaggatcgcgcggttggaggaaaaagtaaaaaccttgaaggcacag |
| | aactctgagctggcgagtactgccaacatgctcagagaacaagtcgcacagctgaagc |
| | agaaagtgatgaaccatgtgaacagcggttgtcagctgatgctgactcagcagctgca |
| | gaccttc |
| c-Jun nucleotide | atgaccgccaagatggagaccacattctacgatgacgctctgaacgcttcctttctgc |
| sequence #6 (SEQ | cttccgagtccggcccctacggctactccaatcccaagattctgaagcagagcatgac |
| ID NO: 6) | actgaatctggctgatcccgtgggatctctgaagcctcatctgagagccaagaattcc |
| | gatctgctgacaagccccgacgtgggactgctcaaactggccagccccgaactggaga |
| | ggctcattatccagagctccaacggccacatcaccacaacacctacccctacccagtt |
| | tctctgtcccaagaacgtgacagacgagcaagagggatttgccgaaggcttcgtgaga |
| | gccctcgccgaactgcatagccagaacacactgccttccgtgaccagcgctgctcaac |
| | ccgtgaacggcgctggcatggtcgctcccgccgtcgccagcgtggctggaggaagcgg |
| | atccggaggcttcagcgcttccctccacagcgaacctcccgtgtacgctaatctgagc |
| | aacttcaaccccggcgctctgagcagcggaggaggagctcctagctatggagctgccg |
| | gactggcttttcccgcccagccccagcagcagcagcagcccccccatcatctgcctca |
| | gcagatgcccgtgcagcatcccagactccaagctctgaaggaggagcctcagaccgtc |
| | cccgagatgcccggcgaaaccccccctctgtcccccatcgacatggaaagccaagaga |
| | ggatcaaggccgagaggaagaggatgaggaatagaatcgccgccagcaagtgtagaaa |
| | gaggaagctggagaggatcgccagactggaggagaaggtgaagaccctcaaggctcag |
| | aattccgagctggccagcacagccaacatgctgagagagcaagtggcccagctcaagc |
| | agaaggtgatgaaccacgtcaacagcggatgccagctgatgctcacccagcagctgca |
| | gaccttc |
| c-Jun nucleotide | atgaccgctaaaatggaaaccactttctatgacgatgccctgaacgcctccttccttc |
| sequence #7 (SEQ | cgtccgagtccggaccctacggatactcaaatcctaagatcctcaaacagtcgatgac |
| ID NO: 7) | cctcaacctggccgaccccgtgggatccctgaagccgcacttgcgcgccaagaactcc |
| | gacctcctgacgagcccagacgtgggcctgctgaagctcgcatcacccgaacttgagc |
| | ggttgatcattcagtcctccaacggacatatcaccaccactcccaccccaactcagtt |
| | tctgtgtccgaagaacgtgaccgatgagcaagagggattcgccgagggattcgtggg |
| | gccctggccgagctgcatagccagaacacccttccatccgtgacctcggcggctcagc |
| | ctgtgaacggcgcgggaatggtcgcgcccgccgtggcctcggtggccgggggcagcgg |
| | cagcgggggattttccgcgtcgctgcactccgagccgccggtgtacgccaacctgtca |
| | aacttcaaccctggggccctgagctccggcggtggagcaccttcgtacggcgccgctg |
| | gcctggcgttccccgcgcaaccacagcagcaacagcagccccctcaccacctccccca |
| | acaaatgcctgtgcagcacccgaggctgcaggccctcaaggaagaaccccagactgtg |
| | ccggaaatgccgggggagactccgccgctgtcccctatcgacatggaatcacaggaac |
| | gcattaaggcagagcggaagcgcatgcggaaccggattgccgcctccaagtgccgcaa |
| | gagaaagctcgaaagaatcgccagattggaagaaaaggtcaagactctgaaggcccag |
| | aactctgagctggcatccaccgctaatatgctgagggaacaagtggcccagctgaaac |
| | agaaggtcatgaaccacgtcaacagcggttgccagctgatgctgacccagcaactcca |
| | gacattc |
| c-Jun nucleotide | atgaccgccaagatggagaccaccttctacgacgacgccctgaacgccagcttcctgc |
| sequence #8 (SEQ | ccagcgagagcggaccctacggctactctaaccccaagatcctgaaacagagcatgac |
| ID NO: 8) | actgaatctggccgaccccgtgggcagcctgaagcctcaccttagagccaagaacagc |
| | gacctgctgaccagccccgacgtgggcctgctgaagctcgcctctccagagttagaga |
| | gactgatcatccagtccagcaacggccacatcacaaccaccccaacccctacccagtt |
| | cctgtgccccaagaacgtgaccgacgagcaggagggcttcgccgagggctttgtgaga |
| | gccctggccgagttgcactctcagaacaccctgccctccgtgaccagcgccgctcaac |
| | ctgtgaacggcgcaggaatggttgctcctgccgtggccagcgttgcaggcggatctgg |
| | aagtggaggcttctccgcctcccttcacagcgagcctcccgtgtacgccaacctgagc |
| | aacttcaaccccggcgccctgagcagtggaggaggcgctcccagctatggagcagctg |
| | gattagccttccccgcccagccacagcagcagcaacagcctccccaccacctgcctca |
| | gcaaatgcctgtgcagcaccctcggctgcaggcccttaaggaggagccccagaccgtt |
| | cctgagatgcctggcgagacccctcccctgagccctatcgacatggagtcccaggagc |
| | ggatcaaggccgagcggaagcggatgcggaaccggatcgctgcttccaagtgccggaa |
| | gagaaagctggagagaatcgcccggctggaggagaaggtgaagaccctgaaggcccag |
| | aactccgagctggcctccaccgccaacatgctgcgggagcaggttgcacagctgaagc |
| | agaaggtcatgaaccacgtgaacagcggctgccagctgatgctgacccagcagctgca |
| | gaccttc |
| c-Jun nucleotide | atgacagcgaagatggagacaaccttctatgacgatgctcttaacgcctccttcctgc |
| sequence #9 (SEQ | cttccgaaagcgggccctacgggtactctaatcctaagatacttaagcaatcgatgac |
| ID NO: 9) | tctcaacctcgctgacccggttggctcactgaaaccacacctgagagctaagaatagt |
| | gacctgctcactagtcccgatgtcgggcttctgaagctggcctctcccgagctggaga |
| | ggcttatcatccaatcatcaaatggccacatcaccactaccccaacaccaactcaatt |
| | cctttgccctaaaaacgtgaccgacgaacaggaaggcttcgccgagggttttgtccgg |
| | gccttggccgagctgcattctcaaaatacactgccaagcgtcacttctgcggcgcagc |
| | cggttaacggagcagggatggtggctcccgccgttgctagcgtggccggcggttccgg |
| | ctccggcggtttctctgcctccttgcattctgagccaccagtctacgcgaacctgtcc |
| | aactttaatccgggggcgctgagtagcggaggcggcgcccctagctatggggcagctg |
| | gactggccttcccggcacaaccccaacaacaacagcaaccgccacaccatcttcctca |
| | acaaatgccagtgcaacatccacgcttacaagccctcaaggaggaaccccagaccgtg |
| | cctgagatgcccggcgaaaccccgccattgagccctattgacatggaaagtcaagaga |
| | gaattaaggcagagcgcaagagaatgaggaaccggatcgcagcatctaagtgccgcaa |
| | acggaaattggagcggatcgctcgcttggaggagaaggtcaagactctcaaggcccag |
| | aactccgagcttgcgagcacagctaatatgctgcgcgagcaggtggcccagttaaaac |
| | aaaaggtcatgaaccatgtgaacagcggctgtcagctgatgcttacgcaacagctgca |
| | aacctttggctccggtgcaacgaacttcagcctgctgaagcaggccggagatgttgag |
| | gaaaatccaggtccc |
| c-Jun nucleotide | atgacggccaaaatggagactacgttctacgatgacgcactcaacgcgtccttcctgc |
| sequence #10 (SEQ | cctctgagagtggaccctatggctactccaatccaaagatcctgaagcagtctatgac |
| ID NO: 10) | cctcaacctggcggacccggtgggctcccttaagccgcacttgcgcgccaagaactcc |
| | gacctgctgacctcccctgatgtgggcctcctcaagctcgctagccctgaattggaga |
| | ggctgatcatccagagctcaaatggccacatcaccaccacacctaccccaacccagtt |
| | cctgtgcccaaaaaacgtgaccgacgagcaggagggcttcgcggagggcttcgtcaga |
| | gctctggccgagctgcactcacagaacacgctcccttccgtgacctccgctgcccagc |
| | cggtcaatggcgctggaatggtggctccggctgtggcctctgttgccggcggctccgg |
| | ctccggaggcttttcagcttctctgcattctgagcccccagtgtacgctaacctgagc |
| | aacttcaaccccggggcgctcagctccggtggcggtgccccgagctacggcgcggctg |
| | ggctggcgttccccgctcagcctcagcagcaacagcaacctccccaccacctgccaca |
| | gcagatgcctgtgcagcacccacgcctgcaggccttgaaggaggaacctcagactgtg |
| | ccagagatgcccggcgagaccccacccctgtccccgattgacatggagagccaggagc |
| | gcatcaaggcagagcgcaagcgtatgcgcaaccgcatcgcggcctccaagtgccgaaa |
| | gcgcaagctggagcggattgctcgcctggaggagaaggtgaagaccctgaaggcccag |
| | aattccgagctggcctcgaccgccaacatgctacgagaacaggtcgcgcagctgaaac |
| | agaaggtcatgaaccatgtcaacagcgggtgccagctgatgttgacccagcagcttca |
| | gaccttc |

The c-Jun nucleotide sequence disclosed herein can be codon-optimized using any methods known in the art. For instance, in some aspects, the codons of a c-Jun nucleotide sequence disclosed herein has been optimized to modify (e.g., increase or decrease) one or more of the following parameters compared to the wild-type nucleotide sequence (e.g., SEQ ID NO: 11): (i) codon adaptation index (i.e., codon usage bias); (ii) guanine-cytosine (GC) nucleotide content; (iii) mRNA secondary structure and unstable motifs; (iv) repeat sequences (e.g., direct repeats, inverted repeats, dyad repeats); (v) restriction enzyme recognition sites; or (vi) combinations thereof.

In some aspects, an exogenous polynucleotide encoding a c-Jun polypeptide provided herein is capable of increasing the expression of the encoded c-Jun protein when transfected, transduced or otherwise introduced into an immune cell (e.g., human immune cell), as compared to a corresponding expression in a cell transfected with the wild-type c-Jun nucleotide sequence (e.g., SEQ ID NO: 11). In some aspects, the expression of the c-Jun protein in the immune cell modified to comprise the exogenous polynucleotide is increased by at least about 0.5-fold, by at least about 1-fold, by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 12-fold, by at least about 14-fold, by at least about 16-fold, by at least about 18-fold, by at least about 20-fold, by at least about 25-fold, by at least about 30-fold, by at least about 35-fold, by at least about 40-fold, by at least about 45-fold, or by at least about 50-fold, compared to the corresponding expression in the cell transfected, transduced, or otherwise genetically modified to express with the wild-type c-Jun nucleotide sequence (e.g., SEQ ID NO: 11).

While certain disclosures provided above generally relate to modifying an immune cell to comprise an exogenous nucleotide sequence encoding a c-Jun protein (wild-type c-Jun or a variant thereof), it will be apparent to those skilled in the art that other suitable methods can be used to induce and/or increase c-Jun protein expression (either wild-type or a variant thereof) in a cell. For instance, as described herein, in some aspects, the endogenous c-Jun protein expression can be increased with a transcriptional activator (e.g., CRISPRa). Unless indicated otherwise, disclosures provided above using exogenous nucleotide sequences equally apply to other approaches of inducing and/or increasing c-Jun protein expression in a cell provided herein (e.g., transcriptional activator, e.g., CRISPRa).

In some aspects, the increased expression of the c-Jun protein can improve and/or enhance one or more properties of the modified immune cells (e.g., T cells, such as CD4+ and/or CD8+ T cells). Non-limiting examples of such properties include: resistance to exhaustion (e.g., as indicated by reduced expression of exhaustion markers, such as PD-1, CD39, TIM-3, and/or LAG-3; increased persistence/survival; delay of the onset of dysfunctional states; and/or increased cytokine (e.g., IFN-γ and/or IL-2) production), increased expansion/proliferation, increased antigen sensitivity, improved effector function, in particular, improved effector function following repeated antigen stimulation (e.g., cytokine production upon antigen stimulation, lysis of cells expressing the target antigen, or both), or combinations thereof.

Assays useful for measuring exhaustion, cell phenotype, persistence, cytotoxicity and/or killing, proliferation, cytokine production/release, and gene expression profiles are known in the art and include, for example flow cytometry, intracellular cytokine staining (ICS), INCUCYTE® immune cell killing analysis, Meso Scale Discovery (MSD) or similar assay, persistent antigen stimulation assays, bulk and single cell RNAseq (see e.g., Fron Genet. 2020; 11:220; 2019 Bioinformatics 35:i436-445; 2019 Annual Review of Biomed. Data Sci. 2:139-173), cytotoxicity/killing assays, ELISA, western blot and other standard molecular and cell biology methods such as described herein or as described, for example, in Current Protocols in Molecular Biology or Current Protocols in Immunology (John Wiley & Sons, Inc., 1999-2021) or elsewhere.

In some aspects, the increased expression of the c-Jun protein increases the resistance of the immune cell to exhaustion. In some aspects, the resistance to exhaustion is increased by at least about 0.5-fold, by at least about 1-fold, by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 12-fold, by at least about 14-fold, by at least about 16-fold, by at least about 18-fold, by at least about 20-fold, by at least about 25-fold, by at least about 30-fold, by at least about 35-fold, by at least about 40-fold, by at least about 45-fold, or by at least about 50-fold, compared to a reference cell (e.g., corresponding cell that was not modified to have increased c-Jun protein expression).

In some aspects, the increased c-Jun protein expression can decrease exhaustion in an exhausted cell. In some aspects, the increased expression of the c-Jun protein can decrease exhaustion by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%, compared to a reference cell (e.g., corresponding exhausted cell that was not modified to have increased c-Jun protein expression), as measured, for example, using one or more assays as described herein.

In some aspects, the increased c-Jun protein expression delays the onset of exhaustion in a cell. In some aspects, the increased expression of the c-Jun protein delays the onset of exhaustion by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%, compared to a reference cell (e.g., corresponding cell that was not modified to have increased c-Jun protein expression), as measured, for example, using one or more assays as described herein. In some aspects, the increased c-Jun protein expression delays the onset of exhaustion by at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days or more.

Accordingly, in some aspects, the expression of one or more exhaustion markers (e.g., TIGIT, PD-1, TIM-3, and/or LAG-3) in a cell described herein is decreased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%, compared to a reference cell (e.g., corresponding cell that was not modified to have increased c-Jun protein expression).

In some aspects, the expression of one or more exhaustion markers (e.g., TIGIT, PD-1, TIM-3, and/or LAG-3) in a cell described herein is decreased by at least about 1.5-fold, at least about 2-fold, at least about 2.5-fold, at least about 3.0-fold, at least about 3.5-fold, at least about 4-fold, at least 4.5-fold, at least about 5-fold, at least about 10-fold, at least about 15-fold, at least about 20-fold, at least about 25-fold, at least about 30-fold, at least about 35-fold, at least about 40-fold, at least about 45-fold, at least about 50-fold, at least about 55-fold, at least about 60-fold, at least about 65-fold, at least about 70-fold, at least about 75-fold, at least about 80-fold, at least about 85-fold, at least about 90-fold, at least about 95-fold, or at least about 100-fold or more, compared to a reference cell (e.g., corresponding cell that has not been engineered to overexpress c-Jun).

In some aspects, the exhaustion state of a population of immune cells (e.g., modified and cultured using the methods provided herein) can be determined by quantifying the amount (e.g., number and/or percentage) of cells within the population of immune cells that express a given exhaustion marker (e.g., TIGIT, PD-1, TIM-3, and/or LAG-3). For instance, when a population of immune cells is modified to express an increased level of a c-Jun protein (e.g., in combination with a chimeric binding protein), the amount (e.g., number and/or percentage) of cells that express a given exhaustion marker is reduced, compared to the amount in a corresponding population of immune cells that was not modified as described herein. Accordingly, in some aspects, the amount of cells that express a given exhaustion marker in a population of modified immune cells described herein is decreased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to the amount in a corresponding population of immune cells that was not modified as described herein.

In some aspects, the increased expression of the c-Jun protein can increase the persistence/survival of the immune cell, e.g., when administered to a subject in vivo. In some aspects, the persistence/survival of the cell is increased by at least about 0.5-fold, by at least about 1-fold, by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 12-fold, by at least about 14-fold, by at least about 16-fold, by at least about 18-fold, by at least about 20-fold, by at least about 25-fold, by at least about 30-fold, by at least about 35-fold, by at least about 40-fold, by at least about 45-fold, or by at least about 50-fold, compared to a reference cell (e.g., corresponding cell that was not modified to have increased c-Jun protein expression).

In some aspects, the persistence/survival of the immune cell described herein is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to the amount in a corresponding population of immune cells that was not modified as described herein.

Accordingly, in some aspects, immune cells of the present disclosure are modified to overexpress c-Jun (e.g., with an exogenous nucleotide sequence encoding a c-Jun polypeptide and/or a transcription activator which is capable of increasing the expression of endogenous c-Jun) and cultured in a medium comprising potassium ion at a concentration higher than 5 mM, such that after the modification and the culturing, the persistence/survival of the immune cells is increased compared to reference cells. As described herein, in some aspects, the reference cells comprise corresponding immune cells that: (i) are not modified to overexpress c-Jun but cultured in a medium comprising potassium ion at a concentration higher than 5 mM; (ii) are modified to overexpress c-Jun but not cultured in a medium comprising potassium ion at a concentration higher than 5 mM; or (iii) both (i) and (ii).

In some aspects, the increased expression of the c-Jun protein can increase the expansion/proliferation of the immune cell, e.g., upon antigen stimulation. In some aspects, the expansion/proliferation of the cell is increased by at least about 0.5-fold, by at least about 1-fold, by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 12-fold, by at least about 14-fold, by at least about 16-fold, by at least about 18-fold, by at least about 20-fold, by at least about 25-fold, by at least about 30-fold, by at least about 35-fold, by at least about 40-fold, by at least about 45-fold, or by at least about 50-fold, compared to a reference cell (e.g., corresponding cell that was not modified to have increased c-Jun protein expression).

In some aspects, the expansion/proliferation of the immune cell, e.g., upon antigen stimulation, is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to the amount in a corresponding population of immune cells that was not modified as described herein.

Accordingly, in some aspects, immune cells of the present disclosure are modified to overexpress c-Jun (e.g., with an exogenous nucleotide sequence encoding a c-Jun polypeptide and/or a transcription activator which is capable of increasing the expression of endogenous c-Jun) and cultured in a medium comprising potassium ion at a concentration higher than 5 mM, such that after the modification and the culturing, the expansion/proliferation of the immune cells is increased compared to reference cells. As described herein, in some aspects, the reference cells comprise corresponding immune cells that: (i) are not modified to overexpress c-Jun but cultured in a medium comprising potassium ion at a concentration higher than 5 mM; (ii) are modified to overexpress c-Jun but not cultured in a medium comprising potassium ion at a concentration higher than 5 mM; or (iii) both (i) and (ii).

In some aspects, the increased expression of the c-Jun protein can increase the effector function of the cell, e.g., increased cytokine (e.g., IFN-7, TNF-α, and/or IL-2) production, granzyme release, and/or cytotoxicity. In some aspects, the increase in effector function is in response to persistent antigen stimulation. As used herein, the term “persistent antigen stimulation” or “chronic antigen stimulation” refers to repeated exposure of an immune cell (e.g., T cell) to its cognate antigen, such that the immune cell is stimulated or activated. In some aspects, persistent antigen stimulation comprises exposing an immune cell (e.g., T cells) to its cognate antigen for at least about 1 day, at least about 2 days, at least about 3 days, at least about 4 days, at least about 5 days, at least about 6 days, at least about 1 week, at least about 2 weeks, at least about 3 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year. In some aspects, the persistent antigen stimulation can be continuous. In some aspects, the persistent antigen stimulation can comprise multiple rounds of antigen stimulation, where each round of antigen stimulation is followed by a period of non-antigen stimulation. In some aspects, persistent antigen stimulation comprises at least about 2, at least about 3, at least about 4, at least about 5, or at least about 6 or more rounds of antigen stimulation. As is apparent from the present disclosure and known in the art, such persistent antigen stimulation of an immune cell can result in the exhaustion of the immune cell.

In some aspects, the effector function of the cell is increased by at least about 0.5-fold, by at least about 1-fold, by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 12-fold, by at least about 14-fold, by at least about 16-fold, by at least about 18-fold, by at least about 20-fold, by at least about 25-fold, by at least about 30-fold, by at least about 35-fold, by at least about 40-fold, by at least about 45-fold, or by at least about 50-fold, compared to a reference cell (e.g., corresponding cell that was not modified to have increased c-Jun protein expression).

In some aspects, the increased expression of the c-Jun protein can increase the effector function of the cell by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%, compared to a reference cell.

Accordingly, in some aspects, immune cells of the present disclosure are modified to overexpress c-Jun (e.g., with an exogenous nucleotide sequence encoding a c-Jun polypeptide and/or a transcription activator which is capable of increasing the expression of endogenous c-Jun) and cultured in a medium comprising potassium ion at a concentration higher than 5 mM, such that after the modification and the culturing, the effector function of the immune cells, e.g., in response to persistent antigen stimulation, is increased compared to reference cells. As described herein, in some aspects, the reference cells comprise corresponding immune cells that: (i) are not modified to overexpress c-Jun but cultured in a medium comprising potassium ion at a concentration higher than 5 mM; (ii) are modified to overexpress c-Jun but not cultured in a medium comprising potassium ion at a concentration higher than 5 mM; or (iii) both (i) and (ii).

In some aspects, a cell modified to express an increased level of c-Jun (e.g., as described herein) retains effector function, e.g., increased cytokine (e.g., IFN-7, TNF-α, and/or IL-2) production, granzyme release, and/or cytotoxicity (e.g., ability to kill relevant target cells) for at least one, at least two, at least three, or more, additional rounds in an antigen stimulation assay, such as a serial, chronic or sequential stimulation assay (such as that described in Example 3 or e.g., in Zhao et al., 2015 Cancer Cell 28(4):415-428; Kunkele et al., 2015 Cancer Immunology Research 3(4):368-379; each of which is incorporated herein by reference in its entirety) as compared to control cells (e.g., cells not overexpressing c-Jun).

In some aspects, as compared to the corresponding immune cells that were cultured in the reference culture medium, immune cells cultured in metabolic reprogramming media of the present disclosure (e.g., comprising potassium ion at a concentration higher than 5 mM) are able to produce higher amounts of cytokines (e.g., IFN-γ and/or IL-2) after at least two rounds of antigen stimulation, after at least three rounds of antigen stimulation, after at least four rounds of antigen stimulation, after at least five rounds of antigen stimulation, after at least six rounds of antigen stimulation. Accordingly, in some aspects, provided herein is a method of increasing the production of a cytokine by immune cells in response to antigen stimulation, wherein the method comprises culturing the immune cells in a medium comprising potassium ion at a concentration higher than 5 mM. As described herein, in some aspects, the immune cells have been modified to have an increased level of a c-Jun polypeptide compared to reference cells (e.g., corresponding immune cells that have not been modified to have an increased level of the c-Jun polypeptide).

In some aspects, after the culturing, the production of the cytokine by the modified immune cells in response to an antigen stimulation is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 25-fold, at least about 30-fold, at least about 35-fold, at least about 40-fold, at least about 45-fold, at least about 50-fold, at least about 75-fold, at least about 100-fold, at least about 200-fold, at least about 300-fold, at least about 400-fold, at least about 500-fold, at least about 750-fold, or at least about 1,000-fold or more, as compared to reference cells (e.g., described herein). In some aspects, after the culturing, the production of the cytokine by the modified immune cells in response to an antigen stimulation is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as compared to the reference cell.

Increased expression of c-Jun in T cells can help sustain the active state of the cells by, e.g., alleviating or preventing T cell dysfunction (e.g., T cell exhaustion). Accordingly, the different approaches to increasing c-Jun protein expression in a cell provided herein (e.g., modifying the cell with an exogenous polynucleotide encoding a c-Jun polypeptide and/or a transcriptional activator that is capable of increasing the expression of endogenous c-Jun) can be used to engineer immune cells, such as T cells, which then exhibit sustained, potent cytotoxicity against desired target cells (e.g., the target of the endogenous TCR or the target of a chimeric binding protein as described herein). As compared to T cells that do not overexpress c-Jun, engineered T cells disclosed herein (which have increased expression of the c-Jun protein) display fewer signs of T cell exhaustion as described above.

Additionally, as is apparent from the present disclosure, in some aspects, when any of the modified immune cells provided herein (e.g., expressing an increased level of a c-Jun protein) are cultured using the methods provided herein (e.g., in metabolic reprogramming media comprising potassium ion at a concentration higher than 5 mM), one or more of the above-described properties are further enhanced. For instance, in some aspects, compared to a reference cell (e.g., modified to express an increased level of a c-Jun protein but not cultured in metabolic reprogramming media described herein and/or cultured in metabolic reprogramming media described herein but not modified to express an increased level of a c-Jun protein), an immune cell of the present disclosure (modified to express an increased level of a c-Jun protein and cultured in metabolic reprogramming media (e.g., comprising potassium ion at a concentration higher than 5 mM) is capable of exhibiting one or more of the following: (i) increased resistance to exhaustion (e.g., as indicated by reduced expression of exhaustion markers, such as PD-1, CD39, TIM-3, and/or LAG-3; increased persistence/survival; delay of the onset of dysfunctional states; and/or increased cytokine production), (ii) increased expansion/proliferation, (iii) increased antigen sensitivity, (iv) increased effector function (particularly following repeated antigen stimulation) (e.g., cytokine production upon antigen stimulation, lysis of cells expressing the target antigen, or both), or (v) any combination thereof.

### II.C.2. Additional Translatable Sequences

In some aspects, an immune cell described herein (e.g., modified and cultured using the methods provided herein) can express one or more additional proteins of interest. For instance, in some aspects, a modified immune cell described herein further comprise one or more exogenous nucleotide sequences encoding additional proteins of interest. Accordingly, in some aspects, an immune cell disclosed herein comprises: (i) a first exogenous nucleotide sequence encoding a c-Jun polypeptide, and one or more exogenous nucleotide sequences encoding additional proteins of interest. Non-limiting examples of such additional translatable sequences are described below.

### Ligand Binding Proteins/Chimeric Binding Proteins

In some aspects, an immune cell useful for the present disclosure (e.g., modified to express an increased level of a c-Jun polypeptide) further comprises a nucleotide sequence encoding a ligand binding protein. As used herein, the term “ligand binding protein” refers to any protein that is able to bind a molecule of interest (i.e., ligand) (e.g., an antigen expressed on a tumor cell or a peptide/MHC complex). In some aspects, a ligand binding protein is a chimeric binding protein. As used herein, the term “chimeric binding protein” refers to proteins that are capable of binding to one or more ligands (e.g., antigens (e.g., comprising an antigen-binding moiety)) and are created through the joining of two or more polynucleotide sequences which originally code for separate proteins. Unless indicated otherwise, the terms can be used interchangeably in the present disclosure.

Non-limiting examples of ligand binding proteins (e.g., chimeric binding proteins) include a chimeric antigen receptor (CAR), T cell receptor (TCR), chimeric antibody-T cell receptor (caTCR), chimeric signaling receptor (CSR), T cell receptor mimic (TCR mimic), and combinations thereof.

As further described elsewhere in the present disclosure, in some aspects, the ligand binding protein can be associated with a gene editing tool (e.g., CRISPR-Cas system), where the activation of the ligand binding protein can induce the activation of the gene-editing tool, such that the expression and/or activity of one or more genes are modulated in the cell. For example, in some aspects, a cell described herein (e.g., T cells) is modified to comprise a chimeric binding protein (e.g., CAR) which is linked to a protease and a single guide RNA targeting a regulatory region (e.g., promoter) of a gene of interest. In some aspects, the cell is modified to further comprise a linker for activation of T cells (LAT), complexed to a gene-editing tool, e.g., via a linker. Activation of the chimeric binding protein (e.g., via antigen stimulation) allows the release of the gene editing tool for nuclear localization and modulation of gene expression. Additional aspects of such chimeric binding proteins are provided elsewhere in the present disclosure. See also Pietrobon et al., Int J Mol Sci 22(19): 10828 (October 2021), which is incorporated herein by reference in its entirety.

Chimeric Antigen Receptor (CAR)

As described herein, in some aspects, a chimeric binding protein useful for the present disclosure comprises a CAR. Accordingly, in some aspects, an immune cell that can be cultured using the methods provided herein has been modified to express a CAR and an increased level of a c-Jun protein. In some aspects, the immune cell is a CD8+ T cell and expresses a CAR and an increased level of a c-Jun protein. In some aspects, the immune cell is a CD4+ T cell and expresses a CAR and an increased level of a c-Jun protein. In some aspects, the immune cells comprise both CD8+ T cells and CD4+ T cells, wherein each of the CD8+ T cells and CD4+ T cells express a CAR and an increased level of a c-Jun protein In some aspects, a CAR-expressing cell disclosed herein is a CAR T cell, e.g., a mono CAR T cell, a genome-edited CAR T cell, a dual CAR T cell, or a tandem CAR T cell. Examples of such CAR T cells are provided in International Publication No. WO2020028400, which is incorporated by reference herein in its entirety.

In some aspects, the CAR (e.g., which can be expressed in combination with a c-Jun protein) is designed as a standard CAR. In a “standard CAR”, the different components (e.g., the extracellular targeting domain, transmembrane domain, and intracellular signaling/activation domain) are linearly constructed as a single fusion protein. In some aspects, the CAR is designed as a first generation CAR. “First generation” CARs are composed of an extracellular binding domain, a hinge region, a transmembrane domain, and one or more intracellular signaling domains. All first generation CARs contain the CD3(chain domain as the intracellular signaling domain. In some aspects, the CAR is designed as a second generation CAR. “Second generation” CARs additionally contain a costimulatory domain (e.g., CD28 or 4-1B). In some aspects, the CAR is designed as a third generation CAR. “Third generation” CARs are similar to the second generation CARs except that they contain multiple costimulatory domains (e.g., CD28-4-1BB or CD28-OX40). In some aspects, the CAR is designed as a fourth generation CAR. “Fourth generation” CARs (also known as TRUCKs or armored CARs) additionally contain additional factors that can further improve function. For example, in some aspects, the fourth generation CARs additionally contain cytokines which can be released upon CAR signaling in the targeted tumor tissue. In some aspects, the fourth generation CARs comprise one or more additional elements such as homing and suicide genes, which can help further regulate the activity of the CAR. In some aspects, the CAR is designed as a split CAR. In a “split CAR” system, one or more components of the CAR (e.g., extracellular targeting domain, transmembrane domain, and intracellular signaling/activation domain) are split into two or more parts such that it is dependent on multiple inputs that promote assembly of the intact functional receptor. In some aspects, the CAR is designed as a switchable CAR. With a “switchable CAR,” the CAR can be switched (e.g., transiently) on (on-switch CAR) or off (off-switch CAR) in the presence of a stimulus. Additional examples of CARs that can be used with the present disclosure are described, e.g., in US 2020/0172879 A1 and US 2019/0183932 A1, each of which is incorporated herein by reference in its entirety.

Engineered T Cell Receptor

In some aspects, a chimeric binding protein that can be used with the present disclosure comprises an engineered T cell receptor (TCR) (also referred to in the art as “transgenic” TCRs). As used herein, the term “engineered TCR” or “engineered T cell receptor” refers to a T cell receptor (TCR) that is isolated or engineered to specifically bind with a desired affinity to a major histocompatibility complex (MHC)/peptide target antigen and that is introduced into a population of immune cells, e.g., T cells and/or NK cells.

Accordingly, in some aspects, an immune cell that can be cultured using the methods provided herein have been modified to express a transgenic TCR and an increased level of a c-Jun protein. For instance, in some aspects, the immune cell comprises a CD8+ T cell and expresses a transgenic TCR and an increased level of a c-Jun protein. In some aspects, the immune cell comprises a CD4+ T cell and expresses a transgenic TCR and an increased level of a c-Jun protein. In some aspects, the immune cells comprise both CD8+ T cells and CD4+ T cells, wherein each of the CD8+ T cells and CD4+ T cells comprises a transgenic TCR and expresses an increased level of a c-Jun protein.

TCR is a molecule found on the surface of T cells which is responsible for recognizing fragments of antigen as peptides bound to major histocompatibility complex (MHC) molecules. The TCR is a heterodimer composed of two different protein chains. In some aspects, the TCR consists of an alpha (a) chain and a beta (p) chain (encoded by TRA and TRB, respectively). In some aspects, the TCR consists of gamma and delta (7/6) chains (encoded by TRG and TRD, respectively). When the TCR engages with an antigenic peptide presented by an MHC molecule (peptide/MHC), the T lymphocyte is activated through signal transduction.

In certain embodiments, an engineered TCR is Class I MHC restricted. In another embodiment, the engineered TCR is Class II MHC restricted. In certain embodiments, the engineered TCR recognizes a tumor antigen peptide:MHC complex. In one embodiment, the engineered TCR recognizes a neoantigen peptide:MHC complex. In certain embodiments, the engineered TCR comprises a transmembrane domain and a TCR domain that facilitates recruitment of at least one TCR-associated signaling molecule. In some embodiments, the engineered TCR further comprises one or more TCR derived constant domains, e.g., a CH1 and a CL.

T Cell Receptor Mimics (TCRm)

In some aspects, the chimeric binding protein which can be used to modify an immune cell comprises a T cell receptor mimic (TCR mimic). As used herein, the term “T cell receptor mimic” or “TCR mimic” refers to an antibody (or a fragment thereof) that has been engineered to recognize tumor antigens, where the tumor antigens are displayed in the context of HLA molecules. As will be apparent to those skilled in the art, these antibodies can mimic the specificity of TCR. Non-limiting examples of TCR mimics are provided, e.g., in US 2009/0226474 A1; US 2019/0092876 A1; and Traneska et al., Front. Immunol. 8(1001):1-12 (2017), each of which is incorporated herein by reference in its entirety. In some aspects, the TCR mimic comprises (i) an antibody moiety that specifically binds to a peptide:MHC complex of interest, and (ii) a T cell receptor module capable of recruiting at least one TCR-associated signaling molecule. In some aspects, the TCR mimic comprises (i) an antibody moiety that specifically binds to a peptide:MHC complex of interest, and (ii) a transmembrane domain, one or more intracellular signaling domains (e.g., the CD3(chain domain) and optionally one or more costimulatory domains (e.g., CD28 or 4-1BB).

Accordingly, in some aspects, an immune cell that can be cultured using the methods provided herein have been modified to express a TCR mimic and an increased level of a c-Jun protein (e.g., transduced with one or more exogenous nucleotide sequences encoding a c-Jun protein and a TCR mimic). In some aspects, the immune cell comprises a CD8+ T cell and expresses a TCR mimic and an increased level of a c-Jun protein. In some aspects, the immune cell comprises a CD4+ T cell and expresses a TCR mimic and an increased level of a c-Jun protein. In some aspects, the immune cells comprise both CD8+ T cells and CD4+ T cells, wherein each of the CD8+ T cells and CD4+ T cells express a TCR mimic and an increased level of a c-Jun protein.

In some aspects, the TCR mimic comprises a chimeric antibody-T cell receptor (caTCR). As used herein, a “chimeric antibody-T cell receptor” or “caTCR” comprises (i) an antibody moiety that specifically binds to an antigen of interest and (ii) a T cell receptor module capable of recruiting at least one TCR-associated signaling molecule. In some aspects, the antibody moiety and the T cell receptor module are fused together. Additional disclosure relating to caTCRs that are useful for the present disclosure is provided in, e.g., U.S. Pat. No. 10,822,413 B2; and Xu et al., Cell Discovery 4:62 (2018), each of which is herein incorporated by reference in its entirety.

Accordingly, in some aspects, an immune cell that can be cultured using the methods provided herein have been modified to express a caTCR and an increased level of a c-Jun protein (e.g., transduced with one or more exogenous nucleotide sequence encoding a c-Jun polypeptide and a caTCR). In some aspects, the immune cells modified to express a caTCR and an increased level of a c-Jun protein are further modified to express a chimeric co-stimulatory receptor. In some aspects, an immune cell (such as a T cell) provided herein expresses an increased level of a c-Jun protein and comprises: a caTCR and a chimeric co-stimulatory receptor, comprising: i) a ligand-binding module that is capable of binding or interacting with a target ligand; ii) a transmembrane module; and iii) a co-stimulatory immune cell signaling module that is capable of providing a co-stimulatory signal to the immune cell, wherein the ligand-binding module and the co-stimulatory immune cell signaling module are not derived from the same molecule, and wherein the chimeric co-stimulatory receptor lacks a functional primary immune cell signaling domain. In some aspects, the chimeric co-stimulatory receptor comprises a ligand-binding module that binds to a tumor antigen. Exemplary chimeric co-stimulatory receptors are described in e.g., U.S. Pat. No. 10,822,413, which is herein incorporated by reference in its entirety. In some aspects, the immune cell described herein comprises a CD8+ T cell and expresses a caTCR and an increased level of a c-Jun protein. In some aspects, the immune cell comprises a CD4+ T cell and expresses a caTCR and an increased level of a c-Jun protein. In some aspects, the immune cells comprise both CD8+ T cells and CD4+ T cells, wherein each of the CD8+ T cells and CD4+ T cells express a caTCR and an increased level of a c-Jun protein.

Chimeric Signaling Receptor (CSR)

In some aspects, a chimeric binding protein comprises a chimeric signaling receptor (CSR). “Chimeric signaling receptor” or “CSR” comprises a ligand-binding domain that specifically binds to a target ligand and a co-stimulatory signaling domain capable of providing a stimulatory signal to an immune cell that expresses the CSR. A chimeric signaling receptor can comprise (1) an extracellular binding domain (e.g., natural/modified receptor extracellular domain, natural/modified ligand extracellular domain, scFv, nanobody, Fab, DARPin, and affibody), (2) a transmembrane domain, and (3) an intracellular signaling domain (e.g., a domain that activates transcription factors, or recruits and/or activates JAK/STAT, kinases, phosphatases, and ubiquitin; SH3; SH2; and PDZ). See, e.g., EP340793B1, US 2021/0253665 A1, U.S. Pat. No. 10,822,413 B2, and Xu et al., Cell Discovery 4:62 (2018), each of which is incorporated herein by reference in its entirety.

In some aspects, an immune cell that can be cultured using the methods provided herein (e.g., modified to express an increased level of a c-Jun protein) expresses a chimeric signaling receptor. In some aspects, the immune cell comprises a CD8+ T cell and expresses a CSR and an increased level of a c-Jun protein. In some aspects, the immune cell comprises a CD4+ T cell and expresses a CSR and an increased level of a c-Jun protein. In some aspects, the immune cells comprise both CD8+ T cells and CD4+ T cells, wherein each of the CD8+ T cells and CD4+ T cells express a CSR and an increased level of a c-Jun protein.

Antigen-Binding Domain

As described herein, a chimeric binding protein useful for the present disclosure (e.g., CAR, TCR, caTCR, CSR, or TCR mimic) comprises an antigen-binding domain, a transmembrane domain, a costimulatory domain, an intracellular signaling domain, or combinations thereof. Additional disclosure relating to the transmembrane domain, costimulatory domain, and intracellular signaling domain are provided elsewhere in the present disclosure.

In some aspects, the antigen-binding domain recognizes and specifically binds to an antigen. In some aspects, the antigen-binding domain of a chimeric binding protein described herein specifically binds to an antigen expressed on a tumor cell.

In some aspects, the antigen-binding domain of a chimeric binding protein specifically binds to an antigen selected from CD19, TRAC, TCRβ, BCMA, CLL-1, CS1, CD38, TSHR, CD123, CD22, CD30, CD70, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-l lRa, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gp100, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WTl, NY-ESO-1, LAGE-la, MAGE-Al, legumain, HPV E6, E7, MAGE Al, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant (e.g., HRAS, KRAS, NRAS), hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3F, CD4, CD5, CD7, the extracellular portion of the APRIL protein, neoantigen, or any combinations thereof.

In some aspects, the antigen-binding domain specifically recognizes and binds to BCMA. In some aspects, the antigen-binding domain specifically recognizes and binds to CD147. In some aspects, the antigen-binding domain specifically recognizes and binds to CD19. In some aspects, the antigen-binding domain specifically recognizes and binds to ROR1. In some aspects, the antigen-binding domain specifically recognizes and binds to GPC3. In some aspects, the antigen-binding domain specifically recognizes and binds to GPC2. In some aspects, the antigen-binding domain specifically recognizes and binds to CD19 and CD22. In some aspects, the antigen-binding domain specifically recognizes and binds to CD19 and CD28. In some aspects, the antigen-binding domain specifically recognizes and binds to CD20. In some aspects, the antigen-binding domain specifically recognizes and binds to CD20 and CD19. In some aspects, the antigen-binding domain specifically recognizes and binds to CD22. In some aspects, the antigen-binding domain specifically recognizes and binds to CD30. In some aspects, the antigen-binding domain specifically recognizes and binds to CEA. In some aspects, the antigen-binding domain specifically recognizes and binds to DLL3. In some aspects, the antigen-binding domain specifically recognizes and binds to EGFRvIII. In some aspects, the antigen-binding domain specifically recognizes and binds to GD2. In some aspects, the antigen-binding domain specifically recognizes and binds to HER2. In some aspects, the antigen-binding domain specifically recognizes and binds to IL-1RAP. In some aspects, the antigen-binding domain specifically recognizes and binds to mesothelin. In some aspects, the antigen-binding domain specifically recognizes and binds to NKG2D. In some aspects, the antigen-binding domain specifically recognizes and binds to PSMA. In some aspects, the antigen-binding domain specifically recognizes and binds to TnMUC1.

In some aspects, the antigen-binding domain of a chimeric binding protein described herein specifically recognizes and binds an antigen in complex with an MHC.

As further described elsewhere in the present disclosure, the antigen-binding domain of a chimeric binding protein (e.g., CAR, TCR, caTCR, CSR, or TCR mimic) can be any polypeptide capable of binding one or more antigens (e.g., tumor antigens). In some aspects, the antigen-binding domain comprises, or is derived from, an Ig NAR, a Fab fragment, a Fab′ fragment, a F(ab)′2 fragment, a F(ab)′3 fragment, an Fv, a single chain variable fragment (scFv), a bis-scFv, a (scFv)2, a minibody, a diabody, a triabody, a tetrabody, an intrabody, a disulfide stabilized Fv protein (dsFv), a unibody, a nanobody, and an antigen binding region derived from an antibody that may specifically bind to any of a protein of interest, a ligand, a receptor, a receptor fragment, a peptide aptamer, or combinations thereof. In some aspects, the antigen-binding domain is a single chain Fv (scFv).

In some aspects, a chimeric binding protein described herein comprises an antigen-binding domain which is a natural ligand. As used herein, the term “natural ligand” refers to a naturally existing moiety that specifically binds to an antigen of interest. For instance, in some aspects, the antigen-binding domain can comprise a NKG2D cell receptor, which is a known natural ligand for NKG2D. NKG2D has been described to be expressed on many tumors. See, e.g., Sentman et. al., Cancer J 20(2): 156-159 (2014).

Signaling (Intracellular), Transmembrane, and Costimulatory Domains

In some aspects, a chimeric binding protein described herein (e.g., CAR, TCR, caTCR, CSR, or TCR mimic) comprises an intracellular signaling domain that transduces the effector function signal upon binding of an antigen to the extracellular domain and directs the cell expressing the chimeric binding protein (e.g., T cell) to perform a specialized function. Non-limiting examples of intracellular signaling domain include an intracellular signaling domain region derived from CD3 zeta, FcR gamma, common FcR gamma (FCER1G), Fc gamma RIIa, FcR beta (Fc Epsilon Rib), CD3 gamma, CD3 delta, CD3 epsilon, CD22, CD79a, CD79b, CD278 (“ICOS”), FcεRI, CD66d, CD32, DAP10, DAP12, or any combination thereof. In some aspects, the intracellular signaling domain comprises a CD3 zeta intracellular signaling domain (e.g., such as that set forth in SEQ ID NO: 90).

In some aspects, the chimeric binding protein comprises the entire intracellular domain of a protein disclosed herein. In some aspects, the intracellular domain is truncated. Truncated portion of an intracellular domain can be used in place of the intact chain as long as it still transduces the effector function signal. The term intracellular domain is thus meant to include any truncated portion of the intracellular domain sufficient to transduce the effector function signal.

In some aspects, a chimeric binding protein useful for the present disclosure (e.g., CAR, TCR, caTCR, CSR, or TCR mimic) further comprises a transmembrane domain. In some aspects, the antigen-binding domain of a chimeric binding protein is linked to the intracellular domain by a transmembrane domain. In some aspects, the antigen-binding domain of a chimeric binding protein is connected to the transmembrane domain by a linker. In some aspects, the inclusion of a linker between the antigen-binding domain and the transmembrane domain can affect flexibility of the antigen-binding domain and thereby, improve one or more properties of a chimeric binding protein.

Any transmembrane domain known in the art can be used in the chimeric binding proteins described herein (e.g., CAR, TCR, caTCR, CSR, or TCR mimic). In some aspects, the transmembrane domain is artificial (e.g., an engineered transmembrane domain). In some aspects, the transmembrane domain is derived from a naturally occurring polypeptide. In some aspects, the transmembrane domain comprises a transmembrane domain from a naturally occurring polypeptide. Non-limiting examples of transmembrane domain include a transmembrane domain region of KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, CD19, CD8, or any combination thereof. In some aspects, the transmembrane domain comprises a CD28 transmembrane domain (e.g., such as that set forth in SEQ ID NO: 75).

As described herein, in some aspects, a chimeric binding protein useful for the present disclosure (e.g., CAR, TCR, caTCR, CSR, or TCR mimic) comprises one or more costimulatory domains (e.g., second and third generation CARs). Not to be bound by any one theory, these costimulatory domains can further improve the expansion, activation, memory, persistence, and/or effector function of an immune cell engineered to express the chimeric binding protein (e.g., in combination with the c-Jun polypeptide). In some aspects, the transmembrane domain is fused to the costimulatory domain, optionally a costimulatory domain is fused to a second costimulatory domain, and the costimulatory domain is fused to a signaling domain, not limited to CD3ζ. Non-limiting examples of costimulatory domain include interleukin-2 receptor (IL-2R), interleukin-12 receptor (IL-12R), IL-7, IL-21, IL-23, IL-15, CD2, CD3, CD4, CD7, CD8, CD27, CD28, CD30, CD40, 4-1BB/CD137, ICOS, lymphocyte function-associated antigen-1 (LFA-1), LIGHT, NKG2C, OX40, DAP10, or any combination thereof. In some aspects, the costimulatory domain comprises a 4-1BB/CD137 costimulatory domain (e.g., such as that set forth in SEQ ID NO: 76).

Truncated EGFR

In some aspects, immune cells disclosed herein (e.g., modified and cultured using the methods provided herein) further comprise an exogenous nucleotide sequence encoding a truncated epidermal growth factor receptor (EGFRt), such that the EGFRt comprises only a partial sequence of the full-length EGFR protein (e.g., SEQ ID NO: 19). In some aspects, the EGFRt comprises EGFR extracellular Domains III and IV and an EGFR transmembrane domain, but lacks EGFR extracellular Domains I and II and EGFR intracellular sequence. Accordingly, in some aspects, an immune cell disclosed herein has been modified to comprise: (i) an exogenous nucleotide sequence encoding a c-Jun polypeptide, (ii) an exogenous nucleotide sequence encoding a chimeric binding protein, and (iii) an exogenous nucleotide sequence encoding an EGFRt. As described herein, in some aspects, a transcriptional activator (e.g., CRISPRa) can be used to increase the expression of the c-Jun protein endogenously. Therefore, in some aspects, an immune cell described herein has been modified to comprise: (i) a transcriptional activator that is capable of increasing the expression of endogenous c-Jun protein, (ii) an exogenous nucleotide sequence encoding a chimeric binding protein, and (iii) an exogenous nucleotide sequence encoding an EGFRt. In each of the above aspects, one or more of the multiple exogenous nucleotide sequences can be part of a single polycistronic polynucleotide.

EGFR is a 180 kDa monomeric glycoprotein comprising a large extracellular region, a single spanning transmembrane domain, an intracellular juxtamembrane region, a tyrosine kinase domain, and a C-terminal regulatory region. The extracellular region comprises four domains: Domains I and III are homologous ligand binding domains, and domains II and IV are cysteine rich domains (Ferguson, Annu Rev Biophys. (2008) 37:353-3). Unless otherwise indicated, EGFR as used herein refers to human EGFR. Due to alternative splicing, there are at least four known isoforms of human EGFR. Sequences for the different EGFR isoforms are provided in Table 4 (below).

TABLE 4
Human EGFR sequences

| | |
|---|---|
| Isoform 1 | MRPSGTAGAALLALLAALCPASRALEEKKVCQGTSNKLTQLGTFEDHFLSLQRMFNNCEV |
| (canonical | VLGNLEITYVQRNYDLSFLKTIQEVAGYVLIALNTVERIPLENLQIIRGNMYYENSYALA |
| sequence) | VLSNYDANKTGLKELPMRNLQEILHGAVRFSNNPALCNVESIQWRDIVSSDFLSNMSMDF |
| (also known | QNHLGSCQKCDPSCPNGSCWGAGEENCQKLTKIICAQQCSGRCRGKSPSDCCHNQCAAGC |
| as “p170”) | TGPRESDCLVCRKFRDEATCKDTCPPLMLYNPTTYQMDVNPEGKYSFGATCVKKCPRNYV |
| (UniProt: | VTDHGSCVRACGADSYEMEEDGVRKCKKCEGPCRKVCNGIGIGEFKDSLSINATNIKHFK |
| P00533-1) | NCTSISGDLHILPVAFRGDSFTHTPPLDPQELDILKTVKEITGFLLIQAWPENRTDLHAF |
| | ENLEIIRGRTKQHGQFSLAVVSLNITSLGLRSLKEISDGDVIISGNKNLCYANTINWKKL |
| | FGTSGQKTKIISNRGENSCKATGQVCHALCSPEGCWGPEPRDCVSCRNVSRGRECVDKCN |
| | LLEGEPREFVENSECIQCHPECLPQAMNITCTGRGPDNCIQCAHYIDGPHCVKTCPAGVM |
| | GENNTLVWKYADAGHVCHLCHPNCTYGCTGPGLEGCPTNGPKIPSIATGMVGALLLLLVV |
| | ALGIGLFMRRRHIVRKRTLRRLLQERELVEPLTPSGEAPNQALLRILKETEFKKIKVLGS |
| | GAFGTVYKGLWIPEGEKVKIPVAIKELREATSPKANKEILDEAYVMASVDNPHVCRLLGI |
| | CLTSTVQLITQLMPFGCLLDYVREHKDNIGSQYLLNWCVQIAKGMNYLEDRRLVHRDLAA |
| | RNVLVKTPQHVKITDFGLAKLLGAEEKEYHAEGGKVPIKWMALESILHRIYTHQSDVWSY |
| | GVTVWELMTFGSKPYDGIPASEISSILEKGERLPQPPICTIDVYMIMVKCWMIDADSRPK |
| | FRELIIEFSKMARDPQRYLVIQGDERMHLPSPTDSNFYRALMDEEDMDDVVDADEYLIPQ |
| | QGFFSSPSTSRTPLLSSLSATSNNSTVACIDRNGLQSCPIKEDSFLQRYSSDPTGALTED |
| | SIDDTFLPVPEYINQSVPKRPAGSVQNPVYHNQPLNPAPSRDPHYQDPHSTAVGNPEYLN |
| | TVQPTCVNSTFDSPAHWAQKGSHQISLDNPDYQQDFFPKEAKPNGIFKGSTAENAEYLRV |
| | APQSSEFIGA (SEQ ID NO: 19) |
| Isoform 2 | MRPSGTAGAALLALLAALCPASRALEEKKVCQGTSNKLTQLGTFEDHFLSLQRMFNNCEV |
| (also known | VLGNLEITYVQRNYDLSFLKTIQEVAGYVLIALNTVERIPLENLQIIRGNMYYENSYALA |
| as “p60”) | VLSNYDANKTGLKELPMRNLQEILHGAVRFSNNPALCNVESIQWRDIVSSDFLSNMSMDF |
| (UniProt: | QNHLGSCQKCDPSCPNGSCWGAGEENCQKLTKIICAQQCSGRCRGKSPSDCCHNQCAAGC |
| P00533-2) | TGPRESDCLVCRKFRDEATCKDTCPPLMLYNPTTYQMDVNPEGKYSFGATCVKKCPRNYV |
| | VTDHGSCVRACGADSYEMEEDGVRKCKKCEGPCRKVCNGIGIGEFKDSLSINATNIKHFK |
| | NCTSISGDLHILPVAFRGDSFTHTPPLDPQELDILKTVKEITGLS (SEQ ID NO: 20) |
| Isoform 3 | MRPSGTAGAALLALLAALCPASRALEEKKVCQGTSNKLTQLGTFEDHFLSLQRMFNNCEV |
| (also known | VLGNLEITYVQRNYDLSFLKTIQEVAGYVLIALNTVERIPLENLQIIRGNMYYENSYALA |
| as “p110”) | VLSNYDANKTGLKELPMRNLQEILHGAVRFSNNPALCNVESIQWRDIVSSDFLSNMSMDF |
| (UniProt: | QNHLGSCQKCDPSCPNGSCWGAGEENCQKLTKIICAQQCSGRCRGKSPSDCCHNQCAAGC |
| P00533-3) | TGPRESDCLVCRKFRDEATCKDTCPPLMLYNPTTYQMDVNPEGKYSFGATCVKKCPRNYV |
| | VTDHGSCVRACGADSYEMEEDGVRKCKKCEGPCRKVCNGIGIGEFKDSLSINATNIKHFK |
| | NCTSISGDLHILPVAFRGDSFTHTPPLDPQELDILKTVKEITGFLLIQAWPENRTDLHAF |
| | ENLEIIRGRTKQHGQFSLAVVSLNITSLGLRSLKEISDGDVIISGNKNLCYANTINWKKL |
| | FGTSGQKTKIISNRGENSCKATGQVCHALCSPEGCWGPEPRDCVSCRNVSRGRECVDKCN |
| | LLEGEPREFVENSECIQCHPECLPQAMNITCTGRGPDNCIQCAHYIDGPHCVKTCPAGVM |
| | GENNTLVWKYADAGHVCHLCHPNCTYGPGNESLKAMLFCLFKLSSCNQSNDGSVSHQSGS |
| | PAAQESCLGWIPSLLPSEFQLGWGGCSHLHAWPSASVIITASSCH (SEQ ID NO: 21) |
| Isoform 4 | MRPSGTAGAALLALLAALCPASRALEEKKVCQGTSNKLTQLGTFEDHFLSLQRMFNNCEV |
| (UniProt: | VLGNLEITYVQRNYDLSFLKTIQEVAGYVLIALNTVERIPLENLQIIRGNMYYENSYALA |
| P00533-4) | VLSNYDANKTGLKELPMRNLQEILHGAVRFSNNPALCNVESIQWRDIVSSDFLSNMSMDF |
| | QNHLGSCQKCDPSCPNGSCWGAGEENCQKLTKIICAQQCSGRCRGKSPSDCCHNQCAAGC |
| | TGPRESDCLVCRKFRDEATCKDTCPPLMLYNPTTYQMDVNPEGKYSFGATCVKKCPRNYV |
| | VTDHGSCVRACGADSYEMEEDGVRKCKKCEGPCRKVCNGIGIGEFKDSLSINATNIKHFK |
| | NCTSISGDLHILPVAFRGDSFTHTPPLDPQELDILKTVKEITGFLLIQAWPENRTDLHAF |
| | ENLEIIRGRTKQHGQFSLAVVSLNITSLGLRSLKEISDGDVIISGNKNLCYANTINWKKL |
| | FGTSGQKTKIISNRGENSCKATGQVCHALCSPEGCWGPEPRDCVSCRNVSRGRECVDKCN |
| | LLEGEPREFVENSECIQCHPECLPQAMNITCTGRGPDNCIQCAHYIDGPHCVKTCPAGVM |
| | GENNTLVWKYADAGHVCHLCHPNCTYGS (SEQ ID NO: 22) |

In the above canonical sequence for EGFR (i.e., isoform 1), the various EGFR domains are delineated as follows. The signal peptide spans amino acids 1-24. The extracellular sequence spans amino acids 25-645, wherein Domain I, Domain II, Domain III, and Domain IV span amino acids 25-188, 189-333, 334-504, and 505-645, respectively. The transmembrane domain spans amino acids 646-668. The intracellular domain spans amino acids 669-1,210, where the juxtamembrane domain spans amino acids 669-703 and the tyrosine kinase domain spans amino acids 704-1,210.

In some aspects, the EGFRt useful for the present disclosure comprises an amino acid sequence having at least about 55%, at least about 60%, at least about 65%, at least about 70%, at least about 75%, at least about 80%, at least about 85%, at least about 90%, at least about 9500 at least about 96%, at least about 97%, at least about 98%, or at least about 99% sequence identity to the amino acid sequence set forth in SEQ TD NO: 19.

In some aspects, the EGFRt that can be used with the present disclosure comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 21. In some aspects, the EGFRt comprises the amino acid sequence set forth in SEQ ID NO: 21 (see Table 5). In some aspects, the EGFRt that can be used with the present disclosure comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 24. In some aspects, the EGFRt comprises the amino acid sequence set forth in SEQ ID NO: 24 (see Table 5).

TABLE 5
Truncated EGFR sequences

| | |
|---|---|
| EGFRt | RKVCNGIGIGEFKDSLSINATNIKHFKNCTSISGDLHILPVAFRGDSFTHTPPLD |
| | PQELDILKTVKEITGFLLIQAWPENRTDLHAFENLEIIRGRTKQHGQFSLAVVSL |
| | NITSLGLRSLKEISDGDVIISGNKNLCYANTINWKKLFGTSGQKTKIISNRGENS |
| | CKATGQVCHALCSPEGCWGPEPRDCVSCRNVSRGRECVDKCNLLEGEPREFVENS |
| | ECIQCHPECLPQAMNITCTGRGPDNCIQCAHYIDGPHCVKTCPAGVMGENNTLVW |
| | KYADAGHVCHLCHPNCTYGCTGPGLEGCPTNGPKIPSIATGMVGALLLLLVVALG |
| | IGLFM (SEQ ID NO: 23) |
| EGFRt + first 3 | RKVCNGIGIGEFKDSLSINATNIKHFKNCTSISGDLHILPVAFRGDSFTHTPPLD |
| amino acids of the | PQELDILKTVKEITGFLLIQAWPENRTDLHAFENLEIIRGRTKQHGQFSLAVVSL |
| intracellular domain | NITSLGLRSLKEISDGDVIISGNKNLCYANTINWKKLFGTSGQKTKIISNRGENS |
| of human EGFR | CKATGQVCHALCSPEGCWGPEPRDCVSCRNVSRGRECVDKCNLLEGEPREFVENS |
| | ECIQCHPECLPQAMNITCTGRGPDNCIQCAHYIDGPHCVKTCPAGVMGENNTLVW |
| | KYADAGHVCHLCHPNCTYGCTGPGLEGCPTNGPKIPSIATGMVGALLLLLVVALG |
| | IGLFMRRR (SEQ ID NO: 24) |

In some aspects, the EGFRt described herein additionally comprises a juxtamembrane domain. As used herein, the term “juxtamembrane domain” refers to an intracellular portion of a cell surface protein (e.g., EGFR) immediately C-terminal to the transmembrane domain. Not to be bound by any one theory, in some aspects, the addition of the juxtamembrane domain can increase the expression of the protein encoded by the polynucleotides of the present disclosure.

In some aspects, the juxtamembrane domain can be from about 1 to about 20 (e.g., 2-20, 3-20, 4-20, 5-20, 2-18, 3-18, 4-18, or 5-18) amino acids long. In some aspects, the juxtamembrane domain can be longer than 20 amino acids. In some aspects, the first 1 or more (e.g., first 2, 3, 4, 5, 6, 7, 8, 9, 10, 11, 12, 13, 14, 15, 16, 17, 18, 19, or 20) amino acids of the juxtamembrane domain is a net-neutral or net-positively charged sequence (e.g., the number of arginine and lysine residues is greater than or equal to the number of aspartic acid and glutamic acid residues). In some aspects, those first amino acids contain more than about 30% (e.g., more than 40, 50, 60, 70, 80, or 90%) hydrophilic amino acids. Non-limiting examples of juxtamembrane domains that are useful for the present disclosure are provided in Table 6 (below).

TABLE 6
Juxtamembrane domain sequences

| Net charge | Sequence |
|---|---|
| +1 | K |
| +2 | KR |
| +3 | KRK |
| +2 | KSR |
| +1 | KSGSGS (SEQ ID NO: 25) |
| +2 | SKR |
| +1 | KRSD (SEQ ID NO: 26) |
| +2 | KRSDK (SEQ ID NO: 27) |
| 0 | SGGGG (SEQ ID NO: 28) |
| 0 | SGAGG (SEQ ID NO: 29) |
| +2 | KRADK (SEQ ID NO: 30) |
| +3 | RRRSGGGGSGGGGS (SEQ ID NO: 31) |
| 0 | SGGGGSGGGGS (SEQ ID NO: 32) |
| 0 | (GGGGS) n, n >1 (SEQ ID NO: 33) |

In some aspects, the juxtamembrane domain that can be used with the present disclosure can be derived from the juxtamembrane region of a natural cell surface protein, such as a juxtamembrane region (e.g., the entire or partial sequence of the first 20 juxtamembrane amino acids) of a human receptor tyrosine kinase that interacts with phosphatidylcholine (PC), phosphatidylserine (PS), or phosphatidylinositol-4,5-bisphosphate (PIP2) (see, e.g., Hedger et al., Sci Rep. (2015) 5: 9198). Non-limiting examples of receptor tyrosine kinases are ERBB1 (EGFR), ERBB2 (HER2), ERBB3 (HER3), ERBB4 (HER4), INSR, IGF1R, INSRR, PGFRA, PGFRB, KIT, CSF1R, FLT3, VGFR1, VGFR2, VGFR3, FGFR1, FGFR2, FGFR3, FGFR4, PTK7, NTRK1, NTRK2, NTRK3, ROR1, ROR2, MUSK, MET, RON, UFO, TYRO3, MERTK, TIE1, TIE2, EPHA1, EPHA2, EPHA3, EPHA4, EPHA5, EPHA6, EPHA7, EPHA8, EPHAA, EPHB1, EPHB2, EPHB3, EPHB4, EPHB6, RET, RYK, DDR1, DDR2, ROS1, LMTK1, LMTK2, LMTK3, LTK, ALK, and STYK1. In some aspects, the juxtamembrane domain can comprise one or more mutations (e.g., substitutions or deletions) that remove residues known to be phosphorylated so as to circumvent any unintended signal transducing ability of the protein encoded by the polynucleotides of the present disclosure.

In some aspects, the juxtamembrane domain is derived from a juxtamembrane region of EGFR. Non-limiting examples of EGFR-derived juxtamembrane domains comprise one of the sequences provided in Table 7 (below). In some aspects, the juxtamembrane domain comprises the amino acid sequence RRR. In some aspects, an EGFRt comprising such a juxtamembrane domain comprises the sequence set forth in SEQ ID NO: 24.

TABLE 7
EGFR-derived juxtamembrane domain sequences

| Net charge | Sequence |
|---|---|
| +6 | RRRHIVRKR (SEQ ID NO: 34) |
| +5 | RRRHIVRK (SEQ ID NO: 35) |
| +4 | RRRHIVR (SEQ ID NO: 36) |
| +3 | RRRHIV (SEQ ID NO: 37) |
| +3 | RRRHI (SEQ ID NO: 38) |
| +3 | RRRH (SEQ ID NO: 39) |
| +3 | RRR |
| +2 | RR |
| +1 | R |

As is apparent from the present disclosure, modifying an immune cell described herein (e.g., expressing an increased level of a c-Jun polypeptide and/or comprising an exogenous nucleotide sequence encoding a chimeric binding protein) to further comprise an exogenous nucleotide sequence encoding EGFRt provides certain advantages. For instance, in some aspects, the EGFRt can function as a kill switch. In some aspects, when the engineered cells described herein are no longer needed in the body, a pharmaceutical grade anti-EGFR antibody, such as cetuximab, panitumumab, nimotuzumab, or necitumumab, can be administered to a subject who had received the engineered cells, thereby removing the engineered cells, e.g., through antibody-dependent cellular cytotoxicity (ADCC), complement-dependent cytotoxicity (CDC), and/or antibody-dependent cellular phagocytosis (ADCP).

Spacers

In some aspects, immune cells described herein (e.g., modified and cultured using the methods provided herein) also comprise an exogenous nucleotide sequence encoding a spacer. Accordingly, in some aspects, an immune cell described herein has been modified to express an increased level of a c-Jun protein (e.g., with an exogenous nucleotide sequence encoding the c-Jun protein and/or a transcriptional activator capable of increasing the expression of endogenous c-Jun protein) and comprise: an exogenous nucleotide sequence encoding a c-Jun protein, an exogenous nucleotide sequence encoding a chimeric binding protein, and an exogenous nucleotide sequence encoding a spacer. In some aspects, an immune cell has been modified to express an increased level of a c-Jun protein and comprise: an exogenous nucleotide sequence encoding a c-Jun protein, an exogenous nucleotide sequence encoding a chimeric binding protein, an exogenous nucleotide sequence encoding an EGFRt, and an exogenous nucleotide sequence encoding a spacer. In some aspects, the one or more exogenous nucleotide sequences are part of a single polycistronic polynucleotide. As used herein, the term “spacer” refers to a polypeptide sequence which is capable of covalently linking together two spaced moieties (e.g., P2A linker and a chimeric binding protein).

In some aspects, the spacer is derived from an immunoglobulin (e.g., derived from hinge regions or loop regions). In some aspects, the spacer comprises IgA1, IgA2, IgG1, IgG2, IgG3, IgG4, IgD, IgE, or IgM hinge regions, fragments thereof (alone or capped by additional sequences, e.g., CH1 or CH2 regions sequences), or combinations of fragments from IgA1, IgA2, IgG1, IgG2, IgG3, IgG4, IgD, IgE, or IgM hinge regions (referred to herein as a “hinge region derived spacer”). In some aspects, the spacer comprises IgA1, IgA2, IgG1, IgG2, IgG3, IgG4, IgD, IgE, or IgM constant domain loop regions, fragments thereof (alone or capped by additional sequences, e.g., from adjacent P-strands), or combinations of fragments from IgA1, IgA2, IgG1, IgG2, IgG3, IgG4, IgD, IgE, or IgM loop regions (referred to herein as a “loop region derived spacer”). In some aspects, the spacer comprises hinge region derived spacer, loop region derived spacer, or both (e.g., two or more concatenated hinge region derived spacers and loop region derived spacers).

Accordingly, in some aspects, a polynucleotide described herein encodes a polypeptide comprising (i) a c-Jun protein, (ii) a first linker (e.g., P2A linker), (iii) signal peptide (e.g., hIgκ), (iv) antigen-binding domain (e.g., scFv), (v) a second linker (e.g., GGGSG; SEQ ID NO: 40), (vi) a spacer (e.g., IgG2 hinge derived spacer), (vii) a transmembrane domain (e.g., CD28), (viii) a costimulatory domain (e.g., 4-1BB), (ix) an intracellular signaling domain (e.g., CD3ζ), (x) a third linker (e.g., P2A linker), and (xi) a EGFRt.

In some aspects, a spacer useful for the present disclosure comprises a subsequence of an immunoglobulin heavy chain selected the group consisting of human IgA1 (Uniprot: P01876, IGHA1_HUMAN, immunoglobulin heavy constant alpha 1; SEQ ID NO: 41), human IgA2 (Uniprot P01877, IGHA2_HUMAN, immunoglobulin heavy constant alpha 2; SEQ ID NO: 42), murine IgG2A (Uniprot P01665, GCAM_MOUSE, immunoglobulin gamma 2A chain C region; SEQ ID NO: 43), human IgG1 (Uniprot P01857, IGHG1_HUMAN, immunoglobulin heavy constant gamma 1; SEQ ID NO: 44), human IgG2 (Uniprot P01859, IGHG2_HUMAN, immunoglobulin heavy constant gamma 2; SEQ ID NO: 45), human IgG3 (Uniprot P01860, IGHG3_HUMAN, immunoglobulin heavy constant gamma 3; SEQ ID NO: 46), human IgG4 (Uniprot P01861, IGHG4, immunoglobulin heavy constant gamma 4; SEQ ID NO: 47), human IgD (Uniprot P01880, IGHD_HUMAN, immunoglobulin heavy constant delta; SEQ ID NO: 48), human IgE (Uniprot P01854, IGHE_HUMAN, immunoglobulin heavy constant chain epsilon; SEQ ID NO: 49), or IgM (Uniprot P01871, IGHM_HUMAN, immunoglobulin heavy constant mu; SEQ ID NO: 50), wherein the subsequence comprises the CH1-CH2 hinge region or a portion thereof. In some aspects, the subsequence further comprises an adjacent portion of a CH1 and/or CH2 constant domain.

In some aspects, a spacer comprises a subsequence of an immunoglobulin heavy chain selected the group consisting of human IgA1 (Uniprot: P01876, IGHA1_HUMAN, immunoglobulin heavy constant alpha 1; SEQ ID NO: 41), human IgA2 (Uniprot P01877, IGHA2_HUMAN, immunoglobulin heavy constant alpha 2; SEQ ID NO: 42), murine IgG2A (Uniprot P01665, GCAM_MOUSE, immunoglobulin gamma 2A chain C region; SEQ ID NO: 43), human IgG1 (Uniprot P01857, IGHG1_HUMAN, immunoglobulin heavy constant gamma 1; SEQ ID NO: 44), human IgG2 (Uniprot P01859, IGHG2_HUMAN, immunoglobulin heavy constant gamma 2; SEQ ID NO: 45), human IgG3 (Uniprot P01860, IGHG3_HUMAN, immunoglobulin heavy constant gamma 3; SEQ ID NO: 46), human IgG4 (Uniprot P01861, IGHG4, immunoglobulin heavy constant gamma 4; SEQ ID NO: 47), human IgD (Uniprot P01880, IGHD_HUMAN, immunoglobulin heavy constant delta; SEQ ID NO: 48), human IgE (Uniprot P01854, IGHE_HUMAN, immunoglobulin heavy constant chain epsilon; SEQ ID NO: 49), or IgM (Uniprot P01871, IGHM_HUMAN, immunoglobulin heavy constant mu; SEQ ID NO: 50), wherein the subsequence comprises a loop region from a constant domain or a portion thereof. In some aspects, the subsequence further comprises an adjacent portion of a P-strand.

In some aspects, a spacer useful for the present disclosure is derived from an IgG, e.g., IgG1, IgG2, IgG3, or IgG4. In some aspects, the spacer is derived from an IgG2 hinge. In some aspects, the IgG2 hinge derived spacer comprises at least five, six, or seven consecutive amino acids of SEQ ID NO: 51 (KPCPPCKCP). In some aspects, the spacer comprises an amino acid sequence that is at least about 70%, at least about 75%, at least about 80%, at least about 85%, at least about 90%, at least about 95%, at least about 96%, at least about 97%, at least about 98%, at least about 99%, or about 100% identical to the sequence set forth in SEQ ID NO: 51 (KPCPPCKCP). In some aspects, the spacer comprises, consists, or consists essentially of the sequence set forth in SEQ ID NO: 51 (KPCPPCKCP). In some aspects, the spacer comprises the sequence set forth in SEQ ID NO: 51 (KPCPPCKCP) except for one, 2, 3, 4, 5, 6, 7, 8, 9, or 10 amino acid substitutions. In some aspects, the amino acid substitutions are conservative amino acid substitutions. In some aspects, the amino acid substitution comprises at least one non-conservative amino acid substitution.

In some aspects, a spacer of the present disclosure comprises of the sequence set forth in SEQ ID NO: 51, wherein the spacer sequence further comprises an optional flexible linker (e.g., the linker of GGGSG (SEQ ID NO: 40)). Thus, in some aspects, a spacer of the present disclosure comprises a spacer sequence (e.g., SEQ ID NO: 51) and an optional C-terminal or N-terminal flexible linker. In some aspects, any optional flexible linkers (e.g., gly/ser rich linker) disclosed herein can be appended to the C-terminus and/or the N-terminus of a spacer.

Signal Peptide

As described herein, in some aspects, an immune cell provided herein has been modified to further express a signal peptide (e.g., comprises an exogenous nucleotide sequence encoding a signal peptide). The signal peptide can facilitate the cell surface expression of the encoded protein and then can be subsequently cleaved from the mature protein. In some aspects, such an immune cell has been modified to have an increased level of a c-Jun protein (e.g., with an exogenous nucleotide sequence encoding the c-Jun protein and/or a transcriptional activator capable of increasing the expression of endogenous c-Jun protein) and comprises: an exogenous nucleotide sequence encoding a chimeric binding protein, and an exogenous nucleotide sequence encoding a signal peptide. In some aspects, an immune cell has been modified to express an increased level of a c-Jun protein and comprise: an exogenous nucleotide sequence encoding a chimeric binding protein, an exogenous nucleotide sequence encoding an EGFRt, and an exogenous nucleotide sequence encoding a signal peptide. In some aspects, an immune cell has been modified to express an increased level of a c-Jun protein and comprise: an exogenous nucleotide sequence encoding a chimeric binding protein, an exogenous nucleotide sequence encoding an EGFRt, an exogenous nucleotide sequence encoding a spacer, and an exogenous nucleotide sequence encoding a signal peptide. In some aspects, the one or more exogenous nucleotide sequences are part of a single polycistronic polynucleotide.

Any suitable signal peptide known in the art can be used with the present disclosure. Non-limiting examples of signal peptides are provided in Table 8 (below). In some aspects, the signal peptide is derived from human Ig kappa. In some aspects, the signal peptide comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 54 (MVLQTQVFISLLLWISGAYG). In some aspects, the signal peptide comprises the amino acid sequence set forth in SEQ ID NO: 54 (MVLQTQVFISLLLWISGAYG). In some aspects, the signal peptide is derived from GM-CSF. In some aspects, such a signal peptide comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 53 (MLLLVTSLLLCELPHPAFLLIP). In some aspects, the signal peptide comprises the amino acid sequence set forth in SEQ ID NO: 53 (MLLLVTSLLLCELPHPAFLLIP).

TABLE 8
Signal Peptide Sequences

| Source | Sequence |
|---|---|
| EGFR | MRPSGTAGAALLALLAALCPASRA (SEQ ID NO: |
| | 52) |
| GM-CSF | MLLLVTSLLLCELPHPAFLLIP (SEQ ID NO: 53) |
| human Ig | MVLQTQVFISLLLWISGAYG (SEQ ID NO: 54) |
| kappa | |
| human CD33 | MPLLLLLPLLWAGALA (SEQ ID NO: 55) |

In some aspects, a polynucleotide that can be used to modify an immune cell described herein comprises a single signal peptide (e.g., SEQ ID NO: 53 or 54). In some aspects, the polynucleotide comprises multiple signal peptides (e.g., at least two, three, four, or more). Where multiple signal peptides are involved, in some aspects, each of the multiple signal peptides are different. In some aspects, two or more of the multiple signal peptides are the same.

Linkers

In some aspects, an immune cell described herein (e.g., modified to express an increased level of a c-Jun protein and cultured using the methods provided herein) has been modified to additionally comprise an exogenous nucleotide sequence encoding a linker. Accordingly, in some aspects, an immune cell described herein has been modified to have an increased level of a c-Jun protein (e.g., with an exogenous nucleotide sequence encoding the c-Jun protein and/or a transcriptional activator capable of increasing the expression of endogenous c-Jun protein) and comprise: an exogenous nucleotide sequence encoding a chimeric binding protein, and an exogenous nucleotide sequence encoding a linker. In some aspects, the immune cell has been modified to have an increased level of a c-Jun protein and comprise: an exogenous nucleotide sequence encoding a chimeric binding protein, an exogenous nucleotide sequence encoding an EGFRt, and an exogenous nucleotide sequence encoding a linker. In some aspects, the immune cell has been modified to have an increased level of a c-Jun protein and comprises: an exogenous nucleotide sequence encoding a chimeric binding protein, an exogenous nucleotide sequence encoding an EGFRt, an exogenous nucleotide sequence encoding a spacer, and an exogenous nucleotide sequence encoding a linker. In some aspects, a modified immune cell described herein has an increased level of a c-Jun protein and comprises: an exogenous nucleotide sequence encoding a chimeric binding protein, an exogenous nucleotide sequence encoding an EGFRt, an exogenous nucleotide sequence encoding a spacer, an exogenous nucleotide sequence encoding a signal peptide, and an exogenous nucleotide sequence encoding a linker.

Where multiple exogenous nucleotide sequences are involved, in some aspects, the one or more exogenous nucleotide sequences are part of a single polycistronic polynucleotide. For such aspects, the linker can be between any of the different components of a polynucleotide described herein. For instance, in some aspects, a polynucleotide (e.g., polycistronic) comprises: (i) a first exogenous nucleotide sequence encoding a c-Jun polypeptide, (ii) a second exogenous nucleotide sequence encoding a linker, and (iii) a third nucleotide sequence encoding a chimeric binding protein (e.g., CAR, TCR, caTCR, CSR, or TCR mimic), wherein the second nucleotide sequence is between the first and third nucleotide sequences, such that the c-Jun protein is linked to the chimeric binding protein by the linker. In some aspects, a polynucleotide of the present disclosure can comprise multiple nucleotide sequences encoding a linker (e.g., at least two separate nucleotide sequences). In some aspects, the multiple linkers are the same. In some aspects, the multiple linkers are different.

In some aspects, the linker is a peptide linker. In some aspect, the linker comprises at least about 1 amino acid, at least about 2 amino acids, at least about 3 amino acids, at least about 4 amino acids, at least about 5 amino acids, at least about 6 amino acids, at least about 7 amino acids, at least about 8 amino acids, at least about 9 amino acids, at least about 10 amino acids, at least about 11 amino acids, at least about 12 amino acids, at least about 13 amino acids, at least about 14 amino acids, at least about 15 amino acids, at least about 16 amino acids, at least about 17 amino acids, at least about 18 amino acids, at least about 19 amino acids, at least about 20 amino acids, at least about 25 amino acids, or at least about 30 amino acids. In some aspects, the linker is rich in glycine (e.g., for flexibility). In some aspects, the linker comprises serine and/or threonine (e.g., for solubility). In some aspects, the linker is a Gly/Ser linker.

In some aspects, the glycine/serine linker is according to the formula [(Gly)n-Ser]m (SEQ ID NO: 77) where n is any integer from 1 to 100 and m is any integer from 1 to 100. In some aspects, the glycine/serine linker is according to the formula [(Gly)x-(Ser)y]z (SEQ ID NO: 78) wherein x in an integer from 1 to 4, y is 0 or 1, and z is an integers from 1 to 50. In some aspects, the Gly/Ser linker comprises the sequence Gn (SEQ ID NO: 79), where n can be an integer from 1 to 100. In some aspects, the optional linker can comprise the sequence (GlyAla)n (SEQ ID NO: 80), wherein n is an integer between 1 and 100.

In some aspects, the sequence of the optional linker is GGGG (SEQ ID NO: 81). In some aspects, the sequence of the optional linker is GGGSG (SEQ ID NO: 82).

In some aspects, the optional linker comprises the sequence (GGGSG)n (SEQ ID NO: 64). In some aspects, the optional linker comprises the sequence (GGGGS)n (SEQ ID NO: 65). In some aspects, the optional linker can comprise the sequence (GGGS)n (SEQ ID NO: 66). In some aspects, the optional linker can comprise the sequence (GGS)n (SEQ ID NO: 67). In these instances, n can be an integer from 1 to 100. In other instances, n can be an integer from one to 20, i.e., 1, 2, 3, 4, 5, 6, 7, 8, 9, 10, 11, 12, 13, 14, 15, 16, 17, 18, 19, or 20. In some aspects n is an integer from 1 to 100.

Examples of the optional linker include, but are not limited to, e.g., GSGSGS (SEQ ID NO: 68), GGSGG (SEQ ID NO: 69), SGGSGGS (SEQ ID NO: 70), GGSGGSGGSGGSGGG (SEQ ID NO: 71), GGSGGSGGGGSGGGGS (SEQ ID NO: 72), GGSGGSGGSGGSGGSGGS (SEQ ID NO: 73), or GGGGSGGGGSGGGGS (SEQ ID NO: 74).

In some aspects, the optional linker comprises the sequence PGG. In some aspects, the optional linker comprises additional amino acids in addition to Glycine and Serine. In some aspects, the optional linker comprises 1, 2, 3, 4, or 5 non-gly/non-ser amino acids. In some aspects, the Gly/Ser-linker comprises at least about 60%, at least about 65%, at least about 70%, at least about 80%, at least about 85%, at least about 90%, or at least 95% glycine or serine amino acids.

In some specific aspects, the optional linker is between 1 and 10 amino acids in length. In some aspects, the optional linker as between about 5 and about 10, between about 10 and about 20, between about 20 and about 30, between about 30 and about 40, between about 40 and about 50, between about 50 and about 60, between about 60 and about 70, between about 70 and about 80, between about 80 and about 90, or between about 90 and about 100 amino acids in length.

In some aspects, the linker is a non-cleavable linker, such that the linker and the different components of a polynucleotide provided herein (e.g., c-Jun protein and chimeric binding protein) are expressed as a single polypeptide. In some aspects, the linker is a cleavable linker. As used herein, the term “cleavable linker” refers to a linker that comprises a cleavage site, such that when expressed can be selectively cleaved to produce two or more products. In some aspects, the linker is selected from a P2A linker, a T2A linker, an F2A linker, an E2A linker, a furin cleavage site, or any combination thereof (see Table 9 below). In some aspects, the linker further comprises a GSG linker sequence. In some aspects, a linker useful for the present disclosure comprises an Internal Ribosome Entry Site (IRES), such that separate polypeptides encoded by the first and second genes are produced during translation. Additional description of linkers that can be used with the present disclosure are provided, e.g., in WO 2020/223625 A1 and US 2019/0276801 A1, each of which is incorporated herein by reference in its entirety.

TABLE 9
Linker Sequences

| | |
|---|---|
| P2A | ATNFSLLKQAGDVEENPGP (SEQ ID NO: 14) |
| T2A | EGRGSLLTCGDVEENPGP (SEQ ID NO: 15) |
| F2A | VKQTLNFDLLKLAGDVESNPGP (SEQ ID NO: 16) |
| E2A | QCTNYALLKLAGDVESNPGP (SEQ ID NO: 17) |
| Furin | RAKR (SEQ ID NO: 18) |
| Cleavage | |
| Site | |

In some aspects, the linker comprises a P2A linker. In some aspects, the linker comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 14. In some aspects, the linker comprises the amino acid sequence set forth in SEQ ID NO: 14.

In some aspects, the linker comprises a T2A linker. In some aspects, the linker comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 15. In some aspects, the linker comprises the amino acid sequence set forth in SEQ ID NO: 15.

In some aspects, the linker comprises an F2A linker. In some aspects, the linker comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 16. In some aspects, the linker comprises the amino acid sequence set forth in SEQ ID NO: 16.

In some aspects, the linker comprises an E2A linker. In some aspects, the linker comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 17. In some aspects, the linker comprises the amino acid sequence set forth in SEQ ID NO: 17.

In some aspects, the linker comprises an amino acid sequence comprising a furin cleavage site. In some aspects, the linker comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 18. In some aspects, the linker comprises the amino acid sequence set forth in SEQ ID NO: 18.

As is apparent from the above disclosure, in some aspects, an immune cell described herein (e.g., modified and cultured using the methods provided herein) comprises an exogenous polynucleotide which comprises (from 5′ to 3′): (i) a first nucleotide sequence encoding a c-Jun polypeptide, (ii) a second nucleotide sequence encoding a first linker (e.g., P2A linker), (iii) a third nucleotide sequence encoding a first signal peptide (e.g., hIgκ), (iv) a fourth nucleotide sequence encoding a chimeric binding protein (e.g., scFv), (v) a fifth nucleotide sequence encoding a second linker (e.g., GGGSG; SEQ ID NO: 40), (vi) a sixth nucleotide sequence encoding a spacer (e.g., IgG2 hinge derived spacer), (vii) a seventh nucleotide sequence encoding a transmembrane domain (e.g., CD28), (viii) an eighth nucleotide sequence encoding a costimulatory domain (e.g., 4-1BB), (ix) a ninth nucleotide sequence encoding an intracellular signaling domain (e.g., CD3ζ), (x) a tenth nucleotide sequence encoding a third linker (e.g., P2A linker), (xi) an eleventh nucleotide sequence encoding a second signal peptide (e.g., GMCSFRαSP), and (xii) a twelfth nucleotide sequence encoding a EGFRt.

Delivery Vectors

In some aspects, provided herein are vectors (e.g., expression vectors) that can be used to modify an immune cell described herein (e.g., cultured using the methods provided herein). In some aspects, a vector described herein comprises multiple (e.g., 2, 3, or 4 or more) polynucleotides, wherein the multiple polynucleotides each encode a protein described herein (e.g., c-Jun protein, ligand binding protein (e.g., chimeric binding protein, e.g., CAR), or EGFRt). Accordingly, in some aspects, a vector comprises a polycistronic vector (e.g., bicistronic vector or tricistronic vector). In some aspects, the polynucleotides described herein are comprised on the same vector (e.g., on a multicistronic expression vector). In some aspects, the polynucleotides encoding the proteins described herein (e.g., c-Jun protein, ligand binding protein (e.g., chimeric binding protein, e.g., CAR), or EGFRt) are provided on one or more separate vectors.

As described herein, such vectors are useful for recombinant expression in host cells and cells targeted for therapeutic intervention. The term “vector,” as used herein, is intended to refer to a nucleic acid molecule capable of transporting another nucleic acid to which it has been linked; or an entity comprising such a nucleic acid molecule capable of transporting another nucleic acid. In some aspects, the vector is a “plasmid,” which refers to a circular double stranded DNA loop into which additional DNA segments can be ligated. In some aspects, the vector is a viral vector, wherein additional DNA segments can be ligated into the viral genome. Certain vectors, or polynucleotides that are part of vectors, are capable of autonomous replication in a host cell into which they are introduced (e.g., bacterial vectors having a bacterial origin of replication, and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present disclosure, “plasmid” and “vector” can sometimes be used interchangeably, depending on the context, as the plasmid is the most commonly used form of vector. However, also disclosed herein are other forms of expression vectors, such as viral vectors (e.g., lentiviruses, replication defective retroviruses, poxviruses, herpesviruses, baculoviruses, adenoviruses, and adeno-associated viruses), which serve equivalent functions.

In some aspects, a vector comprises a polynucleotide described herein (e.g., encoding a c-Jun protein and/or a ligand binding protein) and a regulatory element. For instance, in some aspects, a vector comprises a polynucleotide described herein (e.g., encoding a c-Jun protein and/or a ligand binding protein), operatively linked to a promoter. In some aspects, the vector can comprise multiple promoters (e.g., at least two, at least three, at least four, at least five or more). For instance, in some aspects, the nucleotide sequence encoding the c-Jun protein can be under the control of a first promoter, and the nucleotide sequence encoding one or more of the additional components of the polynucleotide (e.g., chimeric binding protein) can be under the control of a second promoter. In some aspects, each of the multiple promoters are the same. In some aspects, one or more of the multiple promoters are different.

Any suitable promoter known in the art can be used with the present disclosure. In some aspects, the promoters useful for the present disclosure comprises a mammalian or viral promoter, such as a constitutive or inducible promoter. In some aspects, the promoters for the present disclosure comprises at least one constitutive promoter and at least one inducible promoter, e.g., tissue specific promoter.

Constitutive mammalian promoters include, but are not limited to, the promoters for the following genes: hypoxanthine phosphoribosyl transferase (HPRT), adenosine deaminase, pyruvate kinase, beta-actin promoter, and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus, and other retroviruses, and the thymidine kinase promoter of herpes simplex virus. As described herein, in some aspects, promoters that can be used with the present disclosure are inducible promoters. Inducible promoters are expressed in the presence of an inducing agent. For example, the metallothionein promoter is induced to promote transcription and translation in the presence of certain metal ions. When multiple inducible promoters are present, they can be induced by the same inducer molecule or a different inducer.

In some aspects, the promoter comprises a myeloproliferative sarcoma virus enhancer, negative control region deleted, dl587rev primer-binding site substituted (MND) promoter, EF1a promoter, or both.

In some aspects, a vector useful for the present disclosure (e.g., comprising one or more nucleotide sequence encoding a c-Jun protein and/or a ligand binding protein) further comprises one or more additional regulatory elements. Non-limiting examples of regulatory elements include a translation enhancer element (TEE), a translation initiation sequence, a microRNA binding site or seed thereof, a 3′ tailing region of linked nucleosides, an AU rich element (ARE), a post transcription control modulator, a 5′ UTR, a 3′ UTR, a localization sequence (e.g., membrane-localization sequences, nuclear localization sequences, nuclear exclusion sequences, or proteasomal targeting sequences), post-translational modification sequences (e.g., ubiquitination, phosphorylation, or dephosphorylation), or combinations thereof.

In some aspects, the vector can additionally comprise a transposable element. Accordingly, in some aspects, the vector comprises a polynucleotide described herein (e.g., encoding a c-Jun protein and/or a ligand binding protein), which is flanked by at least two transposon-specific inverted terminal repeats (ITRs). In some aspects, the transposon-specific ITRs are recognized by a DNA transposon. In some aspects, the transposon-specific ITRs are recognized by a retrotransposon. Any transposon system known in the art can be used to introduce the nucleic acid molecules into the genome of a host cell, e.g., an immune cell. In some aspects, the transposon is selected from hAT-like Tol2, Sleeping Beauty (SB), Frog Prince, piggyBac (PB), and any combination thereof. In some aspects, the transposon comprises Sleeping Beauty. In some aspects, the transposon comprises piggyBac. See, e.g., Zhao et al., Transl. Lung Cancer Res. 5(1):120-25 (2016), which is incorporated by reference herein in its entirety.

In some aspects, the vector is a transfer vector. The term “transfer vector” refers to a composition of matter which comprises an isolated nucleic acid (e.g., a polynucleotide described herein) and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

In some aspects, the vector is an expression vector. The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

In some aspects, the vector is a viral vector, a mammalian vector, or bacterial vector. In some aspects, the vector is selected from the group consisting of an adenoviral vector, a lentivirus, a Sendai virus vector, a baculoviral vector, an Epstein Barr viral vector, a papovaviral vector, a vaccinia viral vector, a herpes simplex viral vector, a hybrid vector, and an adeno associated virus (AAV) vector.

In some aspects, the adenoviral vector is a third generation adenoviral vector. ADEASY™ is by far the most popular method for creating adenoviral vector constructs. The system consists of two types of plasmids: shuttle (or transfer) vectors and adenoviral vectors. The transgene of interest is cloned into the shuttle vector, verified, and linearized with the restriction enzyme PmeI. This construct is then transformed into ADEASIER-1 cells, which are BJ5183 E. coli cells containing PADEASY™. PADEASY™ is a ˜33 Kb adenoviral plasmid containing the adenoviral genes necessary for virus production. The shuttle vector and the adenoviral plasmid have matching left and right homology arms which facilitate homologous recombination of the transgene into the adenoviral plasmid. One can also co-transform standard BJ5183 with supercoiled PADEASY™ and the shuttle vector, but this method results in a higher background of non-recombinant adenoviral plasmids. Recombinant adenoviral plasmids are then verified for size and proper restriction digest patterns to determine that the transgene has been inserted into the adenoviral plasmid, and that other patterns of recombination have not occurred. Once verified, the recombinant plasmid is linearized with PacI to create a linear dsDNA construct flanked by ITRs. 293 or 911 cells are transfected with the linearized construct, and virus can be harvested about 7-10 days later. In addition to this method, other methods for creating adenoviral vector constructs known in the art at the time the present application was filed can be used to practice the methods disclosed herein.

In some aspects, the viral vector is a retroviral vector, e.g., a lentiviral vector (e.g., a third or fourth generation lentiviral vector). The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

Lentiviral vectors are usually created in a transient transfection system in which a cell line is transfected with three separate plasmid expression systems. These include the transfer vector plasmid (portions of the HIV provirus), the packaging plasmid or construct, and a plasmid with the heterologous envelope gene (env) of a different virus. The three plasmid components of the vector are put into a packaging cell which is then inserted into the HIV shell. The virus portions of the vector contain insert sequences so that the virus cannot replicate inside the cell system. Current third generation lentiviral vectors encode only three of the nine HIV-1 proteins (Gag, Pol, Rev), which are expressed from separate plasmids to avoid recombination-mediated generation of a replication-competent virus. In fourth generation lentiviral vectors, the retroviral genome has been further reduced (see, e.g., TAKARA® LENTI-X™ fourth-generation packaging systems).

In some aspects, non-viral methods can be used to deliver a polynucleotide described herein (e.g., encoding a c-Jun protein and/or a ligand binding protein) into an immune cell. In some aspects, the non-viral method includes the use of a transposon. In some aspects, use of a non-viral method of delivery permits reprogramming of cells, e.g., T or NK cells, and direct infusion of the cells into the subject. In some aspects, the polynucleotide can be inserted into the genome of a target cell (e.g., a T cell) or a host cell (e.g., a cell for recombinant expression of the encoded proteins) by using CRISPR/Cas systems and genome edition alternatives such as zinc-finger nucleases (ZFNs), transcription activator-like effector nucleases (TALENs), and meganucleases (MNs). Non-viral delivery systems also include electroporation, cell squeezing, nanoparticles including lipid nanoparticles, gold nanoparticles, polymer nanoparticles. Illustrative non-viral delivery systems include and are described for example in EbioMedicine 2021 May; 67:103354.

In some aspects, the vector disclosed herein (e.g., lentiviral vector) comprises a polynucleotide comprising one or more nucleotide sequences, which encode (i) a c-Jun protein and (ii) an antigen-binding domain (e.g., scFv). In some aspects, the vector comprises a polynucleotide comprising one or more nucleotide sequences, which encode (i) a c-Jun protein, (ii) an antigen-binding domain (e.g., scFv), and (iii) EGFRt. In some aspects, the vector comprises a polynucleotide comprising one or more nucleotide sequences, which encode (i) a c-Jun protein, (ii) an antigen-binding domain (e.g., scFv), (iii) a transmembrane domain (e.g., CD28), (iv) a costimulatory domain (4-1BB), (v) an intracellular signaling domain (CD3ζ), and (vi) a EGFRt. In some aspects, the one or more nucleotide sequences additionally encode a linker, spacer, signal peptide, or combinations thereof. For instance, in some aspects, a vector described herein comprises a polynucleotide, which comprises (from 5′ to 3′) (i) a first nucleotide sequence encoding a c-Jun protein, (ii) a second nucleotide sequence encoding a first linker (e.g., P2A linker), (iii) a third nucleotide sequence encoding a first signal peptide (e.g., hIgκ), (iv) a fourth nucleotide sequence encoding an antigen-binding domain (e.g., scFv), (v) a fifth nucleotide sequence encoding a second linker (e.g., GGGSG; SEQ ID NO: 40), (vi) a sixth nucleotide sequence encoding a spacer (e.g., IgG2 hinge derived spacer), (vii) a seventh nucleotide sequence encoding a transmembrane domain (e.g., CD28), (viii) an eighth nucleotide sequence encoding a costimulatory domain (e.g., 4-1BB), (ix) a ninth nucleotide sequence encoding an intracellular signaling domain (e.g., CD3ζ), (x) a tenth nucleotide sequence encoding a third linker (e.g., P2A linker), (xi) an eleventh nucleotide sequence encoding a second signal peptide (e.g., GM-CSF), and (xii) a twelfth nucleotide sequence encoding a EGFRt.

In some aspects, the polynucleotides disclosed herein (e.g., encoding a c-Jun protein and/or a ligand binding protein) are DNA (e.g., a DNA molecule or a combination thereof), RNA (e.g., a RNA molecule or a combination thereof), or any combination thereof. In some aspects, the polynucleotides are single stranded or double stranded RNA or DNA (e.g., ssDNA or dsDNA) in genomic or cDNA form, or DNA-RNA hybrids, each of which can include chemically or biochemically modified, non-natural, or derivatized nucleotide bases. As described herein, such nucleic acid sequences can comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleotide sequences will encode the different polypeptides described herein (e.g., c-Jun protein, chimeric binding protein, and/or EGFRt).

### III. Compositions of the Disclosure

Certain aspects of the present disclosure are directed to a cell composition comprising a population of immune cells (e.g., T cell and/or NK cell) cultured according to the methods disclosed herein. Certain aspects of the present disclosure are directed to a cell composition comprising a population of immune cells (e.g., T cell and/or NK cell) modified to express an increased level of a c-Jun polypeptide compared to reference immune cells (e.g., corresponding cells that have not been modified to have increased level of the c-Jun polypeptide) and cultured according to the methods disclosed herein. Cell populations cultured according to the methods and/or in a metabolic reprogramming medium disclosed herein have an increased number of less-differentiated cells as compared to comparable cells cultured according to conventional methods, e.g., in media containing less than 5 mM K⁺. In some aspects, the cells cultured according to the methods disclosed herein exhibit increased expression of one or more marker typical of a stem-like phenotype. In some aspects, cell populations cultured according to the methods and/or in a metabolic reprogramming medium disclosed herein have an increased number of effector-like cells as compared to comparable cells cultured according to conventional methods, e.g., in media containing less than 5 mM K⁺. In some aspects, cell populations cultured according to the methods and/or in a metabolic reprogramming medium disclosed herein have both an increased number of stem-like and effector-like cells as compared to comparable cells cultured according to conventional methods, e.g., in media containing less than 5 mM K⁺. In some aspects, the cells cultured according to the methods disclosed herein exhibit greater proliferative potential compared to cells cultured according to conventional methods. In some aspects, the cells cultured according to the methods disclosed herein exhibit increased transduction efficiency. In some aspects, the cells cultured according to the methods disclosed herein exhibit increased in vivo viability upon transplantation in a subject. In some aspects, the cells cultured according to the methods disclosed herein exhibit increased cell potency. In some aspects, the cells cultured according to the methods disclosed herein exhibit decreased cell exhaustion. In some aspects, the cells cultured according to the methods disclosed herein exhibit increased in vivo persistence upon transplantation in a subject.

In some aspects, the cells cultured according to the methods disclosed herein exhibit increased in vivo activity upon transplantation in a subject. In some aspects, the cells cultured according to the methods disclosed herein exhibit a more durable in vivo response upon transplantation in a subject.

In some aspects, the subject is a human.

In some aspects, at least about 5% of the cells in the cell composition have a stem-like phenotype. In some aspects, at least about 10% of the cells in the cell composition have a stem-like phenotype. In some aspects, at least about 15% of the cells in the cell composition have a stem-like phenotype. In some aspects, at least about 20% of the cells in the cell composition have a stem-like phenotype. In some aspects, at least about 25% of the cells in the cell composition have a stem-like phenotype. In some aspects, at least about 30% of the cells in the cell composition have a stem-like phenotype. In some aspects, at least about 35% of the cells in the cell composition have a stem-like phenotype. In some aspects, at least about 40% of the cells in the cell composition have a stem-like phenotype. In some aspects, at least about 45% of the cells in the cell composition have a stem-like phenotype. In some aspects, at least about 50% of the cells in the cell composition have a stem-like phenotype. In some aspects, at least about 55% of the cells in the cell composition have a stem-like phenotype. In some aspects, at least about 60% of the cells in the cell composition have a stem-like phenotype. In some aspects, at least about 65% of the cells in the cell composition have a stem-like phenotype. In some aspects, at least about 70% of the cells in the cell composition have a stem-like phenotype.

In some aspects, following culture of T cells according to the methods disclosed herein, stem-like T cells constitute at least about 10% to at least about 70% of the total number of T cells in the culture. In some aspects, following culture of T cells according to the methods disclosed herein, stem-like T cells constitute at least about 10%, at least about 20%, at least about 30%, at least about 40%, at least about 50%, at least about 60%, or at least about 70% of the total number of CD8+ T cells in the culture. In some aspects, following culture of T cells according to the methods disclosed herein, stem-like T cells constitute at least about 10%, at least about 20%, at least about 30%, at least about 40%, at least about 50%, at least about 60%, or at least about 70% of the total number of CD4+ T cells in the culture.

In some aspects, following culture of T cells according to the methods disclosed herein, the proportion of progenitor exhausted T cells (i.e., T cells enriched for the TPE gene signature) is increased by between about 1.5 fold and about 20 fold. In some aspects, following culture of T cells according to the methods disclosed herein, the proportion of progenitor exhausted T cells is increased by between about 2 fold and about 10 fold. In some aspects, following culture of T cells according to the methods disclosed herein, the proportion of progenitor exhausted T cells is increased by between about 2 fold and about 5 fold.

In some aspects, following culture of T cells according to the methods disclosed herein, the proportion of progenitor exhausted T cells is increased by at least about 1.5 fold. In some aspects, following culture of T cells according to the methods disclosed herein, the proportion of progenitor exhausted T cells is increased by at least about 2 fold. In some aspects, following culture of T cells according to the methods disclosed herein, the proportion of progenitor exhausted T cells is increased by at least about 2.5 fold. In some aspects, following culture of T cells according to the methods disclosed herein, the proportion of progenitor exhausted T cells is increased by at least about 3 fold. In some aspects, following culture of T cells according to the methods disclosed herein, the proportion of progenitor exhausted T cells is increased by at least about 3.5 fold. In some aspects, following culture of T cells according to the methods disclosed herein, the proportion of progenitor exhausted T cells is increased by at least about 4 fold. In some aspects, following culture of T cells according to the methods disclosed herein, the proportion of progenitor exhausted T cells is increased by at least about 4.5 fold. In some aspects, following culture of T cells according to the methods disclosed herein, the proportion of progenitor exhausted T cells is increased by at least about 5 fold. In some aspects, following culture of T cells according to the methods disclosed herein, the proportion of progenitor exhausted T cells is increased by at least about 5.5 fold. In some aspects, following culture of T cells according to the methods disclosed herein, the proportion of progenitor exhausted T cells is increased by at least about 6 fold. In some aspects, following culture of T cells according to the methods disclosed herein, the proportion of progenitor exhausted T cells is increased by at least about 6.5 fold. In some aspects, following culture of T cells according to the methods disclosed herein, the proportion of progenitor exhausted T cells is increased by at least about 7 fold. In some aspects, following culture of T cells according to the methods disclosed herein, the proportion of progenitor exhausted T cells is increased by at least about 7.5 fold. In some aspects, following culture of T cells according to the methods disclosed herein, the proportion of progenitor exhausted T cells is increased by at least about 8 fold. In some aspects, following culture of T cells according to the methods disclosed herein, the proportion of progenitor exhausted T cells is increased by at least about 8.5 fold. In some aspects, following culture of T cells according to the methods disclosed herein, the proportion of progenitor exhausted T cells is increased by at least about 9 fold. In some aspects, following culture of T cells according to the methods disclosed herein, the proportion of progenitor exhausted T cells is increased by at least about 10 fold.

In some aspects, following culture of T cells according to the methods disclosed herein, the proportion of exhausted T cells (e.g., T cells enriched for the TTE gene signature) is reduced by at least about ¼ and the proportion of progenitor exhausted T cells is increased by at least about 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold or at least about 10 fold. In some aspects, following culture of T cells according to the methods disclosed herein, the proportion of exhausted T cells is reduced by at least about ⅓ and the proportion of progenitor exhausted T cells is increased by at least about 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold or at least about 10 fold. In some aspects, following culture of T cells according to the methods disclosed herein, the proportion of exhausted T cells is reduced by at least about ½ and the proportion of progenitor exhausted T cells is increased by at least about 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold or at least about 10 fold. In some aspects, following culture of T cells according to the methods disclosed herein, the proportion of exhausted T cells is reduced by at least about ¾ and the proportion of progenitor exhausted T cells is increased by at least about 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold or at least about 10 fold.

In some aspects, following culture of T cells according to the methods disclosed herein, the proportion of stem-like T cells is increased by at least about 1.5 fold and the proportion of progenitor exhausted T cells is increased by at least about 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold or at least about 10 fold. In some aspects, following culture of T cells according to the methods disclosed herein, the proportion of stem-like T cells is increased by at least about 2 fold and the proportion of progenitor exhausted T cells is increased by at least about 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold or at least about 10 fold. In some aspects, following culture of T cells according to the methods disclosed herein, the proportion of stem-like T cells is increased by at least about 2.5 fold and the proportion of progenitor exhausted T cells is increased by at least about 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold or at least about 10 fold. In some aspects, following culture of T cells according to the methods disclosed herein, the proportion of stem-like T cells is increased by at least about 3 fold and the proportion of progenitor exhausted T cells is increased by at least about 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold or at least about 10 fold. In some aspects, following culture of T cells according to the methods disclosed herein, the proportion of stem-like T cells is increased by at least about 3.5 fold and the proportion of progenitor exhausted T cells is increased by at least about 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold or at least about 10 fold. In some aspects, following culture of T cells according to the methods disclosed herein, the proportion of stem-like T cells is increased by at least about 4 fold and the proportion of progenitor exhausted T cells is increased by at least about 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold or at least about 10 fold. In some aspects, following culture of T cells according to the methods disclosed herein, the proportion of stem-like T cells is increased by at least about 5 fold and the proportion of progenitor exhausted T cells is increased by at least about 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold or at least about 10 fold. In some aspects, following culture of T cells according to the methods disclosed herein, the proportion of stem-like T cells is increased by at least about 6 fold and the proportion of progenitor exhausted T cells is increased by at least about 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold or at least about 10 fold.

In some aspects, the cell composition comprises an increased percentage of immune cells, e.g., T cells and/or NK cells, which express one or more stem-like markers and an increased percentage of immune cells which express one or more TPE markers. In some aspects, the cell composition comprises an increased percentage of immune cells, e.g., T cells and/or NK cells, which express at least two stem-like markers and an increased percentage of immune cells which express one or more TPE markers. In some aspects, the cell composition comprises an increase percent of immune cells, e.g., T cells and/or NK cells, which express at least three stem-like markers and an increased percentage of immune cells, e.g., T cells and/or NK cells, which express one or more TPE markers. In some aspects, the cell composition comprises an increased percentage of immune cells, e.g., T cells and/or NK cells, which express at least four stem-like markers and an increased percentage of immune cells, e.g., T cells and/or NK cells, which express one or more TPE markers. In some aspects, the cell composition comprises an increased percentage of immune cells, e.g., T cells and/or NK cells, which express one or more stem-like markers and an increased percentage of immune cells, e.g., T cells and/or NK cells, which express at least two TPE markers.

In some aspects, the cell composition comprises an increased percentage of immune cells, e.g., T cells and/or NK cells, which express one or more stem-like markers and an increased percentage of immune cells, e.g., T cells and/or NK cells, which express at least three TPE markers. In some aspects, the cell composition comprises an increased percentage of immune cells, e.g., T cells and/or NK cells, which express one or more stem-like markers and an increased percentage of immune cells, e.g., T cells and/or NK cells, which express at least four TPE markers. In some aspects, the cell composition comprises an increased percentage of immune cells, e.g., T cells and/or NK cells, which express one or more stem-like markers and an increased percentage of immune cells, e.g., T cells and/or NK cells, which express at least five TPE markers.

In some aspects, a cell composition herein comprises a population of immune cells wherein at least about 4% of the cells are progenitor exhausted T cells. In some aspects, a cell composition herein comprises a population of immune cells wherein at least about 4%, about 5%, about 6%, about 7%, about 8%, about 9%, or about 10% of the cells are progenitor exhausted T cells. In some aspects, a cell composition herein comprises a population of immune cells wherein between about 4% and about 10% of the cells are progenitor exhausted T cells. In some aspects, a cell composition herein comprises a population of immune cells wherein between about 4% and about 9% of the cells are progenitor exhausted T cells. In some aspects, a cell composition herein comprises a population of immune cells wherein between about 4% and about 8% of the cells are progenitor exhausted T cells. In some aspects, a cell composition herein comprises a population of immune cells wherein between about 4% and about 7% of the cells are progenitor exhausted T cells. In some aspects, a cell composition herein comprises a population of immune cells wherein between about 4% and about 6% of the cells are progenitor exhausted T cells.

In some aspects, a cell composition herein comprises a population of immune cells wherein at least about 4% of the cells are progenitor exhausted T cells and at least about 4% of the cells are stem-like T cells. In some aspects, a cell composition herein comprises a population of immune cells wherein at least about 4% of the cells are progenitor exhausted T cells and at least about 4%, about 5%, about 6%, about 7%, about 8%, about 9%, or about 10% of the cells are stem-like T cells. In some aspects, a cell composition herein comprises a population of immune cells wherein at least about 4%, about 5%, about 6%, about 7%, about 8%, about 9%, or about 10% of the cells are progenitor exhausted T cells and at least about 4% are stem-like T cells. In some aspects, a cell composition herein comprises a population of immune cells wherein at least about 4%, about 5%, about 6%, about 7%, about 8%, about 9%, or about 10% of the cells are progenitor exhausted T cells and at least about 4%, about 5%, about 6%, about 7%, about 8%, about 9%, or about 10% of the cells are stem-like T cells.

In some aspects, a cell composition herein comprises a population of immune cells wherein at least about 4% of the cells are progenitor exhausted T cells. In some aspects, a cell composition herein comprises a population of immune cells wherein at least about 4%, about 5%, about 6%, about 7%, about 8%, about 9%, or about 10% of the cells are progenitor exhausted T cells and less than about 20% of the cells are terminal exhausted cells (TTE). In some aspects, a cell composition herein comprises a population of immune cells wherein at least about 4% of the cells are progenitor exhausted T cells. In some aspects, a cell composition herein comprises a population of immune cells wherein at least about 4%, about 5%, about 6%, about 7%, about 8%, about 9%, or about 10% of the cells are progenitor exhausted T cells and less than about 20%, about 19%, about 18%, about 17%, about 16% or about 15% of the cells are terminal exhausted cells (TTE).

In some aspects, a cell composition herein comprises a population of immune cells wherein at least about 4% of the cells are progenitor exhausted T cells, at least about 4% of the cells are stem-like T cells and less than about 20% of the cells are TTE. In some aspects, a cell composition herein comprises a population of immune cells wherein at least about 4% of the cells are progenitor exhausted T cells, at least about 4%, about 5%, about 6%, about 7%, about 8%, about 9%, or about 10% of the cells are stem-like T cells and less than about 20% of the cells are TTE. In some aspects, a cell composition herein comprises a population of immune cells wherein at least about 4%, about 5%, about 6%, about 7%, about 8%, about 9%, or about 10% of the cells are progenitor exhausted T cells, at least about 4% are stem-like T cells and less than about 20% of the cells are TTE. In some aspects, a cell composition herein comprises a population of immune cells wherein at least about 4%, about 5%, about 6%, about 7%, about 8%, about 9%, or about 10% of the cells are progenitor exhausted T cells, at least about 4%, about 5%, about 6%, about 7%, about 8%, about 9%, or about 10% of the cells are stem-like T cells and less than about 20% of the cells are TTE.

In some aspects, the number of cells having a stem-like phenotype in the cell composition is increased at least about 1.5-fold as compared to the number of cells in the cell composition prior to the culture. In some aspects, the number of cells having a stem-like phenotype in the cell composition is increased at least about 2.0-fold as compared to the number of cells in the cell composition prior to the culture. In some aspects, the number of cells having a stem-like phenotype in the cell composition is increased at least about 2.5-fold as compared to the number of cells in the cell composition prior to the culture. In some aspects, the number of cells having a stem-like phenotype in the cell composition is increased at least about 3.0-fold as compared to the number of cells in the cell composition prior to the culture. In some aspects, the number of cells having a stem-like phenotype in the cell composition is increased at least about 3.5-fold as compared to the number of cells in the cell composition prior to the culture. In some aspects, the number of cells having a stem-like phenotype in the cell composition is increased at least about 4.0-fold as compared to the number of cells in the cell composition prior to the culture. In some aspects, the number of cells having a stem-like phenotype in the cell composition is increased at least about 4.5-fold as compared to the number of cells in the cell composition prior to the culture. In some aspects, the number of cells having a stem-like phenotype in the cell composition is increased at least about 5.0-fold as compared to the number of cells in the cell composition prior to the culture.

In some aspects, the number of cells having a stem-like phenotype in the cell composition is increased at least about 5.5-fold as compared to the number of cells in the cell composition prior to the culture. In some aspects, the number of cells having a stem-like phenotype in the cell composition is increased at least about 6.0-fold as compared to the number of cells in the cell composition prior to the culture. In some aspects, the number of cells having a stem-like phenotype in the cell composition is increased at least about 6.5-fold as compared to the number of cells in the cell composition prior to the culture. In some aspects, the number of cells having a stem-like phenotype in the cell composition is increased at least about 7.0-fold as compared to the number of cells in the cell composition prior to the culture. In some aspects, the number of cells having a stem-like phenotype in the cell composition is increased at least about 7.5-fold as compared to the number of cells in the cell composition prior to the culture. In some aspects, the number of cells having a stem-like phenotype in the cell composition is increased at least about 8.0-fold as compared to the number of cells in the cell composition prior to the culture. In some aspects, the number of cells having a stem-like phenotype in the cell composition is increased at least about 9.0-fold as compared to the number of cells in the cell composition prior to the culture. In some aspects, the number of cells having a stem-like phenotype in the cell composition is increased at least about 10-fold as compared to the number of cells in the cell composition prior to the culture.

In some aspects, the number of cells having a stem-like phenotype in the cell composition is increased at least about 15-fold as compared to the number of cells in the cell composition prior to the culture. In some aspects, the number of cells having a stem-like phenotype in the cell composition is increased at least about 20-fold as compared to the number of cells in the cell composition prior to the culture. In some aspects, the number of cells having a stem-like phenotype in the cell composition is increased at least about 30-fold as compared to the number of cells in the cell composition prior to the culture. In some aspects, the number of cells having a stem-like phenotype in the cell composition is increased at least about 40-fold as compared to the number of cells in the cell composition prior to the culture. In some aspects, the number of cells having a stem-like phenotype in the cell composition is increased at least about 50-fold as compared to the number of cells in the cell composition prior to the culture. In some aspects, the number of cells having a stem-like phenotype in the cell composition is increased at least about 75-fold as compared to the number of cells in the cell composition prior to the culture. In some aspects, the number of cells having a stem-like phenotype in the cell composition is increased at least about 100-fold as compared to the number of cells in the cell composition prior to the culture. In some aspects, the number of cells having a stem-like phenotype in the cell composition is increased at least about 500-fold as compared to the number of cells in the cell composition prior to the culture.

In some aspects, the number of cells having a stem-like phenotype in the cell composition is increased at least about 1000-fold as compared to the number of cells in the cell composition prior to the culture.

In some aspects, following culture of T cells according to the methods disclosed herein, at least about 10% to at least about 70% of the total number of T cells in the culture are CD39⁻/TCF7⁺ T cells. In some aspects, following culture of T cells according to the methods disclosed herein, at least about 10%, at least about 15%, at least about 20%, at least about 25%, at least about 30%, at least about 35%, or at least about 40% of the total number of T cells in the culture are CD39⁻/TCF7⁺ T cells. In some aspects the T cells are CD4⁺ T cells. In some aspects the T cells are CD8⁺ T cells.

In some aspects, the cell composition comprises immune cells, e.g., T cells and/or NK cells. In some aspects, the cell composition comprises an increase in the percent of immune cells, e.g., T cells and/or NK cells, which express CD95. In some aspects, the cell composition comprises an increase in the percent of immune cells, e.g., T cells and/or NK cells, which do not express CD45RO. In some aspects, the cell composition comprises an increase in the percent of immune cells, e.g., T cells and/or NK cells, which express CD45RA. In some aspects, the cell composition comprises an increase in the percent of immune cells, e.g., T cells and/or NK cells, which express CCR7. In some aspects, the cell composition comprises an increase in the percent of immune cells, e.g., T cells and/or NK cells, which express CD62L. In some aspects, the cell composition comprises an increase in the percent of immune cells, e.g., T cells and/or NK cells, which express TCF7. In some aspects, the cell composition comprises an increase in the percent of immune cells, e.g., T cells and/or NK cells, which express CD3. In some aspects, the cell composition comprises an increase in the percent of immune cells, e.g., T cells and/or NK cells, which express CD27. In some aspects, the cell composition comprises an increase in the percent of immune cells, e.g., T cells and/or NK cells, which express CD95 and CD45RA. In some aspects, the cell composition comprises an increase in the percent of immune cells, e.g., T cells and/or NK cells, which express CD45RA and CCR7. In some aspects, the cell composition comprises an increase in the percent of immune cells, e.g., T cells and/or NK cells, which express CD95, CD45RA, and CCR7. In some aspects, the cell composition comprises an increase in the percent of immune cells, e.g., T cells and/or NK cells, which express CD45RA, CCR7, and CD62L. In some aspects, the cell composition comprises an increase in the percent of immune cells, e.g., T cells and/or NK cells, which express CD95, CD45RA, CCR7, and CD62L. In some aspects, the cell composition comprises an increase in the percent of immune cells, e.g., T cells and/or NK cells, which express CD45RA, CCR7, CD62L, and TCF7. In some aspects, the cell composition comprises an increase in the percent of immune cells, e.g., T cells and/or NK cells, which express CD95, CD45RA, CCR7, CD62L, and TCF7. In some aspects, the cell composition comprises an increase in the percent of immune cells, e.g., T cells and/or NK cells, which express CD45RA, CCR7, CD62L, TCF7, and CD27. In some aspects, the cell composition comprises an increase in the percent of immune cells, e.g., T cells and/or NK cells, which express CD95, CD45RA, CCR7, CD62L, TCF7, and CD27. In some aspects, the cell composition comprises an increase in the percent of immune cells, e.g., T cells and/or NK cells, which express, CD45RA, CCR7, CD62L, TCF7, and CD27, and which do not express CD45RO or which are CD45RO^{low}. In some aspects, the cell composition comprises an increase in the percent of immune cells, e.g., T cells and/or NK cells, which express CD95, CD45RA, CCR7, CD62L, TCF7, and CD27, and which do not express CD45RO or which are CD45RO^{low}.

In some aspects, the cell composition comprises an increase in the percent of immune cells, e.g., T cells and/or NK cells, which do not express CD39 and CD69. In some aspects, the cell composition comprises an increase in the percent of immune cells, e.g., T cells and/or NK cells, which express CD8, and which do not express CD39 and CD69. In some aspects, following culture of T cells according to the methods disclosed herein, at least about 10% to at least about 40% of the total number of T cells in the culture are CD39⁻/CD69⁻ T cells. In some aspects, following culture of T cells according to the methods disclosed herein, at least about 10%, at least about 15%, at least about 20%, at least about 25%, at least about 30%, at least about 35%, or at least about 40% of the total number of T cells in the culture are CD39⁻/CD69⁻ T cells.

In some aspects, the cell composition comprises an increased percentage of immune cells, e.g., T cells and/or NK cells, which express both (i) one or more stem-like markers and (ii) one or more effector-like markers. In some aspects, the cell composition comprises an increased percentage of immune cells, e.g., T cells and/or NK cells, which express at least two stem-like markers and one or more effector-like markers. In some aspects, the cell composition comprises an increase percent of immune cells, e.g., T cells and/or NK cells, which express at least three stem-like markers and one or more effector-like markers. In some aspects, the cell composition comprises an increased percentage of immune cells, e.g., T cells and/or NK cells, which express at least four stem-like markers and one or more effector-like markers. In some aspects, the cell composition comprises an increased percentage of immune cells, e.g., T cells and/or NK cells, which express one or more stem-like markers and at least two effector-like markers.

In some aspects, the stem-like markers are selected from CD45RA+, CD62L+, CCR7+, CD27+, CD28+, BACH2+, LEF1+, TCF7+, and any combination thereof. In some aspects the stem-like markers comprise CD45RA+, CD62L+, CCR7+, and TCF7+, or any combination thereof. In some aspects, the cell expresses CD45RO^{low}. In some aspects, the stem-like markers comprise one or more genes listed herein as part of a gene-signature (see supra; see, e.g., Gattinoni, L., et al., Nat Med 17(10): 1290-97 (2011) or Galletti et al. Nat Immunol 21, 1552-62 (2020)).

In some aspects, the stem-like markers comprise a gene expressed in the WNT signaling pathway. In some aspects, the stem-like markers comprise one or more genes selected from GNG2, PSMC3, PSMB10, PSMC5, PSMB8, PSMB9, AKT1, MYC, CLTB, PSME1, DVL2, PFN1, H2AFJ, LEF1, CTBP1, MOV10, HIST1H2BD, FZD3, ITPR3, PARD6A, LRP5, HIST2H4A, HIST2H3C, HIST1H2AD, HIST2H2BE, HIST3H2BB, DACT1, and any combination thereof. In some aspects, the stem-like markers comprise one or more genes selected from MYC, AKT1, LEF1, and any combination thereof.

In some aspects, the effector-like markers are selected from pSTAT5+, STAT5+, pSTAT3+, STAT3+, and any combination thereof. In some aspects, the effector-like marker comprises a STAT target selected from the group consisting of AKT1, AKT2, AKT3, BCL2L1, CBL, CBLB, CBLC, CCND1, CCND2, CCND3, CISH, CLCF1, CNTF, CNTFR, CREBBP, CRLF2, CSF2, CSF2RA, CSF2RB, CSF3, CSF3R, CSH1, CTF1, EP300, EPO, EPOR, GH1, GH2, GHR, GRB2, IFNA1, IFNA10, IFNA13, IFNA14, IFNA16, IFNA17, IFNA2, IFNA21, IFNA4, IFNA5, IFNA6, IFNA7, IFNA8, IFNAR1, IFNAR2, IFNB1, IFNE, IFNG, IFNGR1, IFNGR2, IFNK, IFNL1, IFNL2, IFNL3, IFNLR1, IFNW1, IL10, IL10RA, IL10RB, IL11, IL1IRA, IL12A, IL12B, IL12RB1, IL12RB2, IL13, IL13RA1, IL13RA2, IL15, IL15RA, IL19, IL2, IL20, IL20RA, IL20RB, IL21, IL21R, IL22, IL22RA1, IL22RA2, IL23A, IL23R, IL24, IL26, IL2RA, IL2RB, IL2RG, IL3, IL3RA, IL4, IL4R, IL5, IL5RA, IL6, IL6R, IL6ST, IL7, IL7R, IL9, IL9R, IRF9, JAK1, JAK2, JAK3, LEP, LEPR, LIF, LIFR, MPL, MYC, OSM, OSMR, PIAS1, PIAS2, PIAS3, PIAS4, PIK3CA, PIK3CB, PIK3CD, PIK3CG, PIK3R1, PIK3R2, PIK3R3, PIK3R5, PIM1, PRL, PRLR, PTPN11, PTPN6, SOCS1, SOCS2, SOCS3, SOCS4, SOCS5, SOCS7, SOS1, SOS2, SPRED1, SPRED2, SPRY1, SPRY2, SPRY3, SPRY4, STAM, STAM2, STAT1, STAT2, STAT3, STAT4, STAT5A, STAT5B, STAT6, TPO, TSLP, TYK2, and any combination thereof.

In some aspects, the effector-like markers are effector memory-associated genes that comprise one or more genes selected from TBCD, ARL4C, KLF6, LPGAT1, LPIN2, WDFY1, PCBP4, PIK343, FAS, LLGL2, PPP2R2B, TTC39C, GGA2, LRP8, PMAIP1, MVD, IL15RA, FHOD1, EML4, PEA15, PLEKHA5, WSB2, PAM, CD68, MSC, TLR3, S1PR5, KLRB1, CYTH3, RAB27B, SCD5, and any combination thereof. In some aspects, the effector-like markers comprise one or more genes selected from KLF6, FAS, KLRB1, TLR3, and any combination thereof.

In some aspects, the cell composition comprises an increase in the percent of immune cells, e.g., T cells and/or NK cells, that are CD45RA+, STAT5+, and STAT3+. In some aspects, the cell composition comprises an increase in the percent of immune cells e.g., T cells and/or NK cells, that are CD62L+, STAT5+, and STAT3+. In some aspects, the cell composition comprises an increase in the percent of immune cells, e.g., T cells and/or NK cells, that are TCF7+, STAT5+, and STAT3+. In some aspects, the cell composition comprises an increase in the percent of immune cells, e.g., T cells and/or NK cells, that are CD45RA+, CD62L+, CCR7+, CD27+, CD28+, BACH2+, LEF1+, TCF7+, STAT5+, and STAT3+. In some aspects, the cell composition comprises an increase in the percent of immune cells, e.g., T cells and/or NK cells, that are CD45RA+, CD62L+, CCR7+, CD27+, CD28+, BACH2+, LEF1+, TCF7+, pSTAT5+, STAT5+, pSTAT3+, and STAT3+. In some aspects, the cell composition comprises an increase in the percent of immune cells, e.g., T cells and/or NK cells, that are CD45RA+, CD45RO−, CD62L+, CCR7+, CD27+, CD28+, BACH2+, LEF1+, TCF7+, pSTAT5+, STAT5+, pSTAT3+, and STAT3+.

In some aspects, an immune cell, e.g., T cells and/or NK cells, comprises one or more markers selected from CD45RA+, CD62L+, CCR7+, CD27+, CD28+, BACH2+, LEF1+, TCF7+, and any combination thereof and one or more markers selected from pSTAT5+, STAT5+, pSTAT3+, STAT3+, and any combination thereof. In some aspects, the immune cell, e.g., T cells and/or NK cells, expresses CD45RO^{low}. In some aspects, an immune cell, e.g., T cells and/or NK cells, comprises one or more markers selected from CD45RA+, CD62L+, CCR7+, CD27+, CD28+, BACH2+, LEF1+, TCF7+, and any combination thereof and one or more effector-like markers. In some aspects, an immune cell, e.g., T cells and/or NK cells, comprises one or more stem-like markers and one or more markers selected from pSTAT5+, STAT5+, pSTAT3+, STAT3+, and any combination thereof. In some aspects, the immune cell, e.g., T cells and/or NK cells, expresses CD45RO^{low}.

Some aspects of the present disclosure are directed to a cell composition comprising a population of immune cells, wherein the population of immune cells comprises (i) a first sub-population of immune cells expressing one or more stem-like markers (e.g., stem-like immune cells) and (ii) a second sub-population of immune cells expressing one or more effector-like marker (e.g., effector-like immune cells), wherein the population of immune cells comprises a higher percentage (i.e., the number of stem-like immune cells/the total number of immune cells) of the first sub-population of immune cells expressing one or more stem-like markers, as compared to a population of immune cells cultured using conventional methods, e.g., in a medium having less than 5 mM potassium ion. In some aspects the immune cells are T cells. In some aspects the immune cells are NK cells. In some aspects, the immune cells, e.g., T cells and/or NK cells, cultured according to the methods disclosed herein result in these cell compositions.

In some aspects, immune cells, e.g., T cells and/or NK cells, cultured according to the methods disclosed herein have increased expression, e.g., a higher percentage of immune cells, e.g., T cells and/or NK cells, that express, GZMB, MHC-II, LAG3, TIGIT, and/or NKG7, and decreased expression, e.g., a lower percentage of immune cells, e.g., T cells and/or NK cells, that express, IL-32. Cells highest for NKG7 have been shown to be better killers (Malarkannan et al. 2020 Nat. Immuno.), whereas cells higher in IL-32 have been shown to have activation-induced cell death (Goda et al., 2006 Int. Immunol). In some aspects the immune cells, e.g., T cells and/or NK cells, with higher expression of GZMB, MHC-II, LAG3, TIGIT, and/or NKG7 are CD8+ T cells expressing effector-like markers. In some aspects the immune cells, e.g., T cells and/or NK cells, with lower expression of IL-32 are CD8+ T cells expressing effector-like markers.

In some aspects, the cell composition, obtained by any method described herein (e.g., the yield of the final cell product for use as a therapy), comprises at least about 1×10⁵, 5×10⁵, 1×10⁶, 5×10⁶, 1×10⁷, 5×10⁷, 1×10⁸, 5×10⁸, 1×10⁹, or 5×10⁹ cells. In some aspects, the cell composition, obtained by any method described herein, comprises at least about 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, or 5×10⁹ stem-like cells. In some aspects, the cell composition, obtained by any method described herein, comprises at least about 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 10×10¹⁰, 11×10¹⁰, 12×10¹⁰, 13×10¹⁰, 14×10¹⁰, or 15×10¹⁰ cells. In some aspects, the cell composition, obtained by any method described herein, comprises at least about 1×10⁶ cells. In some aspects, the cell composition, obtained by any method described herein, comprises at least about 1×10⁶ stem-like cells. In some aspects, the cell composition, obtained by any method described herein, comprises at least about 1×10¹⁰ cells. In some aspects, the cell composition, obtained by any method described herein, comprises at least about 2×10¹⁰ cells. In some aspects, the cell composition, obtained by any method described herein, comprises at least about 3×10¹⁰ cells. In some aspects, the cell composition, obtained by any method described herein, comprises at least about 4×10¹⁰ cells. In some aspects, the cell composition, obtained by any method described herein, comprises at least about 5×10¹⁰ cells. In some aspects, the cell composition, obtained by any method described herein, comprises at least about 6×10¹⁰ cells. In some aspects, the cell composition, obtained by any method described herein, comprises at least about 7×10¹⁰ cells. In some aspects, the cell composition, obtained by any method described herein, comprises at least about 8×10¹⁰ cells. In some aspects, the cell composition, obtained by any method described herein, comprises at least about 9×10¹⁰ cells. In some aspects, the cell composition, obtained by any method described herein, comprises at least about 10×10¹⁰ cells. In some aspects, the cell composition, obtained by any method described herein, comprises at least about 11×10¹⁰ cells. In some aspects, the cell composition, obtained by any method described herein, comprises at least about 12×10¹⁰ cells. In some aspects, the cell composition, obtained by any method described herein, comprises at least about 13×10¹⁰ cells. In some aspects, the cell composition, obtained by any method described herein, comprises at least about 14×10¹⁰ cells. In some aspects, the cell composition, obtained by any method described herein, comprises at least about 15×10¹⁰ cells. In some aspects, cell yield represents the total number of CD3+ cells.

In some aspects, the methods disclosed herein yield a composition comprising at least about 1×10¹⁰, at least about 1.1×10¹⁰, at least about 1.2×10¹⁰, at least about 1.3×10¹⁰, at least about 1.4×10¹⁰, at least about 1.5×10¹⁰, at least about 1.6×10¹⁰, at least about 1.7×10¹⁰, at least about 1.8×10¹⁰, at least about 1.9×10¹⁰, or at least about 2.0×10¹⁰ cells by at least about day 10 of culturing in the presently disclosed medium. In some aspects, the methods disclosed herein yield a composition comprising at least about 1.8×10¹⁰ cells by at least about day 10 of culturing in the presently disclosed medium.

In some aspects, the cell composition comprises at least about 1×10¹⁰, at least about 1.1×10¹⁰, at least about 1.2×10¹⁰, at least about 1.3×10¹⁰, at least about 1.4×10¹⁰, at least about 1.5×10¹⁰, at least about 1.6×10¹⁰, at least about 1.7×10¹⁰, at least about 1.8×10¹⁰, at least about 1.9×10¹⁰, or at least about 2.0×10¹⁰ stem-like cells. In some aspects, the methods disclosed herein yield a composition comprising at least about 1×10¹⁰, at least about 1.1×10¹⁰, at least about 1.2×10¹⁰, at least about 1.3×10¹⁰, at least about 1.4×10¹⁰, at least about 1.5×10¹⁰, at least about 1.6×10¹⁰, at least about 1.7×10¹⁰, at least about 1.8×10¹⁰, at least about 1.9×10¹⁰, or at least about 2.0×10¹⁰ stem-like cells by at least about day 10 of culture. In some aspects, the methods disclosed herein yield a composition comprising at least about 1.8×10¹⁰ stem-like cells by at least about day 10 of culturing in the presently disclosed medium.

In some aspects, the methods disclosed herein yield a composition comprising immune cells that are at least about 80%, at least about 85%, at least about 90%, at least about 94%, at least about 95%, at least about 96%, at least about 97%, at least about 98%, or at least about 99% viable. In some aspects, the methods disclosed herein yield a composition comprising at least about 1.8×10¹⁰ stem-like cells with at least about 94% cell viability.

### IV. Methods of Treatment

Some aspects of the present disclosure are directed to methods of administering an immune cell described herein (e.g., modified to express a chimeric binding protein and an increased level of c-Jun protein, and cultured using the methods provided herein). Some aspects of the present disclosure are directed to methods of treating a disease or disorder in a subject in need thereof, comprising administering to the subject an immune cell described herein. For instance, in some aspects, disclosed herein is a method of treating a disease or disorder in a subject in need thereof, comprising administering to the subject an immune cell that has been engineered to express a chimeric binding protein (e.g., CAR) and overexpress a c-Jun protein. In some aspects, the disease or condition comprises a tumor, i.e., a cancer. In some aspects, the method comprises stimulating a T cell-mediated immune response to a target cell population or tissue in a subject, comprising administering an immune cell described herein. In some aspects, the target cell population comprises a tumor. In some aspects, the tumor is a solid tumor.

In some aspects, administering an immune cell described herein (e.g., modified to express a chimeric binding protein and an increased level of c-Jun protein, and cultured using the methods provided herein) reduces a tumor volume in the subject compared to a reference tumor volume. In some aspects, the reference tumor volume is the tumor volume in the subject prior to the administration. In some aspects, the reference tumor volume is the tumor volume in a corresponding subject that did not receive the administration. In some aspects, the tumor volume in the subject is reduced by at least about 5%, at least about 10%, at least about 15%, at least about 30%, at least about 40%, at least about 50%, at least about 60%, at least about 70%, at least about 80%, at least about 90%, or at least about 100% after the administration compared to the reference tumor volume.

In some aspects, treating a tumor comprises reducing a tumor weight in the subject. In some aspects, administering an immune cell described herein (e.g., modified to express a chimeric binding protein and an increased level of c-Jun protein, and cultured using the methods provided herein) can reduce the tumor weight in a subject when administered to the subject. In some aspects, the tumor weight is reduced by at least about 5%, at least about 10%, at least about 15%, at least about 30%, at least about 40%, at least about 50%, at least about 60%, at least about 70%, at least about 80%, at least about 90%, or at least about 100% after the administration compared to a reference tumor weight. In some aspects, the reference tumor weight is the tumor weight in the subject prior to the administration. In some aspects, the reference tumor weight is the tumor weight in a corresponding subject that did not receive the administration.

In some aspects, administering an immune cell described herein (e.g., modified to express a chimeric binding protein and have increased level of c-Jun protein, and cultured using the methods provided herein) to a subject, e.g., suffering from a tumor, can increase the number and/or percentage of T cells (e.g., CD4⁺ or CD8⁺) in the blood of the subject. In some aspects, the T cells are the modified immune cells. In some aspects, the number and/or percentage of the T cells (e.g., modified to express a chimeric binding protein and have increased level of a c-Jun protein, and cultured using the methods provided herein) in the blood is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least 220%, at least about 230%, at least about 240%, at least about 250%, at least about 260%, at least about 270%, at least about 280%, at least about 290%, or at least about 300% or more compared to a reference (e.g., corresponding value in a subject that did not receive the administration or the same subject prior to the administration). In some aspects, the number and/or percentage of T cells in the blood is increased by at least about 2-fold, at least about 3-fold, at least about 4-fold, at least about 5-fold, at least about 6-fold, at least about 7-fold, at least about 8-fold, at least about 9-fold, or at least about 10-fold or more compared to a reference (e.g., corresponding subject that did not receive the administration).

In some aspects, administering an immune cell described herein (e.g., modified to express a chimeric binding protein and have increased level of c-Jun protein, and cultured using the methods provided herein) to a subject, e.g., suffering from a tumor, can increase the number and/or percentage of T cells (e.g., CD4+ or CD8+) in a tumor and/or a tumor microenvironment (TME) of the subject. In some aspects, the T cells are the modified immune cells. In some aspects, the number and/or percentage of the T cells (e.g., modified to express a chimeric binding protein and have increased level of a c-Jun protein) in a tumor and/or TME is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least 220%, at least about 230%, at least about 240%, at least about 250%, at least about 260%, at least about 270%, at least about 280%, at least about 290%, or at least about 300% or more compared to a reference (e.g., corresponding value in a subject that did not receive the administration or the same subject prior to the administration). In some aspects, the number and/or percentage of T cells in a tumor and/or TME is increased by at least about 2-fold, at least about 3-fold, at least about 4-fold, at least about 5-fold, at least about 6-fold, at least about 7-fold, at least about 8-fold, at least about 9-fold, or at least about 10-fold or more compared to a reference (e.g., corresponding subject that did not receive the administration).

In some aspects, administering an immune cell described herein (e.g., modified to express a chimeric binding protein and an increased level of c-Jun protein, and cultured using the methods provided herein) to a subject, e.g., suffering from a tumor, can increase the duration of an immune response in a subject relative to the duration of an immune response in a corresponding subject that did not receive the administration (e.g., treated with a corresponding cell but lacking c-Jun protein expression). In some aspects, the duration of the immune response is increased by at least about 5%, at least about 10%, at least about 20%, at least about 30%, at least about 40%, at least about 50%, at least about 75%, at least about 100%, at least about 150%, at least about 200%, at least about 300%, at least about 400%, at least about 500%, or at least about 1000% or more compared to a reference (e.g., corresponding subject that did not receive the administration). In some aspects, the duration of the immune response is increased by at least about 2-fold, at least about 3-fold, at least about 4-fold, at least about 5-fold, at least about 6-fold, at least about 7-fold, at least about 8-fold, at least about 9-fold, or at least about 10-fold or more compared to a reference (e.g., corresponding subject that did not receive the administration). In some aspects, the duration of an immune response is increased by at least about 1 day, at least about 2 days, at least about 3 days, at least about 4 days, at least about 5 days, at least about 6 days, at least about 1 week, at least about 2 weeks, at least about 3 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, or at least about 5 years, as compared to a reference (e.g., corresponding subject that did not receive the administration).

As described herein, an immune cell described herein (e.g., modified to express a chimeric binding protein and an increased level of c-Jun protein, and cultured using the methods provided herein) can be used to treat variety of cancers. Non-limiting examples of cancers that can be treated include adrenal cortical cancer, advanced cancer, anal cancer, aplastic anemia, bile duct cancer, bladder cancer, bone cancer, bone metastasis, brain tumors, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, non-small cell lung cancer, small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor, secondary cancers caused by cancer treatment, and combinations thereof. In some aspects, the cancer is associated with a solid tumor.

In some aspects, an immune cell described herein (e.g., modified to express a chimeric binding protein and an increased level of c-Jun protein, and cultured using the methods provided herein) is used in combination with other therapeutic agents (e.g., anti-cancer agents and/or immunomodulating agents). Accordingly, in some aspects, a method of treating a disease or disorder (e.g., tumor) disclosed herein comprises administering an immune cell described herein (e.g., modified to express a chimeric binding protein and an increased level of c-Jun protein, and cultured using the methods provided herein) in combination with one or more additional therapeutic agents. Such agents can include, for example, chemotherapeutic drug, targeted anti-cancer therapy, oncolytic drug, cytotoxic agent, immune-based therapy, cytokine, surgery, radiotherapy, activator of a costimulatory molecule, immune checkpoint inhibitor, a vaccine, a cellular immunotherapy, or any combination thereof.

In some aspects, an immune cell described herein (e.g., modified to express a chimeric binding protein and an increased level of c-Jun protein, and cultured using the methods provided herein) is administered to the subject prior to or after the administration of the additional therapeutic agent. In some aspects, an immune cell described herein (modified to express a chimeric binding protein and an increased level of c-Jun protein, and cultured using the methods provided herein) is administered to the subject concurrently with the additional therapeutic agent. In some aspects, an immune cell described herein (e.g., modified to express a chimeric binding protein and an increased level of c-Jun protein, and cultured using the methods provided herein) and the additional therapeutic agent can be administered concurrently as a single composition in a pharmaceutically acceptable carrier. In some aspects, an immune cell described herein (e.g., modified to express a chimeric binding protein and an increased level of c-Jun protein, and cultured using the methods provided herein) and the additional therapeutic agent are administered concurrently as separate compositions.

In some aspects, an immune cell described herein (e.g., modified to express a ROR1-binding protein and have an increased level of c-Jun protein, and cultured using the methods provided herein) is administered to the subject prior to or after the administration of a BCR-ABL/Src kinase inhibitor, such as dasatinib or ponatinib. In some aspects, dasatinib or ponatinib can be administered to reduce cytotoxicities that can sometimes occur with CAR-T cell therapy (e.g., cytokine storm). Src kinases are known to play an important role in physiological T-cell activation. Consistent with this, dasatinib has been shown to profoundly inhibit antigen specific physiological T-cell activation, proliferation, cytokine production, and degranulation in a dose-dependent manner (Schade et al., Blood 111:1366-77, 2008; Weichsel et al., Clin Cancer Res 14:2484-91, 2008) and has been shown to reduce cytoxicities in CAR-T cell therapy (see e.g., US2021032363).

In some aspects, an immune cell described herein (e.g., modified to express a chimeric binding protein and an increased level of c-Jun protein, and cultured using the methods provided herein) is used in combination with a standard of care treatment (e.g., surgery, radiation, and chemotherapy). Methods described herein can also be used as a maintenance therapy, e.g., a therapy that is intended to prevent the occurrence or recurrence of tumors.

In some aspects, an immune cell provided herein (e.g., modified to express a chimeric binding protein and an increased level of c-Jun protein, and cultured using the methods provided herein) is used in combination with one or more anti-cancer agents, such that multiple elements of the immune pathway can be targeted. Non-limiting examples of such combinations include: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Tregs or other immune suppressing cells (e.g., myeloid-derived suppressor cells); a therapy that stimulates positive immune regulation, e.g., with agonists that stimulate the CD-137, OX-40, and/or CD40 or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically engineered cells, e.g., cells engineered to express a chimeric antigen receptor (CAR-T therapy); a therapy that inhibits a metabolic enzyme such as indoleamine dioxigenase (IDO), dioxigenase, arginase, or nitric oxide synthetase; a therapy that reverses/prevents T cell anergy or exhaustion; a therapy that triggers an innate immune activation and/or inflammation at a tumor site; administration of immune stimulatory cytokines; blocking of immuno repressive cytokines; or any combination thereof.

In some aspects, an anti-cancer agent comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof. Non-limiting examples of such immune checkpoint inhibitors include the following: anti-PD1 antibody (e.g., nivolumab (OPDIVO®), pembrolizumab (KEYTRUDA®; MK-3475), pidilizumab (CT-011), PDR001, MEDIO680 (AMP-514), TSR-042, REGN2810, JS001, AMP-224 (GSK-2661380), PF-06801591, BGB-A317, BI 754091, SHR-1210, and combinations thereof); anti-PD-L1 antibody (e.g., atezolizumab (TECENTRIQ®; RG7446; MPDL3280A; RO5541267), durvalumab (MEDI4736, IMFINZI®), BMS-936559, avelumab (BAVENCIO®), LY3300054, CX-072 (Proclaim-CX-072), FAZ053, KN035, MDX-1105, and combinations thereof); and anti-CTLA-4 antibody (e.g., ipilimumab (YERVOY®), tremelimumab (ticilimumab; CP-675,206), AGEN-1884, ATOR-1015, and combinations thereof).

In some aspects, an anti-cancer agent comprises an immune checkpoint activator (i.e., promotes signaling through the particular immune checkpoint pathway). In some aspects, immune checkpoint activator comprises OX40 agonist (e.g., anti-OX40 antibody), LAG-3 agonist (e.g. anti-LAG-3 antibody), 4-1BB (CD137) agonist (e.g., anti-CD137 antibody), GITR agonist (e.g., anti-GITR antibody), TIM3 agonist (e.g., anti-TIM3 antibody), or combinations thereof.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); Crooks, Antisense drug Technology: Principles, strategies and applications, 2^{nd} Ed. CRC Press (2007) and in Ausubel et al. (1989) Current Protocols in Molecular Biology (John Wiley and Sons, Baltimore, Md.).

All of the references cited above, as well as all references cited herein and the amino acid or nucleotide sequences (e.g., GenBank numbers and/or Uniprot numbers), are incorporated herein by reference in their entireties.

The following examples are offered by way of illustration and not by way of limitation.

### EXAMPLES

### Example 1: Analysis of CAR Transduction Efficiency

To assess the effect that metabolic reprogramming media has on CAR transduction efficiency, human CD4+ and CD8+ T cells were transduced with anti-ROR1 CAR constructs in either metabolic reprogramming media (MRM) or a T-cell conditioned medium (i.e., TCM). Provided below are exemplary methods used in carrying out the present Example.

### Media Preparation

T cell conditioned medium (TCM), which was used as a control, was supplemented with immune Cell Serum Replacement (Thermo Fisher), 2 mM L-glutamine (Gibco), 2 mM Glutamax (Gibco), MEM Non-Essential Amino Acids Solution (Gibco), Sodium pyruvate (Gibco), IL-2, 200 IU/mL; IL-7, 1200 IU/ml; IL-15, 200 IU/ml.

Metabolic reprogramming media (MRM) was produced using TCM supplemented with varying concentrations of sodium, potassium, glucose, and calcium. The final concentrations were in the range of: NaCl (40-80 mM), KCl (40-80 mM), Calcium (0.5-2.8 mM), Glucose (10-24 mM) and osmolality (˜250-260 mOsmol). See Table. 10.

TABLE 10
Media with varying concentrations of potassium,
sodium, glucose, and calcium

| | K | NaCl | Glucose | Ca | Osmolality | Tonicity* |
|---|---|---|---|---|---|---|
| Media | (mM) | (mM) | (mM) | (mM) | (mOsmol) | (mOsmol) |
| Basal Media | 4 | 118.47 | ~24 | ~2.8 | 245 | 245 |
| Condition 1 | 80 | 55.6 | 15 | 1.2 | ~262.26 | 271.2 |
| Condition 2 | 75 | 59.3 | 15.4 | 1.3 | ~260 | 268.6 |
| Condition 3 | 70 | 63.9 | 15.9 | 1.4 | ~259.7 | 267.8 |
| Condition 4 | 65 | 67.6 | 16.3 | 1.5 | ~257.5 | 265.2 |
| Condition 5 | 60 | 72.2 | 16.8 | 1.6 | ~257.2 | 264.4 |
| Condition 6 | 55 | 76 | 17.2 | 1.7 | ~255.2 | 262 |
| Condition 7 | 50 | 80.5 | 17.7 | 1.8 | ~254.7 | 261 |
| RPMI | 5.34 | 103 | 11.1 | 0.4 | | 216.7 |
| Gibco + | | | | | | |
| ICSR | | | | | | |
| RPMI | 55.34 | 103 | | | | 316.7 |
| 1640 + | | | | | | |
| 50 mM K+ | | | | | | |

*Tonicity is calculated based on the following formula: 2 × (concentration of K + concentration of NaCl)

Lentiviral Vector (LVV) Construction and Lentiviral Production

An anti-ROR1 CAR construct comprising the following components was generated: (i) anti-ROR1 CAR (derived from the R12 antibody) (referred to herein as “R12 CAR”) (SEQ ID NO: 83), (ii) truncated EGFR (“EGFRt”) (SEQ ID NO: 24), and (iii) wild-type c-Jun protein (SEQ ID NO: 13) (referred to herein as the “c-Jun-R12 CAR”; SEQ ID NO: 86). See Table 12 (below). The c-Jun-R12 CAR construct was designed, such that when transduced in a cell (e.g., T cell), the transduced cell would exhibit increased c-Jun protein expression along with surface expression of the anti-ROR1 CAR and EGFRt. As a control, a corresponding anti-ROR1 CAR construct comprising truncated CD19 (“CD19t”) instead of c-Jun was also generated (referred to herein as the “control CD19t-R12 CAR”). See Terakura, S. et al., Blood 119(1): 72-82 (2012), which is incorporated herein by reference in its entirety.

Lentiviral vectors were pseudotyped with the VSV-G envelope and produced by transient transfection of HEK293T cells. The final bulk was held at 2-8° C. for no longer than 24 hours prior to filling 1 mL aliquots of LVV and stored at −80° C. The LVV aliquots were thawed on ice prior to T cell transduction.

T Cell Isolation

CD4+ and CD8+ T cells were isolated from three healthy donors and frozen using vendors, BloodWorks (Seattle, WA, USA) and AllCells (Alameda, CA, USA). The vendors obtained and maintained all appropriate consent forms from the donors. To isolate the CD4+ and CD8+ T cells, samples were collected samples via apheresis, from which CD4+ and CD8+ cells were isolated separately in order of CD8+ T cells positively selected first followed by positive selection for CD4+ T cells of the flow-through from the CD8 selection. Isolated CD4+ or CD8+ T cells were frozen either at 20E+06 cells (AllCells) or 50E+06 cells (BloodWorks) per vial.

Cell Culture and Transduction

Healthy donor cryopreserved human CD4+ and CD8+ T cells (i.e., from the vendors) were thawed in the appropriate media (i.e., TCM or MRM) and combined at a 1:1 ratio. The combined donor CD4+ and CD8+ T cells were centrifuged at 300×g for 5 minutes and resuspended in appropriate media (i.e., T cell conditioned media or MRM) supplemented with IL-2, IL-7, and IL-15. The T cells were then activated using CD3/CD28 TRANSACT™ (Miltenyi Biotec Inc.). After 24 hours of activation (i.e., day 1) in either TCM or MRM, the T cells were transduced with the above-described LVVs comprising the anti-ROR1 CAR constructs (i.e., “c-Jun-R12 CAR” and “control CD19t-R12 CAR”). Non-transduced T cells were used as control. The following day after transduction (i.e., day 2), fresh media (i.e., TCM or MRM) were added to dilute the TRANSACT™ and end T cell activation. The transduced T cells were allowed to further expand for five additional days (i.e., day 7), and then either subsequently analyzed or cryopreserved in liquid nitrogen for long-term storage.

Transduction Efficiency Analysis

To compare the CAR transduction efficiency from the different groups (see Table 11), the percentage of CD4+ and CD8+ T cells expressing the following was determined using flow cytometry: (i) c-Jun, anti-ROR1 R12 scFv, and EGFRt comprising sequence set forth in SEQ ID NO: 24 or (ii) c-Jun, anti-ROR1 R12 scFv, and truncated CD19.

TABLE 11
Experimental Groups

| Group No. | Description |
|---|---|
| 1 | Non-transduced T cells cultured in TCM |
| 2 | T cells transduced with control CD19t-R12 CAR and |
| | cultured in TCM |
| 3 | T cells transduced with c-Jun-R12 CAR and cultured |
| | in TCM |
| 4 | Non-transduced T cells cultured in MRM |
| 5 | T cells transduced with control CD19t-R12 CAR |
| | cultured in MRM |
| 6 | T cells transduced with c-Jun-R12 CAR cultured |
| | in MRM |

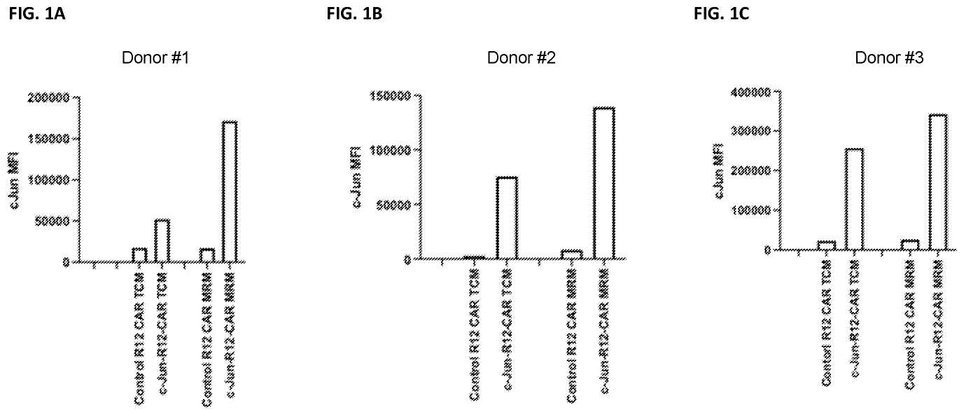

In both the TCM and MRM groups and within each donor, the transduction efficiency between the control CD19t-R12 CAR and c-Jun-R12 CAR was comparable. Similarly, as between the TCM and MRM groups, the percentage of transduced T cells (i.e., expressing both EGFRt and R12 CAR) was comparable. There was also no significant difference observed in the percentage of CD4+ and CD8+ T cells that were transduced from the different groups within each donor. Interestingly, T cells transduced with c-Jun-R12 CAR from the MRM group expressed significantly higher levels of c-Jun protein expression compared to the corresponding transduced T cells from the TCM group (see FIGS. 1A-1C). The increased expression was specific to c-Jun protein and was not global to the other transgenes (i.e., R12 CAR and EGFRt).

These results suggest that the anti-ROR1 CAR constructs described herein are capable of being transduced into T cells with similar degree under both culture conditions. The results further suggest that the metabolic reprogramming media condition could be useful in selectively increasing the expression of the c-Jun protein of the anti-ROR1 CAR constructs provided herein.

### Example 2: Analysis of Stem-Like Phenotypic Expression

To assess the effect of metabolic reprogramming on the stem-like properties of the transduced T cells overexpressing c-Jun, human CD4+ and CD8+ T cells were transduced with anti-ROR1 CAR constructs as described in Example 1 (i.e., c-Jun-R12 CAR or control CD19t-R12 CAR). Then, after the cells were allowed to expand for four to five additional days (i.e., day 6 or 7), the stemness of the transduced T cells was assessed using flow cytometry.

Briefly, the T cells were first washed with cell staining buffer and stained with anti-CCR7 for 15 minutes at 37° C. Next, the T cells were washed again and then a master mix of the antibodies against several other antigens (as detailed below) was added to the cells and incubated for 25 minutes in the dark at room temperature. Cells were then washed with cell staining buffer and permeabilized with the FOXP3 staining kit (ebioscience) as per manufacturers' protocol. After fixing, the cells were blocked with pre-diluted normal mouse serum (Jackson ImmunoResearch-#015-000-120) and normal rabbit serum (Jackson ImmunoResearch-#011-000-120) for 15 minutes in the dark at room temperature. The cells were then stained with a 2× antibody cocktail of TCF7 and c-Jun for 30 minutes in the dark at room temperature. After thoroughly washing the cells, they were analyzed by flow cytometry on Cytek Aurora Spectral Flow Cytometer and analyzed using FlowJo software (TreeStar, Ashland, OR).

The following are the list of antibodies used for assessing the stemness markers: CD8 (Thermo-#58-0088-42), CD4 (BD-#612936), CD27 (BD-#612829), CD3 (Thermo-#612896), CD28 (Biolegend-#302936), CD62L (BD-#740301), R12 Anti-Id (Genscript-#48F6H5E1), EGFR (BioLegend-#98812), CD45RO (BioLegend-#566143), CD39 (BioLegend-#328236), TCF7 (Cell Signaling-#9066S), c-Jun (Cell Signaling-#15683S), CCR7 (BD-#562381), CD45RA (BD-#560673), LAG-3 (Thermo-#67-2239-42), TIM-3 (Thermo-#78-3109-42), TIGIT (Thermo-#46-9500-42), PD-1 (Thermo-#25-2799-42). Specifically, as described herein, “stem-like” cells were defined as: CD45RO⁻CCR7⁺CD45RA⁺CD62L⁺CD27⁺CD28⁺TCF7⁺.

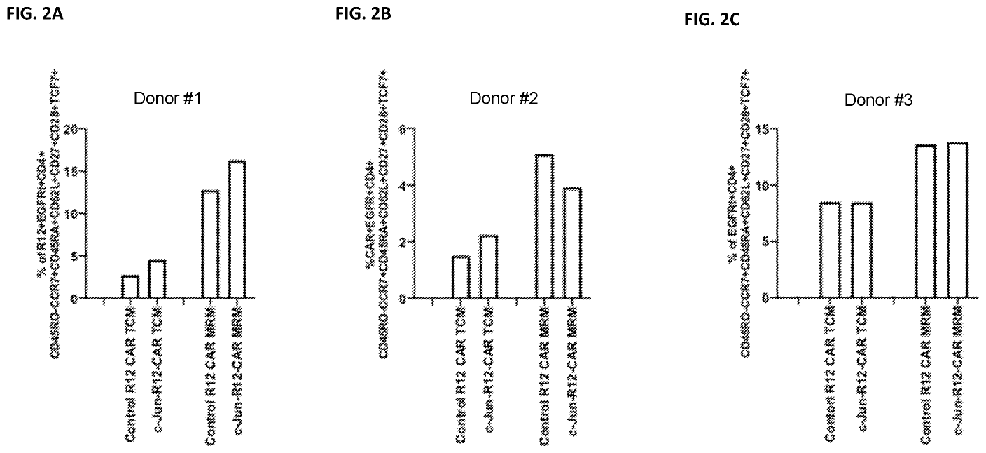

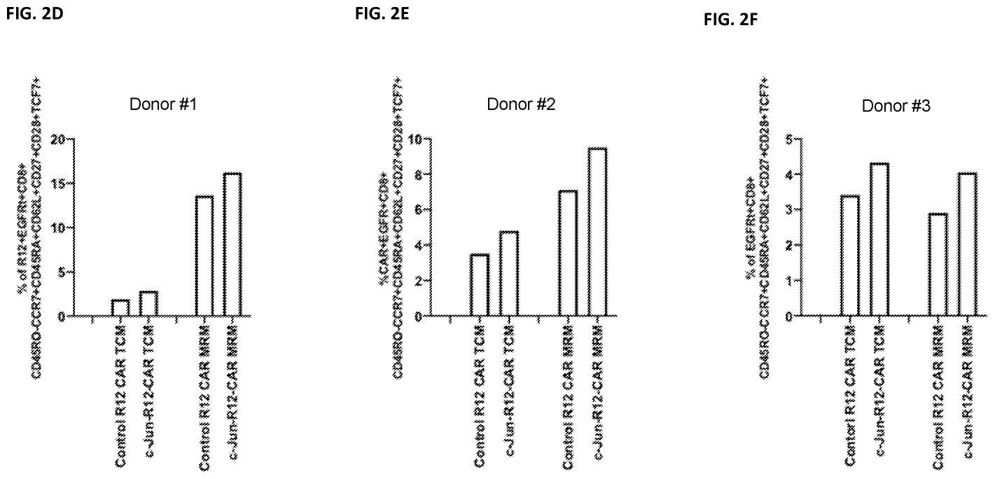

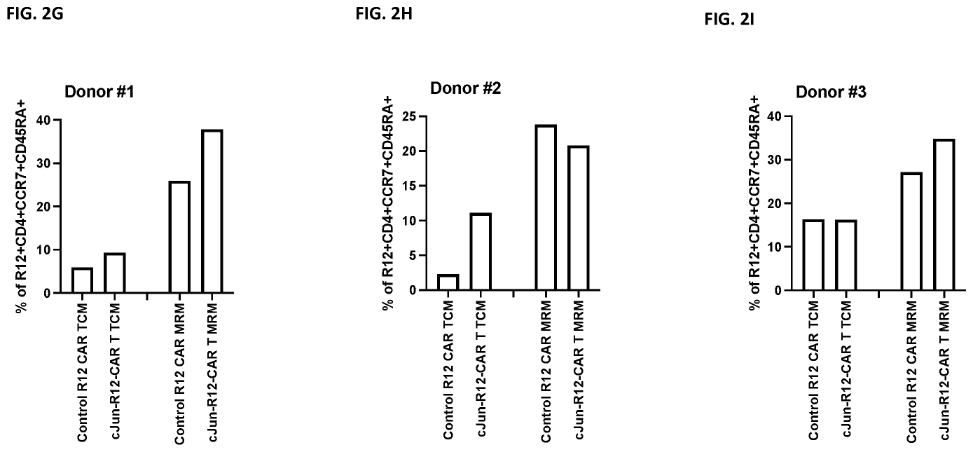

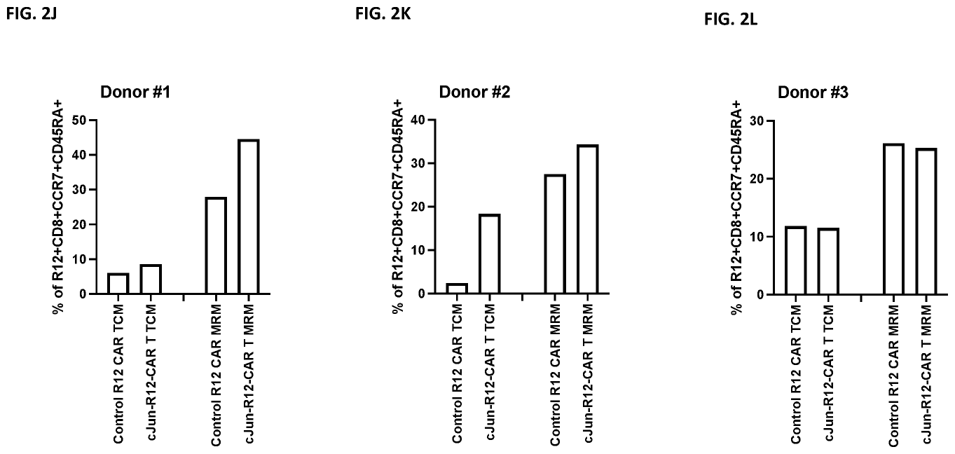

As shown in FIGS. 2A-2C, compared to cells from the T_{CM} groups, CD4+ T cells transduced with an anti-ROR1 CAR construct in MRM were more stem-like as to their phenotypic expression. This was generally true regardless of whether the CD4+ T cells were transduced with the c-Jun-R12 CAR or the control CD19t-R12 CAR (see last two bars in FIGS. 2A-2C). Similarly, CD8+ T cells transduced in MRM were generally more stem-like compared to corresponding cells transduced in TCM (at least for CD8+ T cells derived from donors #1 and #2; see FIGS. 2D and 2E). But, unlike the CD4+ T cells, consistent increase in stem-like cells were observed when CD8+ T cells were transduced with c-Jun-R12 CAR as compared to the control CD19t-R12 CAR. Accordingly, among the CD8+ T cells, the greatest percentage of stem-like cells was observed when CD8+ T cells were transduced with c-Jun-R12 CAR in MRM. As shown in FIGS. 2G-2I, compared to cells from the TCM groups, CD4+ T cells transduced with an anti-ROR1 CAR construct in MRM contained higher proportions of naïve and stem cell memory T cells (as evidenced by CCR7⁺ and CD45RA⁺ expression) (compare first two bars with last two bars, respectively). In general, increase in proportions of naïve and stem cell memory T cells were also observed when CD4+ T cells were transduced with c-Jun-anti-ROR1 CAR as compared to the control anti-ROR1 CAR (compare second and fourth bars to the first and third bars in FIGS. 2G-2I). Similar results were observed in CD8+ T cells (see FIGS. 2J-2L). Accordingly, among both the CD4+ T cells and the CD8+ T cells, the greatest percentage of naïve and stem cell memory T cells was generally observed when transduced with c-Jun-anti-ROR1 CAR in MRM.

These results highlight the usefulness of the metabolic reprogramming media described herein in producing transduced CD4+ and CD8+ T cells that are less differentiated (i.e., more stem-like). And, at least for CD8+ T cells, the results further suggest that overexpressing transcription factors, such as c-Jun, can further improve the stem-like properties of the transduced T cells.

### Example 3: Functional Analysis

To further assess the effect that MRM has on anti-ROR1 CAR T cells, human CD4+ and CD8+ T cells were transduced with anti-ROR1 CAR constructs and expanded as described in Example 1. At day 6 or 7, the transduced CD4+ and CD8+ T cells were analyzed functionally (e.g., IL-2 and/or INF-γ production and in vitro killing after primary and/or chronic antigen stimulation).

Cytotoxicity and Cytokine Secretion

The cytolytic activity of the transduced T cells was measured using an in vitro killing assay. Briefly, the transduced T cells (“effector”) were co-cultured with target tumor cells (“target) at an effector:target ratios of 11:4, 1:16, 1:64, and 1:128 and scanned at 4× magnification every 6 hours using the IncuCyte (cytolytic activity was measured by tracking the number of red nuclei representing the target tumor cells). After 24 hours of co-culture, supernatant was collected from the different conditions and frozen at −80° C. for later cytokine analysis. The culture plates containing the cells were then returned to the IncuCyte for continued periodic scanning.

For cytokine secretion analysis, the previously frozen supernatant was thawed and the levels of certain cytokines (e.g., IL-2 and IFN-g) was assessed using the MesoScaleDiscovery (MSD) multiplex platform and measured on the MSD Meso Sector S 600 machine according to the manufacturer's protocol.

Serial Restimulation Assay

In this assay, the transduced CD4+ and CD8+ T cells were serially restimulated every three or four days with A549 NLR target cells. The T cells were plated at an E:T ratio of 1:1 for a total of 2 to 4 rounds of stimulation. A density of 3×10⁵ transduced T cells/mL was maintained throughout the study. To set up each round of stimulation, the T cells were stained with the following markers and analyzed using flow cytometry to calculate the proportion of transduced T cell population present in the co-culture: CD45, CD3, CD4, CD8, CAR, and EGFRt (SEQ ID NO: 24). An aliquot of each sample was reserved for a titrated Incucyte killing assay, as described above.

Results

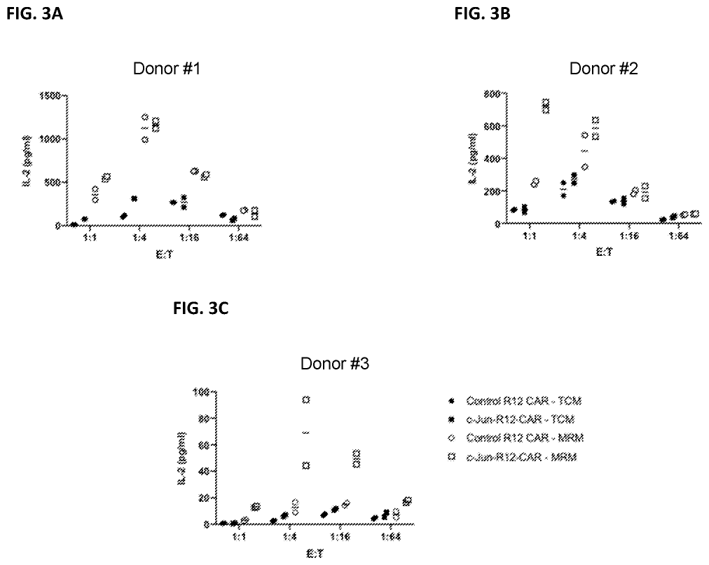

As shown in FIGS. 3A-3C, a clear functional difference was observed in the transduced T cells from the different test groups. After primary antigen stimulation, T cells transduced and cultured in MRM produced higher amounts of IL-2 compared to the corresponding cells transduced and cultured in TCM. And, as observed earlier in Example 1, the increased c-Jun protein expression in the transduced T cells also resulted in greater IL-2 secretion. For instance, in both the TCM and MRM groups, T cells that were transduced with the c-Jun-R12 CAR produced higher levels of IL-2 after primary stimulation, compared to corresponding cells that were transduced with the control CD19t-R12 CAR. Accordingly, greatest IL-2 production was generally observed in T cells modified to overexpress c-Jun and cultured in MRM.

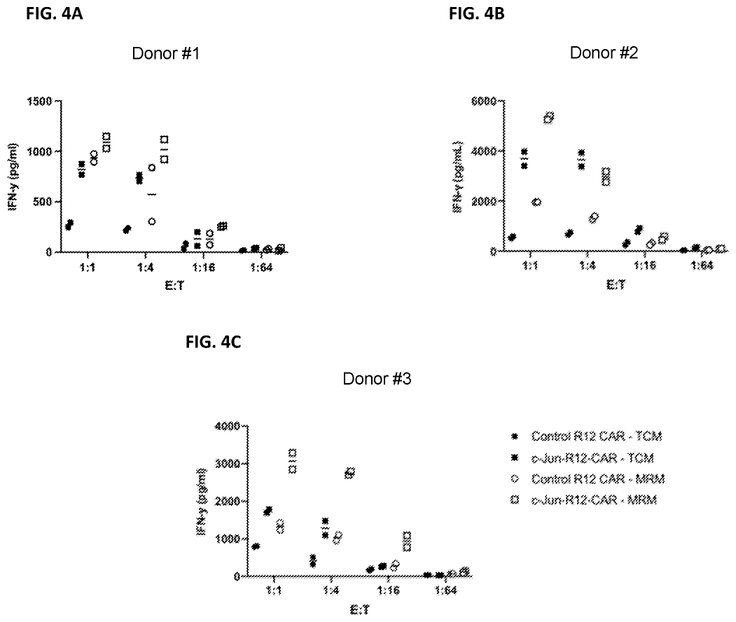

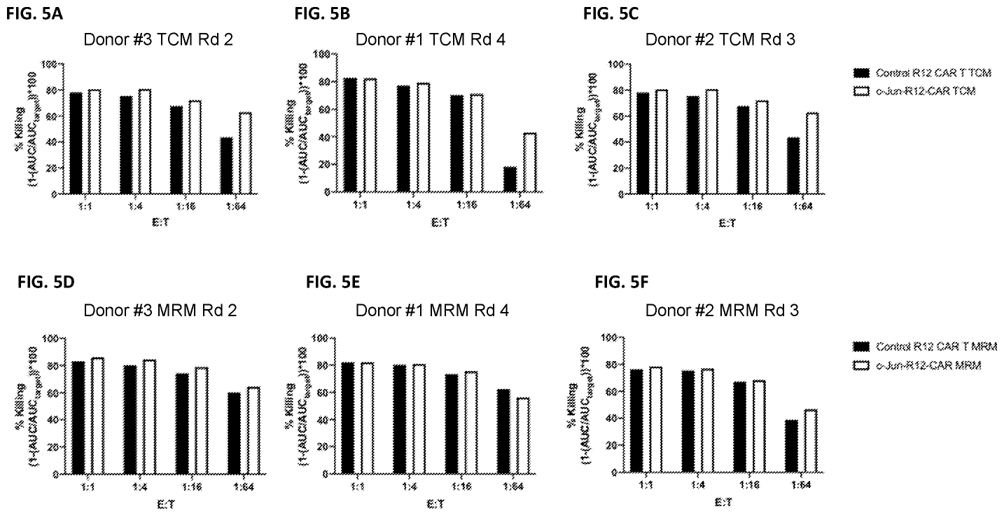

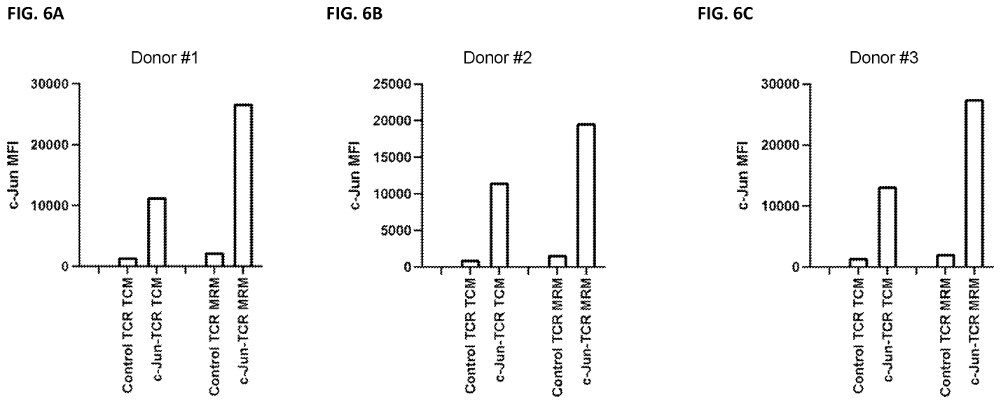

Similar results were observed after serial/chronic antigen stimulation. As shown in FIGS. 4A-4C, following the terminal round of antigen stimulation, T cells transduced and cultured in MRM retained their ability to produce IFN-γ compared to the corresponding cells that were transduced and cultured in TCM. Again, T cells transduced with c-Jun-R12 CAR from the MRM group maintained the ability to produce IFN-γ much longer compared to transduced cells from the other groups. As to the cytotoxicity of the transduced cells after multiple antigen stimulation, T cells transduced and cultured in MRM maintained their ability to kill tumor cells much longer, compared to the corresponding cells from the TCM group (FIGS. 5A-5E).

Collectively, the above results confirm that CAR T cells modified to overexpress c-Jun (e.g., with ROR1 CAR and c-Jun overexpression) cultured in MRM allow for the generation of stem-like transduced T cells that remain functional even after chronic antigen stimulation.

### Example 4: Analysis Of The Effect Of Metabolic Reprogramming Media On Anti-CD19 CAR Bearing Immune Cells Overexpressing C-Jun

To determine whether the improved biological effects observed above in Examples 1-3 are also applicable for immune cells targeting other tumor antigens, human CD4+ and CD8+ T cells will be modified to overexpress c-Jun and to comprise one or more exogenous nucleotide sequences encoding an anti-CD19 chimeric binding protein. The one or more exogenous nucleotide sequences will be introduced into the immune cells using any suitable methods known in the art and/or described herein (e.g., non-viral delivery). As in the above Examples, the immune cells will be modified and cultured in either metabolic reprogramming media or in a control medium that does not comprise potassium ion at a concentration higher than 5 mM (e.g., TCM). Then, the modified immune cells will be assessed for various properties, including but not limited to, transduction efficiency, stemness, effector function (including after repeated antigen stimulation), or resistance to exhaustion.

### Example 5: Analysis Of The Effect Of Metabolic Reprogramming Media On Anti-HER2 CAR-Bearing Immune Cells Overexpressing C-Jun

To determine whether the improved biological effects observed above in Examples 1-3 are also applicable for immune cells targeting other tumor antigens, human CD4+ and CD8+ T cells will be modified to overexpress c-Jun and to comprise one or more exogenous nucleotide sequences encoding an anti-HER2 chimeric binding protein. The one or more exogenous nucleotide sequences will be introduced into the immune cells using any suitable methods known in the art and/or described herein (e.g., non-viral delivery). As in the above Examples, the immune cells will be modified and cultured in either metabolic reprogramming media or in a control medium that does not comprise potassium ion at a concentration higher than 5 mM (e.g., TCM). Then, the modified immune cells will be assessed for various properties, including but not limited to, transduction efficiency, stemness, effector function (including after repeated antigen stimulation), or resistance to exhaustion.

### Example 6: Analysis Of The Effect Of Metabolic Reprogramming Media On Anti-Mesothelin CAR-Bearing Immune Cells Overexpressing C-Jun

To determine whether the improved biological effects observed above in Examples 1-3 are also applicable for immune cells targeting other tumor antigens, human CD4+ and CD8+ T cells will be modified to overexpress c-Jun and to comprise one or more exogenous nucleotide sequences encoding an anti-mesothelin chimeric binding protein. The one or more exogenous nucleotide sequences will be introduced into the immune cells using any suitable methods known in the art and/or described herein (e.g., non-viral delivery). As in the above Examples, the immune cells will be modified and cultured in either metabolic reprogramming media or in a control medium that does not comprise potassium ion at a concentration higher than 5 mM (e.g., TCM). Then, the modified immune cells will be assessed for various properties, including but not limited to, transduction efficiency, stemness, effector function (including after repeated antigen stimulation), or resistance to exhaustion.

### Example 7: Analysis Of The Effect Of Metabolic Reprogramming Media On Anti-PSCA CAR-Bearing Immune Cells Overexpressing C-Jun

To determine whether the improved biological effects observed above in Examples 1-3 are also applicable for immune cells targeting other tumor antigens, human CD4+ and CD8+ T cells will be modified to overexpress c-Jun and to comprise one or more exogenous nucleotide sequences encoding an anti-PSCA chimeric binding protein. The one or more exogenous nucleotide sequences will be introduced into the immune cells using any suitable methods known in the art and/or described herein (e.g., non-viral delivery). As in the above Examples, the immune cells will be modified and cultured in either metabolic reprogramming media or in a control medium that does not comprise potassium ion at a concentration higher than 5 mM (e.g., TCM). Then, the modified immune cells will be assessed for various properties, including but not limited to, transduction efficiency, stemness, effector function (including after repeated antigen stimulation), or resistance to exhaustion.

### Example 8: Analysis of the Effect of Metabolic Reprogramming Medium on Engineered TCR-Bearing Immune Cells Overexpressing C-Jun

To determine whether the improved biological effects observed above in Examples 1-3 are also applicable for immune cells modified to express an engineered TCR, human CD4+ and CD8+ T cells were modified to overexpress c-Jun and to comprise one or more exogenous nucleotide sequences encoding an engineered NY-ESO-1-specific TCR. As is apparent from the present disclosure, the one or more exogenous nucleotide sequences can be introduced into the immune cells using any suitable methods known in the art and/or described herein (e.g., non-viral delivery). As described further below (and similar to the earlier Examples), in the present Example, lentiviral vectors were used to introduce the exogenous nucleotide sequences into the immune cells. Additionally, the immune cells were transduced and cultured in either metabolic reprogramming media or in a control medium that does not comprise potassium ion at a concentration higher than 5 mM. Then, the modified immune cells were assessed for various properties, including but not limited to, transduction efficiency, stemness phenotype, effector function (e.g., the ability of the modified NY-ESO-1+ T cells overexpressing c-Jun to recognize and kill NY-ESO-1-expressing target cells, including after repeated stimulation), or resistance to exhaustion. More specific exemplary methods used are provided below.

### Media Preparation

The T cell conditioned medium (TCM) and metabolic reprogramming medium (MRM) were prepared as described in Example 1.

Lentiviral Vector (LVV) Construction

An NY-ESO1 TCR construct comprising the following components was generated: (i) NY-ESO1 TCR alpha chain (SEQ ID NO: 98) and beta chain (SEQ ID NO: 96), and (ii) wild-type c-Jun protein (SEQ ID NO: 13) (referred to herein as the “c-Jun-NY-ESO1 TCR”; SEQ ID NO: 95). See Table 17 (below). The c-Jun-NY-ESO1 construct was designed, such that when transduced in a cell (e.g., T cell), the transduced cell would exhibit increased c-Jun protein expression along with surface expression of the NY-ESO1 TCR. As a control, a corresponding NY-ESO1 TCR construct lacking c-Jun was also generated (referred to herein as the “control NY-ESO1 TCR”).

Lentiviral vectors were produced as described in Example 1.

T Cell Isolation

CD4+ and CD8+ T cells were isolated from three healthy donors and frozen by AllCells (Alameda, CA, USA). The vendor obtained and maintained all appropriate consent forms from the donors. To isolate the CD4+ and CD8+ T cells, samples were collected samples via apheresis, from which CD4+ and CD8+ cells were isolated separately in order of CD8+ T cells positively selected first followed by positive selection for CD4+ T cells of the flow-through from the CD8 selection. Isolated CD4+ or CD8+ T cells were frozen at 20E+06 cells (AllCells) per vial.

Cell Culture and Transduction

Healthy donor cryopreserved human CD4+ and CD8+ T cells (i.e., from the vendors) were thawed in the appropriate media (i.e., TCM or MRM) and combined at a 1:1 ratio. The combined donor CD4+ and CD8+ T cells were centrifuged at 300×g for 5 minutes and resuspended in appropriate media (i.e., T cell conditioned media or MRM) supplemented with IL-2 (200 IU/ml), IL-7 (1200 IU/ml), and IL-15 (200 IU/ml). The T cells were then activated using CD3/CD28 TRANSACT™ (Miltenyi Biotec Inc.). After 24 hours of activation (i.e., day 1) in either TCM or MRM, the T cells were transduced with the above-described LVVs comprising the NY-ESO1 TCR constructs (i.e., “c-Jun-NY-ESO1 TCR” and “control NY-ESO1 TCR”). Non-transduced T cells were used as control. The following day after transduction (i.e., day 2), fresh media (i.e., TCM or MRM) were added to dilute the TRANSACT™ and end T cell activation. The transduced T cells were allowed to further expand for five additional days (i.e., day 7), and then either subsequently analyzed or cryopreserved in liquid nitrogen for long-term storage.

Transduction Efficiency Analysis

To compare the TCR transduction efficiency from the different groups (see Table 12), the percentage of CD4+ and CD8+ T cells expressing the following was determined using flow cytometry: c-Jun and NY-ESO1 TCR comprising sequence set forth in SEQ ID NO: 95.

TABLE 12
Experimental Groups

| Group No. | Description |
|---|---|
| 1 | Non-transduced T cells cultured in TCM |
| 2 | T cells transduced with control NY-ESO1 TCR |
| | and cultured in TCM |
| 3 | T cells transduced with c-Jun-NY-ESO1 TCR and |
| | cultured in TCM |
| 4 | Non-transduced T cells cultured in MRM |
| 5 | T cells transduced with control NY-ESO1 TCR |
| | cultured in MRM |
| 6 | T cells transduced with c-Jun-NY-ESO1 TCR |
| | cultured in MRM |

There was no consistent difference observed in the percentage of CD4+ and CD8+ T cells that were transduced from the different groups within each donor. Similar to the previous results with the c-Jun-R12 CAR, T cells transduced with c-Jun-NY-ESO1 TCR from the MRM group expressed significantly higher levels of c-Jun protein expression compared to the corresponding transduced T cells from the TCM group (see FIGS. 1A-1C). The increased expression was specific to c-Jun protein and was not global (i.e., NY-ESO1 TCR expression levels remained comparable).

These results confirm the earlier findings (see, e.g., Example 1) that the metabolic reprogramming media condition described herein could be useful in selectively increasing the expression of the c-Jun protein of a ligand binding protein construct (e.g., the NY-ESO1 TCR construct provided herein) without significantly altering the percentage of CD4+ and CD8+ T cells.

### Example 9: Analysis of Phenotypic Expression of Engineered TCR-Bearing Immune Cells Transduced and Cultured in Metabolic Reprogramming Medium

To further assess whether the metabolic reprogramming media has any effect on the differentiation status of transduced T cells, human CD4+ and CD8+ T cells were transduced with NY-ESO1 TCR constructs as described in Example 8 (i.e., c-Jun-NY-ESO1 TCR or control NY-ESO1 TCR). Then, after the cells were allowed to expand for five additional days (i.e., day 7), the phenotype of the transduced T cells was assessed using flow cytometry.

The phenotype of NY-ESO-1 T cell products was assessed by spectral flow cytometry on the final day of production. Briefly, approximately 2×10⁵ cells were washed with FACS buffer and blocked with mouse serum and human IgG in the dark at 37° C. for 10 minutes before staining with anti-CCR7 Ab in the dark at 37° C. for 15 minutes. The cells were then washed with FACS buffer, stained with Ab mix containing the remaining surface markers and live/dead in the dark at RT for 25 minutes. After surface staining, cells were washed with FACS buffer and fixed and permeabilized with Foxp3 fixation/permeabilization buffer in the dark at room temperature (RT) for 30 minutes, and subsequently, washed with Foxp3 permeabilization wash buffer. Cells were then blocked with rabbit and mouse serum in the dark at RT for 10 to 15 minutes before stained with Abs against intracellular markers in the dark at RT for 30 minutes in Foxp3 permeabilization wash buffer. Cells were washed with Foxp3 permeabilization wash buffer followed by a wash in FACS buffer. Samples were re-suspended in FACS buffer and acquired with a Cytek Aurora Spectral Flow Cytometer and analyzed using FlowJo software (TreeStar, Ashland, OR). Specifically, as described in this experiment, “naïve and stem cell memory” cells were defined as: CCR7⁺CD45RA⁺. The list of exemplary antibodies and reagents that can be used for assessing the phenotype of the transduced T cells are provided in Tables 13-14.

TABLE 13
Antibodies Used for Phenotypic Assessment

| Antigen | Fluorochrome | Supplier |
|---|---|---|
| Live/Dead Fixable | eFluor780 | ThermoFisher Scientific |
| TCRvβ13.1 | PE | Beckman Coulter |
| CD3 | BUV805 | BD Bioscience |
| CD8 | AF532 | ThermoFisher Scientific |
| CD4 | BUV496 | BD Bioscience |
| CD27 | BUV737 | BD Bioscience |
| CD28 | BV510 | Biolegend |
| CD45RO | BV480 | BD Bioscience |
| CD62L | BUV395 | BD Bioscience |
| CD127 | PE Cy5 | Biolegend |
| CD39 | BV605 | Biolegend |
| LAG-3 | SB702 | ThermoFisher Scientific |
| TIM3 | SB780 | ThermoFisher Scientific |
| PD-1 | PE -Cy7 | ThermoFisher Scientific |
| TIGIT | PerCP-eF710 | ThermoFisher Scientific |
| CCR7 | PE CF594 | BD Bioscience |
| CD45RA | AF700 | BD Bioscience |
| CD45 | BV570 | Biolegend |
| c-Jun | AF647 | Cell Signaling Technology |
| TCF1/TCF7 | Pacific Blue | Cell Signaling Technology |
| cleaved PARP (Asp214) | AF488 | Cell Signaling Technology |

TABLE 14
Staining Reagents Used for Phenotypic Assessment

| Reagent | Supplier |
|---|---|
| CSB FACS buffer | Biolegend |
| Foxp3/Transcription Factor | ThermoFisher Scientific |
| Staining Buffer Set | |
| Brilliant Stain Buffer Plus | BD Bioscience |
| Chrome Pure Human IgG, | Jackson ImmunoResearch |
| whole molecule | |
| Normal Rabbit Serum | Jackson ImmunoResearch |
| Normal Mouse Serum | Jackson ImmunoResearch |
| AbC Total Antibody | ThermoFisher Scientific |
| Compensation Bead Kit | |

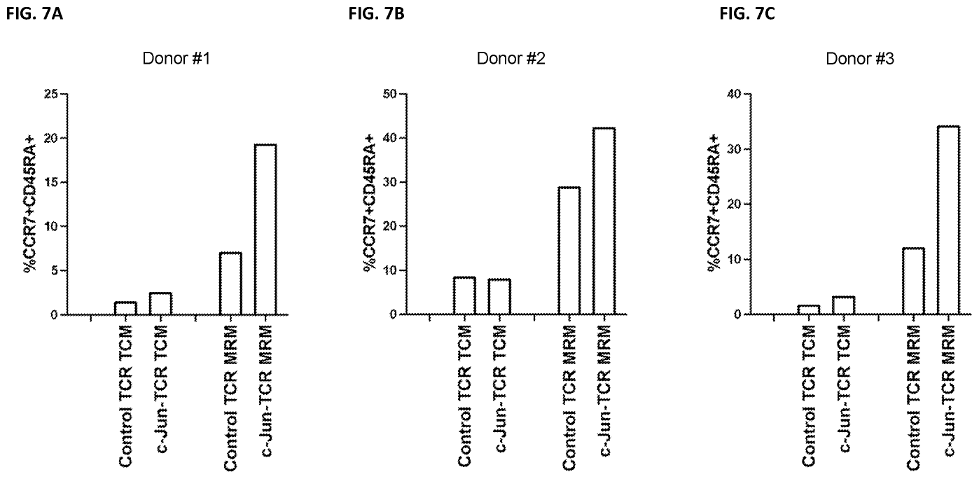

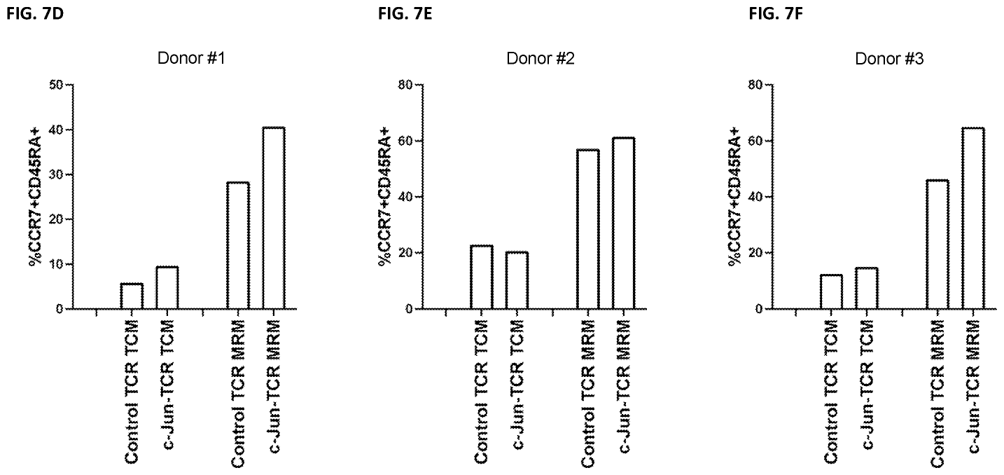

As shown in FIGS. 7A-7C, compared to cells from the TCM groups, CD4+ T cells transduced with an NY-ESO1 TCR construct in MRM contained higher proportions of naïve and stem cell memory T cells (as evidenced by CCR7T and CD45RA⁺ expression) (compare first two bars with last two bars, respectively). Consistent increase in proportions of naïve and stem cell memory T cells were also observed when CD4+ T cells were transduced with c-Jun-NY-ESO1 TCR as compared to the control NY-ESO1 TCR (compare second and fourth bars to the first and third bars in FIGS. 7A-7C). Similar results were observed in CD8+ T cells (see FIGS. 7D-7F). Accordingly, among both the CD4+ T cells and the CD8+ T cells, the greatest percentage of naïve and stem cell memory T cells was observed when transduced with c-Jun-NY-ESO1 TCR in MRM.

These results confirm the usefulness of the metabolic reprogramming media described herein in producing transduced CD4+ and CD8+ T cells that are less differentiated. The results further confirm that overexpressing the transcription factor c-Jun can further improve the naïve and stem cell memory properties of the transduced T cells.

### Example 10: Functional Analysis of Engineered TCR-Bearing Immune Cells Transduced and Cultured in Metabolic Reprogramming Medium

To further assess the effect that MRM has on NY-ESO1 TCR T cells, human CD4+ and CD8+ T cells were transduced with NY-ESO1 TCR constructs and expanded as described in Example 8, and analyzed functionally (e.g., IL-2 and IFN-γ production and in vitro killing after primary and/or chronic antigen stimulation).

Cytotoxicity and Cytokine Secretion

The cytolytic activity of the transduced T cells was measured using an in vitro killing assay. Briefly, the transduced T cells (“effector”) were co-cultured with target tumor cells (“target”) at an effector:target ratios of 1:1 and 1:4 and scanned at 10× magnification every 6 hours using the IncuCyte (cytolytic activity was measured by tracking the number of red nuclei representing the target tumor cells). After 22 hours of co-culture, supernatant was collected from the different conditions and frozen at −80° C. for later cytokine analysis. The culture plates containing the cells were then returned to the IncuCyte for continued periodic scanning.

For cytokine secretion analysis, the previously frozen supernatant was thawed and the levels of certain cytokines (e.g., IL-2 and IFN-7) was assessed using the MesoScaleDiscovery (MSD) multiplex platform and measured on the MSD Meso Sector S 600 machine according to the manufacturer's protocol.

Sequential Restimulation Assay

In this assay, the transduced CD4+ and CD8+ T cells were chronically restimulated every three or four days with A375 target cells or H1703 target cells. Both the A375 and H1703 cells expressed NucLight Red (NLR; nuclear-restricted mKate2), so that the non-lysed cells can be quantified using Incucyte. The different test groups were incubated at an effector-to-target (E:T) cell ratio of 1:1 (H1703) or 1:4 (A375) for 162 (H1703) or 234 (A375) hours. The T cells were plated at an E:T ratio of 1:1 for a total of two (H1703) or three (A375) rounds of stimulation. Following each round, ¼ of the co-culture volume was transferred to fresh target tumor cells to reset.

Results

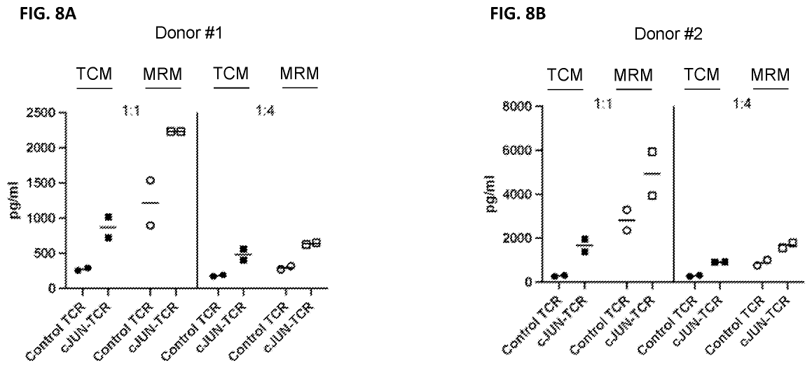

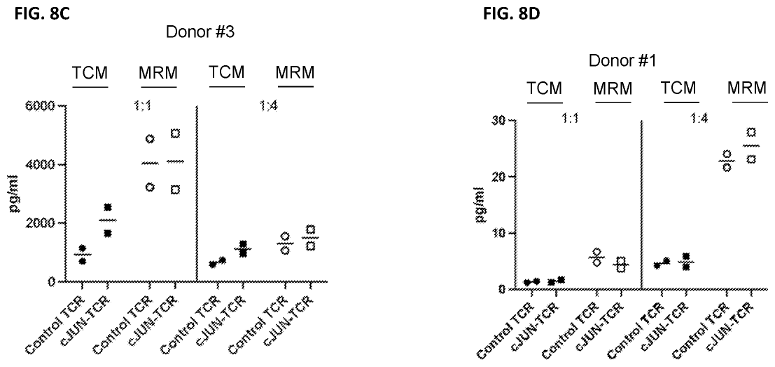

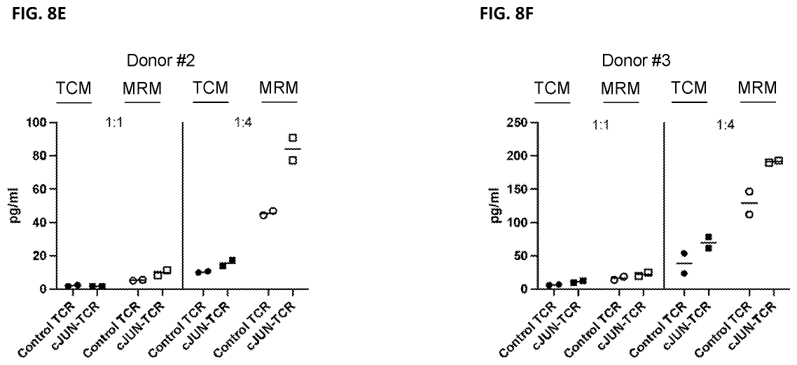

As shown in FIGS. 8A-8F, there was a clear difference in IL-2 production among the transduced T cells from the different test groups. After primary antigen stimulation using A375 cells (which exhibit a high NY-ESO1 antigen density; FIGS. 8A-8C) and H1703 cells (which exhibit a low NY-ESO1 antigen density; FIGS. 8D-8F), T cells transduced with NY-ESO1 TCR (with or without c-Jun) and cultured in MRM produced higher amounts of IL-2 compared to the corresponding cells transduced and cultured in TCM. And in general, the increased c-Jun protein expression in the transduced T cells was also associated with greater IL-2 secretion. For instance, in both the TCM and MRM groups, T cells that were transduced with the c-Jun-NY-ESO1 TCR produced higher levels of IL-2 after primary stimulation, compared to corresponding cells that were transduced with the control NY-ESO1 TCR. Accordingly, greatest IL-2 production was generally observed in T cells modified to overexpress c-Jun and cultured in MRM. The overall production of IL-2 was higher in the presence of A375 tumor cells that express higher density of the cognate antigen compared to H1703.

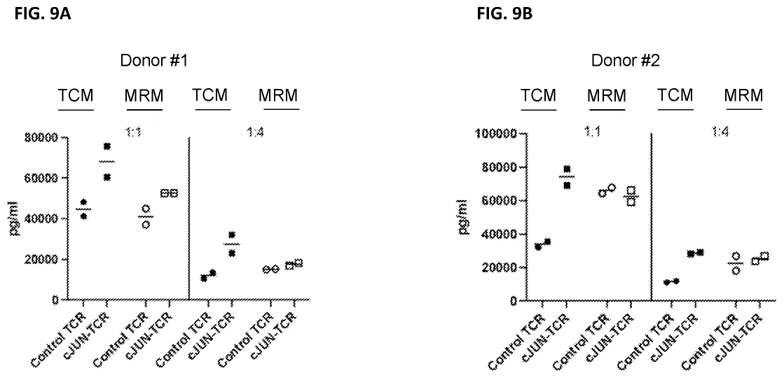

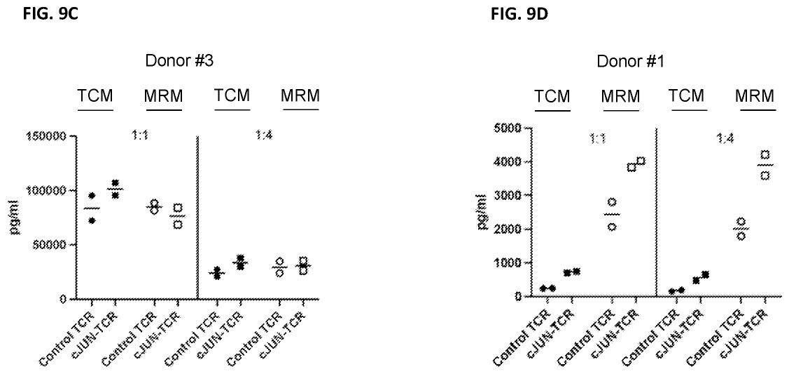

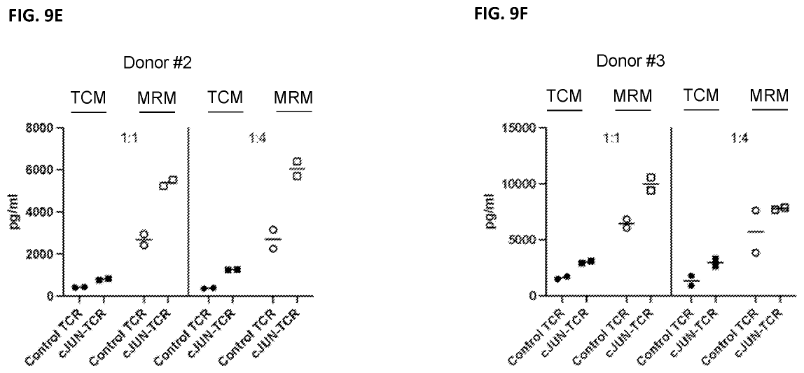

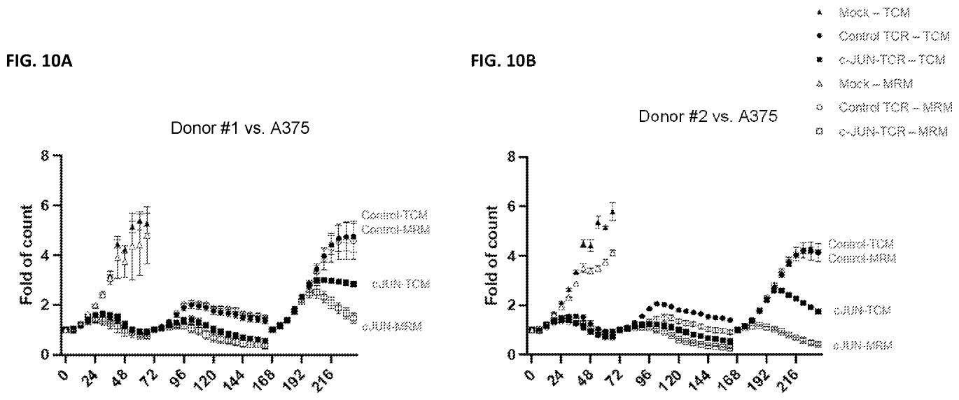

As shown in FIGS. 9A-9F, a similar trend (i.e., increased production in T cells transduced and cultured in MRM as compared to TCM; and increased production in T cells also transduced to overexpress c-Jun as compared to cells transduced with just the NY-ESO1 TCR) in IFN-γ production following primary antigen stimulation with the H1703 cells, which express lower level of the NY-ESO1 antigen (FIGS. 9D-9F). With the A375 cells, which express a higher level of the NY-ESO1 antigen, there was no clear difference in IFN-γ production among the different groups (FIGS. 9A-9C).

In chronic infection and cancer, T cells can become exhausted through persistent antigen exposure leading to progressive loss of T-cell effector functions, such as cytolytic activity and cytokine secretion. Therefore, to assess whether the above-described NY-ESO1 TCR-transduced T cells (with or without c-Jun overexpression) exhibit any differences in their tendency to become exhausted, the transduced T cells from the different groups were chronically stimulated with repeated exposure to target tumor cells (i.e., NY-ESO1 expressing A375 and H1703 cells). Then, lysis of the target cells was evaluated by tracking total NLR intensity every 6 hrs at 10× magnification then normalized to time 0 hr of assay setup for each round of stimulation.

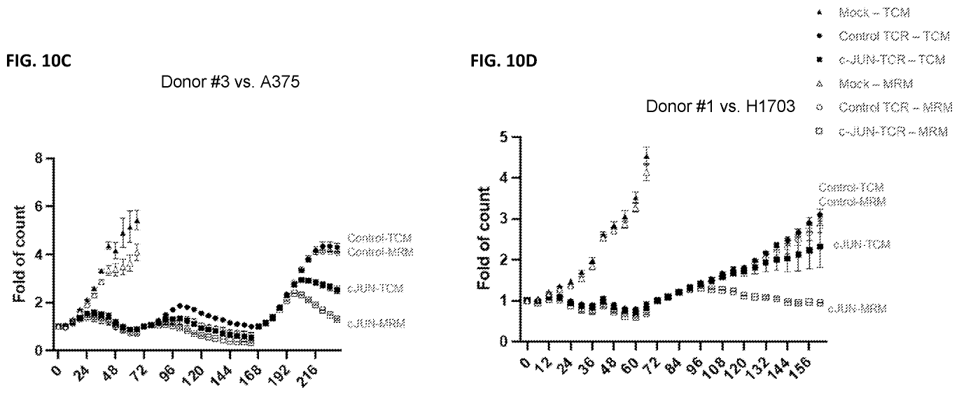

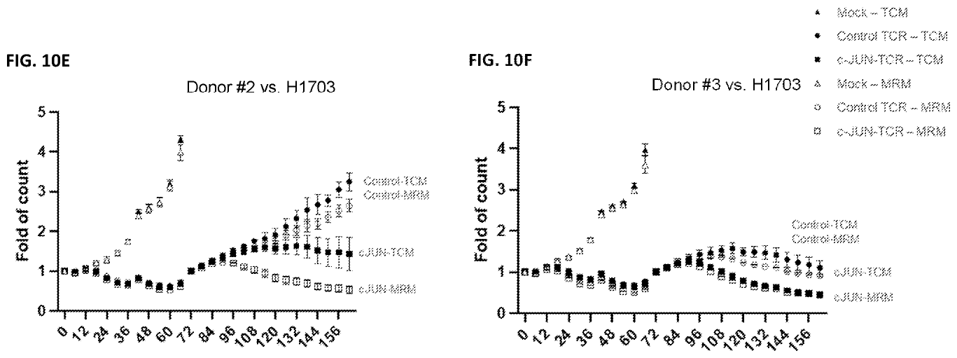

As shown in FIGS. 10A-10C, c-Jun overexpression alone was able to prolong the effector function of the transduced T cells after chronic antigen stimulation (regardless of whether the cells were transduced and cultured in TCM or MRM). However, T cells transduced with c-Jun-NY-ESO1 TCR (i.e., overexpressing c-Jun) from the MRM group maintained the ability to lyse A375 tumor cells much longer compared to the transduced cells from any of the other groups. Similar results were observed using the H1703 cells (see FIGS. 10D-10F).

Collectively, the above results confirm that TCR T cells modified to overexpress c-Jun (e.g., with NY-ESO1 TCR and c-Jun overexpression) and cultured in MRM allow for the generation of less-differentiated transduced T cells that remain functional even after chronic antigen stimulation.

### Example 11: Transcriptome Analysis

To assess the effect of MRM on the T cells at the gene level, single cell RNA-seq analysis was performed on T cells after serial antigen stimulation assay (see, e.g., Example 3). Prior to antigen stimulation, the T cells were transduced with either (i) CD19t-R12 CAR (i.e., R12 CAR without c-Jun) and cultured in control media (“control ROR1 CAR”) or (ii) c-Jun-R12 CAR (i.e., R12 CAR with c-Jun) and cultured in MRM (“c-Jun ROR1 CAR”). At various time points of the serial stimulation assay, RNA was extracted and T cell clusters with enrichment of stem-like genes and T cell terminal exhaustion (TTE) genes were assessed. For the stem-like genes, the gene set described in Caushi et al., Nature 596: 126-132 (2021) was used. For the TTE genes, the gene set described in Oliveira et al., Nature 596: 119-125 (2021) was used. Both Caushi et al. and Oliveira et al. are incorporated herein by reference in their entirety. The identification of stem-like clusters indicates the existence of relatively less differentiated CD8+ T cells, and identification of TTE clusters indicates presence of exhausted/dysfunctional CD8+ T cells particularly after serial antigen stimulation.

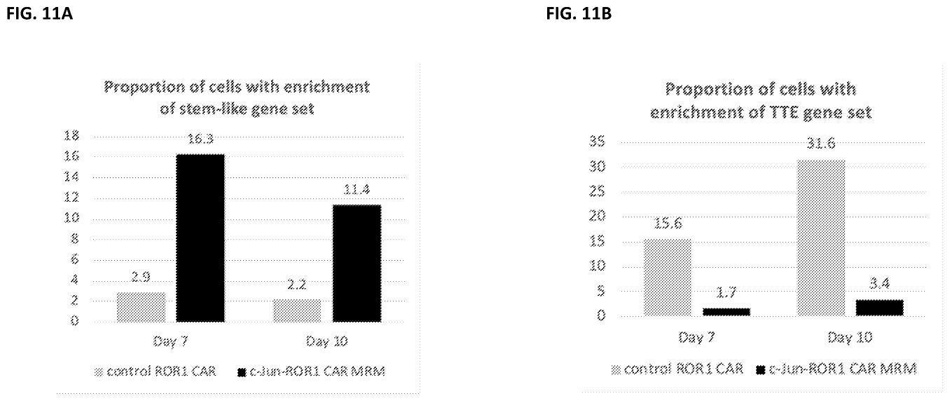

As shown in FIGS. 11A and 11B, among the control ROR1 CAR T cells cultured in control media, the proportion of clusters with enrichment of stem-like gene set remained low at both days 7 and 10 of the serial antigen stimulation, while the proportion of clusters with enrichment of TTE gene set increased with further stimulation (e.g., compare days 7 and 10 in FIG. 11B). These results indicated that after prolonged antigen stimulation, the control ROR1 CAR T cells cultured in control media were more highly differentiated and exhausted/dysfunctional. In contrast, the c-Jun ROR1 CAR T cells cultured in MRM had significantly higher proportion of stem-like enriched clusters (see FIG. 11A) and reduced proportion of TTE enriched clusters (see FIG. 11B) at both days 7 and 10 of the serial antigen stimulation, demonstrating persistent presence of higher stem-like populations.

Collectively, the results provided above further confirm the therapeutic potential of the modified immune cells described herein, e.g., modified to overexpress c-Jun (e.g., with ROR1 CAR and c-Jun overexpression) and cultured in MRM.

### Example 12: Additional Transcriptome Profiling of the Effect of MRM and c-Jun Overexpression on CAR T Cells

Further to Example 6 provided above, single cell RNA-seq analysis was next performed on the transduced T cells described herein (e.g., anti-ROR1 CAR T cells) after adoptive transfer into H1975 xenograft tumor-bearing NSG MHC dKO mice. Specifically, animals received an administration of c-Jun ROR1 CAR T cells (i.e., overexpressing c-Jun) cultured (i.e., transduced and expanded) in MRM (at one of the following doses: 1e6, 2.5e6, and 5e6 cells/animal) or control ROR1 CAR T cells (i.e., not overexpressing c-Jun) cultured in MRM (2.5e6 cells/animal) when tumors reached ˜400 mm³. On day 13 after T cell injection, the mice were sacrificed and the excised tumors were dissociated using the gentleMACS™ Octo Dissociator with heaters and the Tumor Dissociation kit, human (Miltenyi Biotec), as per the manufacturer's protocol, with reduced amount of enzyme R to 20% in the enzyme mix. Dissociated tumor cells were processed for FACS sorting and single cell RNA sequencing was performed on Live mCD45⁻hCD45+ T cells sorted from animals treated with c-Jun ROR1 CAR T cells dosed at 2.5e6 cells/animal (n=5) and 5e6 cells/animal (n=2), and control ROR1 CAR T cells dosed at 2.5e6 cells/animal (n=5).

Samples were processed for single cell RNA sequencing using the Cellular Indexing of Transcriptomes and Epitopes by Sequencing (CITE-Seq) assay. CITE-Seq analysis allows simultaneous measurement of single cell RNA and cell surface protein. Cells were stained with a mix of fluorochrome-conjugated antibodies for FACS sorting, DNA-conjugated Total-SeqC antibodies against cell surface proteins for CITE-seq, and unique hashtag antibodies for cell barcoding. Sorted live mCD45⁻hCD45+ T cells from all uniquely barcoded samples were pooled together and loaded into the Chromium Next GEM Chip K using a Chromium Next GEM Single Cell 5′ v2 Reagent Kit (10× Genomics). After single-cell capture and lysis, cDNA was synthesized and amplified along with DNA conjugated to antibodies bound to cell surface proteins to finally generate 5′ Gene Expression (GEX) and Antibody-derived tag (ADT) libraries according to the manufacturer's protocol (10× Genomics). The GEX and ADT libraries prepared from multiple channels of the Chromium Next GEM Chip K were quantified, pooled, and sequenced together using the NovaSeq 6000 system (Illumina).

Single Cell CITE-Seq data was processed using the 10× Cell Ranger software version 6.1.2 (10× Genomics) with GRCh38 (and control R12 vector sequence and c-Jun sequence from c-Jun ROR1 CAR vector added) as reference genome and default parameters. The cell-gene matrix was further processed using Seurat package (Hao et al. 2021 Cell, 184(13):3573-3587). In brief, cells were first filtered (using thresholds for percent mitochondria, nCount_RNA, nFeature_RNA and hashtag doublets), and then CD8⁺ and CD4⁺ T-cells were separated by gating on CD8 and CD4 protein expression measured by CITE-Seq. CD8+ T-cells from tumors treated control ROR1 CAR T cells or c-Jun ROR1 CAR T cells were combined for single cell transcriptome analysis. The filtered cell-gene matrix was normalized and scaled, with variable feature selection. The effects of cell cycle heterogeneity were corrected by calculating cell cycle phase scores (G2M.Score, S.Score) using CellCycleScoring function in Seurat and then regressing out the cell cycle phase scores. Genes correlated with either of the two cell cycle phase scores (with Pearson correlation coefficient greater than 0.3) were excluded from selected features to further minimize the effects of cell cycle heterogeneity. Mitochondria, ribosome, TCR, and IG complex related genes were also excluded from the selected features. Then, top 50 PCs were calculated by RunPCA function using the filtered features. Afterward, Uniform Manifold Approximation and Projection (UMAP) by RunUMAP function in Seurat was used to map cells to two-dimensional space for visualization with each dot representing a cell. Cells were subjected to cluster analysis using the FindClusters function in Seurat and FindSubCluster function was used for refinement of cluster identification. CITE-Seq analysis is also referred to as single cell RNA-Seq analysis, since the analysis (UMAP, clustering) was done using RNA expression without protein expression in the CD8+ T-cells.

Details of single cell clustering analysis were described in the previous paragraph and in earlier examples (see, e.g., Example 11). Clusters with enrichment of stem-like genes, T cell activation (Tact) genes, T cell progenitor exhaustion (TPE) genes and T cell terminal exhaustion (TTE) genes were assessed. For the stem-like genes, the gene set described in Caushi et al., Nature 596: 126-132 (2021) was used. For the Tact genes, TPE genes and TTE genes, the gene sets described in Oliveira et al., Nature 596: 119-125 (2021) were used. Both Caushi et al. and Oliveira et al. are incorporated herein by reference in their entirety. Non-limiting examples of Tact, TPE, TTE, and stem-like genes are provided elsewhere in the present disclosure. The identification of stem-like clusters indicates the existence of relatively less differentiated CD8⁺ T cells, and identification of Tact clusters indicates presence of activated CD8⁺ T cells in tumors. The identification of TPE cluster indicates the presence of progenitor exhausted CD8⁺ T cells, and identification of TTE clusters indicates presence of exhausted/dysfunctional CD8⁺ T cells in tumors.

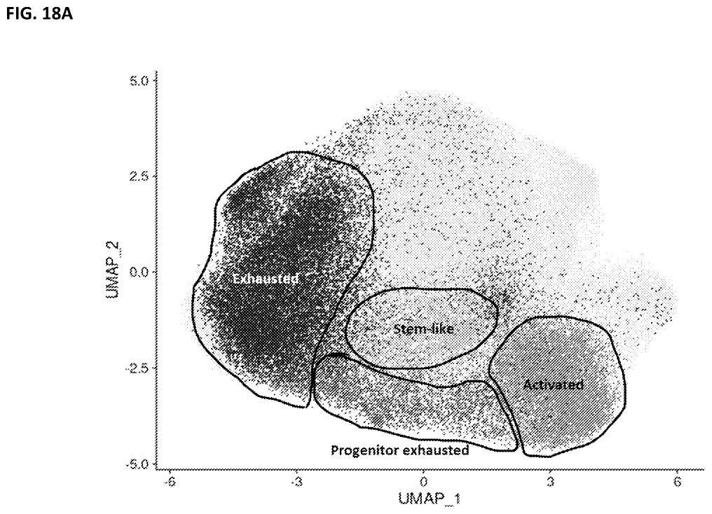

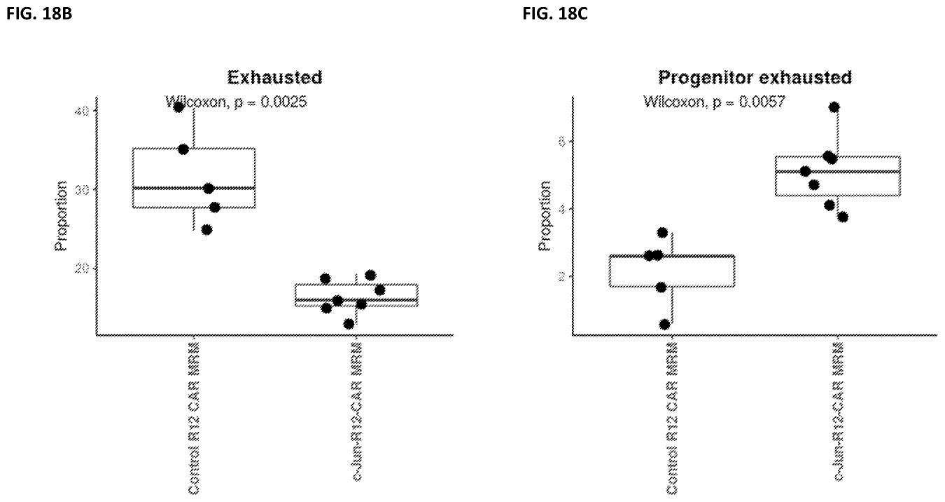

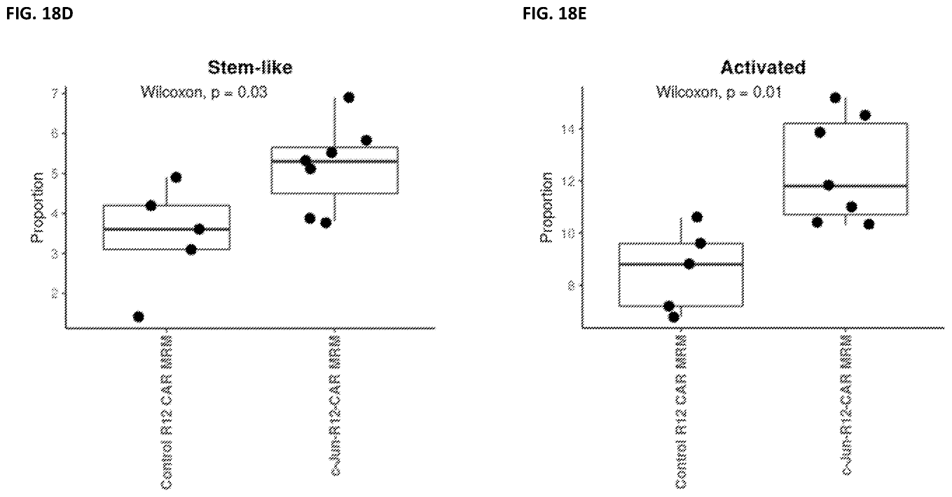

As shown in FIGS. 18A-18E, compared to T cells sorted from tumors treated with the control ROR1 CAR T cells, T cells sorted from tumors treated with the c-Jun ROR1 CAR T cells had significantly reduced proportion of clusters with enrichment of TTE gene set (FIG. 18B) and significantly higher proportion of cluster with enrichment of TPE gene set (FIG. 18C). TPE cells are known to be less exhausted than TTE cells, with expression of memory-associated transcripts (TCF7, CCR7 and IL7R), which have the potential to expand upon activation and to acquire a reinvigorated CD39− memory phenotype that associated with long-term persistence. [Oliveira et al., Nature 596: 119-125 (2021)]. The reduction in the TTE cluster and increase in the TPE cluster demonstrate the effect of c-Jun overexpression in counter exhaustion. Interestingly, T cells sorted from tumors treated with the c-Jun ROR1 CAR T cells had significantly higher proportion of stem-like enriched clusters (FIG. 18D), which indicates the existence and increased persistence of stem-like populations. The proportion of T cell activation enriched clusters was also higher in T cells sorted from tumors treated with the c-Jun ROR1 CAR T cells (FIG. 18E).

These results suggest that overexpression of c-Jun provides an added benefit over the effect of MRM in increasing stem-like populations, thus showing a benefit of the combination of MRM and overexpression of c-Jun.

### Example 13: Further Phenotypic and Functional Analysis of Anti-ROR1 CAR-Bearing Immune Cells Transduced and Cultured in a Range of Metabolic Reprogramming Media

To further assess the effect that MRM has on the modified cells described herein (e.g., anti-ROR1 CAR T cells), several MRMs with varying concentrations of potassium ion were prepared as described in Example 1. Specifically, the final concentrations of the different components of the MRM were in the range of. NaCl (55-90 mM), KCl (40-80 mM), and osmolality (˜250-260 mOsmol). Then, human CD4+ and CD8+ T cells (isolated from three donors) were transduced with anti-ROR1 CAR constructs with and without c-Jun and expanded using the different MRMs or TCM essentially as described in Example 1. Then, as described in the earlier examples (e.g., Examples 2 and 10), the transduced T cells were analyzed for various properties, e.g., transduction efficiency, c-Jun expression, stem-like phenotype expression, and function (e.g., IL-2 and IFN-γ production and in vitro killing after primary and/or chronic antigen stimulation).

### Results:

Similar to the earlier examples (see, e.g., Example 1), after transduction and before analysis, the transduced cells were cultured and expanded for a total of 7 days in either TCM or in the different MRM formulations having different concentrations of potassium ion (i.e., between 40-80 mM).

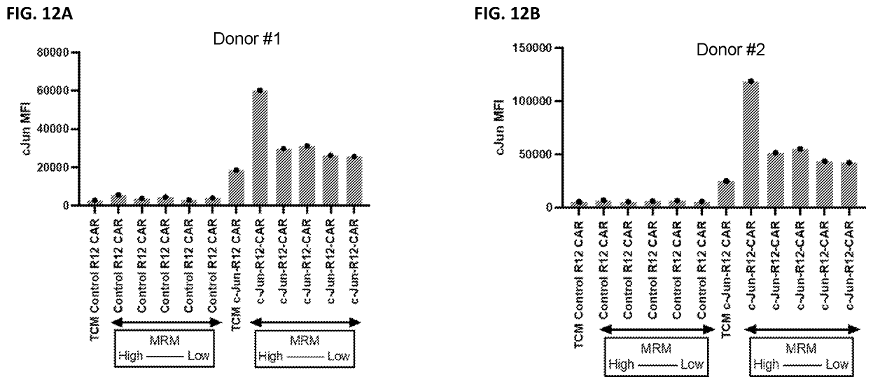

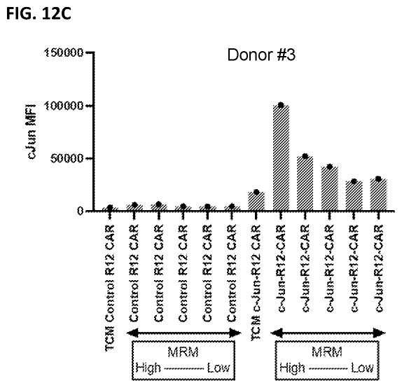

As shown in FIG. 12A-12C, and consistent with the earlier described data (see e.g., FIGS. 1A-1C), for all MRMs tested, T cells transduced with the c-Jun-R12 CAR construct and subsequently cultured in MRM had higher c-Jun expression as compared to corresponding T cells transduced and cultured in TCM. The highest increase in c-Jun expression was observed in MRM with the highest concentration of potassium.

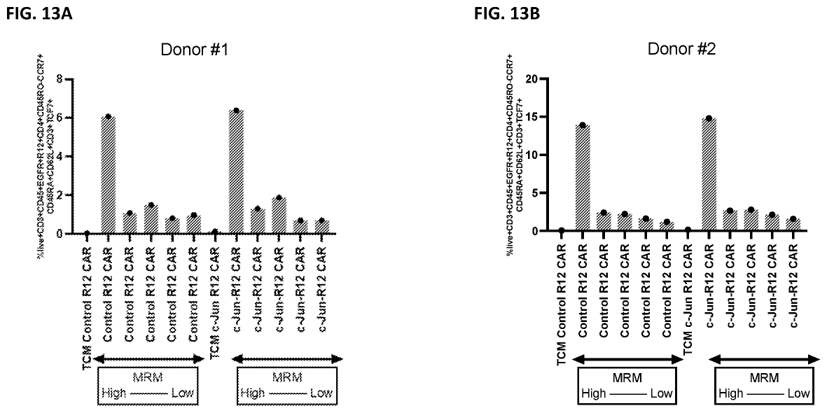

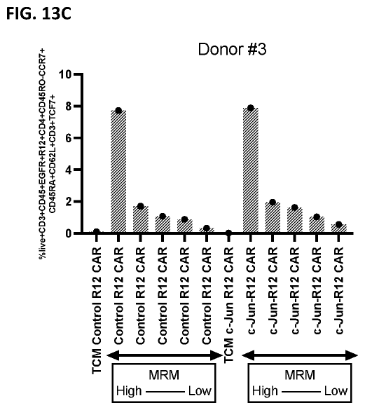

As to the stemness of the modified cells, and, again consistent with the earlier described data (see e.g., FIGS. 2A-2C), compared to cells from the TCM groups, CD4+ T cells transduced with an anti-ROR1 CAR construct in all MRM formulations were more stem-like as to their phenotypic expression (see e.g., FIGS. 13A-13C and Table 15 below). This was generally true regardless of whether the CD4+ T cells were transduced with the c-Jun-R12 CAR or the control CD19t-R12 CAR. The stemness percent in CD4+ T cells was the highest in MRM cultured cells compared to TCM cultured T cells with a dose dependent effect of MRM potassium concentration on stemness (highest stemness in highest potassium concentration).

| | DONOR | DONOR | DONOR | |
|---|---|---|---|---|
| | 1: % | 2: % | 3: % | |
| SAMPLE | STEMNESS | STEMNESS | STEMNESS | Media |
| Control R12 CAR | 0.027636 | 0.0573144 | 0.10542 | TCM |
| Control R12 CAR | 6.0814908 | 13.907619 | 7.7281056 | [K]High |
| Control R12 CAR | 1.0777632 | 2.4080164 | 1.71651375 | |
| Control R12 CAR | 1.496556 | 2.2331232 | 1.0723711 | [symbol] |
| Control R12 CAR | 0.8124688 | 1.6248546 | 0.87793287 | |
| Control R12 CAR | 0.9713664 | 1.1941384 | 0.3202256 | [K]Low |
| c-Jun-R12 CAR | 0.1276288 | 0.15303288 | 0.01848625 | TCM |
| c-Jun-R12 CAR | 6.3925808 | 14.8052124 | 7.8848154 | [K]High |
| c-Jun-R12 CAR | 1.296918 | 2.6763 | 1.95651918 | |
| c-Jun-R12 CAR | 1.884225 | 2.7945 | 1.6268769 | [symbol] |
| c-Jun-R12 CAR | 0.69536456 | 2.13076266 | 1.03421808 | |
| c-Jun-R12 CAR | 0.68810112 | 1.57572768 | 0.56398194 | [K]Low |

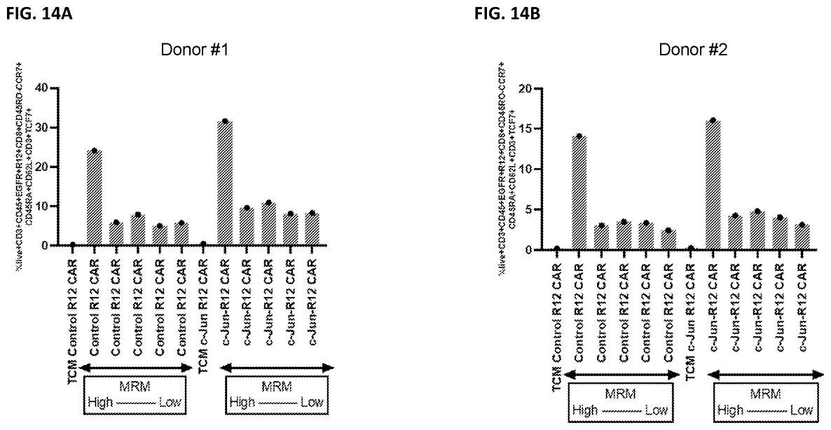

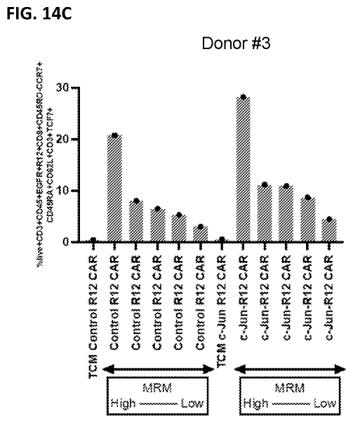

Similarly, CD8+ T cells transduced in MRM were generally more stem-like compared to corresponding cells transduced in TCM (see FIGS. 14A-14C and Table 16). Unlike the CD4+ T cells, consistent increase in stem-like cells was observed when CD8+ T cells were transduced with c-Jun-R12 CAR as compared to the control CD19t-R12 CAR. Accordingly, among the CD8+ T cells, the greatest percentage of stem-like cells was observed when CD8+ T cells were transduced with c-Jun-R12 CAR in MRM across a range of potassium concentrations. Like CD4+ T cells, a dose dependent effect of MRM concentration on stemness was observed with the highest stemness observed in MRM with the highest potassium concentration.

TABLE 16
CD8+ T Cell % Stemness

| | DONOR | DONOR | DONOR | |
|---|---|---|---|---|
| | 1: % | 2: % | 3: % | |
| SAMPLE | STEMNESS | STEMNESS | STEMNESS | Media |
| Control R12 CAR | 0.23411493 | 0.16918502 | 0.43113852 | TCM |
| Control R12 CAR | 24.0826488 | 14.1042504 | 20.7317305 | [K]High |
| Control R12 CAR | 5.9408568 | 3.0264909 | 8.0435576 | |
| Control R12 CAR | 7.8782592 | 3.4642192 | 6.51014 | [symbol] |
| Control R12 CAR | 5.0286663 | 3.3460182 | 5.3109504 | |
| Control R12 CAR | 5.772492 | 2.40669 | 3.003588 | [K]Low |
| c-Jun-R12 CAR | 0.4424251 | 0.23004516 | 0.597632 | TCM |
| c-Jun-R12 CAR | 31.6196316 | 16.076352 | 28.188846 | [K]High |
| c-Jun-R12 CAR | 9.5710475 | 4.2484029 | 11.198784 | |
| c-Jun-R12 CAR | 10.9405647 | 4.7722224 | 10.9214623 | [symbol] |
| c-Jun-R12 CAR | 8.1178136 | 4.0300992 | 8.6918656 | |
| c-Jun-R12 CAR | 8.2869375 | 3.09573 | 4.485132 | [K]Low |

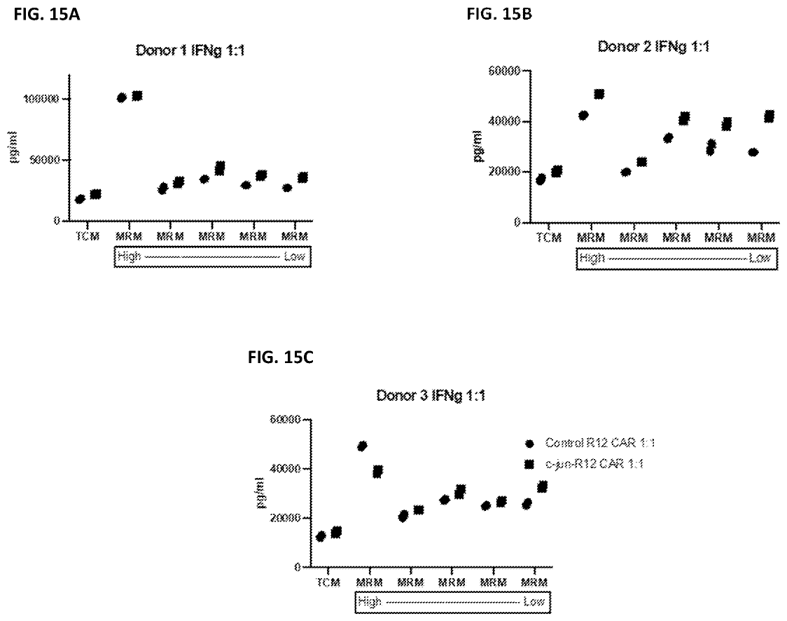

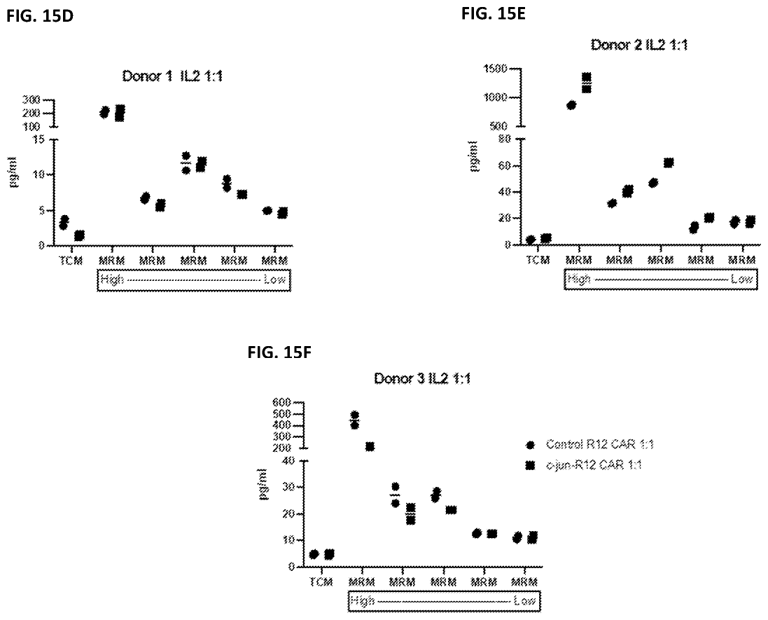

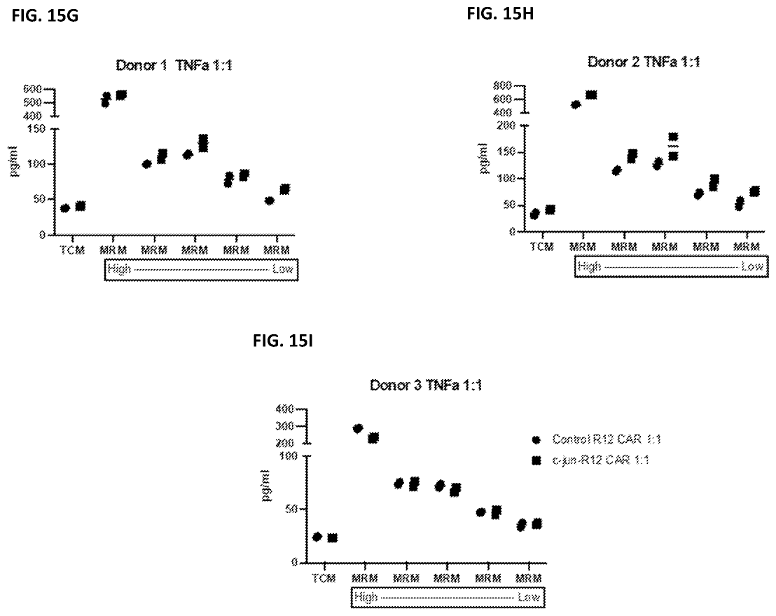

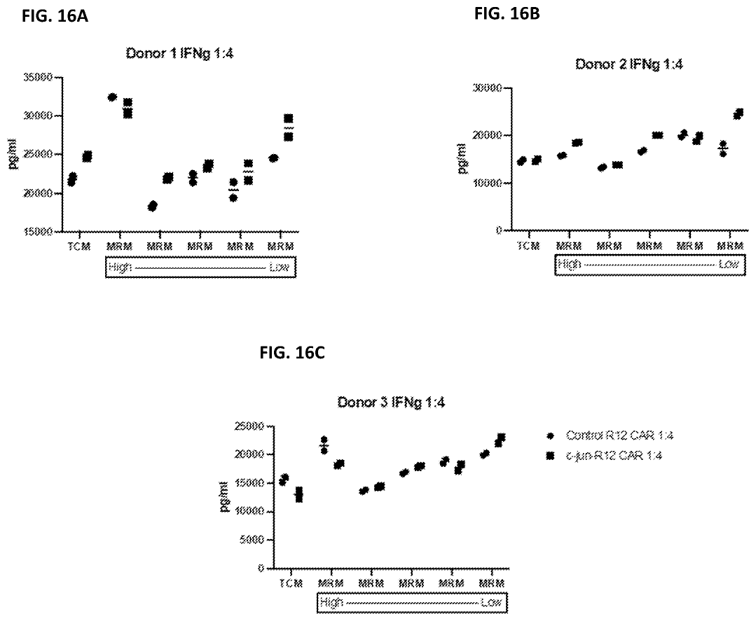

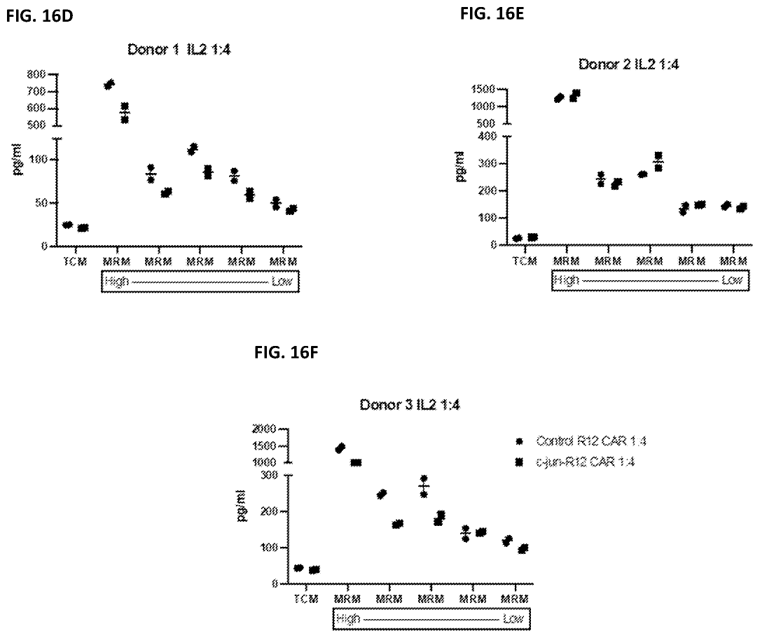

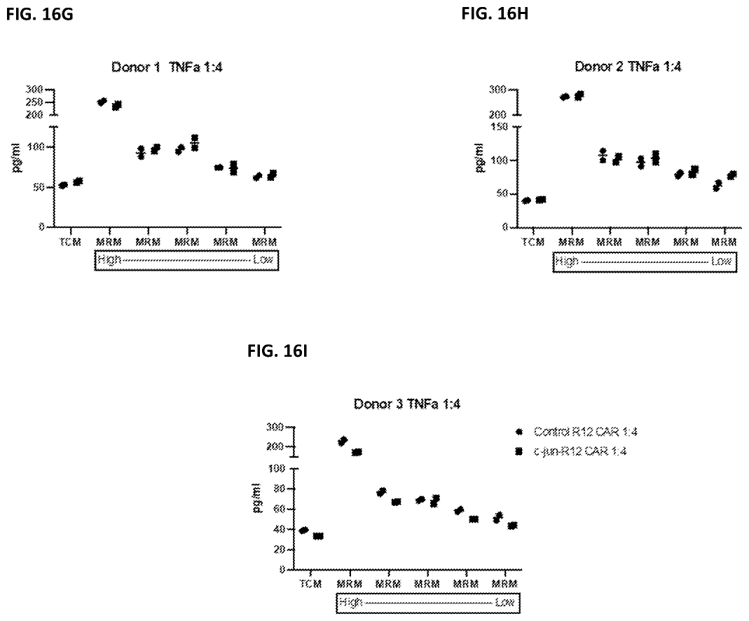

To assess the ability of the T cells transduced and cultured in the different MRMs to produce cytokines after antigen stimulation, IFNγ, IL-2, and TNFα cytokine secretion by untransduced (mock, data not shown), ROR1 CAR with c-Jun (black squares) and without c-Jun (black circles) T cell products (after day 7 of expansion) were assessed after 20 to 22 hours co-culture with A549 NLR cancer cell lines at an E:T of 1:1 and 1:4. As shown in FIGS. 15A-15I (1:1 effector:target ratio) and 16A-16I (1:4 effector:target ratio), anti-ROR1 CAR T cells (with and without c-Jun overexpression) from the MRM groups produced higher levels of IL2, TNFα (from all donors) and IFNγ (from 2 of 3 donors) as compared to T cells modified and cultured in TCM. Again, the highest cytokine production was observed in MRM with the highest potassium concentration.

To assess the cytolytic capability of the modified cells, an in vitro killing assay essentially as described in Example 3 was used. The IncuCyte killing assay was set-up in 96-well flat-bottom moat assay plates using T-cells from either Day 0, 7, or 14 of the serial re-stimulation assay and NucLight Red (NLR) target cell line, A549 at an E:T ratio of 1:1 and 1:4. The tumor viability percentage was calculated using the area under the curve (AUC) (the lower the bar, the higher the cytotoxicity) from IncuCyte killing curves obtained from control R12 CAR and c-Jun-R12 CAR T-cell products cultured in either TCM or MRM (high to low) on Days 0, 7 and 14 (serial stimulation (SS) 1, 3 and 5) of the serial re-stimulation assay after co-culture with A549 NLR target cell lines at an E:T ratio of 1:1 and 1:4. Data for the last round of stimulation (168 hrs to 300 hrs) is shown.

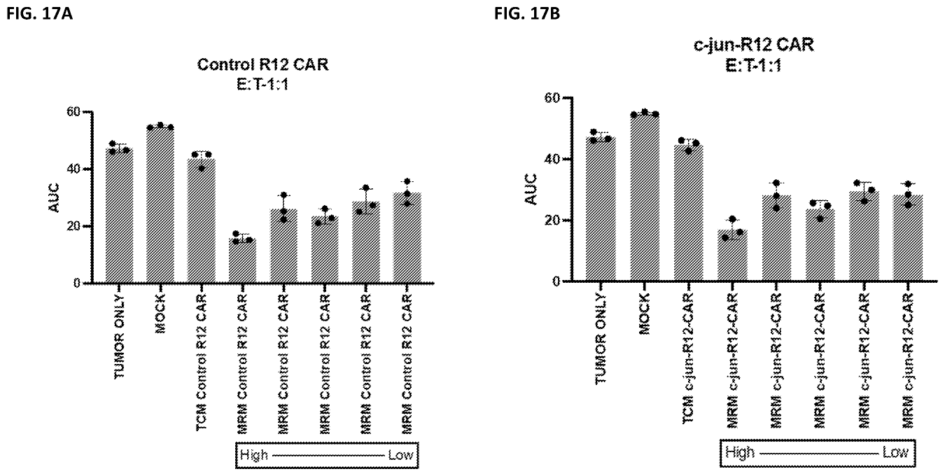

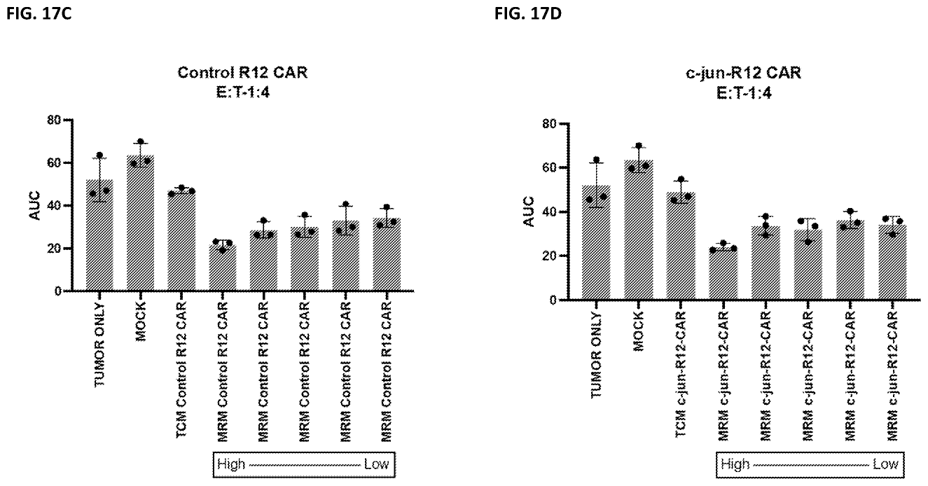

In agreement with the cytokine data described immediately above, anti-ROR1 CAR T cells from the different MRM groups generally exhibited increased killing as compared to those cells from the TCM group. As shown in FIGS. 17A-17D, after 2 rounds of antigen stimulation, much improved cytolytic activity was observed among T cells transduced and subsequently cultured in MRM as compared to the corresponding T cells from the control group. The improved cytolytic activity was observed in both anti-ROR1 CAR T cells overexpressing c-Jun and anti-ROR1 CAR T cells that were not modified to overexpress c-Jun.

Collectively, the above results further confirm the therapeutic benefits of the culturing methods provided herein. More specifically, the above results further demonstrate that modifying T cells (e.g., to express a ligand-binding protein and have increased expression level of a c-Jun protein) in the presence of a medium comprising potassium ion at a concentration higher than 5 mM (e.g., between 40-80 mM) can greatly increase the stemness of the cells and allow the cells to exhibit potent functional activity even in the presence of chronic antigen stimulation.

TABLE 17
c-Jun-anti-ROR1 CAR sequences

| | | |
|---|---|---|
| SEQ | | |
| ID NO | Description | Sequence |

c-Jun-anti-ROR1 CAR

| | | |
|---|---|---|
| 86 | c-Jun anti-ROR1 | MTAKMETTFYDDALNASFLPSESGPYGYSNPKILKQSMTLNLADPVGSLKPH |
| | CAR (Full | LRAKNSDLLTSPDVGLLKLASPELERLIIQSSNGHITTTPTPTQFLCPKNVT |
| | sequence) | DEQEGFAEGFVRALAELHSQNTLPSVTSAAQPVNGAGMVAPAVASVAGGSGS |
| | 1,198aa | GGFSASLHSEPPVYANLSNFNPGALSSGGGAPSYGAAGLAFPAQPQQQQQPP |
| | | HHLPQQMPVQHPRLQALKEEPQTVPEMPGETPPLSPIDMESQERIKAERKRM |
| | | RNRIAASKCRKRKLERIARLEEKVKTLKAQNSELASTANMLREQVAQLKQKV |
| | | MNHVNSGCQLMLTQQLQTFGSGATNFSLLKQAGDVEENPGPMVLQTQVFISL |
| | | LLWISGAYGQEQLVESGGRLVTPGGSLTLSCKASGFDFSAYYMSWVRQAPGK |
| | | GLEWIATIYPSSGKTYYATWVNGRFTISSDNAQNTVDLQMNSLTAADRATYF |
| | | CARDSYADDGALFNIWGPGTLVTISSGGGGSGGGGSGGGGSELVLTQSPSVS |
| | | AALGSPAKITCTLSSAHKTDTIDWYQQLQGEAPRYLMQVQSDGSYTKRPGVP |
| | | DRFSGSSSGADRYLIIPSVQADDEADYYCGADYIGGYVFGGGTQLTVTGGGG |
| | | SGKPCPPCKCPMFWVLVVVGGVLACYSLLVTVAFIIFWVKRGRKKLLYIFKQ |
| | | PFMRPVQTTQEEDGCSCRFPEEEEGGCELRVKFSRSADAPAYQQGQNQLYNE |
| | | LNLGRREEYDVLDKRRGRDPEMGGKPRRKNPQEGLYNELQKDKMAEAYSEIG |
| | | MKGERRRGKGHDGLYQGLSTATKDTYDALHMQALPPRSGATNFSLLKQAGDV |
| | | EENPGPMLLLVTSLLLCELPHPAFLLIPRKVCNGIGIGEFKDSLSINATNIK |
| | | HFKNCTSISGDLHILPVAFRGDSFTHTPPLDPQELDILKTVKEITGFLLIQA |
| | | WPENRTDLHAFENLEIIRGRTKQHGQFSLAVVSLNITSLGLRSLKEISDGDV |
| | | IISGNKNLCYANTINWKKLFGTSGQKTKIISNRGENSCKATGQVCHALCSPE |
| | | GCWGPEPRDCVSCRNVSRGRECVDKCNLLEGEPREFVENSECIQCHPECLPQ |
| | | AMNITCTGRGPDNCIQCAHYIDGPHCVKTCPAGVMGENNTLVWKYADAGHVC |
| | | HLCHPNCTYGCTGPGLEGCPTNGPKIPSIATGMVGALLLLLVVALGIGLFMR |
| | | RR |
| 13 | c-Jun | MTAKMETTFYDDALNASFLPSESGPYGYSNPKILKQSMTLNLADPVGSLKPH |
| | 331 aa | LRAKNSDLLTSPDVGLLKLASPELERLIIQSSNGHITTTPTPTQFLCPKNVT |
| | (aa 1-331 of SEQ | DEQEGFAEGFVRALAELHSQNTLPSVTSAAQPVNGAGMVAPAVASVAGGSGS |
| | ID NO: 86) | GGFSASLHSEPPVYANLSNFNPGALSSGGGAPSYGAAGLAFPAQPQQQQQPP |
| | | HHLPQQMPVQHPRLQALKEEPQTVPEMPGETPPLSPIDMESQERIKAERKRM |
| | | RNRIAASKCRKRKLERIARLEEKVKTLKAQNSELASTANMLREQVAQLKQKV |
| | | MNHVNSGCQLMLTQQLQTF |
| 88 | c-Jun after P2A | MTAKMETTFYDDALNASFLPSESGPYGYSNPKILKQSMTLNLADPVGSLKPH |
| | cleavage | LRAKNSDLLTSPDVGLLKLASPELERLIIQSSNGHITTTPTPTQFLCPKNVT |
| | (remnant boxed) | DEQEGFAEGFVRALAELHSQNTLPSVTSAAQPVNGAGMVAPAVASVAGGSGS |
| | | GGFSASLHSEPPVYANLSNFNPGALSSGGGAPSYGAAGLAFPAQPQQQQQPP |
| | | HHLPQQMPVQHPRLQALKEEPQTVPEMPGETPPLSPIDMESQERIKAERKRM |
| | | RNRIAASKCRKRKLERIARLEEKVKTLKAQNSELASTANMLREQVAQLKQKV |
| | | MNHVNSGCQLMLTQQLQTFGSGATNFSLLKQAGDVEENPG |
| 56 | P2A | GSGATNFSLLKQAGDVEENPGP |
| | 22 aa | |
| | (aa 332-353 of | |
| | SEQ ID NO: 86) | |
| 57 | hIgK | MVLQTQVFISLLLWISGAYG |
| | 20 aa | |
| | (aa 354-373 of | |
| | SEQ ID NO: 86) | |
| 89 | hIgK after P2A | PMVLQTQVFISLLLWISGAYG. (P2A remnant residue double |
| | cleavage | underlined |
| 83 | anti-ROR1 scFv | QEQLVESGGRLVTPGGSLTLSCKASGFDFSAYYMSWVRQAPGKGLEWIATIY |
| | 248 aa | PSSGKTYYATWVNGRFTISSDNAQNTVDLQMNSLTAADRATYFCARDSYADD |
| | (aa 374-621 of | GALFNIWGPGTLVTISSGGGGSGGGGSGGGGSELVLTQSPSVSAALGSPAKI |
| | SEQ ID NO: 86) | TCTLSSAHKTDTIDWYQQLQGEAPRYLMQVQSDGSYTKRPGVPDRFSGSSSG |
| | | ADRYLIIPSVQADDEADYYCGADYIGGYVFGGGTQLTVTG |
| 40 | Linker | GGGSG |
| | 5 aa | |
| | (aa 622-626 of | |
| | SEQ ID NO: 86) | |
| 51 | Spacer | KPCPPCKCP |
| | 9 aa | |
| | (aa 627-635 of | |
| | SEQ ID NO: 86) | |
| 75 | CD28 | MFWVLVVVGGVLACYSLLVTVAFIIFWV |
| | Transmembrane | |
| | Domain | |
| | 28 aa | |
| | (aa 636-663 of | |
| | SEQ ID NO: 86) | |
| 76 | 4-1BB | KRGRKKLLYIFKQPFMRPVQTTQEEDGCSCRFPEEEEGGCEL |
| | 42 aa | |
| | (aa 664-705 of | |
| | SEQ ID NO: 86) | |
| 84 | CD3z | RVKFSRSADAPAYQQGQNQLYNELNLGRREEYDVLDKRRGRDPEMGGKPRRK |
| | 112 aa | NPQEGLYNELQKDKMAEAYSEIGMKGERRRGKGHDGLYQGLSTATKDTYDAL |
| | (aa 706-817 of | HMQALPPR |
| | SEQ ID NO: 86) | |
| 90 | CD3z after P2A | RVKFSRSADAPAYQQGQNQLYNELNLGRREEYDVLDKRRGRDPEMGGKPRRK |
| | cleavage | NPQEGLYNELQKDKMAEAYSEIGMKGERRRGKGHDGLYQGLSTATKDTYDAL |
| | | HMQALPPR [symbol] (linker-P2A remnant |
| | | boxed) |
| 85 | SG linker - P2A | SG-ATNFSLLKQAGDVEENPGP |
| | 21 aa | |
| | (aa 819-838 of | |
| | SEQ ID NO: 86) | |
| 53 | GMCSFR-alpha-SP | MLLLVTSLLLCELPHPAFLLIP |
| | 22 aa | |
| | (aa 839-860 of | |
| | SEQ ID NO: 86) | |
| 91 | GMCSFR-alpha-SP | PMLLLVTSLLLCELPHPAFLLIP (P2A remnant residue double |
| | After P2A | underlined |
| | cleavage | |
| 24 | EGFRt | RKVCNGIGIGEFKDSLSINATNIKHFKNCTSISGDLHILPVAFRGDSFTHTP |
| | 338 aa | PLDPQELDILKTVKEITGFLLIQAWPENRTDLHAFENLEIIRGRTKQHGQFS |
| | (aa 860-1,198 of | LAVVSLNITSLGLRSLKEISDGDVIISGNKNLCYANTINWKKLFGTSGQKTK |
| | SEQ ID NO: 86) | IISNRGENSCKATGQVCHALCSPEGCWGPEPRDCVSCRNVSRGRECVDKCNL |
| | | LEGEPREFVENSECIQCHPECLPQAMNITCTGRGPDNCIQCAHYIDGPHCVK |
| | | TCPAGVMGENNTLVWKYADAGHVCHLCHPNCTYGCTGPGLEGCPTNGPKIPS |
| | | IATGMVGALLLLLVVALGIGLFMRRR |
| 87 | c-Jun anti-ROR1 | TGAAAGACCCCACCTGTAGGTTTGGCAAGCTAGGATCAAGGTTAGGAACAGA |
| | (Full nucleotide | GAGACAGCAGAATATGGGCCAAACAGGATATCTGTGGTAAGCAGTTCCTGCC |
| | sequence (with | CCGGCTCAGGGCCAAGAACAGTTGGAACAGCAGAATATGGGCCAAACAGGAT |
| | promoter) | ATCTGTGGTAAGCAGTTCCTGCCCCGGCTCAGGGCCAAGAACAGATGGTCCC |
| | 4,022 | CAGATGCGGTCCCGCCCTCAGCAGTTTCTAGAGAACCATCAGATGTTTCCAG |
| | nucleotides | GGTGCCCCAAGGACCTGAAATGACCCTGTGCCTTATTTGAACTAACCAATCA |
| | | GTTCGCTTCTCGCTTCTGTTCGCGCGCTTCTGCTCCCCGAGCTCAATAAAAG |
| | | AGCCCACAACCCCTCACTCGGCGCGATCAGAACCTCTTACGAGTCGGCTAGC |
| | | GCCGCCACCATGACAGCCAAGATGGAAACCACATTCTACGACGACGCCCTGA |
| | | ACGCCTCATTCCTGCCTTCTGAGAGCGGACCTTACGGCTACAGCAATCCTAA |
| | | GATCCTGAAACAGAGCATGACCCTTAACCTGGCTGATCCTGTTGGAAGCCTG |
| | | AAACCTCACCTGAGAGCCAAAAACAGCGACCTGCTCACCAGCCCTGATGTGG |
| | | GCCTGCTGAAGCTGGCCTCTCCAGAGCTGGAACGGCTGATCATCCAGAGCAG |
| | | CAACGGCCACATCACAACCACCCCTACCCCTACACAATTCCTGTGCCCTAAG |
| | | AACGTGACCGACGAGCAGGAGGGCTTCGCCGAAGGCTTTGTGCGGGCCCTGG |
| | | CAGAACTGCACTCTCAGAACACCCTGCCTAGCGTGACCTCCGCCGCCCAGCC |
| | | TGTCAACGGCGCCGGAATGGTGGCCCCTGCCGTGGCTTCTGTGGCCGGCGGC |
| | | AGCGGCAGCGGCGGATTCAGCGCCTCTCTGCACTCTGAGCCTCCTGTCTACG |
| | | CCAATCTGTCTAATTTCAACCCCGGAGCCCTGTCCAGCGGCGGCGGAGCTCC |
| | | TAGCTACGGCGCTGCTGGACTGGCCTTCCCCGCCCAGCCCCAGCAACAGCAG |
| | | CAGCCTCCACACCACCTGCCCCAGCAGATGCCCGTGCAGCACCCTAGACTGC |
| | | AGGCCCTGAAGGAAGAACCCCAAACAGTGCCTGAGATGCCTGGCGAGACACC |
| | | TCCACTGAGCCCCATCGACATGGAAAGCCAGGAGCGGATCAAGGCCGAGAGA |
| | | AAGAGAATGCGGAACAGAATCGCCGCTAGCAAGTGCAGAAAGCGGAAGCTGG |
| | | AAAGAATCGCCAGACTGGAAGAGAAGGTGAAGACCCTGAAAGCCCAAAATAG |
| | | CGAGCTGGCCAGCACCGCCAACATGCTGCGGGAACAGGTGGCCCAGCTGAAG |
| | | CAGAAGGTGATGAACCACGTGAACTCTGGTTGTCAGCTGATGCTGACCCAGC |
| | | AGCTCCAGACCTTCGGCTCCGGTGCAACGAACTTCAGCCTGCTGAAGCAGGC |
| | | CGGAGATGTTGAGGAAAATCCAGGTCCCATGGTCTTGCAGACTCAAGTATTT |
| | | ATATCCCTTTTGCTCTGGATCTCTGGAGCTTACGGCCAGGAACAGCTCGTCG |
| | | AAAGCGGCGGCAGACTGGTGACACCTGGCGGCAGCCTGACCCTGAGCTGCAA |
| | | GGCCAGCGGCTTCGACTTCAGCGCCTACTACATGAGCTGGGTCCGCCAGGCC |
| | | CCTGGCAAGGGACTGGAATGGATCGCCACCATCTACCCCAGCAGCGGCAAGA |
| | | CCTACTACGCCACCTGGGTGAACGGACGGTTCACCATCTCCAGCGACAACGC |
| | | CCAGAACACCGTGGACCTGCAGATGAACAGCCTGACAGCCGCCGACCGGGCC |
| | | ACCTACTTTTGCGCTCGGGACAGCTACGCCGACGACGGCGCCCTGTTCAACA |
| | | TCTGGGGCCCTGGCACCCTGGTGACAATCTCTAGCGGCGGAGGCGGATCTGG |
| | | TGGCGGAGGAAGTGGCGGCGGAGGATCTGAGCTGGTGCTGACCCAGAGCCCC |
| | | TCTGTGTCTGCTGCCCTGGGAAGCCCTGCCAAGATCACCTGTACCCTGAGCA |
| | | GCGCCCACAAGACCGACACCATCGACTGGTATCAGCAGCTGCAGGGCGAGGC |
| | | CCCCAGATACCTGATGCAGGTGCAGAGCGACGGCAGCTACACCAAGAGGCCA |
| | | GGCGTGCCCGACAGGTTCAGCGGATCTAGCTCTGGCGCCGACCGCTACCTGA |
| | | TCATCCCCAGCGTGCAGGCCGATGACGAGGCCGATTACTACTGTGGCGCCGA |
| | | CTACATCGGCGGCTACGTGTTCGGCGGAGGCACCCAGCTGACCGTGACCGGT |
| | | GGCGGAGGTTCAGGCAAACCGTGCCCTCCGTGCAAGTGTCCTATGTTCTGGG |
| | | TGCTGGTGGTGGTCGGAGGCGTGCTGGCCTGCTACAGCCTGCTGGTCACCGT |
| | | GGCCTTCATCATCTTTTGGGTGAAACGGGGCAGAAAGAAACTCCTGTATATA |
| | | TTCAAACAACCATTTATGAGACCAGTACAAACTACTCAAGAGGAAGATGGCT |
| | | GTAGCTGCCGATTTCCAGAAGAAGAAGAAGGAGGATGTGAACTGCGGGTGAA |
| | | GTTCAGCAGAAGCGCCGACGCCCCTGCCTACCAGCAGGGCCAGAATCAGCTG |
| | | TACAACGAGCTGAACCTGGGCAGAAGGGAAGAGTACGACGTCCTGGATAAGC |
| | | GGAGAGGCCGGGACCCTGAGATGGGCGGCAAGCCTCGGCGGAAGAACCCCCA |
| | | GGAAGGCCTGTATAACGAACTGCAGAAAGACAAGATGGCCGAGGCCTACAGC |
| | | GAGATCGGCATGAAGGGCGAGCGGAGGCGGGGCAAGGGCCACGACGGCCTGT |
| | | ATCAGGGCCTGTCCACCGCCACCAAGGATACCTACGACGCCCTGCACATGCA |
| | | GGCCCTGCCCCCAAGGTCCGGAGCCACTAACTTCTCCCTGTTGAAACAAGCA |
| | | GGGGATGTCGAAGAGAATCCCGGGCCAATGCTTCTCCTGGTGACAAGCCTTC |
| | | TGCTCTGTGAATTACCACACCCAGCATTCCTCCTGATCCCACGCAAAGTGTG |
| | | CAACGGAATAGGTATTGGTGAATTTAAGGACTCACTCTCCATAAATGCTACG |
| | | AATATTAAACACTTCAAAAACTGCACCTCCATCAGTGGCGATCTCCACATCC |
| | | TGCCGGTGGCATTTAGGGGTGACTCCTTCACACATACTCCTCCTCTGGACCC |
| | | ACAAGAACTGGATATTCTGAAAACCGTAAAGGAAATCACAGGGTTTTTGCTG |
| | | ATTCAAGCTTGGCCTGAAAACAGGACGGACCTCCATGCCTTTGAGAACCTAG |
| | | AAATCATACGCGGCAGGACCAAGCAGCATGGACAGTTTTCTCTTGCTGTCGT |
| | | GAGCCTGAACATAACATCCTTGGGATTACGCTCCCTCAAGGAGATAAGTGAT |
| | | GGAGATGTGATAATTTCAGGAAACAAAAATTTGTGCTATGCAAATACAATAA |
| | | ACTGGAAAAAACTGTTTGGGACCTCCGGCCAGAAAACCAAAATTATAAGCAA |
| | | CAGAGGCGAAAACAGCTGCAAGGCCACAGGCCAGGTCTGCCATGCCTTGTGC |
| | | TCCCCCGAGGGCTGCTGGGGCCCGGAGCCCAGGGATTGCGTGTCTTGCCGGA |
| | | ATGTCAGCCGAGGCAGGGAATGCGTGGACAAGTGCAACCTTCTGGAAGGCGA |
| | | GCCAAGGGAGTTTGTGGAGAACTCTGAGTGCATACAGTGCCACCCAGAGTGC |
| | | CTGCCTCAGGCCATGAACATCACCTGCACAGGACGGGGACCAGACAACTGTA |
| | | TCCAGTGTGCCCACTACATTGACGGCCCCCACTGCGTCAAGACCTGCCCGGC |
| | | AGGAGTCATGGGAGAAAACAACACCCTGGTCTGGAAGTACGCAGACGCCGGC |
| | | CATGTGTGCCACCTGTGCCATCCAAACTGCACCTACGGATGCACTGGGCCAG |
| | | GTCTTGAAGGCTGTCCAACGAACGGGCCTAAGATCCCGTCCATCGCCACTGG |
| | | GATGGTGGGGGCCCTCCTCTTGCTGCTGGTGGTGGCCCTGGGGATCGGCCTC |
| | | TTCATGCGCCGAAGGTGA |
| 64 | MND Promoter | TGAAAGACCCCACCTGTAGGTTTGGCAAGCTAGGATCAAGGTTAGGAACAGA |
| | | GAGACAGCAGAATATGGGCCAAACAGGATATCTGTGGTAAGCAGTTCCTGCC |
| | | CCGGCTCAGGGCCAAGAACAGTTGGAACAGCAGAATATGGGCCAAACAGGAT |
| | | ATCTGTGGTAAGCAGTTCCTGCCCCGGCTCAGGGCCAAGAACAGATGGTCCC |
| | | CAGATGCGGTCCCGCCCTCAGCAGTTTCTAGAGAACCATCAGATGTTTCCAG |
| | | GGTGCCCCAAGGACCTGAAATGACCCTGTGCCTTATTTGAACTAACCAATCA |
| | | GTTCGCTTCTCGCTTCTGTTCGCGCGCTTCTGCTCCCCGAGCTCAATAAAAG |
| | | AGCCCACAACCCCTCACTCGGC |
| 95 | c-Jun anti- | MTAKMETTFYDDALNASFLPSESGPYGYSNPKILKQSMTLNLADPVGSLKPH |
| | NYESO1 TCR (Full | LRAKNSDLLTSPDVGLLKLASPELERLIIQSSNGHITTTPTPTQFLCPKNVT |
| | sequence) | DEQEGFAEGFVRALAELHSQNTLPSVTSAAQPVNGAGMVAPAVASVAGGSGS |
| | 958 aa | GGFSASLHSEPPVYANLSNFNPGALSSGGGAPSYGAAGLAFPAQPQQQQQPP |
| | | HHLPQQMPVQHPRLQALKEEPQTVPEMPGETPPLSPIDMESQERIKAERKRM |
| | | RNRIAASKCRKRKLERIARLEEKVKTLKAQNSELASTANMLREQVAQLKQKV |
| | | MNHVNSGCQLMLTQQLQTFGSGATNFSLLKQAGDVEENPGPMSIGLLCCAAL |
| | | SLLWAGPVNAGVTQTPKFQVLKTGQSMTLQCAQDMNHEYMSWYRQDPGMGLR |
| | | LIHYSVGAGITDQGEVPNGYNVSRSTTEDFPLRLLSAAPSQTSVYFCASSYV |
| | | GNTGELFFGEGSRLTVLEDLRNVTPPKVSLFEPSKAEIANKQKATLVCLARG |
| | | FFPDHVELSWWVNGKEVHSGVCTDPQAYKESNYSYCLSSRLRVSATFWHNPR |
| | | NHFRCQVQFHGLSEEDKWPEGSPKPVTQNISAEAWGRADCGITSASYQQGVL |
| | | SATILYEILLGKATLYAVLVSTLVVMAMVKRKNSRGRAKRSGSGATNFSLLK |
| | | QAGDVEENPGPMETLLGLLILWLQLQWVSSKQEVTQIPAALSVPEGENLVLN |
| | | CSFTDSAIYNLQWFRQDPGKGLTSLLLIQSSQREQTSGRLNASLDKSSGRST |
| | | LYIAASQPGDSATYLCAVRPLYGGSYIPTFGRGTSLIVHPYDIQNPEPAVYQ |
| | | LKDPRSQDSTLCLFTDFDSQINVPKTMESGTFITDKCVLDMKAMDSKSNGAI |
| | | AWSNQTSFTCQDIFKETNATYPSSDVPCDATLTEKSFETDMNLNFQNLLVIV |
| | | LRILLLKVAGFNLLMTLRLWSS |
| 13 | c-Jun | MTAKMETTFYDDALNASFLPSESGPYGYSNPKILKQSMTLNLADPVGSLKPH |
| | 331 aa | LRAKNSDLLTSPDVGLLKLASPELERLIIQSSNGHITTTPTPTQFLCPKNVT |
| | (aa 1-331 of SEQ | DEQEGFAEGFVRALAELHSQNTLPSVTSAAQPVNGAGMVAPAVASVAGGSGS |
| | ID NO: 95) | GGFSASLHSEPPVYANLSNFNPGALSSGGGAPSYGAAGLAFPAQPQQQQQPP |
| | | HHLPQQMPVQHPRLQALKEEPQTVPEMPGETPPLSPIDMESQERIKAERKRM |
| | | RNRIAASKCRKRKLERIARLEEKVKTLKAQNSELASTANMLREQVAQLKQKV |
| | | MNHVNSGCQLMLTQQLQTF |
| 96 | NY-ESO1 TCR beta | MSIGLLCCAALSLLWAGPVNAGVTQTPKFQVLKTGQSMTLQCAQDMNHEYMS |
| | chain | WYRQDPGMGLRLIHYSVGAGITDQGEVPNGYNVSRSTTEDFPLRLLSAAPSQ |
| | 315 aa | TSVYFCASSYVGNTGELFFGEGSRLTVLEDLRNVTPPKVSLFEPSKAEIANK |
| | | QKATLVCLARGFFPDHVELSWWVNGKEVHSGVCTDPQAYKESNYSYCLSSRL |
| | | RVSATFWHNPRNHFRCQVQFHGLSEEDKWPEGSPKPVTQNISAEAWGRADCG |
| | | ITSASYQQGVLSATILYEILLGKATLYAVLVSTLVVMAMVKRKNSRG |
| 18 | Furin cleavage | RAKR |
| | site | |
| 97 | Linker | SGSG |
| 14 | P2A | ATNFSLLKQAGDVEENPGP |
| | 19aa | |
| 98 | NY-ESO1 TCR | METLLGLLILWLQLQWVSSKQEVTQIPAALSVPEGENLVLNCSFTDSAIYNL |
| | alpha chain | QWFRQDPGKGLTSLLLIQSSQREQTSGRLNASLDKSSGRSTLYIAASQPGDS |
| | 271 aa | ATYLCAVRPLYGGSYIPTFGRGTSLIVHPYDIQNPEPAVYQLKDPRSQDSTL |
| | | CLFTDFDSQINVPKTMESGTFITDKCVLDMKAMDSKSNGAIAWSNQTSFTCQ |
| | | DIFKETNATYPSSDVPCDATLTEKSFETDMNLNFQNLLVIVLRILLLKVAGF |
| | | NLLMTLRLWSS |
| 99 | cJun NY-ESO1 TCR | ATGACAGCCAAGATGGAAACCACATTCTACGACGACGCCCTGAACGCCTCAT |
| | | TCCTGCCTTCTGAGAGCGGACCTTACGGCTACAGCAATCCTAAGATCCTGAA |
| | | ACAGAGCATGACCCTTAACCTGGCTGATCCTGTTGGAAGCCTGAAACCTCAC |
| | | CTGAGAGCCAAAAACAGCGACCTGCTCACCAGCCCTGATGTGGGCCTGCTGA |
| | | AGCTGGCCTCTCCAGAGCTGGAACGGCTGATCATCCAGAGCAGCAACGGCCA |
| | | CATCACAACCACCCCTACCCCTACACAATTCCTGTGCCCTAAGAACGTGACC |
| | | GACGAGCAGGAGGGCTTCGCCGAAGGCTTTGTGCGGGCCCTGGCAGAACTGC |
| | | ACTCTCAGAACACCCTGCCTAGCGTGACCTCCGCCGCCCAGCCTGTCAACGG |
| | | CGCCGGAATGGTGGCCCCTGCCGTGGCTTCTGTGGCCGGCGGCAGCGGCAGC |
| | | GGCGGATTCAGCGCCTCTCTGCACTCTGAGCCTCCTGTCTACGCCAATCTGT |
| | | CTAATTTCAACCCCGGAGCCCTGTCCAGCGGCGGCGGAGCTCCTAGCTACGG |
| | | CGCTGCTGGACTGGCCTTCCCCGCCCAGCCCCAGCAACAGCAGCAGCCTCCA |
| | | CACCACCTGCCCCAGCAGATGCCCGTGCAGCACCCTAGACTGCAGGCCCTGA |
| | | AGGAAGAACCCCAAACAGTGCCTGAGATGCCTGGCGAGACACCTCCACTGAG |
| | | CCCCATCGACATGGAAAGCCAGGAGCGGATCAAGGCCGAGAGAAAGAGAATG |
| | | CGGAACAGAATCGCCGCTAGCAAGTGCAGAAAGCGGAAGCTGGAAAGAATCG |
| | | CCAGACTGGAAGAGAAGGTGAAGACCCTGAAAGCCCAAAATAGCGAGCTGGC |
| | | CAGCACCGCCAACATGCTGCGGGAACAGGTGGCCCAGCTGAAGCAGAAGGTG |
| | | ATGAACCACGTGAACTCTGGTTGTCAGCTGATGCTGACCCAGCAGCTCCAGA |
| | | CCTTCGGCTCCGGTGCAACGAACTTCAGCCTGCTGAAGCAGGCCGGAGATGT |
| | | TGAGGAAAATCCAGGTCCCATGTCTATCGGACTGCTGTGCTGTGCCGCCCTG |
| | | AGCCTGCTGTGGGCAGGACCTGTGAACGCAGGAGTGACCCAGACACCAAAGT |
| | | TCCAGGTGCTGAAGACCGGCCAGAGCATGACACTGCAGTGCGCCCAGGATAT |
| | | GAATCACGAGTACATGTCCTGGTATCGGCAGGACCCTGGCATGGGCCTGAGA |
| | | CTGATCCACTACTCCGTGGGAGCAGGAATCACCGATCAGGGAGAGGTGCCAA |
| | | ACGGCTATAACGTGAGCCGGAGCACCACAGAGGACTTCCCACTGAGACTGCT |
| | | GTCCGCCGCACCTTCCCAGACCAGCGTGTACTTTTGCGCCAGCTCCTATGTG |
| | | GGCAACACAGGCGAGCTGTTCTTTGGAGAGGGAAGCAGGCTGACCGTGCTGG |
| | | AGGATCTGCGCAACGTGACACCCCCTAAGGTGTCTCTGTTCGAGCCCAGCAA |
| | | GGCCGAGATCGCCAATAAGCAGAAGGCCACCCTGGTGTGCCTGGCAAGGGGC |
| | | TTCTTTCCTGATCACGTGGAGCTGTCCTGGTGGGTGAACGGCAAGGAGGTGC |
| | | ACTCTGGCGTGTGCACCGACCCACAGGCCTACAAGGAGAGCAATTACTCCTA |
| | | TTGTCTGTCTAGCCGGCTGAGAGTGTCCGCCACATTTTGGCACAACCCACGG |
| | | AATCACTTCAGATGCCAGGTGCAGTTTCACGGCCTGTCTGAGGAGGATAAGT |
| | | GGCCAGAGGGAAGCCCAAAGCCAGTGACCCAGAACATCTCCGCCGAGGCATG |
| | | GGGAAGGGCAGACTGTGGCATCACCTCCGCCTCTTATCAGCAGGGCGTGCTG |
| | | AGCGCCACAATCCTGTACGAGATCCTGCTGGGCAAGGCCACCCTGTATGCCG |
| | | TGCTGGTGTCCACACTGGTGGTCATGGCCATGGTGAAGAGGAAGAATTCTAG |
| | | GGGCCGCGCAAAGCGCAGCGGATCCGGAGCAACCAACTTCTCCCTGCTGAAG |
| | | CAGGCCGGCGATGTGGAGGAGAATCCTGGCCCAATGGAGACACTGCTGGGCC |
| | | TGCTGATCCTGTGGCTGCAGCTGCAGTGGGTGTCCTCTAAGCAGGAGGTGAC |
| | | CCAGATCCCTGCCGCCCTGAGCGTGCCAGAGGGAGAGAACCTGGTGCTGAAT |
| | | TGCTCCTTCACAGATTCTGCCATCTACAACCTGCAGTGGTTTAGGCAGGACC |
| | | CAGGCAAGGGACTGACCTCTCTGCTGCTGATCCAGAGCTCCCAGAGGGAGCA |
| | | GACCAGCGGCCGGCTGAACGCCTCCCTGGATAAGTCTAGCGGCAGAAGCACC |
| | | CTGTACATCGCAGCCTCCCAGCCTGGCGACTCTGCCACATACCTGTGCGCCG |
| | | TGCGGCCACTGTACGGAGGCAGCTATATCCCCACCTTCGGCAGAGGCACATC |
| | | CCTGATCGTGCACCCCTACGACATCCAGAACCCCGAGCCTGCCGTGTATCAG |
| | | CTGAAGGACCCTCGGTCTCAGGATAGCACCCTGTGCCTGTTCACAGACTTTG |
| | | ATTCTCAGATCAATGTGCCCAAGACCATGGAGAGCGGCACCTTTATCACAGA |
| | | CAAGTGCGTGCTGGATATGAAGGCCATGGACTCTAAGAGCAACGGCGCCATC |
| | | GCCTGGAGCAATCAGACCTCCTTCACATGCCAGGATATCTTTAAGGAGACCA |
| | | ACGCCACATACCCATCCTCTGACGTGCCCTGTGATGCCACCCTGACAGAGAA |
| | | GAGCTTCGAGACCGACATGAACCTGAATTTTCAGAACCTGCTGGTCATCGTG |
| | | CTGAGGATCCTGCTGCTGAAGGTGGCCGGCTTTAATCTGCTGATGACACTGC |
| | | GCCTGTGGAGCTCC |
| 100 | EF1a promoter | GGCTCCGGTGCCCGTCAGTGGGCAGAGCGCACATCGCCCACAGTCCCCGAGA |
| | (Long) | AGTTGGGGGGAGGGGTCGGCAATTGAACCGGTGCCTAGAGAAGGTGGCGCGG |
| | | GGTAAACTGGGAAAGTGATGTCGTGTACTGGCTCCGCCTTTTTCCCGAGGGT |
| | | GGGGGAGAACCGTATATAAGTGCAGTAGTCGCCGTGAACGTTCTTTTTCGCA |
| | | ACGGGTTTGCCGCCAGAACACAGGTAAGTGCCGTGTGTGGTTCCCGCGGGCC |
| | | TGGCCTCTTTACGGGTTATGGCCCTTGCGTGCCTTGAATTACTTCCACCTGG |
| | | CTGCAGTACGTGATTCTTGATCCCGAGCTTCGGGTTGGAAGTGGGTGGGAGA |
| | | GTTCGAGGCCTTGCGCTTAAGGAGCCCCTTCGCCTCGTGCTTGAGTTGAGGC |
| | | CTGGCCTGGGCGCTGGGGCCGCCGCGTGCGAATCTGGTGGCACCTTCGCGCC |
| | | TGTCTCGCTGCTTTCGATAAGTCTCTAGCCATTTAAAATTTTTGATGACCTG |
| | | CTGCGACGCTTTTTTTCTGGCAAGATAGTCTTGTAAATGCGGGCCAAGATCT |
| | | GCACACTGGTATTTCGGTTTTTGGGGCCGCGGGCGGCGACGGGGCCCGTGCG |
| | | TCCCAGCGCACATGTTCGGCGAGGCGGGGCCTGCGAGCGCGGCCACCGAGAA |
| | | TCGGACGGGGGTAGTCTCAAGCTGGCCGGCCTGCTCTGGTGCCTGGCCTCGC |
| | | GCCGCCGTGTATCGCCCCGCCCTGGGCGGCAAGGCTGGCCCGGTCGGCACCA |
| | | GTTGCGTGAGCGGAAAGATGGCCGCTTCCCGGCCCTGCTGCAGGGAGCTCAA |
| | | AATGGAGGACGCGGCGCTCGGGAGAGCGGGCGGGTGAGTCACCCACACAAAG |
| | | GAAAAGGGCCTTTCCGTCCTCAGCCGTCGCTTCATGTGACTCCACGGAGTAC |
| | | CGGGCGCCGTCCAGGCACCTCGATTAGTTCTCGAGCTTTTGGAGTACGTCGT |
| | | CTTTAGGTTGGGGGGAGGGGTTTTATGCGATGGAGTTTCCCCACACTGAGTG |
| | | GGTGGAGACTGAAGTTAGGCCAGCTTGGCACTTGATGTAATTCTCCTTGGAA |
| | | TTTGCCCTTTTTGAGTTTGGATCTTGGTTCATTCTCAAGCCTCAGACAGTGG |
| | | TTCAAAGTTTTTTTCTTCCATTTCAGGTGTCGTGAATCGATTG |

1-2. (canceled)

3. A method of preparing a population of immune cells for immunotherapy comprising culturing immune cells in a medium comprising potassium ion at a concentration higher than 5 mM, wherein the immune cells have been modified to have an increased level of a c-Jun polypeptide as compared to corresponding immune cells that have not been modified to have an increased level of the c-Jun polypeptide.

4. A method of increasing a stemness of immune cells and/or increasing a yield of immune cells during ex vivo or in vitro culture comprising culturing immune cells in a medium comprising potassium ion at a concentration higher than 5 mM, wherein the immune cells have been modified to have an increased level of a c-Jun polypeptide as compared to corresponding immune cells that have not been modified to have an increased level of the c-Jun polypeptide.

5. (canceled)

6. A method of improving one or more functions of immune cells in response to an antigen stimulation comprising culturing the immune cells in a medium comprising potassium ion at a concentration higher than 5 mM, wherein the immune cells have been modified to have an increased level of a c-Jun polypeptide as compared to corresponding immune cells that have not been modified to have an increased level of the c-Jun polypeptide.

7-11. (canceled)

12. The method of claim 6, wherein the one or more functions comprise the ability: (i) to kill target cells, (ii) to produce a cytokine upon further antigen stimulation, or (iii) both (i) and (ii).

13-15. (canceled)

16. The method of claim 3, wherein the immune cells have been modified with an exogenous polynucleotide encoding the c-Jun polypeptide, such that after the modification, the immune cells have an increased level of the c-Jun polypeptide as compared to the corresponding immune cells.

17-18. (canceled)

19. A method of increasing the expression of a c-Jun polypeptide in an immune cell comprising modifying the immune cell with an exogenous polynucleotide, which encodes the c-Jun polypeptide, in a medium comprising potassium ion at a concentration higher than 5 mM, wherein after the modification the expression of the c-Jun polypeptide in the immune cell is increased compared to a reference cell.

20. The method of claim 19, wherein the reference cell comprises corresponding immune cells that: (i) have been modified to have an increased level of the c-Jun polypeptide and cultured in a medium that does not comprise potassium ion at a concentration higher than 5 mM; (ii) have not been modified to have an increased level of the c-Jun polypeptide and cultured in the medium that comprises potassium ion at a concentration higher than 5 mM; (iii) have not been modified to have an increased level of the c-Jun polypeptide and cultured in a medium that does not comprise potassium ion at a concentration higher than 5 mM; or (iv) any combination of (i) to (iii).

21-25. (canceled)

26. The method of claim 16, wherein the exogenous polynucleotide encoding the c-Jun polypeptide comprises
a. a nucleotide sequence having at least 89% sequence identity to the nucleic acid sequence as set forth in SEQ ID NO: 1;
b. a nucleotide sequence having at least 90% sequence identity to the nucleic acid sequence as set forth in SEQ ID NO: 2;
c. a nucleotide sequence having at least about 30% sequence identity to the nucleic acid sequence as set forth in SEQ ID NO: 4;
d. a nucleotide sequence having at least 79% sequence identity to the nucleic acid sequence as set forth in SEQ ID NO: 5;
e. a nucleotide sequence having at least 88% sequence identity to the nucleic acid sequence as set forth in SEQ ID NO: 6;
f. a nucleotide sequence having at least 82% sequence identity to the nucleic acid sequence as set forth in SEQ ID NO: 7;
g. a nucleotide sequence having at least 90% sequence identity to the nucleic acid sequence as set forth in SEQ ID NO: 8;
h. a nucleotide sequence having at least 55% sequence identity to the nucleic acid sequence as set forth in SEQ ID NO: 9; or
i. a nucleotide sequence having at least 85% sequence identity to the nucleic acid sequence as set forth in SEQ ID NO: 10.

27-44. (canceled)

45. The method of claim 3, wherein the immune cells further comprise: (a) a ligand binding protein, (b) a truncated EGFR (EGFRt), or (c) both (a) and (b).

46-80. (canceled)

81. The method of claim 3, wherein the concentration of potassium ion is selected from the group consisting of about 40 mM, about 45 mM, about 50 mM, about 55 mM, about 60 mM, about 65 mM, about 70 mM, about 75 mM, and about 80 mM.

82. The method of any claim 3, wherein the concentration of potassium ion is between about 30 mM and about 80 mM.

83. (canceled)

84. The method of claim 3, wherein the medium further comprises: (a) a sodium ion, (b) a cytokine, (c) a cell expansion agent, (d) a calcium ion, (e) a glucose, (f) a CD3 agonist, (g) a CD28 agonist, or (h) any combinations thereof.

85. The method of claim 84, wherein: (a) the sodium ion comprises NaCl; (b) the cytokine comprise an interleukin-2 (IL-2), interleukin-7 (IL-7), interleukin-21 (IL-21), interleukin-15 (IL-15), or combinations thereof; (c) the cell expansion agent comprises a GSK3B inhibitor, ACLY inhibitor, PI3K inhibitor, AKT inhibitor, or combinations thereof; (d) the CD3 agonist comprises an anti-CD3 antibody: (e) the CD28 agonist comprises an anti-CD28 antibody: or (f) any combination of (a) to (e).

86. (canceled)

87. The method of claim 3, wherein the medium is hypotonic or isotonic.

88. (canceled)

89. The method of claim 84, wherein:
(a) the sum of the potassium ion concentration and the sodium ion concentration, multiplied by two is: (i) more than 240 mM and less than 280 mM or (ii) more than or equal to 280 mM and less than 300 mM;
(b) the medium comprises IL-2 at a concentration from about 50 IU/mL to about 500 IU/mL;
(c) the medium comprises IL-21 at a concentration from about 50 IU/mL to about 500 IU/mL;
(d) the medium comprises IL-7 at a concentration from about 500 IU/mL to about 1,500 IU/mL;
(e) the medium comprises IL-15 at a concentration from about 50 IU/mL to about 500 IU/mL;
(f) the medium comprises glucose at a concentration from about 10 mM to about 25 mM;
(g) the medium comprises calcium ion at a concentration from about 0.4 mM to about 2.8 mM; or
(h) any combination of (a) to (g).

90-126. (canceled)

127. A population of immune cells prepared by the method of claim 3.

128-129. (canceled)

130. A pharmaceutical composition comprising the population of immune cells of claim 127, and a pharmaceutically acceptable carrier.

131. A composition comprising a population of immune cells, which comprises CD4+ T cells, CD8+ T cells, or both CD4+ T cells and CD8+ T cells, wherein the CD4+ T cells, the CD8+ T cells or both have been modified to (a) express a ligand binding protein selected from a chimeric antigen receptor (CAR) or an engineered T cell receptor (TCR) and (b) have an increased level of a c-Jun polypeptide as compared to a corresponding immune cells that have not been modified to have an increased level of the c-Jun polypeptide, wherein (i) at least about 15% of the modified CD4+ T cells are surface positive for CCR7 and CD45RA; (ii) at least about 20% of the modified CD8+ T cells are surface positive for CCR7 and CD45RA; (iii) at least about 4% of the immune cells are progenitor exhausted T cells: (iv) at least about 4% of the immune cells are stem-like T cells: or (v) any combination of (i) to (iv).

132-143. (canceled)

144. A method of treating a disease or condition in a subject in need thereof comprising administering the population of immune cells of claim 127 to the subject.

145. The method of claim 144, wherein the disease or condition comprises a cancer.
